(19) United States
(12) Patent Application Publication
(10) Pub. No.: US 2024/0150433 A1
(43) Pub. Date: May 9, 2024

(54) ERT2 MUTANTS AND USES THEREOF

(71) Applicant: Senti Biosciences, Inc., South San Francisco, CA (US)

(72) Inventors: Michelle Elizabeth Hung, South San Francisco, CA (US); Rebecca Tayler Cottman, San Carlos, CA (US); Russell Morrison Gordley, San Francisco, CA (US); Gary Lee, Castro Valley, CA (US); Timothy Kuan-Ta Lu, Cambridge, MA (US); Srinivasaraghavan Kannan, Singapore (SG); Chandra Shekhar Verma, Singapore (SG)

(21) Appl. No.: 18/481,805

(22) Filed: Oct. 5, 2023

Related U.S. Application Data
(63) Continuation of application No. PCT/US2022/023673, filed on Apr. 6, 2022.
(60) Provisional application No. 63/171,227, filed on Apr. 6, 2021.

(51) Int. Cl. C07K 14/72; C07K 14/715; C12N 15/85

(52) U.S. Cl. CPC C07K 14/721; C07K 14/7155; C12N 15/85; C07K 2319/09; C12N 2800/107; C12N 2830/002

(57) ABSTRACT

Provided herein are mutants of estrogen receptor alpha ligand binding domain (ER-LBD), and chimeric proteins including such mutant ER-LBD. Also provided are methods of modulating transcription and modulating localization of such chimeric proteins.

### CROSS REFERENCE TO RELATED APPLICATIONS

This application is a continuation of International Application No. PCT/US2022/023673, filed Apr. 6, 2022, which claims the benefit of U.S. Provisional Patent Application No. 63/171,227, filed Apr. 6, 2021, each of which are hereby incorporated by reference in their entireties for all purposes.

### SEQUENCE LISTING

The instant application contains a Sequence Listing XML which has been submitted electronically and is hereby incorporated by reference in its entirety. Said XML copy, created on Oct. 18, 2023, is named STB-028WOC1, and is 212,713 bytes in size.

### BACKGROUND

Estrogen receptor (ER) is a ligand-dependent transcription factor that binds endogenous hormone ligands such as estrogen and estradiol. Synthetic ligands that bind to ER have been developed for treating ER-positive cancers such as ER-positive breast cancer. For example, active metabolites of the drug tamoxifen induce nuclear translocation of ER and antagonize ER in a tissue-selective manner. Tamoxifen and its active metabolites are also utilized as a tool for controlling nuclear localization in the research setting. For example, an ER ligand binding domain variant known as ERT2 has been used widely as a fusion protein with Cre recombinase to regulate Cre recombinase-based gene editing in animal model systems. The ability to manipulate nuclear localization using a synthetic ligand would also be useful in therapeutic applications, such as for regulation of therapeutic genes. Thus, ERT2-based systems with improved sensitivity to and/or selectivity for synthetic ligands would be useful for employing ERT2-based gene regulation in a clinical setting.

### SUMMARY

Provided herein, in some embodiments, are modified estrogen receptor ligand binding domains (ER-LBD) with improved sensitivity and/or selectivity for non-endogenous ligands, such as tamoxifen and metabolites thereof. Also provided herein, in some embodiments, are chimeric proteins including a modified ER-LBD as described herein, genetic switches, polynucleotide molecules encoding the modified ER-LBD and chimeric protein as described herein, cells encoding the polynucleotide molecules described herein or expressing the modified ER-LBD and chimeric protein as described herein, and methods of using the modified ER-LBD, chimeric protein, polynucleotide molecule, genetic switch, or cells as described herein.

The modified ER-LBD and chimeric proteins described herein have greater sensitivity to and/or selectivity for non-endogenous ligands (e.g., 4-hydroxytamoxifen, also referred to as “4-OHT”) as compared to ERT2. ERT2 is a ligand binding domain of ER which includes a G400V amino acid substitution, an M543A amino acid substitution, and an L544A amino acid substitution (see SEQ ID NO: 2). ERT2 may also include, in addition to G400V/M543A/L544A, a V595A amino acid substitution (see SEQ ID NO: 3). The average peak plasma concentration following a typical clinical dose of tamoxifen is in the nanomolar range (e.g., approximately 40 ng/mL). However, sensitivity of wild-type ERT2 to tamoxifen metabolites (e.g., endoxifen and 4-OHT) is too low for its use to regulate gene expression at nanomolar concentrations of the metabolites. Furthermore, ERT2 may be responsive to endogenous ligands such as estradiol. Thus, the improved sensitivity to and/or selectivity for non-endogenous ligands of the modified ER-LBD and chimeric proteins including a modified ER-LBD allow for use of ER-based systems for controlling gene regulation.

Provided herein are modified estrogen receptor ligand binding domain (ER-LBD) comprising an amino acid sequence corresponding to amino acids 282-595 of SEQ ID NO: 1, wherein the modified ER-LBD comprises a G400V amino acid substitution, an M543A amino acid substitution, and an L544A amino acid substitution, and one or more additional amino acid substitutions, wherein the one or more additional amino acid substitutions are within a region of SEQ ID NO: 1 selected from the group consisting of: positions 343-354, positions 380-392, positions 404-463, and positions 517-540, and position 547, and wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions. In some aspects, the modified ER-LBD further comprises a V595A amino acid substitution. In some aspects, the non-endogenous ligand is selected from the group consisting of: 4-hydroxytamoxifen, N-desmethyltamoxifen, tamoxifen-N-oxide, and endoxifen.

In some aspects, the one or more additional amino acid substitutions are at one or more positions of SEQ ID NO: 1 selected from the group consisting of: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

In some aspects, the one or more positions comprise position 343 of SEQ ID NO: 1. In some aspects, the amino acid substitution at position 343 of SEQ ID NO: 1 is selected from the group consisting of: M343F, M343I, M343L, and M343V.

In some aspects, the one or more positions comprise position 344 of SEQ ID NO: 1. In some aspects, the amino acid substitution at position 344 of SEQ ID NO: 1 is G344M.

In some aspects, the one or more positions comprise position 345 of SEQ ID NO: 1. In some aspects, the amino acid substitution at position 345 of SEQ ID NO: 1 is L345S.

In some aspects, the one or more positions comprise position 346 of SEQ ID NO: 1. In some aspects, the amino acid substitution at position 346 of SEQ ID NO: 1 is selected from the group consisting of: L346I, L346M, L346F, and L346V.

In some aspects, the one or more positions comprise position 347 of SEQ ID NO: 1. In some aspects, the amino acid substitution at position 347 of SEQ ID NO: 1 is selected from the group consisting of: T347D, T347E, T347F, T347I, T347K, T347L, T347M, T347N, T347Q, T347R, T347S, and T347V.

In some aspects, the one or more positions comprise position 348 of SEQ ID NO: 1. In some aspects, the amino acid substitution at position 348 of SEQ ID NO: 1 is N348K.

In some aspects, the one or more positions comprise position 349 of SEQ ID NO: 1. In some aspects, the amino acid substitution at position 349 of SEQ ID NO: 1 is selected from the group consisting of: L349I, L349M, L349F, and L349V.

In some aspects, the one or more positions comprise position 350 of SEQ ID NO: 1. In some aspects, the amino acid substitution at position 350 of SEQ ID NO: 1 is selected from the group consisting of: A350F, A350I, A350L, A350M and A350V.

In some aspects, the one or more positions comprise position 351 of SEQ ID NO: 1. In some aspects, the amino acid substitution at position 351 of SEQ ID NO: 1 is selected from the group consisting of: D351E, D351F, D351I, D351L, D351M, D351N, D351Q, and D351V.

In some aspects, the one or more positions comprise position 352 of SEQ ID NO: 1. In some aspects, the amino acid substitution at position 352 of SEQ ID NO: 1 is R352K.

In some aspects, the one or more positions comprise position 354 of SEQ ID NO: 1. In some aspects, the amino acid substitution at position 354 of SEQ ID NO: 1 is selected from the group consisting of: L354I, L354M, L354F, and L354V.

In some aspects, the one or more positions comprise position 380 of SEQ ID NO: 1. In some aspects, the amino acid substitution at position 380 of SEQ ID NO: 1 is E380Q.

In some aspects, the one or more positions comprise position 384 of SEQ ID NO: 1. In some aspects, the amino acid substitution at position 384 of SEQ ID NO: 1 is selected from the group consisting of: L384I, L384M, L384F, and L384V.

In some aspects, the one or more positions comprise position 386 of SEQ ID NO: 1. In some aspects, the amino acid substitution at position 386 of SEQ ID NO: 1 is I386V.

In some aspects, the one or more positions comprise position 387 of SEQ ID NO: 1. In some aspects, the amino acid substitution at position 387 of SEQ ID NO: 1 is selected from the group consisting of: L387I, L387M, L387F, and L387V.

In some aspects, the one or more positions comprise position 388 of SEQ ID NO: 1. In some aspects, the amino acid substitution at position 388 of SEQ ID NO: 1 is selected from the group consisting of: M388I, M388L, and M388F.

In some aspects, the one or more positions comprise position 389 of SEQ ID NO: 1. In some aspects, the amino acid substitution at position 389 of SEQ ID NO: 1 is I389M.

In some aspects, the one or more positions comprise position 391 of SEQ ID NO: 1. In some aspects, the amino acid substitution at position 391 of SEQ ID NO: 1 is selected from the group consisting of: L391I, L391M, L391F, and L391V.

In some aspects, the one or more positions comprise position 392 of SEQ ID NO: 1. In some aspects, the amino acid substitution at position 392 of SEQ ID NO: 1 is V392M.

In some aspects, the one or more positions comprise position 404 of SEQ ID NO: 1. In some aspects, the amino acid substitution at position 404 of SEQ ID NO: 1 is selected from the group consisting of: F404I, F404L, F404M, and F404V.

In some aspects, the one or more positions comprise position 407 of SEQ ID NO: 1. In some aspects, the amino acid substitution at position 407 of SEQ ID NO: 1 is N407D.

In some aspects, the one or more positions comprise position 409 of SEQ ID NO: 1. In some aspects, the amino acid substitution at position 409 of SEQ ID NO: 1 is L409V.

In some aspects, the one or more positions comprise position 413 of SEQ ID NO: 1. In some aspects, the amino acid substitution at position 413 of SEQ ID NO: 1 is N413D.

In some aspects, the one or more positions comprise position 414 of SEQ ID NO: 1. In some aspects, the amino acid substitution at position 414 of SEQ ID NO: 1 is Q414E.

In some aspects, the one or more positions comprise position 417 of SEQ ID NO: 1. In some aspects, the amino acid substitution at position 417 of SEQ ID NO: 1 is C417S.

In some aspects, the one or more positions comprise position 418 of SEQ ID NO: 1. In some aspects, the amino acid substitution at position 418 of SEQ ID NO: 1 is selected from the group consisting of: V418I, V418L, V418M, and V418F.

In some aspects, the one or more positions comprise position 420 of SEQ ID NO: 1. In some aspects, the amino acid substitution at position 420 of SEQ ID NO: 1 is selected from the group consisting of: G420I, G420M, G420F, and G420V.

In some aspects, the one or more positions comprise position 421 of SEQ ID NO: 1. In some aspects, the amino acid substitution at position 421 of SEQ ID NO: 1 is selected from the group consisting of: M421I, M421L, M421F, and M421V.

In some aspects, the one or more positions comprise position 422 of SEQ ID NO: 1. In some aspects, the amino acid substitution at position 422 of SEQ ID NO: 1 is V422I.

In some aspects, the one or more positions comprise position 424 of SEQ ID NO: 1. In some aspects, the amino acid substitution at position 424 of SEQ ID NO: 1 is selected from the group consisting of: I424L, I424M, I424F, and I424V.

In some aspects, the one or more positions comprise position 428 of SEQ ID NO: 1. In some aspects, the amino acid substitution at position 428 of SEQ ID NO: 1 is selected from the group consisting of: L428I, L428M, L428F, and L428V.

In some aspects, the one or more positions comprise position 463 of SEQ ID NO: 1. In some aspects, the amino acid substitution at position 463 of SEQ ID NO: 1 is S463P.

In some aspects, the one or more positions comprise position 517 of SEQ ID NO: 1. In some aspects, the amino acid substitution at position 517 of SEQ ID NO: 1 is M517A.

In some aspects, the one or more positions comprise position 521 of SEQ ID NO: 1. In some aspects, the amino acid substitution at position 521 of SEQ ID NO: 1 is selected from the group consisting of: G521A, G521F, G521I, G521L, G521M, and G521V.

In some aspects, the one or more positions comprise position 522 of SEQ ID NO: 1, In some aspects, the amino acid substitution at position 522 of SEQ ID NO: 1 is selected from the group consisting of: M522I, M522L, and M522V.

In some aspects, the one or more positions comprise position 524 of SEQ ID NO: 1. In some aspects, the amino acid substitution at position 524 of SEQ ID NO: 1 is selected from the group consisting of: H524A, H524I, H524L, H524F, and H524V.

In some aspects, the one or more positions comprise position 525 of SEQ ID NO: 1. In some aspects, the amino acid substitution at position 525 of SEQ ID NO: 1 is selected from the group consisting of: L525F, L525I, L525M, L525N, L525Q, L525S, L525T, and L525V.

In some aspects, the one or more positions comprise position 526 of SEQ ID NO: 1. In some aspects, the amino acid substitution at position 526 of SEQ ID NO: 1 is Y526L.

In some aspects, the one or more positions comprise position 527 of SEQ ID NO: 1. In some aspects, the amino acid substitution at position 527 of SEQ ID NO: 1 is S527N.

In some aspects, the one or more positions comprise position 528 of SEQ ID NO: 1. In some aspects, the amino acid substitution at position 528 of SEQ ID NO: 1 is selected from the group consisting of: M528F, M528I, and M528V.

In some aspects, the one or more positions comprise position 533 of SEQ ID NO: 1. In some aspects, the amino acid substitution at position 533 of SEQ ID NO: 1 is selected from the group consisting of: V533F and V533W.

In some aspects, the one or more positions comprise position 534 of SEQ ID NO: 1. In some aspects, the amino acid substitution at position 534 of SEQ ID NO: 1 is selected from the group consisting of: V534Q and V534R.

In some aspects, the one or more positions comprise position 536 of SEQ ID NO: 1. In some aspects, the amino acid substitution at position 536 of SEQ ID NO: 1 is selected from the group consisting of: L536F, and L536M, L536R, and L536Y.

In some aspects, the one or more positions comprise position 537 of SEQ ID NO: 1. In some aspects, the amino acid substitution at position 537 of SEQ ID NO: 1 is selected from the group consisting of: Y537E and Y537S.

In some aspects, the one or more positions comprise position 538 of SEQ ID NO: 1. In some aspects, the amino acid substitution at position 538 of SEQ ID NO: 1 is selected from the group consisting of: D538G and D538K.

In some aspects, the one or more positions comprise position 539 of SEQ ID NO: 1. In some aspects, the amino acid substitution at position 539 of SEQ ID NO: 1 is selected from the group consisting of: L539A and L539R.

In some aspects, the one or more positions comprise position 540 of SEQ ID NO: 1. In some aspects, the amino acid substitution at position 540 of SEQ ID NO: 1 is selected from the group consisting of: L540A and L540F.

In some aspects, the one or more positions comprise position 547 of SEQ ID NO: 1. In some aspects, the amino acid substitution at position 547 of SEQ ID NO: 1 is H547A.

In some aspects, the one or more additional amino acid substitutions are two amino acid substitutions. In some aspects, each of the two amino acid substitutions are at a position of SEQ ID NO: 1 selected from the group consisting of: 343, 345, 347, 348, 351, 354, 384, 387, 388, 389, 391, 392, 404, 418, 421, 521, 524, and 525. In some aspects, the two amino acid substitutions are at positions 345 and 348 of SEQ ID NO: 1 and wherein the amino acid substitution at position 345 of SEQ ID NO: 1 is L345S and the amino acid substitution at position 348 of SEQ ID NO: 1 is N348K. In some aspects, the two amino acid substitutions are at positions 384 and 389 of SEQ ID NO: 1 and wherein the amino acid substitution at position 384 of SEQ ID NO: 1 is L384M and the amino acid substitution at position 389 of SEQ ID NO: 1 is I389M. In some aspects, the two amino acid substitutions are at positions 421 and 392 of SEQ ID NO: 1 and wherein the amino acid substitution at position 421 of SEQ ID NO: 1 is M421I and the amino acid substitution at position 392 of SEQ ID NO: 1 is V392M. In some aspects, the two amino acid substitutions are at positions 354 and 391 of SEQ ID NO: 1 and wherein the amino acid substitution at position 354 of SEQ ID NO: 1 is L354I and the amino acid substitution at position 391 of SEQ ID NO: 1 is L391F. In some aspects, the two amino acid substitutions are at positions 354 and 384 of SEQ ID NO: 1 and wherein the amino acid substitution at position 354 of SEQ ID NO: 1 is L354I and the amino acid substitution at position 384 of SEQ ID NO: 1 is L384M. In some aspects, the two amino acid substitutions are at positions 354 and 387 of SEQ ID NO: 1 and wherein the amino acid substitution at position 354 of SEQ ID NO: 1 is L354I and the amino acid substitution at position 387 of SEQ ID NO: 1 is L387M. In some aspects, the two amino acid substitutions are at positions 387 and 391 and wherein the amino acid substitution at position 387 of SEQ ID NO: 1 is L387M and the amino acid substitution at position 391 of SEQ ID NO: 1 is L391F. In some aspects, the two amino acid substitutions are at positions 384 and 387 of SEQ ID NO: 1 and wherein the amino acid substitution at position 384 of SEQ ID NO: 1 is L384M and the amino acid substitution at position 387 of SEQ ID NO: 1 is L387M. In some aspects, the two amino acid substitutions are at positions 384 and 391 of SEQ ID NO: 1 and wherein the amino acid substitution at position 384 of SEQ ID NO: 1 is L384M and the amino acid substitution at position 391 of SEQ ID NO: 1 is L391F.

In some aspects, the one or more additional amino acid substitutions are three amino acid substitutions. In some aspects, each of the three amino acid substitutions are at a position of SEQ ID NO: 1 selected from the group consisting of: 343, 347, 351, 354, 388, 391, 404, 414, 418, 463, 521, 524, and 525. In some aspects, the three amino acid substitutions are at positions 354, 384, and 391 of SEQ ID NO: 1 and wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F. In some aspects, the three amino acid substitutions are at positions 414, 463, and 524 of SEQ ID NO: 1 and wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L.

In some aspects, the one or more additional amino acid substitutions are four amino acid substitutions. In some aspects, each of the four amino acid substitutions are at a position of SEQ ID NO: 1 selected from the group consisting of: 343, 347, 351, 354, 384, 388, 391, 404, 413, 418, 463, 521, 524, and 525. In some aspects, the four amino acid substitutions are at positions 354, 384, 391, and 418 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I. In some aspects, the four amino acid substitutions are at positions 343, 388, 521, and 404 of SEQ ID NO: 1 and wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L. In some aspects, the four amino acid substitutions are at positions 524, 347, 351, and 525 of SEQ ID NO: 1 and wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N. In some aspects, the four amino acid substitutions are at positions 354, 384, 391, and 463 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P. In some aspects, the four amino acid substitutions are at positions 384, 391, 413, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F.

In some aspects, the one or more additional amino acid substitutions are five amino acid substitutions. In some aspects, each of the five amino acid substitutions are at a position of SEQ ID NO: 1 selected from the group consisting of: 354, 384, 391, 409, 413, 414, 421, 463, and 524, In some aspects, the five amino acid substitutions are at positions 384, 409, 413, 463, and 524 of SEQ ID NO: 1 and wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L. In some aspects, the five amino acid substitutions are at positions 391, 413, 414, 463, and 524 of SEQ ID NO: 1 and wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F. In some aspects, the five amino acid substitutions are at positions 391, 414, 421, 463, and 524 of SEQ ID NO: 1 and wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F. In some aspects, the five amino acid substitutions are at positions 354, 409, 413, 421, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L. In some aspects, the five amino acid substitutions are at positions 354, 409, 421, 463, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.

In some aspects, the one or more additional amino acid substitutions are six amino acid substitutions. In some aspects, each of the six amino acid substitutions are at a position of SEQ ID NO: 1 selected from the group consisting of: 354, 384, 391, 409, 413, 414, 421, 463, and 524. In some aspects, the six amino acid substitutions are at positions 384, 391, 413, 421, 463, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L. In some aspects, the six amino acid substitutions are at positions 409, 413, 414, 421, 463, and 524 of SEQ ID NO: 1 and wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L. In some aspects, the six amino acid substitutions are at positions 354, 391, 409, 413, 414, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L.

In some aspects, the one or more additional amino acid substitutions are seven amino acid substitutions. In some aspects, each of the seven amino acid substitutions are at a position of SEQ ID NO: 1 selected from the group consisting of: 354, 384, 391, 409, 413, 414, 421, 463, 517, and 524. In some aspects, the seven amino acid substitutions are at positions 354, 384, 409, 413, 421, 463, and 524 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F. In some aspects, the seven amino acid substitutions are at positions 354, 391, 413, 421, 463, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L. In some aspects, the seven amino acid substitutions are at positions 354, 391, 413, 414, 421, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F.

In some aspects, the one or more additional amino acid substitutions are eight amino acid substitutions. In some aspects, the eight amino acid substitutions are at positions 384, 391, 409, 413, 421, 463, 517, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

Also provided herein is a chimeric protein comprising a polypeptide of interest fused to a modified ER-LBD as described herein. In some aspects, the polypeptide of interest comprises a nucleic acid binding domain. In some aspects, the nucleic acid binding domain comprises a zinc finger domain. In some aspects, the nucleic acid binding domain comprises a zinc finger domain. In some aspects, the zinc finger domain comprises the sequence

(SEQ ID NO: 62)
MSRPGERPFQCRICMRNFSNMSNLTRHTRTHTGEKPFQCRICMRNFSDR
SVLRRHLRTHTGSQKPFQCRICMRNFSDPSNLARHTRTHTGEKPFQCRI
CMRNFSDRSSLRRHLRTHTGSQKPFQCRICMRNFSQSGTLHRHTRTHTG
EKPFQCRICMRNFSQRPNLTRHLRTHLRGS.

In some aspects, the chimeric protein comprises a chimeric transcription factor, and wherein the polypeptide of interest comprises a nucleic acid binding domain and a transcriptional modulator domain. In some aspects, the transcriptional modular domain is a transcriptional activator. In some aspects, the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain). In some aspects, the transcriptional modular domain is a p65 transcriptional activator comprising the amino acid sequence of

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

Also provided for herein is an isolated polynucleotide molecule comprising a nucleotide sequence encoding a modified ER-LBD as described herein, or encoding the chimeric protein as described herein.

Also provided for herein is a heterologous construct comprising a promoter operatively linked to a polynucleotide molecule as described herein.

Also provided for herein is a plasmid comprising a heterologous construct as described herein.

Also provided for herein is a cell comprising a heterologous construct as described herein or a plasmid as described herein.

Also provided for herein is a genetic switch for modulating transcription of a gene of interest, comprising: (a) a chimeric protein as described herein, wherein the chimeric protein binds to a chimeric transcription factor-responsive (CTF-responsive) promoter operably linked to the gene of interest; and (b) a non-endogenous ligand, wherein binding of the non-endogenous ligand to the modified ER-LBD induces the chimeric protein to modulate transcription of the gene of interest.

In some aspects, the non-endogenous ligand is selected from the group consisting of: 4-hydroxytamoxifen, N-desmethyltamoxifen, tamoxifen-N-oxide, and endoxifen.

In some aspects, the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme.

In some aspects, the gene of interest encodes a cytokine selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha; and/or

In some aspects, the gene of interest encodes an IL12p70 fusion protein comprising the amino acid sequence of

(SEQ ID NO: 58)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSGGGSGGGSGGGSGGG
SRNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS.

Also provided for herein is a method of modulating transcription of a gene of interest, comprising: transforming a cell with (i) a heterologous construct encoding a chimeric protein as described herein and (ii) a target expression cassette comprising a chimeric transcription factor-responsive (CTF-responsive) promoter operably linked to the gene of interest, and inducing the chimeric protein to modulate transcription of the gene of interest by contacting the transformed cell with a non-endogenous ligand. In some aspects, the method further comprises culturing the transformed cell under conditions suitable for expression of the chimeric protein prior to inducing the chimeric protein to modulate transcription. In some aspects, modulating transcription comprises activating transcription of the gene of interest. In some aspects, the target expression cassette is encoded by the heterologous construct encoding a chimeric protein as described herein or the target expression cassette is encoded by a second heterologous construct. In some aspects, the non-endogenous ligand is selected from the group consisting of: 4-hydroxytamoxifen, N-desmethyltamoxifen, tamoxifen-N-oxide, and endoxifen.

Also provided for herein is a method of modulating localization of a chimeric protein comprising transforming a cell with a heterologous construct encoding a chimeric protein as described herein, and inducing nuclear localization of the chimeric protein by contacting the transformed cell with a non-endogenous ligand. In some aspects, the method further comprising culturing the transformed cell under conditions suitable for expression of the chimeric protein prior to inducing the nuclear localization. In some aspects, the non-endogenous ligand is selected from the group consisting of: 4-hydroxytamoxifen, N-desmethyltamoxifen, tamoxifen-N-oxide, and endoxifen.

In some aspects, the transformed cell is in a human or animal, and wherein contacting the transformed cell with the non-endogenous ligand comprises administering a pharmacological dose of the ligand to the human or animal.

In some aspects, the non-endogenous ligand is administered at a concentration at which the non-endogenous ligand is substantially inactive on a wild-type estrogen receptor alpha of SEQ ID NO: 1.

Provided herein are modified ER-LBD comprising an amino acid sequence corresponding to amino acids 282-595 of SEQ ID NO: 1 and one or more additional amino acid substitutions within a region of SEQ ID NO: 1 selected from the group consisting of: positions 343-354, positions 380-392, positions 404-463, and positions 517-540, and position 547. In some aspects, the modified ER-LBD as described herein further comprises a G400V amino acid substitution, an M543A amino acid substitution, and an L544A amino acid substitution. In some aspects, the modified ER-LBD further comprises a G400V amino acid substitution, an M543A amino acid substitution, an L544A, and a V595A amino acid substitution.

In some aspects, the modified ER-LBD comprises a G400V amino acid substitution, an M543A amino acid substitution, and an L544A amino acid substitution, and one or more additional amino acid substitutions. In some aspects, the modified ER-LBD has greater sensitivity to a non-endogenous ligand as compared to an ER-LBD comprising the amino acid sequence of SEQ ID NO: 2. In some aspects, the modified ER-LBD has greater selectivity to a non-endogenous ligand as compared to an ER-LBD comprising the amino acid sequence of SEQ ID NO: 2.

In some aspects, the modified ER-LBD comprises a G400V amino acid substitution, an M543A amino acid substitution, an L544A amino acid substitution, and a V595A amino acid substitution, and one or more additional amino acid substitutions. In some aspects, the modified ER-LBD has greater sensitivity to a non-endogenous ligand as compared to an ER-LBD comprising the amino acid sequence of SEQ ID NO: 3. In some aspects, the modified ER-LBD has greater selectivity to a non-endogenous ligand as compared to an ER-LBD comprising the amino acid sequence of SEQ ID NO: 3.

In some aspects, a modified ER-LBD of the present disclosure has greater sensitivity to a non-endogenous ligand as compared to an endogenous ligand as a result of the one or more additional amino acid substitutions.

In some aspects, a modified ER-LBD of the present disclosure has greater sensitivity to a non-endogenous ligand as compared to an ER-LBD comprising the amino acid sequence of SEQ ID NO: 2 or SEQ ID NO: 3.

In some aspects, a modified ER-LBD of the present disclosure has greater selectivity to a non-endogenous ligand as compared to an ER-LBD comprising the amino acid sequence of SEQ ID NO: 2 or SEQ ID NO: 3.

In some aspects, the one or more additional amino acid substitutions are at one or more positions of SEQ ID NO: 1 selected from the group consisting of: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

In some aspects, the one or more positions include position 343 of SEQ ID NO: 1. In some aspects the amino acid substitution at position 343 is selected from the group consisting of: M343F, M343I, M343L, and M343V.

In some aspects, the one or more positions include position 344 of SEQ ID NO: 1. In some aspects the amino acid substitution at position 344 is G344M.

In some aspects, the one or more positions include position 345 of SEQ ID NO: 1. In some aspects the amino acid substitution at position 345 is L345S.

In some aspects, the one or more positions include position 346 of SEQ ID NO: 1. In some aspects the amino acid substitution at position 346 is selected from the group consisting of: L346I, L346M, L346F, and L346V.

In some aspects, the one or more positions include position 347 of SEQ ID NO: 1. In some aspects the amino acid substitution at position 347 is selected from the group consisting of: T347D, T347E, T347F, T347I, T347K, T347L, T347M, T347N, T347Q, T347R, T347S, and T347V.

In some aspects, the one or more positions include position 348 of SEQ ID NO: 1. In some aspects the amino acid substitution at position 348 is N348K.

In some aspects, the one or more positions include position 349 of SEQ ID NO: 1. In some aspects the amino acid substitution at position 349 is selected from the group consisting of: L349I, L349M, L349F, and L349V.

In some aspects, the one or more positions include position 350 of SEQ ID NO: 1. In some aspects the amino acid substitution at position 350 is selected from the group consisting of: A350F, A350I, A350L, A350M and A350V.

In some aspects, the one or more positions include position 351 of SEQ ID NO: 1. In some aspects the amino acid substitution at position 351 is selected from the group consisting of: D351E, D351F, D351I, D351L, D351M, D351N, D351Q, and D351V.

In some aspects, the one or more positions include position 352 of SEQ ID NO: 1. In some aspects the amino acid substitution at position 352 is R352K.

In some aspects, the one or more positions include position 354 of SEQ ID NO: 1. In some aspects the amino acid substitution at position 354 is selected from the group consisting of: L354I, L354M, L354F, and L354V.

In some aspects, the one or more positions include position 380 of SEQ ID NO: 1. In some aspects the amino acid substitution at position 380 is E380Q.

In some aspects, the one or more positions include position 384 of SEQ ID NO: 1. In some aspects the amino acid substitution at position 384 is selected from the group consisting of: L384I, L384M, L384F, and L384V.

In some aspects, the one or more positions include position 386 of SEQ ID NO: 1. In some aspects the amino acid substitution at position 386 is I386V.

In some aspects, the one or more positions include position 387 of SEQ ID NO: 1. In some aspects the amino acid substitution at position 387 is selected from the group consisting of: L387I, L387M, L387F, and L387V.

In some aspects, the one or more positions include position 388 of SEQ ID NO: 1. In some aspects the amino acid substitution at position 388 is selected from the group consisting of: M388I, M388L, and M388F.

In some aspects, the one or more positions include position 389 of SEQ ID NO: 1. In some aspects the amino acid substitution at position 389 is I389M.

In some aspects, the one or more positions include position 391 of SEQ ID NO: 1. In some aspects the amino acid substitution at position 391 is selected from the group consisting of: L391I, L391M, L391F, and L391V.

In some aspects, the one or more positions include position 392 of SEQ ID NO: 1. In some aspects the amino acid substitution at position 392 is V392M.

In some aspects, the one or more positions include position 404 of SEQ ID NO: 1. In some aspects the amino acid substitution at position 404 is selected from the group consisting of: F404I, F404L, F404M, and F404V.

In some aspects, the one or more positions include position 407 of SEQ ID NO: 1. In some aspects the amino acid substitution at position 407 is N407D.

In some aspects, the one or more positions include position 409 of SEQ ID NO: 1. In some aspects the amino acid substitution at position 409 is L409V.

In some aspects, the one or more positions include position 413 of SEQ ID NO: 1. In some aspects the amino acid substitution at position 413 is N413D.

In some aspects, the one or more positions include position 414 of SEQ ID NO: 1. In some aspects the amino acid substitution at position 414 is Q414E.

In some aspects, the one or more positions include position 417 of SEQ ID NO: 1. In some aspects the amino acid substitution at position 417 is C417S.

In some aspects, the one or more positions include position 418 of SEQ ID NO: 1. In some aspects the amino acid substitution at position 418 is selected from the group consisting of: V418I, V418L, V418M, and V418F.

In some aspects, the one or more positions include position 420 of SEQ ID NO: 1. In some aspects the amino acid substitution at position 420 is selected from the group consisting of: G420I, G420M, G420F, and G420V.

In some aspects, the one or more positions include position 421 of SEQ ID NO: 1. In some aspects the amino acid substitution at position 421 is selected from the group consisting of: M421I, M421L, M421F, and M421V.

In some aspects, the one or more positions include position 422 of SEQ ID NO: 1. In some aspects the amino acid substitution at position 422 is V422I.

In some aspects, the one or more positions include position 424 of SEQ ID NO: 1. In some aspects the amino acid substitution at position 424 is selected from the group consisting of: I424L, I424M, I424F, and I424V.

In some aspects, the one or more positions include position 428 of SEQ ID NO: 1. In some aspects the amino acid substitution at position 428 is selected from the group consisting of: L428I, L428M, L428F, and L428V.

In some aspects, the one or more positions include position 463 of SEQ ID NO: 1. In some aspects the amino acid substitution at position 463 is S463P.

In some aspects, the one or more positions include position 517 of SEQ ID NO: 1. In some aspects the amino acid substitution at position 517 is M517A.

In some aspects, the one or more positions include position 521 of SEQ ID NO: 1. In some aspects the amino acid substitution at position 521 is selected from the group consisting of: G521A, G521F, G521I, G521L, G521M, and G521V.

In some aspects, the one or more positions include position 522 of SEQ ID NO: 1. In some aspects the amino acid substitution at position 522 is selected from the group consisting of: M522I, M522L, and M522V.

In some aspects, the one or more positions include position 524 of SEQ ID NO: 1. In some aspects the amino acid substitution at position 524 is selected from the group consisting of: H524A, H524I, H524L, H524F, and H524V.

In some aspects, the one or more positions include position 525 of SEQ ID NO: 1. In some aspects the amino acid substitution at position 525 is selected from the group consisting of: L525F, L525I, L525M, L525N, L525Q, L525S, L525T, and L525V.

In some aspects, the one or more positions include position 526 of SEQ ID NO: 1. In some aspects the amino acid substitution at position 526 is Y526L.

In some aspects, the one or more positions include position 527 of SEQ ID NO: 1. In some aspects the amino acid substitution at position 527 is S527N.

In some aspects, the one or more positions include position 528 of SEQ ID NO: 1. In some aspects the amino acid substitution at position 528 is selected from the group consisting of: M528F, M528I, and M528V.

In some aspects, the one or more positions include position 533 of SEQ ID NO: 1. In some aspects the amino acid substitution at position 533 is selected from the group consisting of: V533F and V533W.

In some aspects, the one or more positions include position 534 of SEQ ID NO: 1. In some aspects the amino acid substitution at position 534 is selected from the group consisting of: V534Q and V534R.

In some aspects, the one or more positions include position 536 of SEQ ID NO: 1. In some aspects the amino acid substitution at position 536 is selected from the group consisting of: L536F, and L536M, L536R, and L536Y.

In some aspects, the one or more positions include position 537 of SEQ ID NO: 1. In some aspects the amino acid substitution at position 537 is selected from the group consisting of: Y537E and Y537S.

In some aspects, the one or more positions include position 538 of SEQ ID NO: 1. In some aspects the amino acid substitution at position 538 is selected from the group consisting of: D538G and D538K.

In some aspects, the one or more positions include position 539 of SEQ ID NO: 1. In some aspects the amino acid substitution at position 539 is selected from the group consisting of: L539A and L539R.

In some aspects, the one or more positions include position 540 of SEQ ID NO: 1. In some aspects the amino acid substitution at position 540 is selected from the group consisting of: L540A and L540F.

In some aspects, the one or more positions include position 547 of SEQ ID NO: 1. In some aspects the amino acid substitution at position 547 is H547A.

In some aspects, the one or more additional amino acid substitutions include two amino acid substitutions. In some aspects, each of the two amino acid substitutions are at a position of SEQ ID NO: 1 selected from the group consisting of: 343, 345, 347, 348, 351, 354, 384, 387, 388, 389, 391, 392, 404, 418, 421, 521, 524, and 525.

In some aspects, the two amino acid substitutions are at positions 345 and 348 of SEQ ID NO: 1. In some aspects, the amino acid substitution at position 345 of SEQ ID NO: 1 is L345S and the amino acid substitution at position 348 of SEQ ID NO: 1 is N348K.

In some aspects, the two amino acid substitutions are at positions 384 and 389 of SEQ ID NO: 1. In some aspects, the amino acid substitution at position 384 of SEQ ID NO: 1 is L384M and the amino acid substitution at position 389 of SEQ ID NO: 1 is I389M.

In some aspects, the two amino acid substitutions are at positions 421 and 392 of SEQ ID NO: 1. In some aspects, the amino acid substitution at position 421 of SEQ ID NO: 1 is M421I and the amino acid substitution at position 392 of SEQ ID NO: 1 is V392M.

In some aspects, the two amino acid substitutions are at positions 354 and 391 of SEQ ID NO: 1. In some aspects, the amino acid substitution at position 354 of SEQ ID NO: 1 is L354I and the amino acid substitution at position 391 of SEQ ID NO: 1 is L391F.

In some aspects, the two amino acid substitutions are at positions 354 and 384 of SEQ ID NO: 1. In some aspects, the amino acid substitution at position 354 of SEQ ID NO: 1 is L354I and the amino acid substitution at position 384 of SEQ ID NO: 1 is L384M.

In some aspects, the two amino acid substitutions are at positions 354 and 387 of SEQ ID NO: 1. In some aspects, the amino acid substitution at position 354 of SEQ ID NO: 1 is L354I and the amino acid substitution at position 387 of SEQ ID NO: 1 is L387M.

In some aspects, the two amino acid substitutions are at positions 387 and 391. In some aspects, the amino acid substitution at position 387 of SEQ ID NO: 1 is L387M and the amino acid substitution at position 391 of SEQ ID NO: 1 is L391F.

In some aspects, the two amino acid substitutions are at positions 384 and 387 of SEQ ID NO: 1. In some aspects, the amino acid substitution at position 384 of SEQ ID NO: 1 is L384M and the amino acid substitution at position 387 of SEQ ID NO: 1 is L387M.

In some aspects, the two amino acid substitutions are at positions 384 and 391 of SEQ ID NO: 1. In some aspects, the amino acid substitution at position 384 of SEQ ID NO: 1 is L384M and the amino acid substitution at position 391 of SEQ ID NO: 1 is L391F.

In some aspects, the one or more additional amino acid substitutions include three amino acid substitutions. In some aspects, the three amino acid substitutions are each at a position of SEQ ID NO: 1 selected from the group consisting of: 343, 347, 351, 354, 388, 391, 404, 418, 521, 524, and 525.

In some aspects, the three amino acid substitutions are at positions 354, 384, and 391 of SEQ ID NO: 1. In some aspects, the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F.

In some aspects, the one or more additional amino acid substitutions include four amino acid substitutions.

In some aspects, the four amino acid substitutions are at positions 354, 384, 391, and 418 of SEQ ID NO: 1. In some aspects, the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I.

In some aspects, the four amino acid substitutions are at positions 343, 388, 521, and 404 of SEQ ID NO: 1. In some aspects, the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L.

In some aspects, the four amino acid substitutions are at positions 524, 347, 351, and 525 of SEQ ID NO: 1. In some aspects, the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N.

In some aspects, the non-endogenous ligand is selected from the group consisting of: 4-hydroxytamoxifen (4-OHT), N-desmethyltamoxifen, tamoxifen-N-oxide, and endoxifen.

Also provided are chimeric proteins including a polypeptide of interest fused to a modified ER-LBD as described herein. In some aspects, the polypeptide of interest includes a nucleic acid binding domain. In some aspects, the nucleic acid binding domain includes a zinc finger (ZF) domain. In some aspects, the chimeric protein is a transcription factor and the polypeptide of interest includes a transcriptional modulator domain.

Also provided are isolated polynucleotide molecules encoding modified ER-LBD as described herein or the chimeric protein as described herein.

Also provided are heterologous constructs including a promoter operably linked to a polynucleotide molecule encoding a modified ER-LBD as described herein or a chimeric protein as described herein.

Also provided are plasmids comprising the heterologous constructs as described herein.

Also provided are cells (such as an isolated cell or a population of cells) including a heterologous construct as described herein or a plasmid as described herein.

Also provided is a genetic switch for modulating transcription of a gene of interest. In some aspects, the genetic switch includes a chimeric protein including a modified ER-LBD as described herein and a transcription modulator, and a non-endogenous ligand, wherein binding of the non-endogenous ligand to the modified ER-LBD induces the chimeric protein to modulate transcription of the gene of interest. In some aspects, the non-endogenous ligand of the genetic switch is selected from the group consisting of: 4-hydroxytamoxifen (4-OHT), N-desmethyltamoxifen, tamoxifen-N-oxide, and endoxifen.

Also provided herein is a method of modulating transcription of a gene of interest. In some aspects, the method includes (a) transforming a cell with (i) a heterologous construct encoding the chimeric protein including a modified ER-LBD and a transcriptional modulator domain, and (ii) a target expression cassette comprising a gene of interest; (b) culturing the transformed call under conditions suitable for expression of the chimeric protein; and (c) inducing the chimeric protein to modulate transcription of the gene of interest by contacting the transformed cell with a non-endogenous ligand.

In some aspects, the method of modulating transcription is a method of activating transcription.

In some aspects, the method of modulating transcription is a method of repressing transcription.

In some aspects, the target expression cassette is encoded by the heterologous construct encoding the chimeric.

In some aspects, the target expression cassette is encoded by a different heterologous construct from the heterologous construct encoding the chimeric.

Also provided is a method of modulating localization of a polypeptide of interest. In some aspects, the method includes (a) transforming a cell with a heterologous construct encoding a chimeric protein including a polypeptide of interest fused to a modified ER-LBD as described herein; (b) culturing the transformed cell under conditions suitable for expression of the chimeric protein; and (c) inducing nuclear localization of the chimeric protein by contacting the transformed cell with a non-endogenous ligand.

In some aspects, the transformed cell of any of the methods described herein is in a human or an animal. In some aspects, contacting the transformed cell with the non-endogenous ligand comprises administering a pharmacological dose of the ligand to the human or animal.

In some aspects, the non-endogenous ligand of step (c) of the previously described methods is selected from the group consisting of: 4-hydroxytamoxifen, N-desmethyltamoxifen, tamoxifen-N-oxide, and endoxifen.

In some embodiments, the non-endogenous ligand is administered at a concentration at which the non-endogenous ligand is substantially inactive on wild-type estrogen receptor alpha.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1A and FIG. 1B, provide binding energy calculations for the first set of mutations analyzed in silico. FIG. 1A provides binding energy calculations for binding to estradiol, FIG. 1B provides binding energy calculations for binding to 4-OHT.

FIG. 2 provides binding energy calculations for 4-OHT binding, for the second set of mutations analyzed in silico.

FIG. 3 provides binding energy calculations for 4-OHT binding, for the third set of mutations analyzed in silico.

FIG. 4 provides binding energy calculations for 4-OHT binding, for the fourth set of mutations analyzed in silico.

FIG. 5 provides binding energy calculations for 4-OHT binding, for the fifth set of mutations analyzed in silico.

FIG. 6 shows structural differences between the estradiol-bound and non-endogenous ligand-bound conformations in the orientation and docking site of helix 12.

FIG. 7 provides binding energy calculations for the agonist-bound versus the antagonist-bound conformation, for the sixth set of mutations analyzed in silico.

FIG. 8A, FIG. 8B, and FIG. 8C show the effect of various modified ER-LBDs on reporter expression over various concentrations of 4-OHT, as assayed in a first transfection screen.

FIG. 9A, FIG. 9B, and FIG. 9C show the effect of various modified ER-LBDs on reporter expression over various concentrations of 4-OHT, as assayed in a second transfection screen.

FIG. 10A, FIG. 10B, and FIG. 10C show the effect of various modified ER-LBDs on reporter expression over various concentrations of 4-OHT, as assayed in a third transfection screen.

FIG. 11A and FIG. 11B show the effect of various modified ER-LBDs on reporter expression over various concentrations of 4-OHT, as assayed in a first transduction screen.

FIG. 12 shows the effect of various modified ER-LBDs on reporter expression over various concentrations of 4-OHT, as assayed in a second transduction screen.

FIG. 13 shows the effect of various modified ER-LBDs on reporter expression over various concentrations of 4-OHT, as assayed in a second transduction screen.

FIG. 14A and FIG. 14B show a backbone for high-throughput protein engineering of ERT2 (SB04401) and an OFF mCherry reporter construct (SB01066).

FIG. 15A and FIG. 15B show the effect of various modified ER-LBDs on reporter expression over various concentrations of endoxifen and 4-OHT, as assayed in a combinatorial library screen.

FIG. 16A, FIG. 16B, FIG. 16C, and FIG. 16D show the effect of various modified ER-LBDs on reporter expression over various concentrations of endoxifen, 4-OHT, and estradiol, as assayed in a validation screen. SB03422 is the wild-type ER-LBD and is included as a benchmark for performance.

FIG. 17A and FIG. 17B shows the effect of various modified ER-LBDs on reporter expression over various concentrations of endoxifen and 4-OHT, as assayed in NK cells. Arrow indicates estimated pharmacologically relevant 4-OHT or endoxifen concentrations in humans.

FIG. 18A and FIG. 18B show the effect of various modified ER-LBDs on IL-12 expression over various concentrations of endoxifen, as assayed in NK cells.

### DETAILED DESCRIPTION

Terms used in the claims and specification are defined as set forth below unless otherwise specified.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Modified Estrogen Receptor Ligand Binding Domains (ER-LBD)

The present disclosure provides a modified estrogen receptor ligand binding domain (ER-LBD) comprising an amino acid sequence corresponding to amino acids 282-595 of SEQ ID NO: 1 (human Estrogen Receptor, UniProt ID No: P03372), comprising amino acid substitutions G400V, M543A, and L544A or amino acid substitutions G400V, M543A, L544A, and V595A, and comprising one or more additional amino acid substitutions to ligand binding residues within a region of SEQ ID NO: 1 selected from positions 343-354, positions 380-392, positions 404-463, and positions 517-540, and position 547. In some aspects, the one or more amino acid substitutions result in: (a) greater sensitivity to a non-endogenous ligand as compared to an endogenous ligand, (b) greater sensitivity to a non-endogenous ligand as compared to an ER-LBD of SEQ ID NO: 2 or SEQ ID NO: 3, and/or (c) greater selectivity to a non-endogenous ligand as compared to an ER-LBD of SEQ ID NO: 2 or SEQ ID NO: 3.

The one or more additional amino acid substitutions may result in: (a) greater sensitivity to a non-endogenous ligand as compared to an endogenous ligand, (b) greater sensitivity to a non-endogenous ligand as compared to an ER-LBD of SEQ ID NO: 2 or SEQ ID NO: 3 and/or (c) greater selectivity to a non-endogenous ligand as compared to an ER-LBD of SEQ ID NO: 2 or SEQ ID NO: 3. In some embodiments, the one or more additional amino acid substitutions results in greater sensitivity to a non-endogenous ligand as compared to an ER-LBD of SEQ ID NO: 2. In some embodiments, the one or more additional amino acid substitutions results in greater sensitivity to a non-endogenous ligand as compared to an ER-LBD of SEQ ID NO: 2. In some embodiments, the one or more additional amino acid substitutions results in greater selectivity to a non-endogenous ligand as compared to an ER-LBD of SEQ ID NO: 2. In some embodiments, the one or more additional amino acid substitutions results in greater selectivity to a non-endogenous ligand as compared to an ER-LBD of SEQ ID NO: 3.

“Ligand binding residues” refers to residues located at the ligand binding pocket of estrogen receptor (ER) or an ER-ligand binding domain, and includes the pocket for binding to an endogenous ligand (e.g., estradiol) and the pocket for binding to a non-endogenous ligand such as 4-OHT. Residues within positions 343-354, positions 380-392 and positions 404-463 corresponding to SEQ ID NO: 1 are involved in binding to both endogenous and non-endogenous ligands. Residues within positions 517-547 (e.g., residues 517-40 and residue 547) corresponding to SEQ ID NO: 1 are located within a helix referred to as helix 12 and are involved in endogenous ligand binding.

Greater sensitivity to a non-endogenous ligand as compared to sensitivity to a non-endogenous ligand means that the modified ER-LBD binds to a non-endogenous ligand (e.g., endoxifen) with a higher affinity as compared to the affinity of its binding to an endogenous ligand (e.g., estradiol).

Greater sensitivity to a non-endogenous ligand as compared to sensitivity an ER-LBD not including the one or more amino acid substitutions (e.g., an ER-LBD comprising the amino acid sequence of SEQ ID NO: 2 or SEQ ID NO: 3) means that the modified ER-LBD binds to a non-endogenous ligand (e.g., endoxifen) with a higher affinity as compared to the affinity of binding of ER-LBD not including the one or more additional amino acid substitutions to the non-endogenous ligand. In some embodiments, the greater sensitivity is at least a 1.5-fold, at least a 2-fold, at least a 3-fold, at least a 4-fold, or at least a 5-fold improvement in binding affinity to a non-endogenous ligand, as compared to binding of an ER-LBD not including the one or more additional amino acid substitutions. In some embodiments, greater sensitivity is demonstrated by greater transcriptional modulation (e.g., greater transcriptional activation or greater transcriptional repression) of a chimeric transcription factor including a modified ER-LBD, as compared to a chimeric transcription factor including an ER-LBD that lacks the one or more additional amino acid substitutions. In some embodiments, in a transfection of transduction assay, a chimeric transcription factor including a modified ER-LBD is capable of inducing at least 10%, at least 15%, at least 20%, at least 25%, at least 30%, or at least 35% greater expression of a reporter under control of a chimeric transcription factor-responsive promoter in response to a non-endogenous ligand (e.g., 4-OHT) (as measured by % of cells positive for the reporter, or as measured by geometric mean fluorescent intensity) as compared to the expression of the reporter under the same conditions but with an ER-LBD that lacks the one or more additional amino acid substitutions.

Greater selectivity to a non-endogenous ligand refers to preferential binding to a non-endogenous ligand (e.g., 4-OHT or endoxifen) as compared to an endogenous ligand (e.g., estradiol). Selectivity may be measured using a selectivity coefficient, which is the equilibrium constant for the reaction of displacement by one ligand (e.g., a non-endogenous ligand) of another ligand (e.g., an endogenous ligand) in a complex with the substrate (e.g., a modified ER-LBD). The greater the selectivity coefficient, the more a competing ligand (e.g., an endogenous ligand) will displace the initial ligand (e.g., a non-endogenous ligand) from the complex formed with the substrate (e.g., a modified ER-LBD). In some embodiments, greater selectivity is demonstrated by improved transcriptional modulation of a chimeric transcription factor in the presence of a non-endogenous ligand as compared to transcriptional modulation in the presence of an endogenous ligand. In some embodiments, in a transfection of transduction assay, a chimeric transcription factor including a modified ER-LBD is capable of inducing at least 10%, at least 15%, at least 20%, at least 25%, at least 30%, or at least 35% greater expression of a reporter under control of a chimeric transcription factor-responsive promoter in response to a non-endogenous ligand (e.g., 4-OHT) (as measured by % of cells positive for the reporter, or as measured by geometric mean fluorescent intensity) as compared to the expression of the reporter under the same conditions but in response to an endogenous ligand (e.g., estradiol).

In some aspects, the one or more amino acid substitutions to ligand binding residues include one or more amino acid substitutions within helix 12. Helix 12 of an ER-LBD includes residue positions 533-547 of SEQ ID NO: 1. In some embodiments, the one or more amino acid substitutions within helix 12 are at one or more positions selected from 538, 536, 539, 540, 547, 534, 533, and 537.

“Non-endogenous ligand” may refer to, for example, a synthetic estrogen receptor binding ligand that is not naturally expressed by an organism that expresses an estrogen receptor. Non-endogenous estrogen receptor binding ligands include, without limitation, tamoxifen and metabolites thereof, such as 4-hydroxytamoxifen, N-desmethyltamoxifen, tamoxifen-N-oxide, and endoxifen.

The one or more additional amino acid substitutions may be at one or more positions of SEQ ID NO:1 selected from 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547. In some embodiments, the one or more additional amino acid substitutions include substitutions at one of the above-listed positions, two of the above-listed positions, three of the above-listed positions, four of the above-listed positions, or five of the above-listed positions.

In some aspects, the one or more additional amino acids substitutions are selected from one or more of the substitutions listed in Table 1.

TABLE 1
Amino Acid Substitutions

| | | | | | |
|---|---|---|---|---|---|
| M343I | L349I | L384M | N413D | L428F | L525S |
| M343L | L349M | L384V | Q414E | M517A | Y526L |
| M343V | L349V | L384F | C417S | G521A | S527N |
| M343F | L349F | I386V | V418I | G521I | M528I |
| G344M | A350L | L387I | V418L | G521L | M528V |
| L345S | A350M | L387M | V418M | G521M | M528F |
| L346I | A350V | L387V | V418F | G521V | V533F |
| L346M | A350F | L387F | G420I | G521F | V533W |
| L346V | A350I | M388I | G420M | M522I | V534R |
| L346F | D351I | M388L | G420V | M522L | V534Q |
| T347I | D351L | M388F | G420F | M522V | L536F |
| T347L | D351M | I389M | M421I | H524A | L536M |
| T347M | D351N | L391I | M421L | H524I | L536Y |
| T347N | D351V | L391M | M421V | H524L | Y537E |
| T347R | D351E | L391V | M421F | H524V | D538K |
| T347V | D351F | L391F | V422I | H524F | L539R |
| T347D | D351Q | V392M | I424L | L525I | L539A |
| T347E | R352K | F404I | I424M | L525M | L540A |
| T347F | L354I | F404L | I424V | L525N | L540F |
| T347K | L354M | F404M | I424F | L525T | H547A |
| T347Q | L354V | F404V | L428I | L525V | |
| T347S | L354F | N407D | L428M | L525F | |
| N348K | L384I | L409V | L428V | L525Q | |

In some aspects, the one or more additional mutations comprise at least two mutations, at least three mutations, at least four mutations, at least five mutations, at least six mutations, at least seven mutations, or at least eight mutations. In some aspects, the one or more additional mutations comprise two to ten mutations, two to nine mutations, two to eight mutations, two to seven mutations, two to six mutations, two to five mutations, two to four mutations, two to three mutations, three to ten mutations, three to nine mutations, three to eight mutations, three to seven mutations, three to six mutations, three to five mutations, three to four mutations, four to ten mutations, four to nine mutations, four to eight mutations, four to seven mutations, four to six mutations, four to five mutations, five to ten mutations, five to nine mutations, five to eight mutations, five to seven mutations, five to six mutations, six to ten mutations, six to nine mutations, six to eight mutations, six to seven mutations, seven to ten mutations, seven to nine mutations, seven to eight mutations, eight to ten mutations, eight to nine mutations, or nine to ten mutations.

In some aspects, the one or more additional mutations comprise at least two mutations that are selected from the mutations listed in Table 2.

TABLE 2
Combination Amino Acid Substitutions

| | |
|---|---|
| L345S_N348K | L384M_L391F |
| L384M_I389M | L354I_L384M |
| M421I_V392M | L354I_L384M_L391F |
| L354I_L391F | L354I_L384M_L391F_V418I |
| L354I_L387M | M343I_M388I_G521I_F404L |
| L387M_L391F | H524V_T347R_D351Q_L525N |
| L384M_L387M | L354I_L384M_L391F_V418I |
| Q414E_S463P_H524L | L354I_L384M_L391V_S463P |
| L384M_L391V_N413D_H524F | L384M_L409V_N413D_S463P_H524L |
| L391V_N413D_Q414E_S463P_H524F | L391V_Q414E_M421L_S463P_H524F |
| L354I_L409V_N413D_M421L_H524L | L354I_L409V_M421L_S463P_H524L |
| L384M_L391V_N413D_M421L_S463P_H524L | L409V_N413D_Q414E_M421L_S463P_H524L |
| L354I_L391V_L409V_N413D_Q414E_H524L | L354I_L384M_L409V_N413D_M421L_S463P_H524F |
| L354I_L391V_N413D_M421L_S463P_M517A_H524L | L354I_L391V_N413D_Q414E_M421L_M517A_H524F |
| L384M_L391V_L409V_N413D_M421L_S463P_M517A_H524F | |

In some embodiments, provided herein is a modified ER-LBD variant having an amino acid sequence that is at least 85%, at least 90%, at least 91%, at least 92%, at least 93%, at least 94%, at least 95%, at least 96%, at least 97%, at least 98%, at least 99%, or 100% identical to a modified ER-LBD as described herein, provided that the variant includes the G400V/MS43A/L544A triple amino acid substitution or the G400V/M543A/L544A/V595A quadruple amino acid substitution, and includes the one or more additional amino acid substitutions that confer greater sensitivity and/or greater selectivity for a non-endogenous ligand (e.g., one or more of the amino acid substitutions shown in Table 1 and Table 2).

Chimeric Proteins

In some aspects, the present disclosure provides chimeric proteins including a polypeptide of interest fused to the modified ER-LBD. The modified ER-LBD is capable of nuclear localization upon binding to a non-endogenous ligand. Thus, fusion of a modified ER-LBD to a polypeptide of interest may allow for control of cellular localization of the polypeptide of interest.

In some embodiments, the polypeptide of interest includes a linker. One or more linkers can be used between various domains of chimeric proteins, such as between an ER-LBD and a polypeptide of interest. For example, a polypeptide linker can include an amino acid sequence such as one or more of: GGGGSGGGGSGGGGSVDGF (SEQ ID NO: 4) and ASGGGGSAS (SEQ ID NO: 5).

In some embodiments, the polypeptide of interest includes at least one nucleic acid binding domain. In some embodiments, the nucleic acid binding domain is a zinc-finger domain. In some embodiments, the chimeric protein includes a transcription modulator, such as a transcription activator or a transcription repressor. Inclusion of a nucleic acid binding domain may allow for targeted nucleic acid binding by the chimeric protein that is inducible by a non-endogenous ligand (e.g., 4-OHT or endoxifen).

In some aspects, the nucleic acid binding domain comprises a DNA binding zinc finger protein domain (ZF protein domain). In some aspects, the ZF protein domain is modular in design and is composed of zinc finger arrays (ZFA). In some aspects, the transcriptional effector domain is selected from a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif (SEQ ID NO: 82) of the hairy-related basic helix-loop-helix repressor proteins, the motif is known as a WRPW (SEQ ID NO: 82) repression domain; a DNA (cytosine-5)-methyltransferase 3B (DNMT3B) repression domain; and an HP1 alpha chromoshadow repression domain.

In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

An exemplary ZF protein domain is shown in the sequence

(SEQ ID NO: 6)
SRPGERPFQCRICMRNFSRRHGLDRHTRTHTGEKPFQCRICMRNFSDHS
SLKRHLRTHTGSQKPFQCRICMRNFSVRHNLTRHLRTHTGEKPFQCRIC
MRNFSDHSNLSRHLKTHTGSQKPFQCRICMRNFSQRSSLVRHLRTHTGE
KPFQCRICMRNFSESGHLKRHLRTHLRGS.

In some embodiments, a ZF protein domain is a ZF5-7 DNA binding domain. An exemplary ZF5-7 DNA binding domain is shown in the sequence

(SEQ ID NO: 62)
MSRPGERPFQCRICMRNFSNMSNLTRHTRTHTGEKPFQCRICMRNFSDR
SVLRRHLRTHTGSQKPFQCRICMRNFSDPSNLARHTRTHTGEKPFQCRI
CMRNFSDRSSLRRHLRTHTGSQKPFQCRICMRNFSQSGTLHRHTRTHTG
EKPFQCRICMRNFSQRPNLTRHLRTHLRGS.

In some embodiments, the chimeric protein is a chimeric transcription factor and includes, in addition to the modified ER-LBD, a nucleic acid binding domain and a transcriptional modulator domain. In some aspects, the nucleic acid binding domain and the transcriptional modulator domain are part of the same naturally occurring protein. In some aspects, the nucleic acid binding domain and the transcriptional modulator domain are heterologous and do not exist naturally within the same protein.

“Transcriptional modulator domain” as used herein refers to a polypeptide domain that, when targeted to a promoter region of a gene (e.g., by a nucleic acid binding domain that specifically binds to a promoter of interest), is capable of modulating the transcription of the gene. In some aspects, the transcriptional modulator domain comprises a transcriptional repressor. In some aspects, the transcriptional repressor comprises a transcriptional repressor domain selected from a Kruppel associated box (KRAB) repression domain; a Repressor Element Silencing Transcription Factor (REST) repression domain; a WRPW motif (SEQ ID NO: 82) of the hairy-related basic helix-loop-helix repressor proteins, the motif is known as a WRPW (SEQ ID NO: 82) repression domain; a DNA (cytosine-5)-methyltransferase 3B (DNMT3B) repression domain; and an HP1 alpha chromoshadow repression domain.

In some aspects, the transcriptional modulator domain comprises a transcriptional activator. In some aspects, the transcriptional activator comprises a transcriptional activator domain selected from a Herpes Simplex Virus Protein 16 (VP16) activation domain; an activation domain comprising four tandem copies of VP16; a VP64 activation domain; a p65 activation domain of NFκB (i.e., p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain). In some aspects, the transcriptional modulator domain comprises a p65 transcriptional activator. In some aspects, a p65 transcriptional activator comprises the amino acid sequence

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

Genetic Switches

Also provided herein are genetic switches for modulating transcription. A genetic switch may include (a) a chimeric transcription factor that includes a modified ER-LBD and is capable of binding to a chimeric transcription factor-responsive promoter (CTF-responsive promoter) operably linked to a gene of interest, and (b) a non-endogenous ligand that binds to the modified ER-LBD of the chimeric protein. Upon binding of the non-endogenous ligand to the modified ER-LBD, the chimeric protein may modulate transcription of a gene of interest.

In some embodiments, the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme. In some embodiments, the gene of interest encodes a cytokine. In some embodiments, the gene of interest encodes a cytokine selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha. In some embodiments, the gene of interest encodes an IL12p70 fusion protein comprising the amino acid sequence of

(SEQ ID NO: 58)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSGGGSGGGSGGGSGGG
SRNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS.

In some embodiments, the non-endogenous ligand is selected from 4-hydroxytamoxifen (4-OHT), N-desmethyltamoxifen, tamoxifen-N-oxide, and endoxifen.

In particular embodiments, the non-endogenous ligand is 4-hydroxytamoxifen (4-OHT, also referred to as afimoxifene).

In particular embodiments, the non-endogenous ligand is endoxifen.

Isolated Polynucleotide Molecules and Heterologous Constructs

Also provided herein are isolated polynucleotide molecules and heterologous constructs encoding a modified ER-LBD or chimeric protein as described herein. In some aspects the present disclosure provides an isolated polynucleotide molecule comprising a nucleotide sequence encoding a modified ER-LBD or chimeric protein as described herein. In some aspects, the present disclosure provides a heterologous construct comprising a promoter operatively linked to the polynucleotide molecule encoding the modified ER-LBD or chimeric protein.

In some aspects, the present disclosure further provides isolated polynucleotides and/or heterologous constructs including a target gene expression cassette.

“Isolated” nucleic acid molecule or polynucleotide refers to a nucleic acid molecule, such as DNA or RNA, which has been removed from its native environment. For example, a polynucleotide encoding a modified ER-LBD or chimeric protein contained in a heterologous construct is considered isolated. Further examples of an isolated polynucleotide include recombinant polynucleotides maintained in heterologous host cells or purified (partially or substantially) polynucleotides in solution. An isolated polynucleotide also includes a polynucleotide molecule contained in cells that ordinarily contain the polynucleotide molecule, but the polynucleotide molecule is present extrachromosomally or at a chromosomal location that is different from its natural chromosomal location.

Isolated polynucleotide molecules include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

In some embodiments, the isolated polynucleotide molecule is selected from: a DNA, a cDNA, an RNA, an mRNA, and a naked plasmid (linear or circular).

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs.

In some aspects, the chimeric protein encoded by the polynucleotide molecule is a chimeric transcription factor, and the polynucleotide molecule further includes a target expression cassette including a gene of interest operably linked to a chimeric transcription factor-responsive (CTF-responsive) promoter. In some embodiments, the target expression cassette is present in the same heterologous construct as the chimeric protein. In some embodiments, the chimeric protein and the target expression cassette are present in separate heterologous constructs.

The term “expression cassette” refers to a polynucleotide generated recombinantly or synthetically, with a series of nucleic acid elements that permit transcription of a particular polynucleotide in a target cell. The expression cassette can be incorporated into a plasmid, chromosome, mitochondrial DNA, plastid DNA, virus, or nucleic acid fragment. Typically, the expression cassette portion of an expression vector includes, among other sequences, a nucleic acid sequence to be transcribed and a promoter. In some aspects, the present disclosure provides an expression cassette including a polynucleotide encoding a modified ER-LBD or a chimeric protein including a modified ER-LBD.

The isolated polynucleotide molecules and heterologous constructs including a modified ER-LBD as described herein are engineered polynucleotide molecules. An “engineered polynucleotide” is a polynucleotide that does not occur in nature. It should be understood, however, that while an engineered polynucleotide as a whole is not naturally-occurring, it may include nucleotide sequences that occur in nature. In some embodiments, an engineered polynucleotide comprises nucleotide sequences from different organisms (e.g., from different species). For example, in some embodiments, an engineered polynucleotide includes a murine nucleotide sequence, a bacterial nucleotide sequence, a human nucleotide sequence, and/or a viral nucleotide sequence. The term “engineered polynucleotide” includes recombinant nucleic acids and synthetic nucleic acids. A “recombinant polynucleotide” refers to a molecule that is constructed by joining nucleotide molecules and, in some embodiments, can replicate in a live cell. A “synthetic polynucleotide” refers to a molecule that is amplified or chemically, or by other means, synthesized. Synthetic polynucleotides include those that are chemically modified, or otherwise modified, but can base pair with naturally-occurring nucleotide molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant polynucleotides and synthetic polynucleotides also include those molecules that result from the replication of either of the foregoing. Engineered polynucleotides of the present disclosure may be encoded by a single molecule (e.g., included in the same plasmid or other vector) or by multiple different molecules (e.g., multiple different independently-replicating molecules).

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5’ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

In some embodiments, the polynucleotide molecules as described herein are included in a heterologous construct. The term “vector” or “expression vector” is synonymous with “heterologous construct” and refers to a polynucleotide molecule that is used to introduce and direct the expression of one or more genes that are operably associated with the construct in a target cell. The term includes the construct as a self-replicating nucleic acid structure as well as the vector incorporated into the genome of a host cell into which it has been introduced. A heterologous construct as described herein includes an expression cassette. In some aspects, provided herein is a heterologous construct comprising an expression cassette that comprises a promoter operably linked to a polynucleotide molecule that encodes a modified ER-LBD or a chimeric protein including a modified ER-LBD

As used herein, a “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

As used herein, an “inducible promoter” refers to a promoter characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., a chimeric transcription factor as described herein) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

As used herein, a promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA binding domain (DBD), an interferon-gamma-activated sequence (GAS) (Decker, T. et al. J Interferon Cytokine Res. 1997 Mar; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 3, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Non-limiting examples of components of inducible promoters include those shown in Table 4.

TABLE 3

| | | | | Response | |
|---|---|---|---|---|---|
| | Promoter and | Transcription | Inducer | to inducer | |
| System | operator | factor (TF) | molecule | TF | T |
| Transcriptional activator-responsive promotors | | | | | |
| AIR | PAIR | AlcR | Acetaldehyde | n.d. | A |
| | (OalcA-PhCMVmin) | | | | |
| ART | PART | ArgR-VP16 | 1-Arginine | B | A |
| | (OARG-PhCMVmin) | | | | |
| BIT | PBIT3 | BIT (BirA-VP16) | Biotin | B | A |
| | (OBirA3-PhCMVmin) | | | | |
| Cumate - | PCR5 | cTA (CymR-VP16) | Cumate | D | DA |
| activator | (OCuO6-PhCMVmin) | | | | |
| Cumate - | PCR5 | rcTA (rCymR-VP16) | Cumate | B | A |
| reverse activator | (OCuO6-PhCMVmin) | | | | |
| E-OFF | PETR | ET (E-VP16) | Erythromycin | D | DA |
| | (OETR-PhCMVmin) | | | | |
| NICE-OFF | PNIC | NT (HdnoR-VP16) | 6-Hydroxy-nicotine | D | DA |
| | (ONIC-PhCMVmin) | | | | |
| PEACE | PTtgR1 | TtgA1 (TtgR-VP16) | Phloretin | D | DA |
| | (OTtgR-PhCMVmin) | | | | |
| PIP-OFF | PPIR | PIT (PIP-VP16) | Pristinamycin I | D | DA |
| | (OPIR-Phsp70min) | | | | |
| QuoRex | PSCA (OscbR- | SCA (ScbR-VP16) | SCB1 | D | DA |
| | PhCMVmin)PSPA | | | | |
| | (OpapRI-PhCMVmin) | | | | |
| Redox | PROP | REDOX (REX-VP16) | NADH | D | DA |
| | (OROP-PhCMVmin) | | | | |
| TET-OFF | PhCMV*-1 | tTA (TetR-VP16) | Tetracycline | D | DA |
| | (OtetO7-PhCMVmin) | | | | |
| TET-ON | PhCMV*-1 | rtTA (rTetR-VP16) | Doxycycline | B | A |
| | (OtetO7-PhCMVmin) | | | | |
| TIGR | PCTA | CTA (RheA-VP16) | Heat | D | DA |
| | (OrheO-PhCMVmin) | | | | |
| TraR | O7x(tra box)- | p65-TraR | 3-Oxo-C8-HSL | B | A |
| | PhCMVmin | | | | |
| VAC-OFF | P1VanO2 | VanA1 (VanR-VP16) | Vanillic acid | D | DA |
| | (OVanO2-PhCMVmin) | | | | |
| Transcriptional repressor-responsive promoters | | | | | |
| Cumate - | PCuO | CymR | Cumate | D | DR |
| repressor | (PCMV5-OCuO) | | | | |
| E-ON | PETRON8 | E-KRAB | Erythromycin | D | DR |
| | (PSV40-OETR8) | | | | |
| NICE-ON | PNIC | NS (HdnoR-KRAB) | 6-Hydroxy-nicotine | D | DR |
| | (PSV40-ONIC8) | | | | |
| PIP-ON | PPIRON | PIT3 (PIP-KRAB) | Pristinamycin I | D | DR |
| | (PSV40-OPIR3) | | | | |
| Q-ON | PSCAON8 | SCS (ScbR-KRAB) | SCB1 | D | DR |
| | (PSV40-OscbR8) | | | | |
| TET-ON<comma> | OtetO-PHPRT | tTS-H4 (TetR-HDAC4) | Doxycycline | D | DR |
| repressor-based | | | | | |
| T-REX | PTetO | TetR | Tetracycline | D | DR |
| | (PhCMV-OtetO2) | | | | |
| UREX | PUREX8 | mUTS (KRAB-HucR) | Uric acid | D | DR |
| | (PSV40-OhucO8) | | | | |
| VAC-ON | PVanON8 | VanA4 (VanR-KRAB) | Vanillic acid | D | DR |
| | (PhCMV-OVanO8) | | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | Tetracycline | DD | DADR |
| ON(NOT IF gate) | PhCMVmin | | Erythromycin | | |
| TET-OFFPIP- | OtetO7-OPIR3- | tTAPIT3E-KRAB | Tetracycline | DDD | DADR |
| ONE-ON | OETR8-PhCMVmin | | Pristinamycin | | DR |
| | | | IErythromycin | | |

TABLE 4

| Name | DNA SEQUENCE | Source |
|---|---|---|
| minimal promoter; minP | AGAGGGTATATAATGGAAGCTCGAC | EU581860.1 |
| | TTCCAG (SEQ ID NO: 7) | (Promega) |
| NFKB response element | GGGAATTTCCGGGGACTTTCCGGGA | EU581860.1 |
| protein promoter; 5x | ATTTCCGGGGACTTTCCGGGAATTTC | (Promega) |
| NFKB-RE | C (SEQ ID NO: 8) | |
| CREB response element | CACCAGACAGTGACGTCAGCTGCCA | DQ904461.1 |
| protein promoter; 4x | GATCCCATGGCCGTCATACTGTGAC | (Promega) |
| CRE | GTCTTTCAGACACCCCATTGACGTCA | |
| | ATGGGAGAA (SEQ ID NO: 9) | |
| NFAT response element | GGAGGAAAAACTGTTTCATACAGAA | DQ904462.1 |
| protein promoter; 3x | GGCGTGGAGGAAAAACTGTTTCATA | (Promega) |
| NFAT binding sites | CAGAAGGCGTGGAGGAAAAACTGTT | |
| | TCATACAGAAGGCGT (SEQ ID NO: | |
| | 10) | |
| SRF response element | AGGATGTCCATATTAGGACATCTAG | FJ773212.1 |
| protein promoter; 5x | GATGTCCATATTAGGACATCTAGGA | (Promega) |
| SRF | TGTCCATATTAGGACATCTAGGATGT | |
| | CCATATTAGGACATCTAGGATGTCC | |
| | ATATTAGGACATCT (SEQ ID NO: 11) | |
| SRF response element | AGTATGTCCATATTAGGACATCTACC | FJ773213.1 |
| protein promoter 2; 5x | ATGTCCATATTAGGACATCTACTATG | (Promega) |
| SRF-RE | TCCATATTAGGACATCTTGTATGTCC | |
| | ATATTAGGACATCTAAAATGTCCAT | |
| | ATTAGGACATCT (SEQ ID NO: 12) | |
| AP1 response element | TGAGTCAGTGACTCAGTGAGTCAGT | JQ858516.1 |
| protein promoter; 6x | GACTCAGTGAGTCAGTGACTCAG | (Promega) |
| AP1-RE | (SEQ ID NO: 13) | |
| TCF-LEF response | AGATCAAAGGGTTTAAGATCAAAGG | JX099537.1 |
| element promoter; 8x | GCTTAAGATCAAAGGGTATAAGATC | (Promega) |
| TCF-LEF-RE | AAAGGGCCTAAGATCAAAGGGACTA | |
| | AGATCAAAGGGTTTAAGATCAAAGG | |
| | GCTTAAGATCAAAGGGCCTA (SEQ ID | |
| | NO: 14) | |
| SBEx4 | GTCTAGACGTCTAGACGTCTAGACG | Addgene Cat No: 16495 |
| | TCTAGAC (SEQ ID NO: 15) | |
| SMAD2/3-CAGACA | CAGACACAGACACAGACACAGACA | Jonk et al. (J Biol |
| x4 | (SEQ ID NO: 16) | Chem. 1998 Aug |
| | | 14;273(33):21145-52. |
| STAT3 binding site | Ggatccggtactcgagatctgcgatctaagtaagcttggca | Addgene Sequencing |
| | ttccggtactgttggtaaagccac (SEQ ID NO: 17) | Result #211335 |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter.

In some aspects, the present disclosure provides a heterologous construct comprising a promoter operatively linked to a polynucleotide molecule encoding a modified ER-LBD or chimeric protein as described herein.

In some embodiments, the promoter operatively linked to a polynucleotide molecule encoding a modified ER-LBD or chimeric protein is a constitutive promoter, an inducible promoter, or a synthetic promoter.

In some embodiments, the promoter operatively linked to a polynucleotide molecule encoding a modified ER-LBD or chimeric protein is a constitutive promoter. Examples of constitutive promoters are shown in Table 5. In some embodiments, the constitutive promoter is selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

TABLE 5

| Name | DNA SEQUENCE |
|---|---|
| CMV | GTTGACATTGATTATTGACTAGTTATTAATAGTAATCAATTACGGGGTCAT |
| | TAGTTCATAGCCCATATATGGAGTTCCGCGTTACATAACTTACGGTAAATG |
| | GCCCGCCTGGCTGACCGCCCAACGACCCCCGCCCATTGACGTCAATAATG |
| | ACGTATGTTCCCATAGTAACGCCAATAGGGACTTTCCATTGACGTCAATGG |
| | GTGGAGTATTTACGGTAAACTGCCCACTTGGCAGTACATCAAGTGTATCAT |
| | ATGCCAAGTACGCCCCCTATTGACGTCAATGACGGTAAATGGCCCGCCTG |
| | GCATTATGCCCAGTACATGACCTTATGGGACTTTCCTACTTGGCAGTACAT |
| | CTACGTATTAGTCATCGCTATTACCATGGTGATGCGGTTTTGGCAGTACAT |
| | CAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTC |
| | CAAAATGTCGTAACAACTCCGCCCCATTGACGCAAATGGGCGGTAGGCGT |
| | GTACGGTGGGAGGTCTATATAAGCAGAGCTC (SEQ ID NO: 18) |
| EF1a | GGCTCCGGTGCCCGTCAGTGGGCAGAGCGCACATCGCCCACAGTCCCCGA |
| | GAAGTTGGGGGGAGGGGTCGGCAATTGAACCGGTGCCTAGAGAAGGTGG |
| | CGCGGGGTAAACTGGGAAAGTGATGCCGTGTACTGGCTCCGCCTTTTTCCC |
| | GAGGGTGGGGGAGAACCGTATATAAGTGCAGTAGTCGCCGTGAACGTTCT |
| | TTTTCGCAACGGGTTTGCCGCCAGAACACAGGTAAGTGCCGTGTGTGGTTC |
| | CCGCGGGCCTGGCCTCTTTACGGGTTATGGCCCTTGCGTGCCTTGAATTAC |
| | TTCCACCTGGCTGCAGTACGTGATTCTTGATCCCGAGCTTCGGGTTGGAAG |
| | TGGGTGGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGAATCTGG |
| | TGGCACCTTCGCGCCTGTCTCGCTGCTTTCGATAAGTCTCTAGCCATTTAA |
| | AATTTTTGATGACCTGCTGCGACGCTTTTTTTCTGGCAAGATAGTCTTGTA |
| | AATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCC |
| | TGCGAGCGCGACCACCGAGAATCGGACGGGGGTAGTCTCAAGCTGGCCG |
| | GCCTGCTCTGGTGCCTGTCCTCGCGCCGCCGTGTATCGCCCCGCCCCGGGC |
| | GGCAAGGCTGGCCCGGTCGGCACCAGTTGCGTGAGCGGAAAGATGGCCG |
| | CTTCCCGGTCCTGCTGCAGGGAGCTCAAAATGGAGGACGCGGCGCTCGGG |
| | AGAGCGGGCGGGTGAGTCACCCACACAAAGGAAAAGGGCCTTTCCGTCCT |
| | CAGCCGTCGCTTCATGTGACTCCACGGAGTACCGGGCGCCGTCCAGGCAC |
| | CTCGATTAGTTCTCGAGCTTTTGGAGTACGTCGTCTTTAGGTTGGGGGGAG |
| | GGGTTTTATGCGATGGAGTTTCCCCACACTGAGTGGGTGGAGACTGAAGT |
| | TAGGCCAGCTTGGCACTTGATGTAATTCTCCTTGGAATTTGCCCTTTTTGA |
| | GTTTGGATCTTGGTTCATTCTCAAGCCTCAGACAGTGGTTCAAAGTTTTTTT |
| | CTTCCATTTCAGGTGTCGTGA (SEQ ID NO: 19) |
| EFS | GGATCTGCGATCGCTCCGGTGCCCGTCAGTGGGCAGAGCGCACATCGCCC |
| | ACAGTCCCCGAGAAGTTGGGGGGAGGGGTCGGCAATTGAACCGGTGCCTA |
| | GAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCC |
| | GTGAACGTTCTTTTTCGCAACGGGTTTGCCGCCAGAACACAGCTGAAGCTT |
| | CGAGGGGCTCGCATCTCTCCTTCACGCGCCCGCCGCCCTACCTGAGGCCGC |
| | CATCCACGCCGGTTGAGTCGCGTTCTGCCGCCTCCCGCCTGTGGTGCCTCC |
| | TGAACTGCGTCCGCCGTCTAGGTAAGTTTAAAGCTCAGGTCGAGACCGGG |
| | CCTTTGTCCGGCGCTCCCTTGGAGCCTACCTAGACTCAGCCGGCTCTCCAC |
| | GCTTTGCCTGACCCTGCTTGCTCAACTCTACGTCTTTGTTTCGTTTTCTGTT |
| | CTGCGCCGTTACAGATCCAAGCTGTGACCGGCGCCTAC (SEQ ID NO: 20) |
| MND | TTTATTTAGTCTCCAGAAAAAGGGGGGAATGAAAGACCCCACCTGTAGGT |
| | TTGGCAAGCTAGGATCAAGGTTAGGAACAGAGAGACAGCAGAATATGGG |
| | CCAAACAGGATATCTGTGGTAAGCAGTTCCTGCCCCGGCTCAGGGCCAAG |
| | AACAGTTGGAACAGCAGAATATGGGCCAAACAGGATATCTGTGGTAAGC |
| | AGTTCCTGCCCCGGCTCAGGGCCAAGAACAGATGGTCCCCAGATGCGGTC |
| | CCGCCCTCAGCAGTTTCTAGAGAACCATCAGATGTTTCCAGGGTGCCCCA |
| | AGGACCTGAAATGACCCTGTGCCTTATTTGAACTAACCAATCAGTTCGCTT |
| | CTCGCTTCTGTTCGCGCGCTTCTGCTCCCCGAGCTCAATAAAAGAGCCCA |
| | (SEQ ID NO: 21) |
| PGK | GGGGTTGGGGTTGCGCCTTTTCCAAGGCAGCCCTGGGTTTGCGCAGGGAC |
| | GCGGCTGCTCTGGGCGTGGTTCCGGGAAACGCAGCGGCGCCGACCCTGGG |
| | TCTCGCACATTCTTCACGTCCGTTCGCAGCGTCACCCGGATCTTCGCCGCT |
| | ACCCTTGTGGGCCCCCCGGCGACGCTTCCTGCTCCGCCCCTAAGTCGGGAA |
| | GGTTCCTTGCGGTTCGCGGCGTGCCGGACGTGACAAACGGAAGCCGCACG |
| | TCTCACTAGTACCCTCGCAGACGGACAGCGCCAGGGAGCAATGGCAGCGC |
| | GCCGACCGCGATGGGCTGTGGCCAATAGCGGCTGCTCAGCGGGGCGCGCC |
| | GAGAGCAGCGGCCGGGAAGGGGCGGTGCGGGAGGCGGGGTGTGGGGCGG |
| | TAGTGTGGGCCCTGTTCCTGCCCGCGCGGTGTTCCGCATTCTGCAAGCCTC |
| | CGGAGCGCACGTCGGCAGTCGGCTCCCTCGTTGACCGAATCACCGACCTC |
| | TCTCCCCAG (SEQ ID NO: 22) |
| SFFV | GTAACGCCATTTTGCAAGGCATGGAAAAATACCAAACCAAGAATAGAGA |
| | AGTTCAGATCAAGGGCGGGTACATGAAAATAGCTAACGTTGGGCCAAACA |
| | GGATATCTGCGGTGAGCAGTTTCGGCCCCGGCCCGGGGCCAAGAACAGAT |
| | GGTCACCGCAGTTTCGGCCCCGGCCCGAGGCCAAGAACAGATGGTCCCCA |
| | GATATGGCCCAACCCTCAGCAGTTTCTTAAGACCCATCAGATGTTTCCAGG |
| | CTCCCCCAAGGACCTGAAATGACCCTGCGCCTTATTTGAATTAACCAATCA |
| | GCCTGCTTCTCGCTTCTGTTCGCGCGCTTCTGCTTCCCGAGCTCTATAAAA |
| | GAGCTCACAACCCCTCACTCGGCGCGCCAGTCCTCCGACAGACTGAGTCG |
| | CCCGGG (SEQ ID NO: 23) |
| SV40 | CTGTGGAATGTGTGTCAGTTAGGGTGTGGAAAGTCCCCAGGCTCCCCAGC |
| | AGGCAGAAGTATGCAAAGCATGCATCTCAATTAGTCAGCAACCAGGTGTG |
| | GAAAGTCCCCAGGCTCCCCAGCAGGCAGAAGTATGCAAAGCATGCATCTC |
| | AATTAGTCAGCAACCATAGTCCCGCCCCTAACTCCGCCCATCCCGCCCCTA |
| | ACTCCGCCCAGTTCCGCCCATTCTCCGCCCCATGGCTGACTAATTTTTTTTA |
| | TTTATGCAGAGGCCGAGGCCGCCTCTGCCTCTGAGCTATTCCAGAAGTAGT |
| | GAGGAGGCTTTTTTGGAGGCCTAGGCTTTTGCAAAAAGCT (SEQ ID NO: 24) |
| UbC | GCGCCGGGTTTTGGCGCCTCCCGCGGGCGCCCCCCTCCTCACGGCGAGCG |
| | CTGCCACGTCAGACGAAGGGCGCAGGAGCGTTCCTGATCCTTCCGCCCGG |
| | ACGCTCAGGACAGCGGCCCGCTGCTCATAAGACTCGGCCTTAGAACCCCA |
| | GTATCAGCAGAAGGACATTTTAGGACGGGACTTGGGTGACTCTAGGGCAC |
| | TGGTTTTCTTTCCAGAGAGCGGAACAGGCGAGGAAAAGTAGTCCCTTCTC |
| | GGCGATTCTGCGGAGGGATCTCCGTGGGGCGGTGAACGCCGATGATTATA |
| | TAAGGACGCGCCGGGTGTGGCACAGCTAGTTCCGTCGCAGCCGGGATTTG |
| | GGTCGCGGTTCTTGTTTGTGGATCGCTGTGATCGTCACTTGGTGAGTTGCG |
| | GGCTGCTGGGCTGGCCGGGGCTTTCGTGGCCGCCGGGCCGCTCGGTGGGA |
| | CGGAAGCGTGTGGAGAGACCGCCAAGGGCTGTAGTCTGGGTCCGCGAGC |
| | AAGGTTGCCCTGAACTGGGGGTTGGGGGGAGCGCACAAAATGGCGGCTGT |
| | TCCCGAGTCTTGAATGGAAGACGCTTGTAAGGCGGGCTGTGAGGTCGTTG |
| | AAACAAGGTGGGGGGCATGGTGGGCGGCAAGAACCCAAGGTCTTGAGGC |
| | CTTCGCTAATGCGGGAAAGCTCTTATTCGGGTGAGATGGGCTGGGGCACC |
| | ATCTGGGGACCCTGACGTGAAGTTTGTCACTGACTGGAGAACTCGGGTTT |
| | GTCGTCTGGTTGCGGGGGCGGCAGTTATGCGGTGCCGTTGGGCAGTGCAC |
| | CCGTACCTTTGGGAGCGCGCGCCTCGTCGTGTCGTGACGTCACCCGTTCTG |
| | TTGGCTTATAATGCAGGGTGGGGCCACCTGCCGGTAGGTGTGCGGTAGGC |
| | TTTTCTCCGTCGCAGGACGCAGGGTTCGGGCCTAGGGTAGGCTCTCCTGAA |
| | TCGACAGGCGCCGGACCTCTGGTGAGGGGAGGGATAAGTGAGGCGTCAG |
| | TTTCTTTGGTCGGTTTTATGTACCTATCTTCTTAAGTAGCTGAAGCTCCGGT |
| | TTTGAACTATGCGCTCGGGGTTGGCGAGTGTGTTTTGTGAAGTTTTTTAGG |
| | CACCTTTTGAAATGTAATCATTTGGGTCAATATGTAATTTTCAGTGTTAGA |
| | CTAGTAAAGCTTCTGCAGGTCGACTCTAGAAAATTGTCCGCTAAATTCTGG |
| | CCGTTTTTGGCTTTTTTGTTAGAC (SEQ ID NO: 25) |
| hEF1aV1 | GGCTCCGGTGCCCGTCAGTGGGCAGAGCGCACATCGCCCACAGTCCCCGA |
| | GAAGTTGGGGGGAGGGGTCGGCAATTGAACCGGTGCCTAGAGAAGGTGG |
| | CGCGGGGTAAACTGGGAAAGTGATGTCGTGTACTGGCTCCGCCTTTTTCCC |
| | GAGGGTGGGGGAGAACCGTATATAAGTGCAGTAGTCGCCGTGAACGTTCT |
| | TTTTCGCAACGGGTTTGCCGCCAGAACACAGGTAAGTGCCGTGTGTGGTTC |
| | CCGCGGGCCTGGCCTCTTTACGGGTTATGGCCCTTGCGTGCCTTGAATTAC |
| | TTCCACCTGGCTGCAGTACGTGATTCTTGATCCCGAGCTTCGGGTTGGAAG |
| | TGGGTGGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGAATCTGG |
| | TGGCACCTTCGCGCCTGTCTCGCTGCTTTCGATAAGTCTCTAGCCATTTAA |
| | AATTTTTGATGACCTGCTGCGACGCTTTTTTTCTGGCAAGATAGTCTTGTA |
| | AATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCC |
| | TGCGAGCGCGGCCACCGAGAATCGGACGGGGGTAGTCTCAAGCTGGCCG |
| | GCCTGCTCTGGTGCCTGGTCTCGCGCCGCCGTGTATCGCCCCGCCCTGGGC |
| | GGCAAGGCTGGCCCGGTCGGCACCAGTTGCGTGAGCGGAAAGATGGCCG |
| | CTTCCCGGCCCTGCTGCAGGGAGCTCAAAATGGAGGACGCGGCGCTCGGG |
| | AGAGCGGGGGGTGAGTCACCCACACAAAGGAAAAGGGCCTTTCCGTCCT |
| | CAGCCGTCGCTTCATGTGACTCCACGGAGTACCGGGCGCCGTCCAGGCAC |
| | CTCGATTAGTTCTCGAGCTTTTGGAGTACGTCGTCTTTAGGTTGGGGGGAG |
| | GGGTTTTATGCGATGGAGTTTCCCCACACTGAGTGGGTGGAGACTGAAGT |
| | TAGGCCAGCTTGGCACTTGATGTAATTCTCCTTGGAATTTGCCCTTTTTGA |
| | GTTTGGATCTTGGTTCATTCTCAAGCCTCAGACAGTGGTTCAAAGTTTTTTT |
| | CTTCCATTTCAGGTGTCGTGA (SEQ ID NO: 26) |
| hCAGG | ACTAGTTATTAATAGTAATCAATTACGGGGTCATTAGTTCATAGCCCATAT |
| | ATGGAGTTCCGCGTTACATAACTTACGGTAAATGGCCCGCCTGGCTGACC |
| | GCCCAACGACCCCCGCCCATTGACGTCAATAATGACGTATGTTCCCATAGT |
| | AACGCCAATAGGGACTTTCCATTGACGTCAATGGGTGGAGTATTTACGGT |
| | AAACTGCCCACTTGGCAGTACATCAAGTGTATCATATGCCAAGTACGCCC |
| | CCTATTGACGTCAATGACGGTAAATGGCCCGCCTGGCATTATGCCCAGTA |
| | CATGACCTTATGGGACTTTCCTACTTGGCAGTACATCTACGTATTAGTCAT |
| | CGCTATTACCATGGTCGAGGTGAGCCCCACGTTCTGCTTCACTCTCCCCAT |
| | CTCCCCCCCCTCCCCACCCCCAATTTTGTATTTATTTATTTTTTAATTATTTT |
| | GTGCAGCGATGGGGGCGGGGGGGGGGGGGGGGCGCGCGCCAGGCGGGGC |
| | GGGGCGGGGCGAGGGGCGGGGCGGGGCGAGGCGGAGAGGTGCGGCGGC |
| | AGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGGAGTCG |
| | CTGCGACGCTGCCTTCGCCCCGTGCCCCGCTCCGCCGCCGCCTCGCGCCGC |
| | CCGCCCCGGCTCTGACTGACCGCGTTACTCCCACAGGTGAGCGGGCGGGA |
| | CGGCCCTTCTCCTCCGGGCTGTAATTAGCGCTTGGTTTAATGACGGCTTGT |
| | TTCTTTTCTGTGGCTGCGTGAAAGCCTTGAGGGGCTCCGGGAGGGCCCTTT |
| | GTGCGGGGGGAGCGGCTCGGGGGGTGCGTGCGTGTGTGTGTGCGTGGGGA |
| | GCGCCGCGTGCGGCTCCGCGCTGCCCGGCGGCTGTGAGCGCTGCGGGCGC |
| | GGCGCGGGGCTTTGTGCGCTCCGCAGTGTGCGCGAGGGGAGCGCGGCCGG |
| | GGGCGGTGCCCCGCGGTGCGGGGGGGGCTGCGAGGGGAACAAAGGCTGC |
| | GTGCGGGGTGTGTGCGTGGGGGGGTGAGCAGGGGGTGTGGGCGCGTCGG |
| | TCGGGCTGCAACCCCCCCTGCACCCCCCTCCCCGAGTTGCTGAGCACGGCC |
| | CGGCTTCGGGTGCGGGGCTCCGTACGGGGCGTGGCGCGGGGCTCGCCGTG |
| | CCGGGCGGGGGGTGGCGGCAGGTGGGGGTGCCGGGCGGGGCGGGGCCGC |
| | CTCGGGCCGGGGAGGGCTCGGGGGAGGGGCGCGGCGGCCCCCGGAGCGC |
| | CGGCGGCTGTCGAGGCGCGGCGAGCCGCAGCCATTGCCTTTTATGGTAAT |
| | CGTGCGAGAGGGCGCAGGGACTTCCTTTGTCCCAAATCTGTGCGGAGCCG |
| | AAATCTGGGAGGCGCCGCCGCACCCCCTCTAGCGGGCGCGGGGCGAAGC |
| | GGTGCGGCGCCGGCAGGAAGGAAATGGGCGGGGAGGGCCTTCGTGCGTC |
| | GCCGCGCCGCCGTCCCCTTCTCCCTCTCCAGCCTCGGGGCTGTCCGCGGGG |
| | GGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCT |
| | TTTTCCTACAGCTCCTGGGCAACGTGCTGGTTATTGTGCTGTCTCATCATTT |
| | TGGCAAAGAATTC (SEQ ID NO: 27) |
| hEF1aV2 | GGGCAGAGCGCACATCGCCCACAGTCCCCGAGAAGTTGGGGGGAGGGGT |
| | CGGCAATTGAACCGGTGCCTAGAGAAGGTGGCGCGGGGTAAACTGGGAA |
| | AGTGATGTCGTGTACTGGCTCCGCCTTTTTCCCGAGGGTGGGGGAGAACC |
| | GTATATAAGTGCAGTAGTCGCCGTGAACGTTCTTTTTCGCAACGGGTTTGC |
| | CGCCAGAACACAG (SEQ ID NO: 28) |
| hACTb | CCACTAGTTCCATGTCCTTATATGGACTCATCTTTGCCTATTGCGACACAC |
| | ACTCAATGAACACCTACTACGCGCTGCAAAGAGCCCCGCAGGCCTGAGGT |
| | GCCCCCACCTCACCACTCTTCCTATTTTTGTGTAAAAATCCAGCTTCTTGTC |
| | ACCACCTCCAAGGAGGGGGAGGAGGAGGAAGGCAGGTTCCTCTAGGCTG |
| | AGCCGAATGCCCCTCTGTGGTCCCACGCCACTGATCGCTGCATGCCCACCA |
| | CCTGGGTACACACAGTCTGTGATTCCCGGAGCAGAACGGACCCTGCCCAC |
| | CCGGTCTTGTGTGCTACTCAGTGGACAGACCCAAGGCAAGAAAGGGTGAC |
| | AAGGACAGGGTCTTCCCAGGCTGGCTTTGAGTTCCTAGCACCGCCCCGCC |
| | CCCAATCCTCTGTGGCACATGGAGTCTTGGTCCCCAGAGTCCCCCAGCGGC |
| | CTCCAGATGGTCTGGGAGGGCAGTTCAGCTGTGGCTGCGCATAGCAGACA |
| | TACAACGGACGGTGGGCCCAGACCCAGGCTGTGTAGACCCAGCCCCCCCG |
| | CCCCGCAGTGCCTAGGTCACCCACTAACGCCCCAGGCCTGGTCTTGGCTG |
| | GGCGTGACTGTTACCCTCAAAAGCAGGCAGCTCCAGGGTAAAAGGTGCCC |
| | TGCCCTGTAGAGCCCACCTTCCTTCCCAGGGCTGCGGCTGGGTAGGTTTGT |
| | AGCCTTCATCACGGGCCACCTCCAGCCACTGGACCGCTGGCCCCTGCCCTG |
| | TCCTGGGGAGTGTGGTCCTGCGACTTCTAAGTGGCCGCAAGCCACCTGAC |
| | TCCCCCAACACCACACTCTACCTCTCAAGCCCAGGTCTCTCCCTAGTGACC |
| | CACCCAGCACATTTAGCTAGCTGAGCCCCACAGCCAGAGGTCCTCAGGCC |
| | CTGCTTTCAGGGCAGTTGCTCTGAAGTCGGCAAGGGGGAGTGACTGCCTG |
| | GCCACTCCATGCCCTCCAAGAGCTCCTTCTGCAGGAGCGTACAGAACCCA |
| | GGGCCCTGGCACCCGTGCAGACCCTGGCCCACCCCACCTGGGCGCTCAGT |
| | GCCCAAGAGATGTCCACACCTAGGATGTCCCGCGGTGGGTGGGGGGCCCG |
| | AGAGACGGGCAGGCCGGGGGCAGGCCTGGCCATGCGGGGCCGAACCGGG |
| | CACTGCCCAGCGTGGGGCGCGGGGGCCACGGCGCGCGCCCCCAGCCCCCG |
| | GGCCCAGCACCCCAAGGCGGCCAACGCCAAAACTCTCCCTCCTCCTCTTCC |
| | TCAATCTCGCTCTCGCTCTTTTTTTTTTTCGCAAAAGGAGGGGAGAGGGGG |
| | TAAAAAAATGCTGCACTGTGCGGCGAAGCCGGTGAGTGAGCGGCGCGGG |
| | GCCAATCAGCGTGCGCCGTTCCGAAAGTTGCCTTTTATGGCTCGAGCGGCC |
| | GCGGCGGCGCCCTATAAAACCCAGCGGCGCGACGCGCCACCACCGCCGA |
| | GACCGCGTCCGCCCCGCGAGCACAGAGCCTCGCCTTTGCCGATCCGCCGC |
| | CCGTCCACACCCGCCGCCAGGTAAGCCCGGCCAGCCGACCGGGGCAGGCG |
| | GCTCACGGCCCGGCCGCAGGCGGCCGCGGCCCCTTCGCCCGTGCAGAGCC |
| | GCCGTCTGGGCCGCAGCGGGGGGCGCATGGGGGGGGAACCGGACCGCCG |
| | TGGGGGGCGCGGGAGAAGCCCCTGGGCCTCCGGAGATGGGGGACACCCC |
| | ACGCCAGTTCGGAGGCGCGAGGCCGCGCTCGGGAGGCGCGCTCCGGGGG |
| | TGCCGCTCTCGGGGCGGGGGCAACCGGCGGGGTCTTTGTCTGAGCCGGGC |
| | TCTTGCCAATGGGGATCGCAGGGTGGGCGCGGCGGAGCCCCCGCCAGGCC |
| | CGGTGGGGGCTGGGGCGCCATTGCGCGTGCGCGCTGGTCCTTTGGGCGCT |
| | AACTGCGTGCGCGCTGGGAATTGGCGCTAATTGCGCGTGCGCGCTGGGAC |
| | TCAAGGCGCTAACTGCGCGTGCGTTCTGGGGCCCGGGGTGCCGCGGCCTG |
| | GGCTGGGGCGAAGGCGGGCTCGGCCGGAAGGGGTGGGGTCGCCGCGGCT |
| | CCCGGGCGCTTGCGCGCACTTCCTGCCCGAGCCGCTGGCCGCCCGAGGGT |
| | GTGGCCGCTGCGTGCGCGCGCGCCGACCCGGCGCTGTTTGAACCGGGCGG |
| | AGGCGGGGCTGGCGCCCGGTTGGGAGGGGGTTGGGGCCTGGCTTCCTGCC |
| | GCGCGCCGCGGGGACGCCTCCGACCAGTGTTTGCCTTTTATGGTAATAAC |
| | GCGGCCGGCCCGGCTTCCTTTGTCCCCAATCTGGGCGCGCGCCGGCGCCCC |
| | CTGGCGGCCTAAGGACTCGGCGCGCCGGAAGTGGCCAGGGCGGGGGCGA |
| | CCTCGGCTCACAGCGCGCCCGGCTAT (SEQ ID NO: 29) |
| heIF4A1 | GTTGATTTCCTTCATCCCTGGCACACGTCCAGGCAGTGTCGAATCCATCTC |
| | TGCTACAGGGGAAAACAAATAACATTTGAGTCCAGTGGAGACCGGGAGC |
| | AGAAGTAAAGGGAAGTGATAACCCCCAGAGCCCGGAAGCCTCTGGAGGC |
| | TGAGACCTCGCCCCCCTTGCGTGATAGGGCCTACGGAGCCACATGACCAA |
| | GGCACTGTCGCCTCCGCACGTGTGAGAGTGCAGGGCCCCAAGATGGCTGC |
| | CAGGCCTCGAGGCCTGACTCTTCTATGTCACTTCCGTACCGGCGAGAAAG |
| | GCGGGCCCTCCAGCCAATGAGGCTGCGGGGGGGGCCTTCACCTTGATAGG |
| | CACTCGAGTTATCCAATGGTGCCTGCGGGCCGGAGCGACTAGGAACTAAC |
| | GTCATGCCGAGTTGCTGAGCGCCGGCAGGCGGGGCCGGGGCGGCCAAAC |
| | CAATGCGATGGCCGGGGCGGAGTCGGGCGCTCTATAAGTTGTCGATAGGC |
| | GGGCACTCCGCCCTAGTTTCTAAGGACCATG (SEQ ID NO: 30) |
| hGAPDH | AGTTCCCCAACTTTCCCGCCTCTCAGCCTTTGAAAGAAAGAAAGGGGAGG |
| | GGGCAGGCCGCGTGCAGTCGCGAGCGGTGCTGGGCTCCGGCTCCAATTCC |
| | CCATCTCAGTCGCTCCCAAAGTCCTTCTGTTTCATCCAAGCGTGTAAGGGT |
| | CCCCGTCCTTGACTCCCTAGTGTCCTGCTGCCCACAGTCCAGTCCTGGGAA |
| | CCAGCACCGATCACCTCCCATCGGGCCAATCTCAGTCCCTTCCCCCCTACG |
| | TCGGGGCCCACACGCTCGGTGCGTGCCCAGTTGAACCAGGCGGCTGCGGA |
| | AAAAAAAAAGCGGGGAGAAAGTAGGGCCCGGCTACTAGCGGTTTTACGG |
| | GCGCACGTAGCTCAGGCCTCAAGACCTTGGGCTGGGACTGGCTGAGCCTG |
| | GCGGGAGGCGGGGTCCGAGTCACCGCCTGCCGCCGCGCCCCCGGTTTCTA |
| | TAAATTGAGCCCGCAGCCTCCCGCTTCGCTCTCTGCTCCTCCTGTTCGACA |
| | GTCAGCCGCATCTTCTTTTGCGTCGCCAGGTGAAGACGGGCGGAGAGAAA |
| | CCCGGGAGGCTAGGGACGGCCTGAAGGCGGCAGGGGCGGGCGCAGGCCG |
| | GATGTGTTCGCGCCGCTGCGGGGTGGGCCCGGGCGGCCTCCGCATTGCAG |
| | GGGCGGGCGGAGGACGTGATGCGGCGCGGGCTGGGCATGGAGGCCTGGT |
| | GGGGGAGGGGAGGGGAGGCGTGGGTGTCGGCCGGGGCCACTAGGCGCTC |
| | ACTGTTCTCTCCCTCCGCGCAGCCGAGCCACATCGCTGAGACAC (SEQ ID |
| | NO: 31) |
| hGRP78 | AGTGCGGTTACCAGCGGAAATGCCTCGGGGTCAGAAGTCGCAGGAGAGA |
| | TAGACAGCTGCTGAACCAATGGGACCAGCGGATGGGGCGGATGTTATCTA |
| | CCATTGGTGAACGTTAGAAACGAATAGCAGCCAATGAATCAGCTGGGGGG |
| | GCGGAGCAGTGACGTTTATTGCGGAGGGGGCCGCTTCGAATCGGCGGCGG |
| | CCAGCTTGGTGGCCTGGGCCAATGAACGGCCTCCAACGAGCAGGGCCTTC |
| | ACCAATCGGCGGCCTCCACGACGGGGCTGGGGGAGGGTATATAAGCCGA |
| | GTAGGCGACGGTGAGGTCGACGCCGGCCAAGACAGCACAGACAGATTGA |
| | CCTATTGGGGTGTTTCGCGAGTGTGAGAGGGAAGCGCCGCGGCCTGTATT |
| | TCTAGACCTGCCCTTCGCCTGGTTCGTGGCGCCTTGTGACCCCGGGCCCCT |
| | GCCGCCTGCAAGTCGGAAATTGCGCTGTGCTCCTGTGCTACGGCCTGTGGC |
| | TGGACTGCCTGCTGCTGCCCAACTGGCTGGCAC (SEQ ID NO: 32) |
| hGRP94 | TAGTTTCATCACCACCGCCACCCCCCCGCCCCCCCGCCATCTGAAAGGGTT |
| | CTAGGGGATTTGCAACCTCTCTCGTGTGTTTCTTCTTTCCGAGAAGCGCCG |
| | CCACACGAGAAAGCTGGCCGCGAAAGTCGTGCTGGAATCACTTCCAACGA |
| | AACCCCAGGCATAGATGGGAAAGGGTGAAGAACACGTTGCCATGGCTAC |
| | CGTTTCCCCGGTCACGGAATAAACGCTCTCTAGGATCCGGAAGTAGTTCC |
| | GCCGCGACCTCTCTAAAAGGATGGATGTGTTCTCTGCTTACATTCATTGGA |
| | CGTTTTCCCTTAGAGGCCAAGGCCGCCCAGGCAAAGGGGCGGTCCCACGC |
| | GTGAGGGGCCCGCGGAGCCATTTGATTGGAGAAAAGCTGCAAACCCTGAC |
| | CAATCGGAAGGAGCCACGCTTCGGGCATCGGTCACCGCACCTGGACAGCT |
| | CCGATTGGTGGACTTCCGCCCCCCCTCACGAATCCTCATTGGGTGCCGTGG |
| | GTGCGTGGTGCGGCGCGATTGGTGGGTTCATGTTTCCCGTCCCCCGCCCGC |
| | GAGAAGTGGGGGTGAAAAGCGGCCCGACCTGCTTGGGGTGTAGTGGGCG |
| | GACCGCGCGGCTGGAGGTGTGAGGATCCGAACCCAGGGGTGGGGGGTGG |
| | AGGCGGCTCCTGCGATCGAAGGGGACTTGAGACTCACCGGCCGCACGTC |
| | (SEQ ID NO: 33) |
| hHSP70 | GGGCCGCCCACTCCCCCTTCCTCTCAGGGTCCCTGTCCCCTCCAGTGAATC |
| | CCAGAAGACTCTGGAGAGTTCTGAGCAGGGGGCGGCACTCTGGCCTCTGA |
| | TTGGTCCAAGGAAGGCTGGGGGGCAGGACGGGAGGCGAAAACCCTGGAA |
| | TATTCCCGACCTGGCAGCCTCATCGAGCTCGGTGATTGGCTCAGAAGGGA |
| | AAAGGCGGGTCTCCGTGACGACTTATAAAAGCCCAGGGGCAAGCGGTCCG |
| | GATAACGGCTAGCCTGAGGAGCTGCTGCGACAGTCCACTACCTTTTTCGA |
| | GAGTGACTCCCGTTGTCCCAAGGCTTCCCAGAGCGAACCTGTGCGGCTGC |
| | AGGCACCGGCGCGTCGAGTTTCCGGCGTCCGGAAGGACCGAGCTCTTCTC |
| | GCGGATCCAGTGTTCCGTTTCCAGCCCCCAATCTCAGAGCGGAGCCGACA |
| | GAGAGCAGGGAACCC (SEQ ID NO: 34) |
| hKINb | GCCCCACCCCCGTCCGCGTTACAACCGGGAGGCCCGCTGGGTCCTGCACC |
| | GTCACCCTCCTCCCTGTGACCGCCCACCTGATACCCAAACAACTTTCTCGC |
| | CCCTCCAGTCCCCAGCTCGCCGAGCGCTTGCGGGGAGCCACCCAGCCTCA |
| | GTTTCCCCAGCCCCGGGCGGGGCGAGGGGCGATGACGTCATGCCGGCGCG |
| | CGGCATTGTGGGGCGGGGCGAGGCGGGGCGCCGGGGGGAGCAACACTGA |
| | GACGCCATTTTCGGCGGCGGGAGCGGCGCAGGCGGCCGAGCGGGACTGG |
| | CTGGGTCGGCTGGGCTGCTGGTGCGAGGAGCCGCGGGGCTGTGCTCGGCG |
| | GCCAAGGGGACAGCGCGTGGGTGGCCGAGGATGCTGCGGGGCGGTAGCT |
| | CCGGCGCCCCTCGCTGGTGACTGCTGCGCCGTGCCTCACACAGCCGAGGC |
| | GGGCTCGGCGCACAGTCGCTGCTCCGCGCTCGCGCCCGGCGGCGCTCCAG |
| | GTGCTGACAGCGCGAGAGAGCGCGGCCTCAGGAGCAACAC (SEQ ID NO: |
| | 35) |
| hUBIb | TTCCAGAGCTTTCGAGGAAGGTTTCTTCAACTCAAATTCATCCGCCTGATA |
| | ATTTTCTTATATTTTCCTAAAGAAGGAAGAGAAGCGCATAGAGGAGAAGG |
| | GAAATAATTTTTTAGGAGCCTTTCTTACGGCTATGAGGAATTTGGGGCTCA |
| | GTTGAAAAGCCTAAACTGCCTCTCGGGAGGTTGGGCGCGGCGAACTACTT |
| | TCAGCGGCGCACGGAGACGGCGTCTACGTGAGGGGTGATAAGTGACGCA |
| | ACACTCGTTGCATAAATTTGCGCTCCGCCAGCCCGGAGCATTTAGGGGCG |
| | GTTGGCTTTGTTGGGTGAGCTTGTTTGTGTCCCTGTGGGTGGACGTGGTTG |
| | GTGATTGGCAGGATCCTGGTATCCGCTAACAGGTACTGGCCCACAGCCGT |
| | AAAGACCTGCGGGGGCGTGAGAGGGGGGAATGGGTGAGGTCAAGCTGGA |
| | GGCTTCTTGGGGTTGGGTGGGCCGCTGAGGGGAGGGGAGGGCGAGGTGA |
| | CGCGACACCCGGCCTTTCTGGGAGAGTGGGCCTTGTTGACCTAAGGGGGG |
| | CGAGGGCAGTTGGCACGCGCACGCGCCGACAGAAACTAACAGACATTAA |
| | CCAACAGCGATTCCGTCGCGTTTACTTGGGAGGAAGGCGGAAAAGAGGTA |
| | GTTTGTGTGGCTTCTGGAAACCCTAAATTTGGAATCCCAGTATGAGAATGG |
| | TGTCCCTTCTTGTGTTTCAATGGGATTTTTACTTCGCGAGTCTTGTGGGTTT |
| | GGTTTTGTTTTCAGTTTGCCTAACACCGTGCTTAGGTTTGAGGCAGATTGG |
| | AGTTCGGTCGGGGGAGTTTGAATATCCGGAACAGTTAGTGGGGAAAGCTG |
| | TGGACGCTTGGTAAGAGAGCGCTCTGGATTTTCCGCTGTTGACGTTGAAAC |
| | CTTGAATGACGAATTTCGTATTAAGTGACTTAGCCTTGTAAAATTGAGGGG |
| | AGGCTTGCGGAATATTAACGTATTTAAGGCATTTTGAAGGAATAGTTGCT |
| | AATTTTGAAGAATATTAGGTGTAAAAGCAAGAAATACAATGATCCTGAGG |
| | TGACACGCTTATGTTTTACTTTTAAACTAGGTCACC (SEQ ID NO: 36) |

### Expression Systems Further Including a Target Expression Cassette

In some aspects, the chimeric protein is a chimeric transcription factor and the present disclosure further provides a target expression cassette including a chimeric transcription factor-responsive (CTF-responsive) promoter.

“Target expression cassette” refers to an expression cassette including a gene with chimeric transcription factor-controllable expression. The expression is controlled by the chimeric transcription factor based on the presence of a non-endogenous ligand (e.g., 4-OHT or endoxifen).

In some aspects, the present disclosure provides polynucleotide molecules encoding a gene of interest operably linked to a chimeric transcription factor-responsive promoter (CTF-responsive promoter). CTF-responsive promoters are synthetic, inducible promoters that are responsive to a chimeric transcription factor including a modified ER-LBD, and are inducible in response to a non-endogenous ligand such as 4-OHT.

In some embodiments, the CTF-responsive promoter comprises a core promoter sequence and a binding domain that binds to a chimeric transcription factor as described herein.

The binding domain may include one or more zinc finger binding sites. The binding domain can comprise 1, 2, 3, 4, 5, 6 7, 8, 9, 10, or more zinc finger binding sites. In some embodiments, the binding domain comprises one zinc finger binding site. In some embodiments, the binding domain comprises two zinc finger binding sites. In some embodiments, the binding domain comprises three zinc finger binding sites. In some embodiments, the binding domain comprises four zinc finger binding sites. An exemplary binding domain comprising zinc finger binding sites is shown in the sequence:

(SEQ ID NO: 37)
cgggtttcgtaacaatcgcatgaggattcgcaacgccttcGGCGTAGCCGATGTCGCGctcccgtctcagtaaaggtc
GGCGTAGCCGATGTCGCGcaatcggactgccttcgtacGGCGTAGCCGATGTCGCGcgtatcagtcg
cctcggaacGGCGTAGCCGATGTCGCGcattcgtaagaggctcactctcccttacacggagtggataACTAGTT
CTAGAGGGTATATAATGGGGGCCA.

The core promoter sequence may include a minimal promoter. Examples of minimal promoters include minP, minCMV, YB_TATA, and minTK

In some aspects, the chimeric protein including the modified ER-LBD is a chimeric transcription factor, and the heterologous construct further includes a target expression cassette including a chimeric-transcription factor responsive promoter. In some aspects, provided herein is a first heterologous construct comprising an expression cassette that comprises a polynucleotide molecule that encodes chimeric transcription factor including a modified ER-LBD, and a second heterologous construct comprising a target expression cassette including a chimeric transcription factor-responsive (CTF-responsive) promoter.

In some embodiments, engineered polynucleotides or constructs of the present disclosure are configured to produce multiple polypeptides. For example, polynucleotides may be configured to produce 2 different polypeptides. The polynucleotide molecule may be configured to produce a polypeptide including a chimeric protein as described herein and a polypeptide of interest, which expressed under control of a promoter that is responsive to the chimeric protein.

In some embodiments, an ER-LBD or chimeric protein as described herein and a gene of interest that can be transcriptionally modulated by the ER-LBD or chimeric protein may be encoded by the same polynucleotide molecule or heterologous construct.

In some embodiments, engineered nucleic acids can be multicistronic, i.e., more than one separate polypeptide (e.g., multiple exogenous polynucleotides) can be produced from a single transcript. Engineered nucleic acids can be multicistronic through the use of various linkers, e.g., a polynucleotide sequence encoding a first exogenous polynucleotide can be linked to a nucleotide sequence encoding a second exogenous polynucleotide, such as in a first gene:linker:second gene 5′ to 3′ orientation. A linker polynucleotide sequence can encode one or more 2A ribosome skipping elements, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third polypeptide molecule, each separated by linkers such that separate polypeptides encoded by the first, second, and third polypeptides are produced).

“Linkers,” as used herein, can refer to polypeptides that link a first polypeptide sequence and a second polypeptide sequence or the multicistronic linkers described above.

Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element. Examples of WPRE sequences include SEQ ID NO: 38 and SEQ ID NO: 39.

Engineered Cells

Also provided herein are cells, and methods of producing cells, that comprise one or more polynucleotide molecules or constructs of the present disclosure. These cells are referred to herein as “engineered cells.” These cells, which typically contain one or more engineered nucleic acids, do not occur in nature. In some embodiments, the cells are isolated cells that recombinantly express the one or more engineered polynucleotides. In some embodiments, the engineered polynucleotides are expressed from one or more vectors or a selected locus from the genome of the cell. In some embodiments, the cells are engineered to include a polynucleotide comprising a promoter operably linked to a nucleotide sequence.

An engineered cell of the present disclosure can comprise an engineered polynucleotide integrated into the cell's genome. An engineered cell can comprise an engineered polynucleotide capable of expression without integrating into the cell's genome, for example, engineered with a transient expression system such as a plasmid or mRNA.

Engineered Cell Types

An engineered cell of the present disclosure can be a human cell. An engineered cell can be a human primary cell. An engineered primary cell can be any somatic cell. An engineered primary cell can be any stem cell. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered cell of the present disclosure is selected from: a T cell (e.g., a CD8+ T cell, a CD4+ T cell, or a gamma-delta T cell), a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage (e.g., an M1 macrophage or an M2 macrophage), a monocyte, a dendritic cell, an erythrocyte, a platelet cell, a neuron, an oligodendrocyte, an astrocyte, a placode-derived cell, a Schwann cell, a cardiomyocyte, an endothelial cell, a nodal cell, a microglial cell, a hepatocyte, a cholangiocyte, a beta cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, an engineered cell of the present disclosure is a T cell (e.g., a CD8+ T cell, a CD4+ T cell, or a gamma-delta T cell). In some embodiments, an engineered of the present disclosure is a cytotoxic T lymphocyte (CTL). In some embodiments, an engineered cell of the present disclosure is a regulatory T cell. In some embodiments, an engineered cell of the present disclosure is a Natural Killer T (NKT) cell. In some embodiments, an engineered cell of the present disclosure is a Natural Killer (NK) cell. In some embodiments, an engineered cell of the present disclosure is a B cell. In some embodiments, an engineered cell of the present disclosure is a tumor-infiltrating lymphocyte (TIL). In some embodiments, an engineered cell of the present disclosure is an innate lymphoid cell. In some embodiments, an engineered cell of the present disclosure is a mast cell. In some embodiments, an engineered cell of the present disclosure is an eosinophil. In some embodiments, an engineered cell of the present disclosure is a basophil. In some embodiments, an engineered cell of the present disclosure is a neutrophil. In some embodiments, an engineered cell of the present disclosure is a myeloid cell. In some embodiments, an engineered cell of the present disclosure is a macrophage e.g., an M1 macrophage or an M2 macrophage). In some embodiments, an engineered cell of the present disclosure is a monocyte. In some embodiments, an engineered or isolated cell of the present disclosure is a dendritic cell. In some embodiments, an engineered cell of the present disclosure is an erythrocyte. In some embodiments, an engineered cell of the present disclosure is a platelet cell. In some embodiments, a cell of the present disclosure is a neuron. In some embodiments, a cell of the present disclosure is an oligodendrocyte. In some embodiments, a cell of the present disclosure is an astrocyte. In some embodiments, a cell of the present disclosure is a placode-derived cell. In some embodiments, an engineered cell of the present disclosure is a Schwann cell. In some embodiments, an engineered cell of the present disclosure is a cardiomyocyte. In some embodiments, an engineered cell of the present disclosure is an endothelial cell. In some embodiments, an engineered cell of the present disclosure is a nodal cell. In some embodiments, an engineered cell of the present disclosure is a microglial cell. In some embodiments, an engineered cell of the present disclosure is a hepatocyte. In some embodiments, an engineered cell of the present disclosure is a cholangiocyte. In some embodiments, an engineered cell of the present disclosure is a beta cell. In some embodiments, an engineered cell of the present disclosure is a human embryonic stem cell (ESC). In some embodiments, an engineered cell of the present disclosure is an ESC-derived cell. In some embodiments, an engineered cell of the present disclosure is a pluripotent stem cell. In some embodiments, an engineered cell of the present disclosure is a mesenchymal stromal cell (MSC). In some embodiments, an engineered cell of the present disclosure is an induced pluripotent stem cell (iPSC). In some embodiments, an engineered cell of the present disclosure is an iPSC-derived cell. In some embodiments, an engineered cell is autologous. In some embodiments, an engineered cell is allogeneic. In some embodiments, an engineered cell of the present disclosure is a CD34+ cell, a CD3+ cell, a CD8+ cell, a CD16+ cell, and/or a CD4+ cell.

In some embodiments, a cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Methods of Engineering Cells

Also provided herein are compositions and methods for engineering cells with any polynucleotide molecule or construct as described herein.

In general, cells are engineered through introduction (i.e., delivery) of one or more polynucleotides of the present disclosure. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally derived nucleic acid. Such engineered virally derived nucleic acids can also be referred to as recombinant viruses or engineered viruses.

A viral vector-based delivery platform can encode more than one engineered nucleic acid, gene, or transgene within the same nucleic acid. For example, an engineered virally derived nucleic acid, e.g., a recombinant virus or an engineered virus, can encode one or more transgenes, including, but not limited to, any of the engineered nucleic acids described herein. The one or more transgenes can be configured to express polypeptides described herein (e.g., a modified ER-LBD). A viral vector-based delivery platform can encode one or more genes in addition to the transgene encoding the modified ER-LBD, such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the modified ER-LBD. One viral vector can deliver more than one engineered polynucleotides, such as one vector that delivers an engineered polynucleotide configured to produce a modified ER-LBD and an engineered polynucleotide configured produce a gene of interest. More than one viral vector can deliver more than one engineered nucleic acids, such as a first vector that delivers an engineered polynucleotide configured to produce a modified ER-LBD and a second vector that delivers an additional engineered polynucleotide. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, or used in vivo and ex vivo gene therapy procedures, e.g., for in vivo delivery. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e., delivery) into a host cell, infected cells (i.e., an engineered cell) can express, and in some case secrete, the modified ER-LBD (or chimeric polypeptide including the modified ER-LBD). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid described herein). The transgenes can be configured to express a modified ER-LBD (or chimeric polypeptide including the modified ER-LBD) and optionally a gene of interest.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., a transgene encoding the modified ER-LBD) into the target cell to provide permanent transgene expression. Retroviral-based delivery systems include, but are not limited to, those based upon murine leukemia, virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of a modified ER-LBD (or chimeric polypeptide including the modified ER-LBD), or used in vivo and ex vivo gene therapy procedures, e.g., for in vivo delivery of the modified ER-LBD (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Lipid Structure Delivery Systems

Engineered nucleic acids of the present disclosure (e.g., a nucleic acid encoding a modified ER-LBD or chimeric protein described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Feigner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

Nanoparticle Delivery

Nanomaterials can be used to deliver engineered nucleic acids (e.g., a nucleic acid encoding a modified ER-LBD or chimeric protein described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

Genomic Editing Systems

Genomic editing systems can be used to engineer a host genome to encode an engineered nucleic acid, such as a nucleic acid encoding a modified ER-LBD of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an isolated polynucleotide or heterologous construct of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids described herein).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The cells described herein can be engineered using non-viral methods, e.g., the nuclease and/or CRISPR mediated gene editing systems described herein can be delivered to a cell using non-viral methods. The cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

Other Engineering Delivery Systems

Various additional means to introduce engineered nucleic acids (e.g., an isolated polynucleotide encoding a modified ER-LBD or chimeric protein described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., an isolated polynucleotide encoding a modified ER-LBD or chimeric protein described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

Methods of Use

Methods of using a modified ER-LBD, chimeric protein, or cell as described herein are also encompassed by this disclosure.

In some aspects, the methods include modulating transcription of a gene of interest. Methods of modulating transcription may include: transforming a cell with (i) a heterologous construct encoding a chimeric transcription factor that includes a modified ER-LBD, and (ii) a target expression cassette comprising a chimeric transcription factor-responsive (CTF-responsive) promoter operably linked to a gene of interest; culturing the transformed cell under conditions suitable for expression of the chimeric protein; and inducing the chimeric protein to modulate transcription of the gene of interest by contacting the transformed cell with a non-endogenous ligand.

In some embodiments, the method of modulating transcription is a method of activating transcription. Activating transcription may be achieved using a chimeric protein

In some embodiments, the methods include activating transcription. Activating transcription may be achieved, for example, using a chimeric protein that includes a modified ER-LBD, an DNA binding domain, and a transcriptional activation domain.

In some embodiments, the methods include repressing transcription. Repressing transcription may be achieved, for example, using a chimeric protein that includes a modified ER-LBD, an DNA binding domain, and a transcriptional repressor domain.

In some aspects, the methods include modulating localization of a chimeric protein. Methods of modulating localization may include transforming a cell with a heterologous construct encoding a chimeric protein including a modified ER-LBD domain and a polypeptide of interest; culturing the transformed call under conditions suitable for expression of the chimeric protein; and inducing nuclear localization of the chimeric protein by contacting the transformed cell with a non-endogenous ligand. In some embodiments, modulating localization comprises inducing nuclear localization.

In some embodiments, the non-endogenous ligand is administered at a concentration at which the non-endogenous ligand is substantially inactive on wild-type estrogen receptor alpha.

In Vivo Methods

The methods provided herein also include modifying localization or modulating transcription in vivo, e.g., by delivering a non-endogenous ligand to a cell expressing the modified ER-LBD or chimeric protein in vivo.

In some embodiments, the transformed cell is in a human or animal, and contacting the transformed cell with the non-endogenous ligand comprises administering a pharmacological dose of the ligand to the human or animal. In some embodiments, the non-endogenous ligand administered to the subject comprises tamoxifen. Upon oral administration of tamoxifen, the drug is converted in the liver to an active tamoxifen metabolite. In some embodiments, the active tamoxifen metabolite is selected from 4-hydroxytamoxifen (“4-OHT”), N-desmethyltamoxifen, tamoxifen-N-oxide, and endoxifen. In some embodiments, the non-endogenous ligand is administered to the subject at a concentration of between about 1 mg per day and about 100 mg per day. In particular embodiments, the non-endogenous ligand is administered to the subject at a concentration of about 40 mg per day.

In some aspects, methods provided herein also include modulating transcription of a gene of interest in vivo, e.g., by delivering to a subject (i) a cell transformed with a chimeric transcription factor as described herein and (ii) a non-endogenous ligand. In some embodiments, the transformed cell comprises a target gene expression cassette comprising a chimeric-transcription factor responsive promoter operably linked the gene of interest.

In some embodiments, the subject a human or animal, and contacting the transformed cell with the non-endogenous ligand comprises administering a pharmacological dose of the non-endogenous ligand to the human or animal.

In some aspects, methods provided herein also include delivering a composition in vivo capable of producing the engineered cells described herein, e.g., capable of delivering a polynucleotide molecules described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the modified ER-LBD, chimeric proteins, or chimeric transcription factors (and in some embodiments, a gene regulated by the chimeric transcription factor) as described herein. Compositions capable of in vivo production of the modified ER-LBD, chimeric protein, or chimeric transcription factor (and in some embodiments, a gene regulated by the chimeric transcription factor) include, but are not limited to, any of the engineered nucleic acids described herein. Compositions capable of in vivo production of inducible transcription factors (and in some embodiments, a gene regulated by the inducible transcription factor) can be a naked mRNA or a naked plasmid.

Pharmaceutical Compositions

The modified ER-LBD, chimeric proteins, and cells of the present disclosure can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Whether it is a cell, polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### ADDITIONAL EMBODIMENTS

The paragraphs below provide additional enumerated embodiments.
1. A modified estrogen receptor ligand binding domain (ER-LBD) comprising an amino acid sequence corresponding to amino acids 282-595 of SEQ ID NO: 1, wherein the modified ER-LBD comprises a G400V amino acid substitution, an M543A amino acid substitution, and an L544A amino acid substitution, and one or more additional amino acid substitutions, wherein the one or more additional amino acid substitutions are within a region of SEQ ID NO: 1 selected from the group consisting of: positions 343-354, positions 380-392, positions 404-463, and positions 517-540, and position 547, and wherein the modified ER-LBD has greater sensitivity to a non-endogenous ligand as compared to an ER-LBD comprising the amino acid sequence of SEQ ID NO: 2.
2. A modified estrogen receptor ligand binding domain (ER-LBD) comprising an amino acid sequence corresponding to amino acids 282-595 of SEQ ID NO: 1, wherein the modified ER-LBD comprises a G400V amino acid substitution, an M543A amino acid substitution, and an L544A amino acid substitution and one or more additional amino acid substitutions, wherein the one or more additional amino acid substitutions are within a region of SEQ ID NO: 1 selected from the group consisting of: positions 343-354, positions 380-392, positions 404-463, and positions 517-540, and position 547 and wherein the modified ER-LBD has greater sensitivity to a non-endogenous ligand as compared to an endogenous ligand as a result of the one or more additional amino acid substitutions.
3. A modified estrogen receptor ligand binding domain (ER-LBD) comprising an amino acid sequence corresponding to amino acids 282-595 of SEQ ID NO: 1, wherein the modified ER-LBD comprises a G400V amino acid substitution, an M543A amino acid substitution, and an L544A amino acid substitution and one or more additional amino acid substitutions, wherein the one or more additional amino acid substitutions are within a region of SEQ ID NO: 1 selected from the group consisting of: positions 343-354, positions 380-392, positions 404-463, and positions 517-540, and position 547, and wherein the modified ER-LBD has greater selectivity to a non-endogenous ligand as compared to an ER-LBD comprising the amino acid sequence of SEQ ID NO: 2.
4. The modified ER-LBD of any one of paragraphs 1 to 3, wherein the one or more additional amino acid substitutions are at one or more positions of SEQ ID NO: 1 selected from the group consisting of: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.
5. The modified ER-LBD of paragraph 4, wherein the one or more positions comprise position 343 of SEQ ID NO: 1.
6. The modified ER-LBD of paragraph 5, wherein the amino acid substitution at position 343 of SEQ ID NO: 1 is selected from the group consisting of: M343F, M343I, M343L, and M343V.
7 The modified ER-LBD of paragraph 4, wherein the one or more positions comprise position 344 of SEQ ID NO: 1.
8. The modified ER-LBD of paragraph 7, wherein the amino acid substitution at position 344 of SEQ ID NO: 1 is G344M.
9. The modified ER-LBD of paragraph 4, wherein the one or more positions comprise position 345 of SEQ ID NO: 1.
10. The modified ER-LBD of paragraph 9, wherein the amino acid substitution at position 345 of SEQ ID NO: 1 is L345S.
11. The modified ER-LBD of paragraph 4, wherein the one or more positions comprise position 346 of SEQ ID NO: 1.
12. The modified ER-LBD of paragraph 11, wherein the amino acid substitution at position 346 of SEQ ID NO: 1 is selected from the group consisting of: L346I, L346M, L346F, and L346V.
13. The modified ER-LBD of paragraph 4, wherein the one or more positions comprise position 347 of SEQ ID NO: 1.
14. The modified ER-LBD of paragraph 13, wherein the amino acid substitution at position 347 of SEQ ID NO: 1 is selected from the group consisting of: T347D, T347E, T347F, T347I, T347K, T347L, T347M, T347N, T347Q, T347R, T347S, and T347V.
15. The modified ER-LBD of paragraph 4, wherein the one or more positions comprise position 348 of SEQ ID NO: 1.
16. The modified ER-LBD of paragraph 15, wherein the amino acid substitution at position 348 of SEQ ID NO: 1 is N348K.
17. The modified ER-LBD of paragraph 4, wherein the one or more positions comprise position 349 of SEQ ID NO: 1.
18. The modified ER-LBD of paragraph 17, wherein the amino acid substitution at position 349 of SEQ ID NO: 1 is selected from the group consisting of: L349I, L349M, L349F, and L349V.
19. The modified ER-LBD of paragraph 4, wherein the one or more positions comprise position 350 of SEQ ID NO: 1.
20. The modified ER-LBD of paragraph 19, wherein the amino acid substitution at position 350 of SEQ ID NO: 1 is selected from the group consisting of: A350F, A350I, A350L, A350M and A350V.
21. The modified ER-LBD of paragraph 4, wherein the one or more positions comprise position 351 of SEQ ID NO: 1.
22. The modified ER-LBD of paragraph 21, wherein the amino acid substitution at position 351 of SEQ ID NO: 1 is selected from the group consisting of: D351E, D351F, D351I, D351L, D351M, D351N, D351Q, and D351V.
23. The modified ER-LBD of paragraph 4, wherein the one or more positions comprise position 352 of SEQ ID NO: 1.
24. The modified ER-LBD of paragraph 23, wherein the amino acid substitution at position 352 of SEQ ID NO: 1 is R352K.
25. The modified ER-LBD of paragraph 4, wherein the one or more positions comprise position 354 of SEQ ID NO: 1.
26. The modified ER-LBD of paragraph 25, wherein the amino acid substitution at position 354 of SEQ ID NO: 1 is selected from the group consisting of: L354I, L354M, L354F, and L354V.
27. The modified ER-LBD of paragraph 4, wherein the one or more positions comprise position 380 of SEQ ID NO: 1.
28. The modified ER-LBD of paragraph 27, wherein the amino acid substitution at position 380 of SEQ ID NO: 1 is E380Q.
29. The modified ER-LBD of paragraph 4, wherein the one or more positions comprise position 384 of SEQ ID NO: 1.
30. The modified ER-LBD of paragraph 29, wherein the amino acid substitution at position 384 of SEQ ID NO: 1 is selected from the group consisting of: L384I, L384M, L384F, and L384V.
31. The modified ER-LBD of paragraph 4, wherein the one or more positions comprise position 386 of SEQ ID NO: 1.
32. The modified ER-LBD of paragraph 31, wherein the amino acid substitution at position 386 of SEQ ID NO: 1 is I386V.
33. The modified ER-LBD of paragraph 4, wherein the one or more positions comprise position 387 of SEQ ID NO: 1.
34. The modified ER-LBD of paragraph 33, wherein the amino acid substitution at position 387 of SEQ ID NO: 1 is selected from the group consisting of: L387I, L387M, L387F, and L387V.
35. The modified ER-LBD of paragraph 4, wherein the one or more positions comprise position 388 of SEQ ID NO: 1.
36. The modified ER-LBD of paragraph 35, wherein the amino acid substitution at position 388 of SEQ ID NO: 1 is selected from the group consisting of: M388I, M388L, and M388F.
37. The modified ER-LBD of paragraph 4, wherein the one or more positions comprise position 389 of SEQ ID NO: 1.
38. The modified ER-LBD of paragraph 37, wherein the amino acid substitution at position 389 of SEQ ID NO: 1 is I389M.
39. The modified ER-LBD of paragraph 4, wherein the one or more positions comprise position 391 of SEQ ID NO: 1.
40. The modified ER-LBD of paragraph 39, wherein the amino acid substitution at position 391 of SEQ ID NO: 1 is selected from the group consisting of: L391I, L391M, L391F, and L391V.
41. The modified ER-LBD of paragraph 4, wherein the one or more positions comprise position 392 of SEQ ID NO: 1.
42. The modified ER-LBD of paragraph 41, wherein the amino acid substitution at position 392 of SEQ ID NO: 1 is V392M.
43. The modified ER-LBD of paragraph 4, wherein the one or more positions comprise position 404 of SEQ ID NO: 1.
44. The modified ER-LBD of paragraph 43, wherein the amino acid substitution at position 404 of SEQ ID NO: 1 is selected from the group consisting of: F404I, F404L, F404M, and F404V.
45. The modified ER-LBD of paragraph 4, wherein the one or more positions comprise position 407 of SEQ ID NO: 1.
46. The modified ER-LBD of paragraph 45, wherein the amino acid substitution at position 407 of SEQ ID NO: 1 is N407D.
47. The modified ER-LBD of paragraph 4, wherein the one or more positions comprise position 409 of SEQ ID NO: 1.
48. The modified ER-LBD of paragraph 47, wherein the amino acid substitution at position 409 of SEQ ID NO: 1 is L409V.
49. The modified ER-LBD of paragraph 4, wherein the one or more positions comprise position 413 of SEQ ID NO: 1.
50. The modified ER-LBD of paragraph 49, wherein the amino acid substitution at position 413 of SEQ ID NO: 1 is N413D.
51. The modified ER-LBD of paragraph 4, wherein the one or more positions comprise position 414 of SEQ ID NO: 1.
52. The modified ER-LBD of paragraph 51, wherein the amino acid substitution at position 414 of SEQ ID NO: 1 is Q414E.
53. The modified ER-LBD of paragraph 4, wherein the one or more positions comprise position 417 of SEQ ID NO: 1.
54. The modified ER-LBD of paragraph 53, wherein the amino acid substitution at position 417 of SEQ ID NO: 1 is C417S.
55. The modified ER-LBD of paragraph 4, wherein the one or more positions comprise position 418 of SEQ ID NO: 1.
56. The modified ER-LBD of paragraph 55, wherein the amino acid substitution at position 418 of SEQ ID NO: 1 is selected from the group consisting of: V418I, V418L, V418M, and V418F.
57. The modified ER-LBD of paragraph 4, wherein the one or more positions comprise position 420 of SEQ ID NO: 1.
58. The modified ER-LBD of paragraph 57, wherein the amino acid substitution at position 420 of SEQ ID NO: 1 is selected from the group consisting of: G420I, G420M, G420F, and G420V.
59. The modified ER-LBD of paragraph 4, wherein the one or more positions comprise position 421 of SEQ ID NO: 1.
60. The modified ER-LBD of paragraph 59, wherein the amino acid substitution at position 421 of SEQ ID NO: 1 is selected from the group consisting of: M421I, M421L, M421F, and M421V.
61. The modified ER-LBD of paragraph 4, wherein the one or more positions comprise position 422 of SEQ ID NO: 1.
62. The modified ER-LBD of paragraph 61, wherein the amino acid substitution at position 422 of SEQ ID NO: 1 is V422I.
63. The modified ER-LBD of paragraph 4, wherein the one or more positions comprise position 424 of SEQ ID NO: 1.
64. The modified ER-LBD of paragraph 63, wherein the amino acid substitution at position 424 of SEQ ID NO: 1 is selected from the group consisting of: I424L, I424M, I424F, and I424V.
65. The modified ER-LBD of paragraph 4, wherein the one or more positions comprise position 428 of SEQ ID NO: 1.
66. The modified ER-LBD of paragraph 65, wherein the amino acid substitution at position 428 of SEQ ID NO: 1 is selected from the group consisting of: L428I, L428M, L428F, and L428V.
67. The modified ER-LBD of paragraph 4, wherein the one or more positions comprise position 463 of SEQ ID NO: 1.
68. The modified ER-LBD of paragraph 67, wherein the amino acid substitution at position 463 of SEQ ID NO: 1 is S463P.
69. The modified ER-LBD of paragraph 4, wherein the one or more positions comprise position 517 of SEQ ID NO: 1.
70. The modified ER-LBD of paragraph 69, wherein the amino acid substitution at position 517 of SEQ ID NO: 1 is M517A.
71. The modified ER-LBD of paragraph 4, wherein the one or more positions comprise position 521 of SEQ ID NO: 1.
72. The modified ER-LBD of paragraph 71, wherein the amino acid substitution at position 521 of SEQ ID NO: 1 is selected from the group consisting of: G521A, G521F, G521I, G521L, G521M, and G521V.
73. The modified ER-LBD of paragraph 4, wherein the one or more positions comprise position 522 of SEQ ID NO: 1.
74. The modified ER-LBD of paragraph 73, wherein the amino acid substitution at position 522 of SEQ ID NO: 1 is selected from the group consisting of: M522I, M522L, and M522V.
75. The modified ER-LBD of paragraph 4, wherein the one or more positions comprise position 524 of SEQ ID NO: 1.
76. The modified ER-LBD of paragraph 75, wherein the amino acid substitution at position 524 of SEQ ID NO: 1 is selected from the group consisting of: H524A, H524I, H524L, H524F, and H524V.
77. The modified ER-LBD of paragraph 4, wherein the one or more positions comprise position 525 of SEQ ID NO: 1.
78. The modified ER-LBD of paragraph 77, wherein the amino acid substitution at position 525 of SEQ ID NO: 1 is selected from the group consisting of: L525F, L525I, L525M, L525N, L525Q, L525S, L525T, and L525V.
79. The modified ER-LBD of paragraph 4, wherein the one or more positions comprise position 526 of SEQ ID NO: 1.
80. The modified ER-LBD of paragraph 79, wherein the amino acid substitution at position 526 of SEQ ID NO: 1 is Y526L.
81. The modified ER-LBD of paragraph 4, wherein the one or more positions comprise position 527 of SEQ ID NO: 1.
82. The modified ER-LBD of paragraph 81, wherein the amino acid substitution at position 527 of SEQ ID NO: 1 is S527N.
83. The modified ER-LBD of paragraph 4, wherein the one or more positions comprise position 528 of SEQ ID NO: 1.
84. The modified ER-LBD of paragraph 83, wherein the amino acid substitution at position 528 of SEQ ID NO: 1 is selected from the group consisting of: M528F, M528I, and M528V.
85. The modified ER-LBD of paragraph 4, wherein the one or more positions comprise position 533 of SEQ ID NO: 1.
86. The modified ER-LBD of paragraph 85, wherein the amino acid substitution at position 533 of SEQ ID NO: 1 is selected from the group consisting of: V533F and V533W.
87. The modified ER-LBD of paragraph 4, wherein the one or more positions comprise position 534 of SEQ ID NO: 1.
88. The modified ER-LBD of paragraph 87, wherein the amino acid substitution at position 534 of SEQ ID NO: 1 is selected from the group consisting of: V534Q and V534R.
89. The modified ER-LBD of paragraph 4, wherein the one or more positions comprise position 536 of SEQ ID NO: 1.
90. The modified ER-LBD of paragraph 89, wherein the amino acid substitution at position 536 of SEQ ID NO: 1 is selected from the group consisting of: L536F, and L536M, L536R, and L536Y.
91. The modified ER-LBD of paragraph 4, wherein the one or more positions comprise position 537 of SEQ ID NO: 1.
92. The modified ER-LBD of paragraph 91, wherein the amino acid substitution at position 537 of SEQ ID NO: 1 is selected from the group consisting of: Y537E and Y537S.
93. The modified ER-LBD of paragraph 4, wherein the one or more positions comprise position 538 of SEQ ID NO: 1.
94. The modified ER-LBD of paragraph 93, wherein the amino acid substitution at position 538 of SEQ ID NO: 1 is selected from the group consisting of: D538G and D538K.
95. The modified ER-LBD of paragraph 4, wherein the one or more positions comprise position 539 of SEQ ID NO: 1.
96. The modified ER-LBD of paragraph 95, wherein the amino acid substitution at position 539 of SEQ ID NO: 1 is selected from the group consisting of: L539A and L539R.
97. The modified ER-LBD of paragraph 4, wherein the one or more positions comprise position 540 of SEQ ID NO: 1.
98. The modified ER-LBD of paragraph 97, wherein the amino acid substitution at position 540 of SEQ ID NO: 1 is selected from the group consisting of: L540A and L540F.
99. The modified ER-LBD of paragraph 4, wherein the one or more positions comprise position 547 of SEQ ID NO: 1.
100. The modified ER-LBD of paragraph 99, wherein the amino acid substitution at position 547 of SEQ ID NO: 1 is H547A.
101. The modified ER-LBD of any one of paragraphs 1-100, wherein the one or more additional amino acid substitutions are two amino acid substitutions.
102. The modified ER-LBD of paragraph 101 wherein each of the two amino acid substitutions are at a position of SEQ ID NO: 1 selected from the group consisting of: 343, 345, 347, 348, 351, 354, 384, 387, 388, 389, 391, 392, 404, 418, 421, 521, 524, and 525.
103. The modified ER-LBD of paragraph 102, wherein the two amino acid substitutions are at positions 345 and 348 of SEQ ID NO: 1.
104. The modified ER-LBD of paragraph 103, wherein the amino acid substitution at position 345 of SEQ ID NO: 1 is L345S and the amino acid substitution at position 348 of SEQ ID NO: 1 is N348K.
105. The modified ER-LBD of paragraph 102, wherein the two amino acid substitutions are at positions 384 and 389 of SEQ ID NO: 1.
106. The modified ER-LBD of paragraph 105, wherein the amino acid substitution at position 384 of SEQ ID NO: 1 is L384M and the amino acid substitution at position 389 of SEQ ID NO: 1 is I389M.
107. The modified ER-LBD of paragraph 102, wherein the two amino acid substitutions are at positions 421 and 392 of SEQ ID NO: 1.
108. The modified ER-LBD of paragraph 107, wherein the amino acid substitution at position 421 of SEQ ID NO: 1 is M421I and the amino acid substitution at position 392 of SEQ ID NO: 1 is V392M.
109. The modified ER-LBD of paragraph 102, wherein the two amino acid substitutions are at positions 354 and 391 of SEQ ID NO: 1.
110. The modified ER-LBD of paragraph 109, wherein the amino acid substitution at position 354 of SEQ ID NO: 1 is L354I and the amino acid substitution at position 391 of SEQ ID NO: 1 is L391F.
111. The modified ER-LBD of paragraph 102, wherein the two amino acid substitutions are at positions 354 and 384 of SEQ ID NO: 1.
112. The modified ER-LBD of paragraph 111, wherein the amino acid substitution at position 354 of SEQ ID NO: 1 is L354I and the amino acid substitution at position 384 of SEQ ID NO: 1 is L384M.
113. The modified ER-LBD of paragraph 102, wherein the two amino acid substitutions are at positions 354 and 387 of SEQ ID NO: 1.
114. The modified ER-LBD of paragraph 113, wherein the amino acid substitution at position 354 of SEQ ID NO: 1 is L354I and the amino acid substitution at position 387 of SEQ ID NO: 1 is L387M.
115. The modified ER-LBD of paragraph 102, wherein the two amino acid substitutions are at positions 387 and 391.
116. The modified ER-LBD of paragraph 115, wherein the amino acid substitution at position 387 of SEQ ID NO: 1 is L387M and the amino acid substitution at position 391 of SEQ ID NO: 1 is L391F.
117. The modified ER-LBD of paragraph 102, wherein the two amino acid substitutions are at positions 384 and 387 of SEQ ID NO: 1.
118. The modified ER-LBD of paragraph 117, wherein the amino acid substitution at position 384 of SEQ ID NO: 1 is L384M and the amino acid substitution at position 387 of SEQ ID NO: 1 is L387M.
119. The modified ER-LBD of paragraph 102, wherein the two amino acid substitutions are at positions 384 and 391 of SEQ ID NO: 1.
120. The modified ER-LBD of paragraph 117, wherein the amino acid substitution at position 384 of SEQ ID NO: 1 is L384M and the amino acid substitution at position 391 of SEQ ID NO: 1 is L391F.
121. The modified ER-LBD of any one of paragraphs 1 to 120, wherein the one or more additional amino acid substitutions are three amino acid substitutions.
122. The modified ER-LBD of paragraph 121, wherein each of the three amino acid substitutions are at a position of SEQ ID NO: 1 selected from the group consisting of: 343, 347, 351, 354, 388, 391, 404, 414, 418, 463, 521, 524, and 525.
123. The modified ER-LBD of paragraph 122, wherein the three amino acid substitutions are at positions 354, 384, and 391 of SEQ ID NO: 1.
124. The modified ER-LBD of paragraph 123, wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F.
125. The modified ER-LBD of paragraph 122, wherein the three amino acid substitutions are at positions 414, 463, and 524 of SEQ ID NO: 1.
126. The modified ER-LBD of paragraph 125, wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L.
127. The modified ER-LBD of any one of paragraphs 1 to 126, wherein the one or more additional amino acid substitutions are four amino acid substitutions.
128. The modified ER-LBD of paragraph 127, wherein each of the four amino acid substitutions are at a position of SEQ ID NO: 1 selected from the group consisting of: 343, 347, 351, 354, 384, 388, 391, 404, 413, 418, 463, 521, 524, and 525.
129. The modified ER-LBD of paragraph 128, wherein the four amino acid substitutions are at positions 354, 384, 391, and 418 of SEQ ID NO: 1.
130. The modified ER-LBD of paragraph 129, wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I.
131. The modified ER-LBD of paragraph 128, wherein the four amino acid substitutions are at positions 343, 388, 521, and 404 of SEQ ID NO: 1.
132. The modified ER-LBD of paragraph 131, wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L.
133. The modified ER-LBD of paragraph 128, wherein the four amino acid substitutions are at positions 524, 347, 351, and 525 of SEQ ID NO: 1.
134. The modified ER-LBD of paragraph 133, wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N.
135. The modified ER-LBD of paragraph 128, wherein the four amino acid substitutions are at positions 354, 384, 391, and 463 of SEQ ID NO: 1.
136. The modified ER-LBD of paragraph 135, wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P.
137. The modified ER-LBD of paragraph 128, wherein the four amino acid substitutions are at positions 384, 391, 413, and 524 of SEQ ID NO: 1.
138. The modified ER-LBD of paragraph 137, wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F.
139. The modified ER-LBD of any one of paragraphs 1 to 138, wherein the one or more additional amino acid substitutions are five amino acid substitutions.
140. The modified ER-LBD of paragraph 139, wherein each of the five amino acid substitutions are at a position of SEQ ID NO: 1 selected from the group consisting of: 354, 384, 391, 409, 413, 414, 421, 463, and 524.
141. The modified ER-LBD of paragraph 140, wherein the five amino acid substitutions are at positions 384, 409, 413, 463, and 524 of SEQ ID NO: 1.
142. The modified ER-LBD of paragraph 141, wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L.
143. The modified ER-LBD of paragraph 140, wherein the five amino acid substitutions are at positions 391, 413, 414, 463, and 524 of SEQ ID NO: 1.
144. The modified ER-LBD of paragraph 143, wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F.
145. The modified ER-LBD of paragraph 140, wherein the five amino acid substitutions are at positions 391, 414, 421, 463, and 524 of SEQ ID NO: 1.
146. The modified ER-LBD of paragraph 145, wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F.
147. The modified ER-LBD of paragraph 140, wherein the five amino acid substitutions are at positions 354, 409, 413, 421, and 524 of SEQ ID NO: 1.
148. The modified ER-LBD of paragraph 147, wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L.
149. The modified ER-LBD of paragraph 140, wherein the five amino acid substitutions are at positions 354, 409, 421, 463, and 524 of SEQ ID NO: 1.
150. The modified ER-LBD of paragraph 149, wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.
151. The modified ER-LBD of any one of paragraphs 1 to 150, wherein the one or more additional amino acid substitutions are six amino acid substitutions.
152. The modified ER-LBD of paragraph 151, wherein each of the six amino acid substitutions are at a position of SEQ ID NO: 1 selected from the group consisting of: 354, 384, 391, 409, 413, 414, 421, 463, and 524.
153. The modified ER-LBD of paragraph 152, wherein the six amino acid substitutions are at positions 384, 391, 413, 421, 463, and 524 of SEQ ID NO: 1.
154. The modified ER-LBD of paragraph 153, wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.
155. The modified ER-LBD of paragraph 152, wherein the six amino acid substitutions are at positions 409, 413, 414, 421, 463, and 524 of SEQ ID NO: 1.
156. The modified ER-LBD of paragraph 155, wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.
157. The modified ER-LBD of paragraph 152, wherein the six amino acid substitutions are at positions 354, 391, 409, 413, 414, and 524 of SEQ ID NO: 1.
158. The modified ER-LBD of paragraph 157, wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L.
159. The modified ER-LBD of any one of paragraphs 1 to 158, wherein the one or more additional amino acid substitutions are seven amino acid substitutions.
160. The modified ER-LBD of paragraph 159, wherein each of the seven amino acid substitutions are at a position of SEQ ID NO: 1 selected from the group consisting of: 354, 384, 391, 409, 413, 414, 421, 463, 517, and 524.
161. The modified ER-LBD of paragraph 160, wherein the seven amino acid substitutions are at positions 354, 384, 409, 413, 421, 463, and 524 of SEQ ID NO: 1.
162. The modified ER-LBD of paragraph 161, wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F.
163. The modified ER-LBD of paragraph 160, wherein the seven amino acid substitutions are at positions 354, 391, 413, 421, 463, 517, and 524 of SEQ ID NO: 1.
164. The modified ER-LBD of paragraph 163, wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L.
165. The modified ER-LBD of paragraph 160, wherein the seven amino acid substitutions are at positions 354, 391, 413, 414, 421, 517, and 524 of SEQ ID NO: 1.
166. The modified ER-LBD of paragraph 165, wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F.
167. The modified ER-LBD of any one of paragraphs 1 to 166, wherein the one or more additional amino acid substitutions are eight amino acid substitutions.
168. The modified ER-LBD of paragraph 167, wherein the eight amino acid substitutions are at positions 384, 391, 409, 413, 421, 463, 517, and 524 of SEQ ID NO: 1.
169. The modified ER-LBD of paragraph 168, wherein the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.
170. The modified ER-LBD of any one of paragraphs 1 to 169, wherein the modified ER-LBD further comprises a V595A amino acid substitution.
171. The modified ER-LBD of any one of paragraphs 1 to 170, wherein the non-endogenous ligand is selected from the group consisting of: 4-hydroxytamoxifen, N-desmethyltamoxifen, tamoxifen-N-oxide, and endoxifen.
172. A chimeric protein comprising a polypeptide of interest fused to the modified ER-LBD of any one of paragraphs 1 to 171.
173. The chimeric protein of paragraph 172, wherein the polypeptide of interest comprises a nucleic acid binding domain.
174. The chimeric protein of paragraph 173, wherein the nucleic acid binding domain comprises a zinc finger domain.
175. The chimeric protein of paragraph 174, wherein the zinc finger domain comprises the sequence

(SEQ ID NO: 62)
MSRPGERPFQCRICMRNFSNMSNLTRHTRTHTGEKPFQCRICMRNFSDR
SVLRRHLRTHTGSQKPFQCRICMRNFSDPSNLARHTRTHTGEKPFQCRI
CMRNFSDRSSLRRHLRTHTGSQKPFQCRICMRNFSQSGTLHRHTRTHTG
EKPFQCRICMRNFSQRPNLTRHLRTHLRGS.

176. The chimeric protein of any one of paragraphs 172 to 175, wherein the chimeric protein comprises a chimeric transcription factor, and wherein the polypeptide of interest comprises a nucleic acid binding domain and a transcriptional modulator domain.
177. The chimeric protein of paragraph 176, wherein the transcriptional modular domain is a transcriptional activator.
178. The chimeric protein of paragraph 177, wherein the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain).
179. The chimeric protein of paragraph 178, wherein the transcriptional activator is a p65 transcriptional activator comprising the amino acid sequence of

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

180. An isolated polynucleotide molecule comprising a nucleotide sequence encoding the modified ER-LBD of any one of paragraphs 1 to 171.
181. An isolated polynucleotide molecule comprising a nucleotide sequence encoding the chimeric protein of any one of paragraphs 172 to 179.
182. A heterologous construct comprising a promoter operatively linked to the polynucleotide molecule of paragraph 180.
183. A heterologous construct comprising a promoter operatively linked to the polynucleotide molecule of paragraph 181.
184. A plasmid comprising the heterologous construct of paragraph 182.
185. A plasmid comprising the heterologous construct of paragraph 183.
186. A cell comprising the heterologous construct of paragraph 182 or the plasmid of paragraph 184.
187. A cell comprising the heterologous construct of paragraph 183 or the plasmid of paragraph 185.
188. A genetic switch for modulating transcription of a gene of interest, comprising:
(a) the chimeric protein of paragraph 176, wherein the chimeric protein binds to a chimeric transcription factor-responsive (CTF-responsive) promoter operably linked to the gene of interest; and
(b) a non-endogenous ligand, wherein binding of the non-endogenous ligand to the modified ER-LBD induces the chimeric protein to modulate transcription of the gene of interest.
189. The genetic switch of paragraph 188, wherein the non-endogenous ligand is selected from the group consisting of: 4-hydroxytamoxifen, N-desmethyltamoxifen, tamoxifen-N-oxide, and endoxifen.
190. The genetic switch of paragraph 188 or paragraph 189, wherein the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme.
191. The genetic switch of paragraph 190, wherein gene of interest encodes a cytokine selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
192. The genetic switch of paragraph 191, wherein the gene of interest encodes an IL12p70 fusion protein comprising the amino acid sequence of

(SEQ ID NO: 58)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSGGGSGGGSGGGSGGG
SRNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS.

193. A method of modulating transcription of a gene of interest, comprising: transforming a cell with (i) a heterologous construct encoding the chimeric protein of paragraph 176 and (ii) a target expression cassette comprising a chimeric transcription factor-responsive (CTF-responsive) promoter operably linked to the gene of interest, and inducing the chimeric protein to modulate transcription of the gene of interest by contacting the transformed cell with a non-endogenous ligand.
194. The method of paragraph 193, wherein the method further comprises culturing the transformed cell under conditions suitable for expression of the chimeric protein prior to inducing the chimeric protein to modulate transcription.
195. The method of paragraph 193 or 194, wherein modulating transcription comprises activating transcription of the gene of interest.
196. The method of paragraph 193 or 194, wherein modulating transcription comprises repressing transcription of the gene of interest.
197. The method of any one of paragraphs 193 to 196, wherein the target expression cassette is encoded by the heterologous construct encoding the chimeric protein of paragraph 174.
198. The method of any one of paragraphs 193 to 196, wherein the target expression cassette is encoded by a second heterologous construct.
199. A method of modulating localization of a chimeric protein comprising transforming a cell with a heterologous construct encoding the chimeric protein of any one of paragraphs 172 to 179, and inducing nuclear localization of the chimeric protein by contacting the transformed cell with a non-endogenous ligand.
200. The method of paragraph 199, the method further comprising culturing the transformed cell under conditions suitable for expression of the chimeric protein prior to inducing the nuclear localization.
201. The method of any one of paragraphs 193, or 195 to 200, wherein the transformed cell is in a human or animal, and wherein contacting the transformed cell with the non-endogenous ligand comprises administering a pharmacological dose of the ligand to the human or animal.
202. The method of any one of paragraphs 193 to 201, wherein the non-endogenous ligand is selected from the group consisting of: 4-hydroxytamoxifen, N-desmethyltamoxifen, tamoxifen-N-oxide, and endoxifen.
203. The method of paragraph 201 or paragraph 202, wherein the non-endogenous ligand is administered at a concentration at which the non-endogenous ligand is substantially inactive on a wild-type estrogen receptor alpha of SEQ ID NO: 1.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: ERT2 Mutations Predicted to Modulate Ligand Binding

In silico modeling was conducted for 4-OHT, endoxifen and estradiol binding to a mutant form of estrogen receptor alpha known as ERT2, to identify mutations predicted to have increased sensitivity to 4-OHT, as compared to an ERT2 of SEQ ID NO: 2.

### Materials and Methods

Available crystal structures of a complex between Estradiol and ERα (PDB: 1QKU, resolution 3.2 Å) and between 4-OHT and ERα (PDB: 3ERT, resolution 1.9 Å) were used to generate models of the complexes between Estradiol and ERT2, 4-OHT and ERT2, and Endoxifen and ERT2. The ERT2 sequence differs from ERα by three residues (G400V/M543A/L544A). Only residues from 306 to 551 were used in the structural models as the available structures were all resolved with only this region.

Using standard protocols (Kannan et al. ACS Omega. 2017 Nov. 30; 2(11): 7881-7891.), MD simulations were carried out for apo ERT2, ERT2—Estradiol, ERT2—4-OHT and ERT2—Endoxifen complexes (each simulation was carried out for 100 ns in triplicate each). Both the ERT2 and the bound ligand/drug remained stable during the simulations (using standard measures). The conformations generated during the last half (50 ns) of the simulations (the simulations are deemed to have equilibrated) were used for subsequent analyses.

### Results

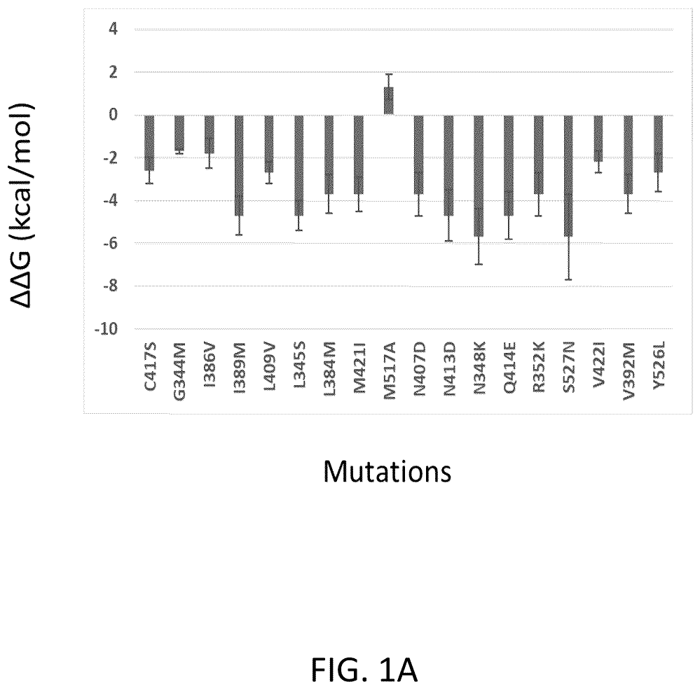

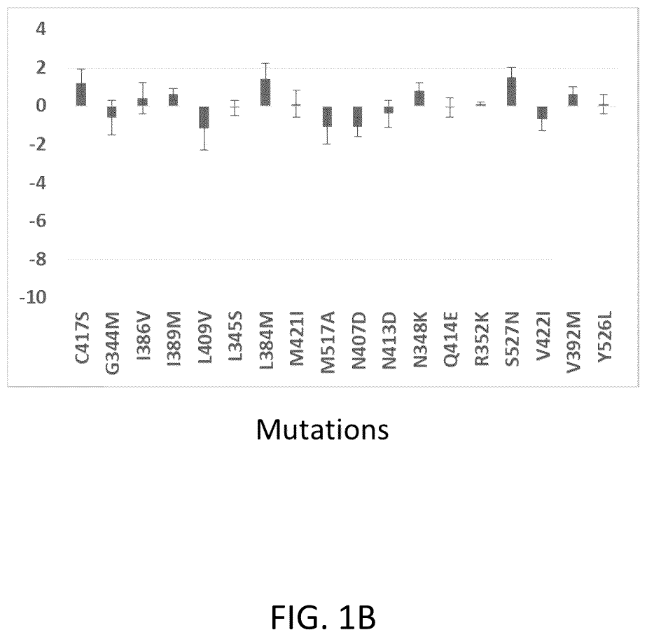

A first set of mutations was analyzed in silico for improved 4-OHT binding. Eighteen mutations to residues in the ligand binding pocket were selected based on amino acids present at the homologous position for other estrogen receptor proteins. Of the 18 selected mutations, 17 of the mutants bind tighter than wild type ERT2 by at least 1.8 kcal/mol; only the M517A mutation appears to destabilize the binding of 4-OHT (FIG. 1A). Next, binding energy calculations were carried out to see the effect of the mutation on the binding of estradiol. Compared to 4-OHT (in which all mutations except M517A favor the binding as indicated by negative ΔΔG values) most mutations (FIG. 1B) had negligible effect on the binding of estradiol (ΔΔG values for all the mutations are within 2 kcal/mol, as compared to the ΔΔG values of 4-OHT which is >2 kcal/mol for most of the mutations). Only mutations L409V, M517A and N407D exhibited increased binding to estradiol of greater than 1 kcal/mol, but both L409V and N407D bind tighter to 4-OHT by 3 and 4 kcal/mol respectively. The first set of mutations is shown below in Table 6.

TABLE 6
Mutations

| | | |
|---|---|---|
| G344M | I389M | C417S |
| L345S | V392M | M421I |
| N348K | N407D | V422I |
| R352K | L409V | M517A |
| L384M | N413D | Y526L |
| I386V | Q414E | S527N |

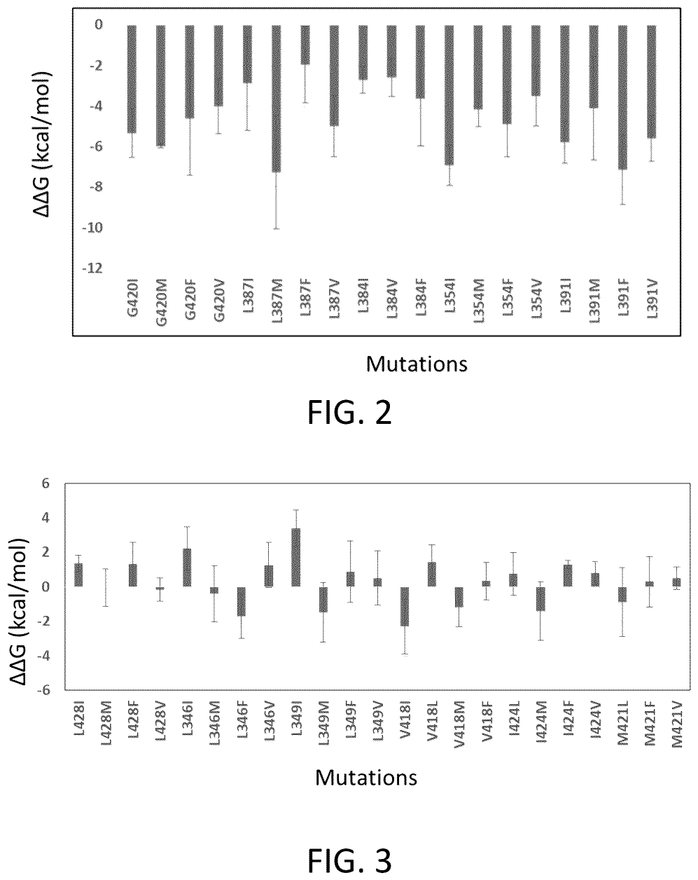

A second set of mutations was analyzed in silico for improved 4-OHT binding. Molecular docking simulations were conducted for 4-OHT and estradiol binding to ERT2, for nineteen different mutations at five additional sites at the ligand binding pocket (in addition to those shown in Table 6), to identify further mutants with increased sensitivity to 4-OHT, as compared to wild-type ERT2. Binding energy calculations were carried out consistent with the calculations performed for the first set of mutations. All nineteen of the mutations exhibited improved binding to 4-OHT in the range of 1.8 kcal/mol to 7 kcal/mol (see FIG. 2). The second set of mutations is shown in Table 7.

TABLE 7
Mutations

| | | |
|---|---|---|
| L354I | L387I | L391V |
| L354M | L387M | G420I |
| L354F | L387F | G420M |
| L354V | L387V | G420F |
| L384I | L391I | G420V |
| L384V | L391M | |
| L384F | L391F | |

A third set of mutations was analyzed in silico for improved 4-OHT binding. A total of 23 mutations at an additional six residue positions in the ligand binding pocket (residues 428, 346, 349, 418, 421, and 424) were chosen for molecular docking simulations. Binding energy calculations were carried out consistent with the calculations performed for the first set of mutations. Six of the mutations (L346F, L349M, V418I, V418M, I424M, and M421L) exhibited improved binding to 4-OHT by at least about 1.5 kcal/mol (FIG. 3). These 23 mutations are shown in Table 8.

TABLE 8
Mutations

| | | |
|---|---|---|
| L346I | V418I | I424M |
| L346M | V418L | I424F |
| L346F | V418M | I424V |
| L346V | V418F | L428I |
| L349I | M421L | L428M |
| L349M | M421F | L428F |
| L349F | M421V | L428V |
| L349V | I424L | |

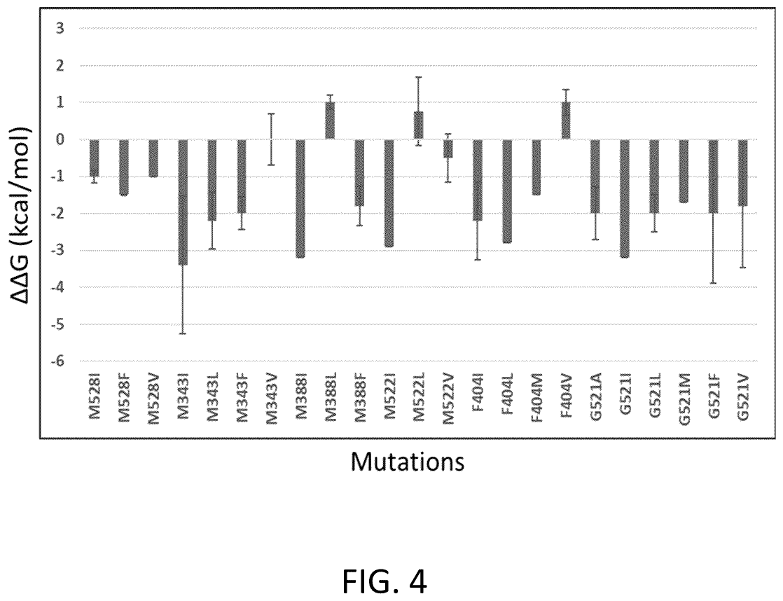

A fourth set of mutations was analyzed in silico for improved 4-OHT binding. A total of 23 mutations at an additional six residue positions in the ligand binding pocket (residues 528, 343, 388, 522, 414, and 521) were chosen for molecular docking simulations. Binding energy calculations were carried out consistent with the calculations performed for the first set of mutations. 18 of the 23 mutations exhibited improved binding to 4-OHT by at least about 1.0 kcal/mol (FIG. 4). The fourth set of mutations is shown in Table 9.

TABLE 9
Mutations

| | | |
|---|---|---|
| M343I | F404L | G521V |
| M343L | F404M | M522I |
| M343F | F404V | M522L |
| M343V | G521A | M522V |
| M388I | G521I | M528I |
| M388L | G521L | M528F |
| M388F | G521M | M528V |
| F404I | G521F | |

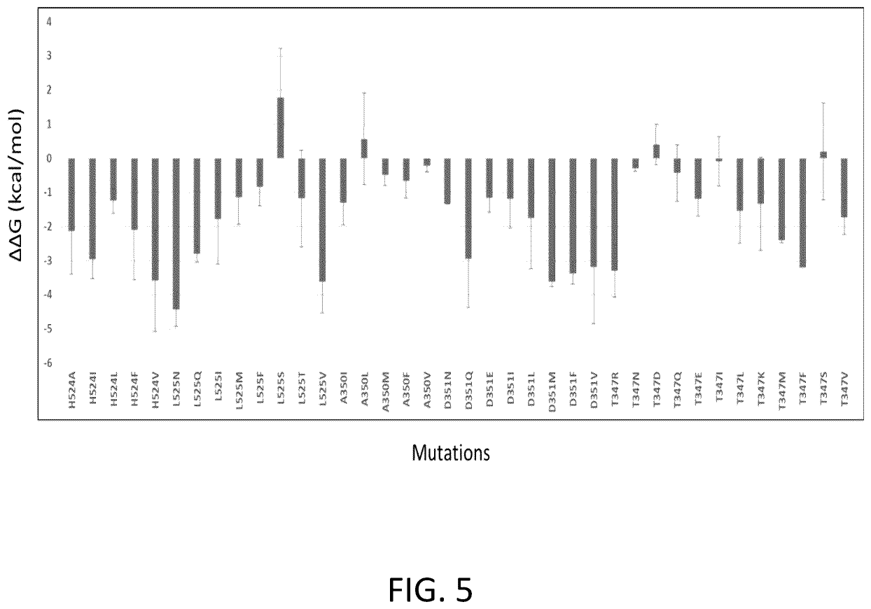

A fifth set of mutations was analyzed in silico for improved 4-OHT binding. A total of 38 mutations at five additional residue positions (residues 524, 525, 347, 350, and 351) were chosen for molecular docking simulations. Binding energy calculations were carried out consistent with the calculations performed for the first set of mutations. 28 of the 38 mutations exhibited improved binding to 4-OHT by at least about 1.0 kcal/mol and up to about 4.5 kcal/mol (FIG. 5). The fifth set of mutations is shown in Table 10.

TABLE 10
Mutations

| | |
|---|---|
| H524A | T347S |
| H524I | A350I |
| H524L | A350L |
| H524F | A350M |
| H524V | A350F |
| L525N | A350V |
| L525Q | D351N |
| L525I | D351Q |
| L525M | D351E |
| L525F | D351I |
| L525S | D351L |
| L525T | D351M |
| L525V | D351F |
| T347V | D351V |

All of the mutations for sets 1-5 were further analyzed with molecular docking simulations for binding to endoxifen and estradiol to determine the energy of binding to endoxifen and estradiol (calculated as ΔΔG in kcal/mol). Additionally, the difference between the binding energy of endoxifen binding as compared to estradiol binding was calculated as AΔΔG values. A summary of the binding energies for each of 4-OHT, endoxifen, and estradiol, and of the binding energy differences of 4-OHT and endoxifen as compared to estradiol binding is shown in Table 11.

TABLE 11

| | 4-OHT | END | EST | 4-OHT-EST | END-EST |
|---|---|---|---|---|---|
| | (ΔΔG) | (ΔΔG) | (ΔΔG) | (ΔΔΔG) | (ΔΔΔG) |
| | (kcal/mol) | (kcal/mol) | (kcal/mol) | (kcal/mol) | (kcal/mol) |
| M343I | −3.4 | −2.2 | 1.5 | −4.9 | −3.7 |
| M343L | −2.2 | −1.6 | 0.9 | −3.1 | −2.6 |
| M343V | 0 | −0.7 | 1.8 | −1.8 | −2.5 |
| M343F | −2 | −1.6 | 2.2 | −4.2 | −3.8 |
| G344M | −1.6 | −1.4 | −0.6 | −1 | −0.8 |
| L345S | −4.3 | 1.1 | −0.1 | −4.2 | 1.2 |
| L346I | 2.2 | −0.8 | −0.9 | 3.1 | 0.1 |
| L346M | −0.4 | −0.8 | −0.5 | 0.1 | −0.3 |
| L346V | 1.3 | 0.8 | 0.5 | 0.8 | 0.3 |
| L346F | −1.7 | −0.8 | −0.1 | −1.6 | −0.6 |
| T347I | −0.1 | −0.7 | 0.4 | −0.5 | −1.1 |
| T347L | −1.6 | −1.2 | 0.5 | −2 | −1.7 |
| T347M | −2.4 | −2.3 | −1.9 | −0.5 | −0.3 |
| T347N | −0.3 | −2.7 | 1.1 | −1.4 | −3.8 |
| T347R | −3.3 | −4.3 | −0.8 | −2.5 | −3.5 |
| T347V | −1.7 | −0.9 | 0 | −1.7 | −0.9 |
| T347D | 0.4 | 3.8 | 0.4 | 0 | 3.4 |
| T347E | −1.2 | 2.5 | 1.1 | −2.3 | 1.4 |
| T347F | −3.2 | −2.2 | 0.1 | −3.3 | −2.2 |
| T347K | −1.3 | −2.5 | 1.4 | −2.7 | −3.9 |
| T347Q | −0.4 | 0.4 | 1.3 | −1.8 | −0.9 |
| T347S | 0.2 | −2.7 | 1.1 | −0.9 | −3.7 |
| N348K | −5.5 | −3.6 | 0.8 | −6.3 | −4.4 |
| L349I | 3.4 | 1.4 | 2 | 1.4 | −0.6 |
| L349M | −1.5 | −2 | 2.6 | −4.1 | −4.6 |
| L349V | 0.5 | 1.5 | 1.7 | −1.2 | −0.2 |
| L349F | 0.9 | −0.8 | −0.7 | 1.6 | −0.1 |
| A350L | 0.6 | −0.6 | 1 | −0.4 | −1.6 |
| A350M | −0.5 | −2 | 1.3 | −1.8 | −3.3 |
| A350V | −0.2 | −1.1 | 0.5 | −0.7 | −1.5 |
| A350F | −0.7 | −2.3 | −1 | 0.4 | −1.3 |
| A350I | −1.3 | −3.8 | 0.6 | −1.9 | −4.4 |
| D351I | −1.2 | −3.5 | 2 | −3.2 | −5.5 |
| D351L | −1.7 | −3.2 | 0.4 | −2.1 | −3.6 |
| D351M | −3.6 | −3 | −0.2 | −3.4 | −2.9 |
| D351N | −1.3 | −2.1 | 1.8 | −3.2 | −4 |
| D351V | −3.2 | −3.8 | 0.6 | −3.8 | −4.4 |
| D351E | −1.2 | 1 | 0.7 | −1.8 | 0.3 |
| D351F | −3.4 | −2.2 | 1.2 | −4.6 | −3.4 |
| D351Q | −3 | 0.1 | 0.3 | −3.2 | −0.2 |
| R352K | −3.8 | −2.5 | 0.1 | −3.9 | −2.6 |
| L354I | −6.9 | −2.5 | 0.9 | −7.8 | −3.4 |
| L354M | −4.2 | −1.3 | 0.9 | −5.1 | −2.2 |
| L354V | −3.5 | −2.5 | 1.5 | −5 | −4 |
| L354F | −4.9 | −2.6 | 0.7 | −5.6 | −3.3 |
| L384I | −2.7 | −4.6 | 0.3 | −3 | −4.9 |
| L384M | −3.3 | 0 | 1.4 | −4.7 | −1.4 |
| L384V | −3.6 | −2.5 | 3.2 | −6.9 | −5.7 |
| L384F | −2.6 | −0.4 | 3 | −5.5 | −3.3 |
| I386V | −1.8 | −3.4 | 0.4 | −2.2 | −3.8 |
| L387I | −2.9 | −2.6 | 0.4 | −3.2 | −3 |
| L387M | −7.3 | −1.9 | 0.3 | −7.5 | −2.2 |
| L387V | −5 | −2.4 | −0.7 | −4.3 | −1.6 |
| L387F | −2 | −0.4 | 1.1 | −3 | −1.4 |
| M388I | −3.2 | −1.7 | 0.3 | −3.5 | −2 |
| M388L | 1 | −0.7 | 1.9 | −0.9 | −2.6 |
| M388F | −1.8 | −1.6 | 1.2 | −3 | −2.8 |
| I389M | −4.6 | −2.1 | 0.6 | −5.2 | −2.7 |
| L391I | −5.8 | −2.8 | 2 | −7.8 | −4.8 |
| L391M | −4.1 | −4.5 | 0 | −4.1 | −4.5 |
| L391V | −5.6 | −3.4 | 1.5 | −7.1 | −4.9 |
| L391F | −7.2 | −1.5 | 0.1 | −7.3 | −1.6 |
| V392M | −3.9 | −2.6 | 0.6 | −4.5 | −3.2 |
| F404I | −2.2 | −1.3 | 2 | −4.2 | −3.4 |
| F404L | −2.8 | −1.7 | −0.5 | −2.3 | −1.2 |
| F404M | −1.5 | −0.3 | 2.2 | −3.7 | −2.4 |
| F404V | 1 | −1 | 1.4 | −0.4 | −2.4 |
| N407D | −3.5 | −2.2 | −1.1 | −2.4 | −1.1 |
| L409V | −2.5 | −1.8 | −1.2 | −1.3 | −0.6 |
| N413D | −4.7 | −0.8 | −0.4 | −4.3 | −0.4 |
| Q414E | −4.5 | −1.8 | −0.1 | −4.4 | −1.7 |
| C417S | −2.6 | −1 | 1.2 | −3.8 | −2.2 |
| V418I | −2.3 | −1.1 | 1.9 | −4.2 | −3.1 |
| V418L | 1.5 | −1.3 | 2 | −0.5 | −3.2 |
| V418M | −1.2 | −1.8 | 1.4 | −2.6 | −3.2 |
| V418F | 0.4 | −2.6 | 0.6 | −0.3 | −3.2 |
| G420I | −5.3 | −2.8 | −0.2 | −5.1 | −2.6 |
| G420M | −6 | −3.9 | −1.2 | −4.8 | −2.7 |
| G420V | −4 | −2.5 | 0.1 | −4.1 | −2.6 |
| G420F | −4.6 | −4 | 3.3 | −7.9 | −7.3 |
| M421I | −3.3 | −2.1 | 0.1 | −3.4 | −2.2 |
| M421L | −0.9 | −1.7 | 0 | −0.9 | −1.7 |
| M421V | 0.5 | 0.4 | 0.4 | 0.1 | 0 |
| M421F | 0.3 | −1.4 | −0.7 | 1 | −0.8 |
| V422I | −2.4 | −2.2 | −0.7 | −1.7 | −1.5 |
| I424L | 0.8 | −0.8 | −0.6 | 1.3 | −0.2 |
| I424M | −1.4 | −1.8 | 0.3 | −1.7 | −2 |
| I424V | 0.8 | −1.8 | 3.1 | −2.3 | −4.9 |
| I424F | 1.3 | −1.4 | 0.8 | 0.5 | −2.2 |
| L428I | 1.3 | −0.5 | 0.5 | 0.8 | −1.1 |
| L428M | 0 | −1.9 | 0.9 | −0.9 | −2.8 |
| L428V | −0.2 | −0.1 | 2.3 | −2.4 | −2.4 |
| L428F | 1.3 | 0.7 | 1.3 | 0 | −0.6 |
| M517A | 1.2 | 0.8 | −1.1 | 2.3 | 1.9 |
| G521A | −2 | 0 | 0.2 | −2.2 | −0.2 |
| G521I | 3.2 | −0.3 | 2.3 | −5.5 | −2.6 |
| G521L | −2 | −1 | 1.9 | −3.9 | −2.9 |
| G521M | −1.7 | −2.5 | 3.3 | −5 | −5.8 |
| G521V | −1.8 | −1.1 | 0.5 | −2.3 | −1.6 |
| G521F | −2 | −1.8 | 3.4 | −5.4 | −5.3 |
| M522I | −2.9 | −2 | 1.7 | −4.6 | −3.7 |
| M522L | 0.8 | −0.7 | 0.1 | 0.7 | −0.8 |
| M522V | −0.5 | −1 | −0.7 | 0.2 | −0.3 |
| H524A | −2.1 | −3 | −0.4 | −1.7 | −2.6 |
| H524I | −3 | −3 | 2.8 | −5.8 | −5.8 |
| H524L | −1.2 | −1.3 | 2 | −3.3 | −3.3 |
| H524V | −3.6 | −3.3 | 0.6 | −4.2 | −4 |
| H524F | −2.1 | −3.9 | 2.7 | −4.8 | −6.6 |
| L525I | −1.8 | −2.1 | 0.5 | −2.3 | −2.6 |
| L525M | −1.2 | −1.8 | −0.2 | −0.9 | −1.5 |
| L525N | −4.4 | −1.4 | −0.4 | −4 | −0.9 |
| L525T | −1.2 | −2.3 | 0.6 | −1.7 | −2.9 |
| L525V | −3.6 | −4.5 | −1 | −2.7 | −3.5 |
| L525F | −0.8 | −1.1 | −0.4 | −0.4 | −0.6 |
| L525Q | −2.8 | −3.4 | −1.4 | −1.4 | −2 |
| L525S | 1.8 | −2.4 | 0.3 | 1.5 | −2.7 |
| Y526L | −2.8 | −2 | 0.1 | −2.9 | −2.1 |
| S527N | −5.8 | −3 | 1.5 | −7.3 | −4.5 |
| M528I | −1 | −2.5 | 0.5 | −1.5 | −2.9 |
| M528V | −1 | −1.1 | 3 | −4 | −4.1 |
| M528F | −1.5 | −1 | 2.5 | −4 | −3.5 |

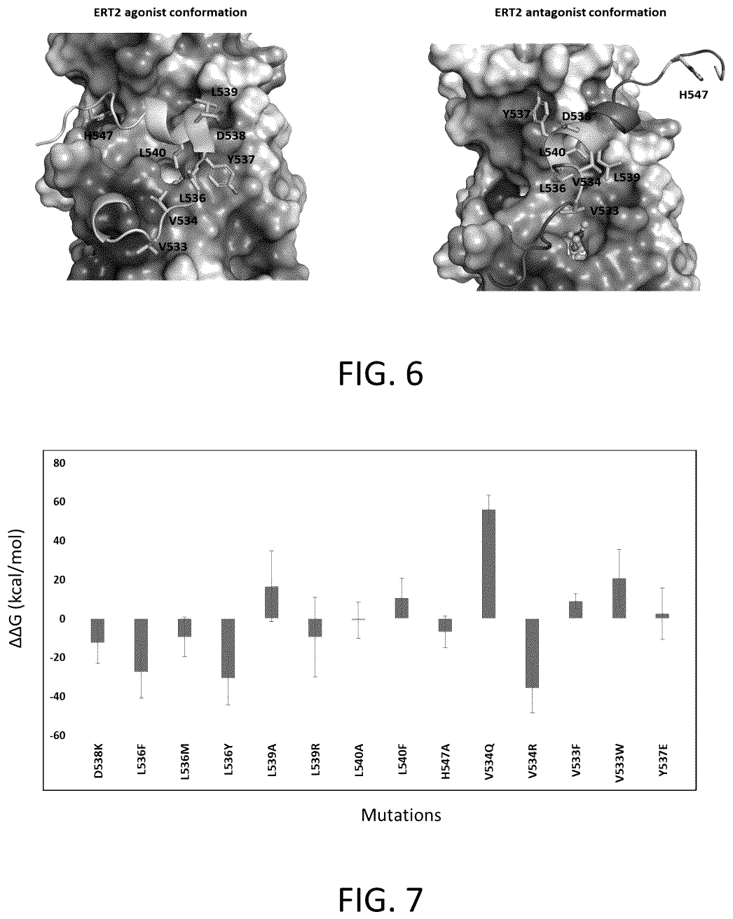

A sixth set of mutations was analyzed in silico to identify mutants that destabilize the agonist-bound confirmation (i.e., the estradiol-bound conformation) and/or stabilize the antagonist-bound confirmation (i.e., the 4-OHT or endoxifen-bound conformation). A major structural difference between the agonist-bound and antagonist-bound conformations lies in the orientation and docking site of helix 12 (H12, see FIG. 6). A total of 14 mutations at eight residue positions in helix 12 (residues 538, 536, 539, 540, 547, 534, 533, and 537) were chosen for analysis. The difference in free energy of the mutant ERT2 and the wild-type ERT2 in the antagonist-bound conformation, and the difference in free energy of the mutant ERT2 and wild-type ERT2 in the agonist-bound conformation were calculated as AG values. Next, ΔΔG values were calculated, with a negative value indicating that the antagonist-bound conformation of the ERT2 mutant is favored over the agonist-bound conformation of the mutant, and a positive value indicating that the agonist conformation is favored over the antagonist-bound conformation. Seven of the fourteen mutations (D538K, L536F, L536Y, L536M, L539R, H547A, and V534R) stabilized the antagonist confirmation (see FIG. 7). The sixth set of mutations is shown in Table 12.

TABLE 12
Mutations

| | |
|---|---|
| V533F | Y537E |
| V533W | D538K |
| V534R | L539R |
| V534Q | L539A |
| L536F | L540A |
| L536M | L540F |
| L536Y | H547A |

### Example 2: ERT2 Mutants with Increased Drug Sensitivity Identified by Transfection Screen

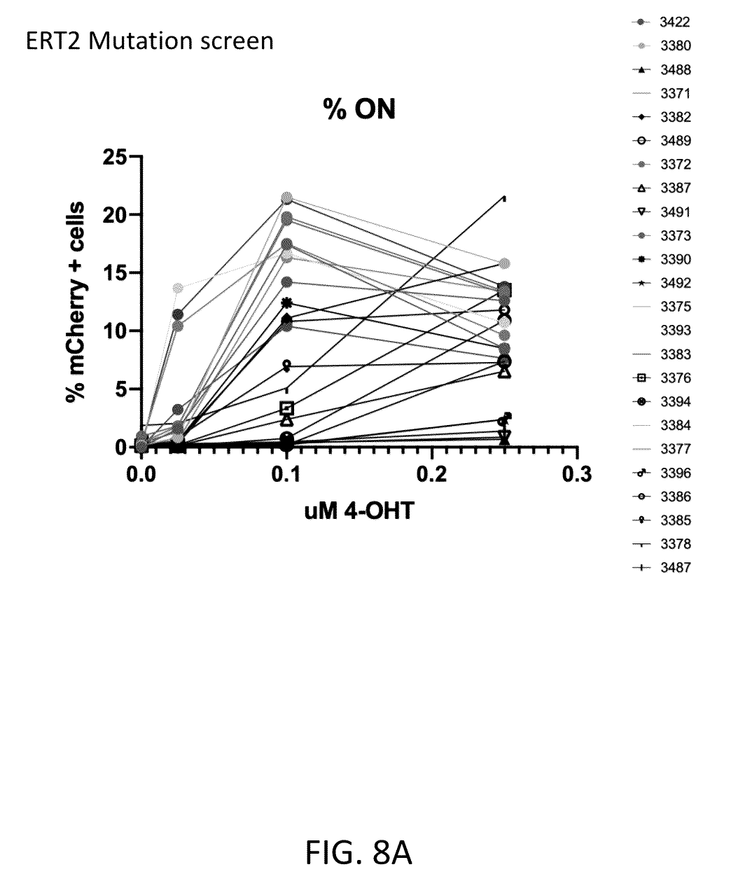

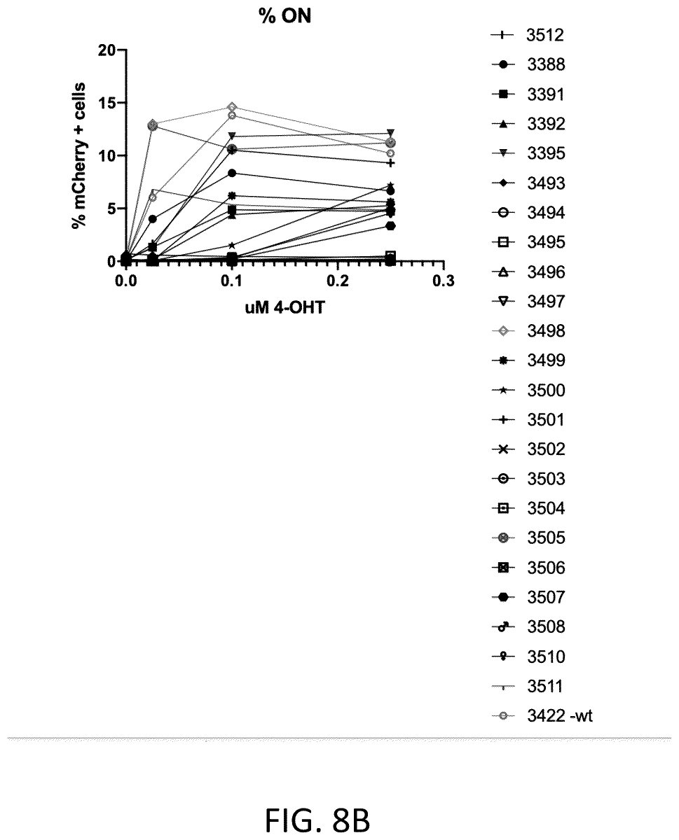

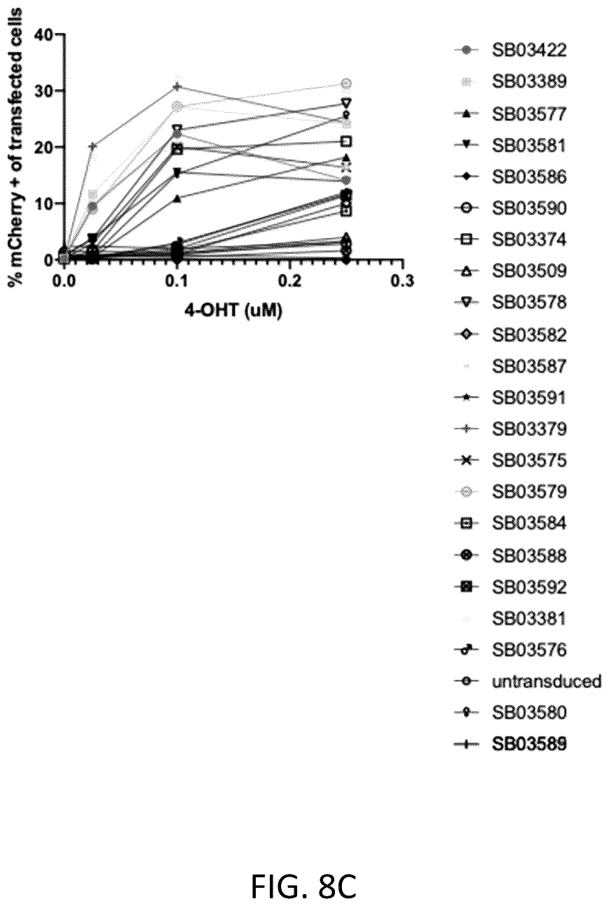

ERT2 mutants were analyzed by transfection assays for the ability to induce reporter expression in response to 4-OHT. In a first transfection screen, constructs encoding ERT2 having mutations described in Example 1 were produced in the background of a “wild-type” ERT2 as shown in SEQ ID NO: 3 (including the G400V/M543A/L544A/V595A quadruple amino acid substitution). Each ERT2 construct included a ZF10-1 domain for DNA binding, a p65 transcriptional activation domain, and the ERT2 mutant. Each construct was tested for sensitivity to 4-OHT. Each mutant was cloned into an expression construct for transfection in a HEK293T+YBTATA mCherry reporter cell line. In a second transfection screen, constructs encoding additional ERT2 mutants described in Example 1 were produced and tested for sensitivity to 4-OHT. For the screens, the cells were treated with three different concentrations of 4-OHT (0.025, 0.1, and 0.25 uM) and then assayed for mCherry expression by fluorescence-activated cell sorting (FACS) (FIGS. 8A-8C, FIGS. 9A-9C, and FIGS. 10A-10C).

### Materials and Methods

HEK293T cells were transduced with a lentivirus encoding a synthetic promoter comprised of 4 ZF10-1 binding sites linked to a YBTATA minimal promoter. This synthetic promoter drives expression of mCherry. Cells from this cell line were called “reporter cells.”

On day 1, reporter cells were plated at 1.5e5 cells/well in a 24 well plate. On day 2, cells were transfected with ERT mutants. A mix of 0.6 ug DNA, 1.8 uL Fugene, and 30 uL Optimem was made for each well where the DNA encodes ZF10-1 fused to p65 and the ERT2 mutant. In some screens a plasmid encoding GFP was included as a control to select transfected cells by flow cytometry. On day 3, cells were split at a ratio of 1:20 and seeded in a 96 well plate. Cells were treated with 0, 0.025, 0.1, or 0.25 uM 4-OHT. On day 5, media was removed and cells were trypsinized and then resuspended in FACS buffer plus Sytox Red (fluoresces in APC channel) viability dye. Cells were run on a flow cytometer and gated by FSC/SSC for cells, FSC/Sytox Red—for live cells, FSC/FSC-Width for single cells, and where possible GFP+ for transfected cells (if transfection control was included). The percent of mCherry positive cells at each drug concentration was plotted and compared to wildtype ERT2, and mutants that were more sensitive to 4-OHT were identified.

### Results

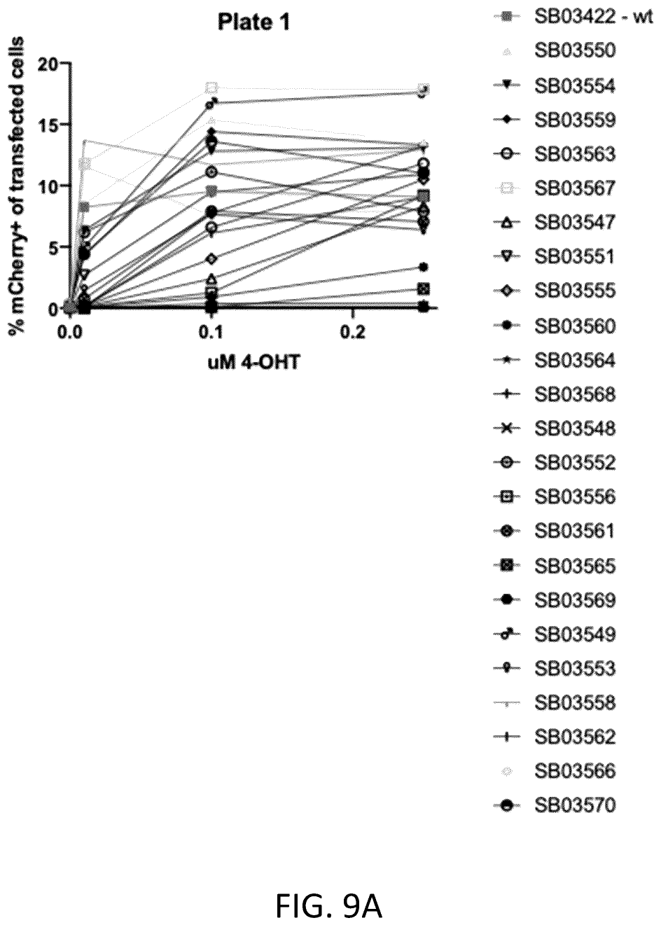

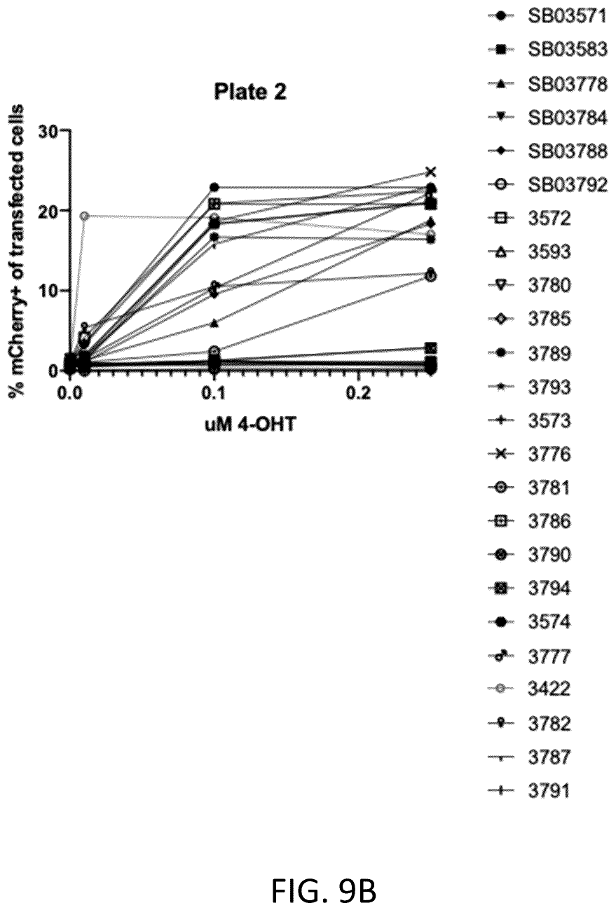

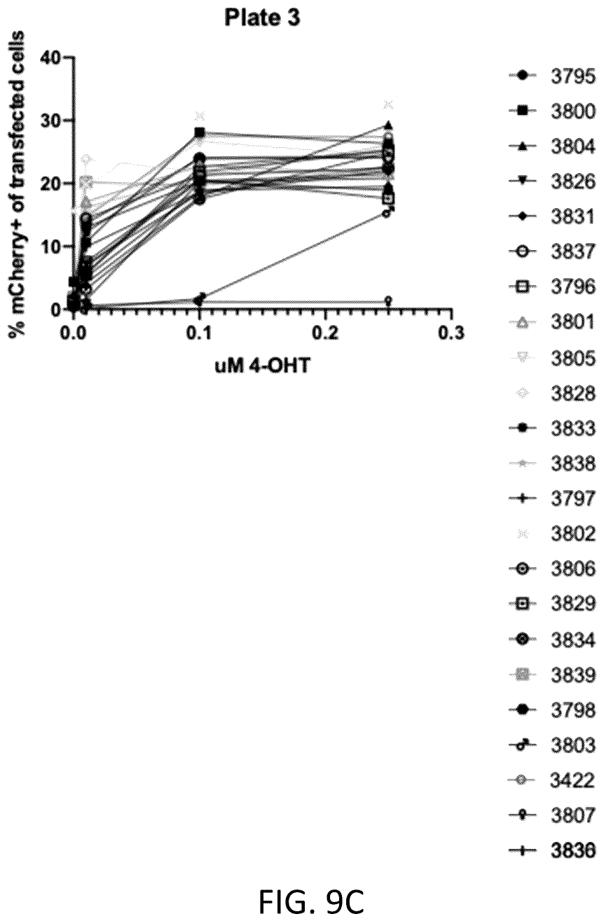

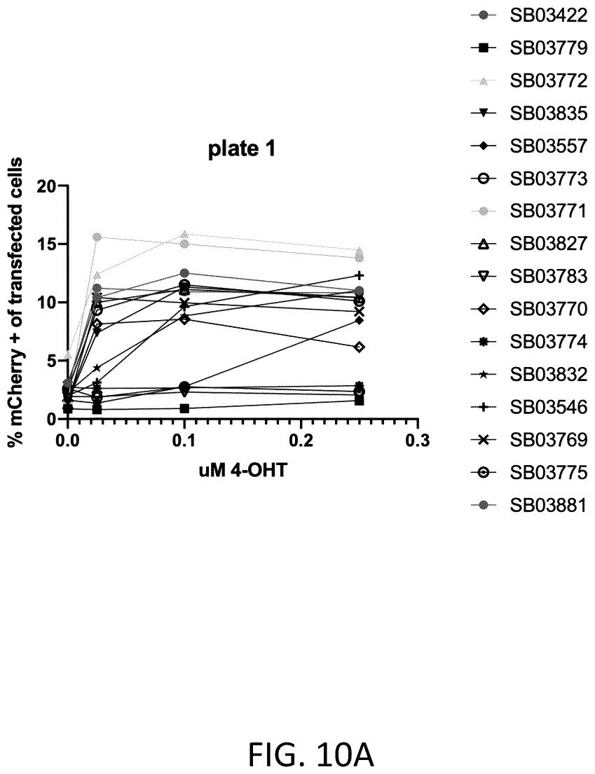

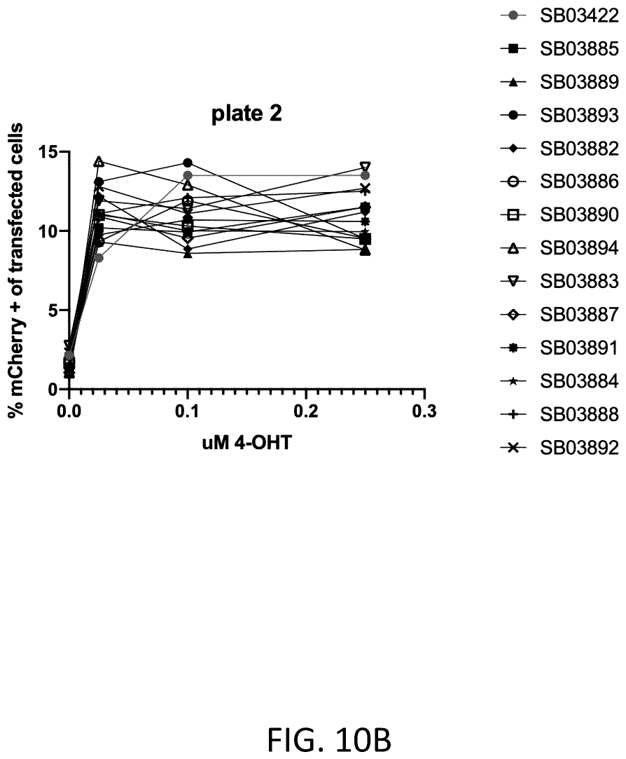

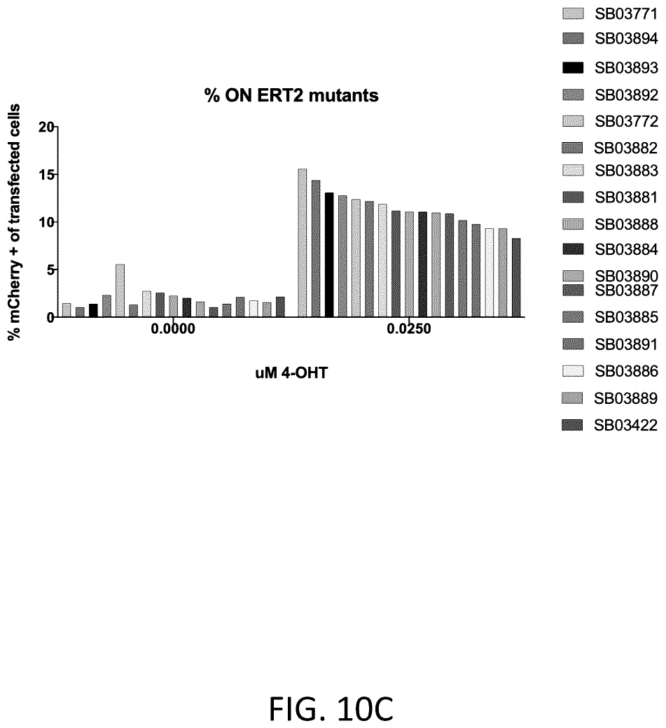

As shown in FIGS. 8A-8C, FIGS. 9A-9C, and FIGS. 10A-10C, the transfection screens identified mutants with improved induction of mCherry expression as compared to an ERT2 (SEQ ID NO: 3). In the first transfection screen, improved expression induction was observed for seventeen of the mutants: L354I (SB03498), L391V (SB03505), Q414E (SB03383), L409V (SB03375), S463P (SB03393), L384M (SB03377), L354I+L384M (SB03511), N413D (SB03381), M517A (SB03379), G344M (SB03372), I386V (SB03373), N407D (SB03380), C417S (SB03371), R352K (SB03384), Y537S (SB03389), M388F (SB03579), and G521A (SB03587), with the greatest improvement of expression induced by the following seven mutants: L354I (SB03498), L391V (SB03505), Q414E (SB03383), L409V (SB03375), and S463P (SB03393), (FIGS. 8A-8C). In the second transfection screen, improved expression induction was observed for ten of the mutants: I424L (SB03558), M421L (SB03566), M421F (SB03567), T347E (SB03801), L536M (SB03828), Y537E (SB03839), T347I (SB03802), and T347M (SB03805), V418I (SB03550), and V533W (SB03838), with strong improvement of expression induced by the following six mutants: I424L (SB03558), M421L (SB03566), M421F (SB03567), T347E (SB03801), L536M (SB03828), and Y537E (SB03839) (FIGS. 9A-9C). In the third transfection screen (FIGS. 10A-10C), improved expression induction was observed for the mutants shown in Table 13. Table 13.

TABLE 13

| Construct | Description |
|---|---|
| SB03771 | H524L |
| SB03894 | L354I + Q414E |
| SB03893 | L354I + L384M + Q414E |
| SB03892 | L391V + Q414E |
| SB03772 | H524F |
| SB03882 | L409V + L391V |
| SB03883 | L409V + L354I + L384M |
| SB03881 | L409V + S463P |
| SB03888 | S463P + L354I |
| SB03884 | L409V + L354I |
| SB03890 | L391V + L354I + L384M |
| SB03887 | S463P + L354I + L384M |
| SB03885 | L409V + Q414E |
| SB03891 | L391V + L354I |
| SB03886 | S463P + L391V |
| SB03889 | S463P + Q414E |

### Example 3: Mutants with Increased Drug Sensitivity Identified by Transduction Screen

ERT2 mutants were analyzed by three transduction screens for the ability to induce reporter expression in response to 4-OHT. The mutants L354I+L384M (identified in the first transfection screen) was included in all three transduction screens. Lentiviral vectors were cloned encoding the ERT2 mutants that demonstrated improved response to 4-OHT in the transfection screen from Example 2. The reporter cell line as described in Example 2 was transduced with lentiviruses encoding the ERT2 mutants, and the ability of the mutants to induce mCherry expression in response to a variety of 4-OHT concentrations was assessed.

### Materials and Methods

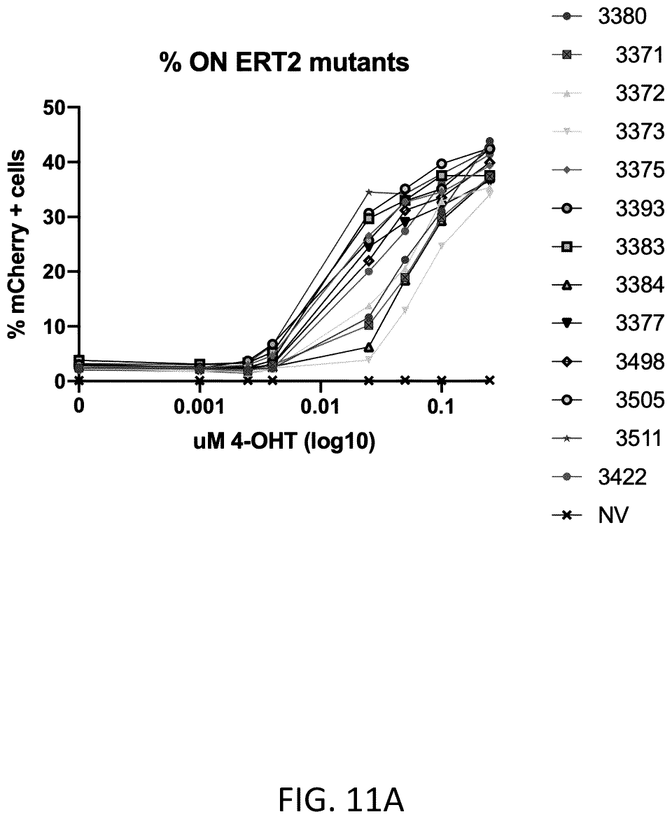

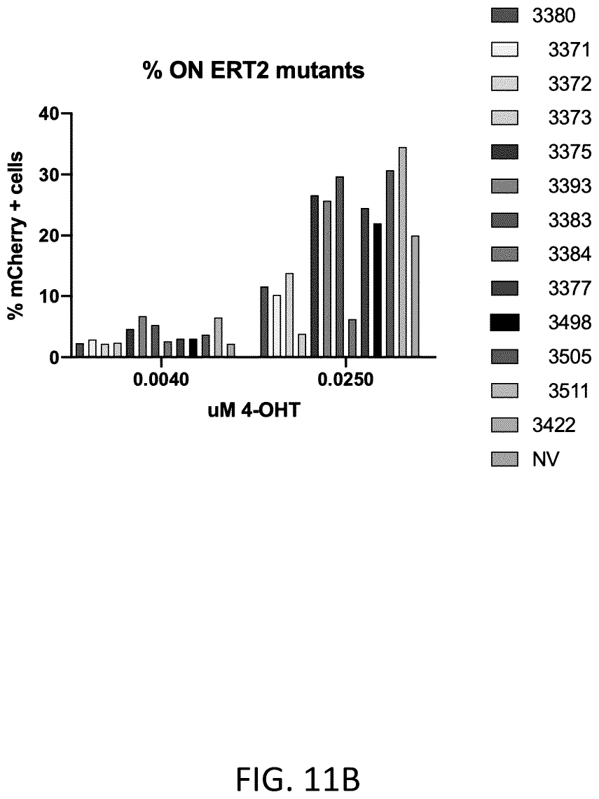

For the transduction screens, on day 1, reporter cells were plated at 2e5 cells/well in a 12 well plate. On day 2, cells were transduced with lentivirus encoding lead ERT mutants from the transfection screen. On days 3 and 4, cells were passaged to maintain <90% confluency on the plate. On day 5, cells were seeded into 96 well plates and treated with 0, 0.001, 0.0025, 0.004, 0.025, 0.05, 0.1, or 0.25 uM 4-OHT. On day 8, media was removed and cells were trypsinized and then resuspended in FACS buffer plus Sytox Red (fluoresces in APC channel) viability dye. Cells were run on a flow cytometer and gated by FSC/SSC for cells, FSC/Sytox Red—for live cells, the percent of mCherry positive cells at each drug concentration was plotted and compared to wildtype ERT2 to find more sensitive mutants (FIG. 11A). The percent of mCherry positive cells at two of the drug concentrations, 0.004 and 0.025 uM 4-OHT, are also shown in a bar graph (FIG. 11B).

### Results

As shown in FIGS. 11A and 11B, the first transduction screen confirmed improved 4-OHT response for several mutants identified as having improved 4-OHT binding in a transfection screen from Example 2. In particular, the mutants L354I, L391V, Q414E, L409V, S463P, L384M, and L354I+L384M all demonstrated an improved 4-OHT response as compared to a wild-type ERT2 (construct 3422, SEQ ID NO: 3). ERT2 mutants demonstrating improved 4-OHT binding in the first transduction screen are shown in Table 14.

TABLE 14

| Construct | Description |
|---|---|
| SB03498 | L354I |
| SB03505 | L391V |
| SB03383 | Q414E |
| SB03375 | L409V |
| SB03393 | S463P |
| SB03377 | L384M |
| SB03511 | L354I + L384M |

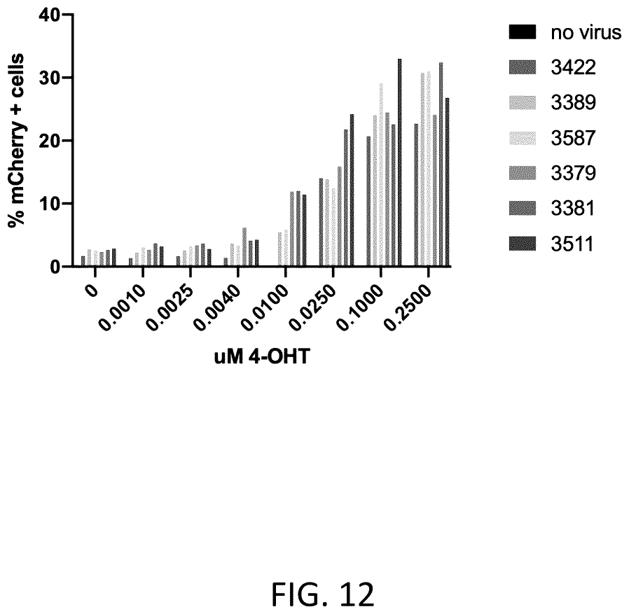

As shown in FIG. 12, the second transduction screen confirmed improved 4-OHT response for mutants identified in a transfection screen from Example 2. In particular, the mutants M517A, and N413D, and L354I+L384M demonstrated an improved 4-OHT response as compared to wild-type ERT2 (construct 3422). Notably, the improved 4-OHT response of the L354I+L384M mutant was confirmed in both the first and the second transduction screens. ERT2 mutants demonstrating improved 4-OHT binding in the second transduction screen are shown in Table 15.

TABLE 15

| Construct | Description |
|---|---|
| SB03379 | M517A |
| SB03381 | N413D |
| SB03511 | L354I + L384M |

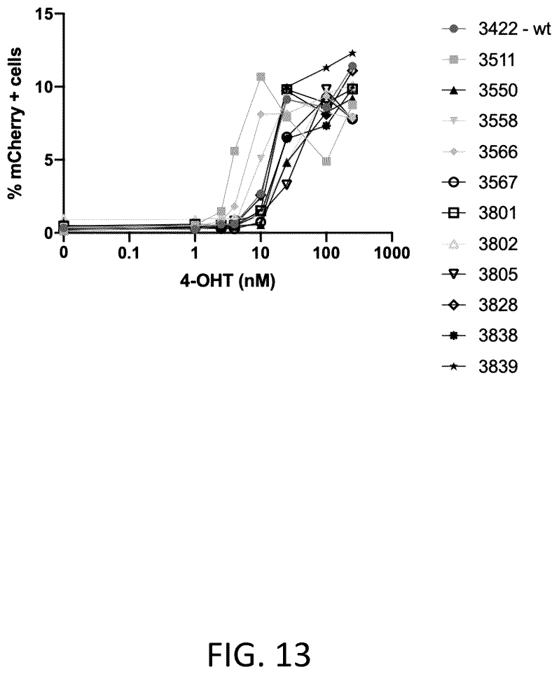

As shown in FIG. 13, the third transduction screen confirmed improved 4-OHT response for mutants identified in a transfection screen from Example 2. In particular, the mutants I524L, M421L, and L354I+L384M demonstrated an improved 4-OHT response as compared to wild-type ERT2 (construct 3422). ERT2 mutants demonstrating improved 4-OHT binding in the third transduction screen are shown in Table 16.

TABLE 16

| Construct | Description |
|---|---|
| SB03558 | I524L |
| SB03566 | M421L |
| SB03511 | L354I + L384M |

### Example 4: ERT2 Mutant Library Screen for Developing Endoxifen ON Switches

### Materials and Methods

### ERT2 Mutant Combinatorial Library Screen

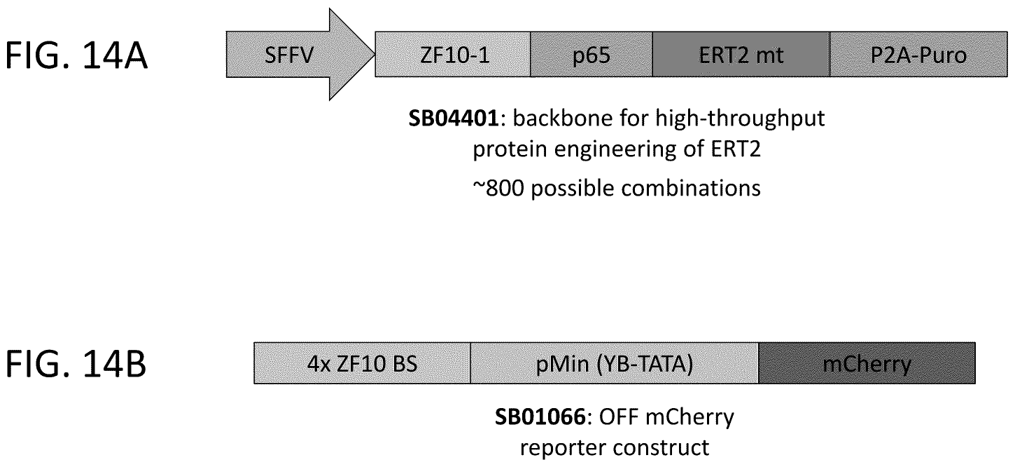

HEK293T cells were transduced with SB04401, a combinatorial ERT2 mutant library (FIG. 14A) comprised of −800 unique ER-LBD variants, each of which are a unique combination of the mutants given in Table 18 along with rationale for their inclusion. To generate a homogenous cell line where each cell had a unique ER-LBD variant per cell, viral integration of transduced HEK293T cells were quantified by copy number assay. A cell line with an average viral integration of <1 copy of the ERT2 mutant library per cell was identified and subjected to puromycin selection. The selected cell line was then transduced with a SB01066 mCherry reporter (FIG. 14B). Transduced cells were then tested for sensitivity to endoxifen and 4-OHT via the mCherry reporter which will express if an ER-LBD variant is sensitive to tested concentrations as low as about 0.1 nM up to about 1 uM. Cells expressing mCherry and therefor responsive to treatment of endoxifen were sorted followed by isolation of genomic DNA from said sorted cells. Variants were identified from the isolated genomic DNA by PCR amplification of the ERT2 coding sequence followed by insertion of the PCR product into per4 TOPO vector (Life Technologies). Colonies obtained were then submitted for Sanger Sequencing. Identified mutants were cloned into constructs, e.g., SB06136-SB06153 (ZF10-105 ERT2 mutant).

TABLE 18

| Mutation | Rationale |
|---|---|
| L391V | Set 2: improve the affinity of 4-OHT/endoxifen to ERT2, single point mutation in the |
| | ligand binding pocket |
| L409V | Set 1: improve the affinity of 4-OHT, mutation in the ligand binding pocket, selected |
| | based on a substitution present in an ER homologue, increased binding to estradiol |
| Q414E | Set 1: improve the affinity of 4-OHT, mutation in the ligand binding pocket, selected |
| | based on a substitution present in an ER homologue |
| N413D | Set 1: improve the affinity of 4-OHT, mutation in the ligand binding pocket, selected |
| | based on a substitution present in an ER homologue |
| S463P | Negative control, clinical mutation |
| M421L | Set 1: improve the affinity of 4-OHT, mutation in the ligand binding pocket, selected |
| | based on a substitution present in an ER homologue |
| M517A | Set 1: mutation in the ligand binding pocket, selected based on a substitution present in |
| | an ER homologue, binds to Estradiol with^{~}1 kcal/mol (in comparison 4-OHT binding |
| | is disfavoured by^{~}1.5 kcal/mol) |
| L354I | Set 2: improve the affinity of 4-OHT/endoxifen to ERT2, single point mutation in the |
| | ligand binding pocket |
| L384M | Set 1: improve the affinity of 4-OHT, mutation in the ligand binding pocket, selected |
| | based on a substitution present in an ER homologue |
| H524L | Set 5: rational optimization of ERT2 to improve its binding to 4-OHT and Endoxifen, |
| | focused on MD simulations in the ligand binding pocket |

ERT2 Mutant Validation in U87MG with mCherry Reporter

About 16 hours before transduction 100k U87MG:1066 cells were seeded into 16 wells in 12 well plate format. During transduction, cells in each well were transduced with 100k pg of virus of ERT2 variant constructs. After transduction, cells were split in to 50k cells per well in a 24 well plate format, and treated with drug-free media or a range of endoxifen or 4-OHT drug conditions. About two days after treatment of cells with endoxifen or 4-OHT, cells were collected and mCherry reporter expression was quantified by flow cytometry.

### Results

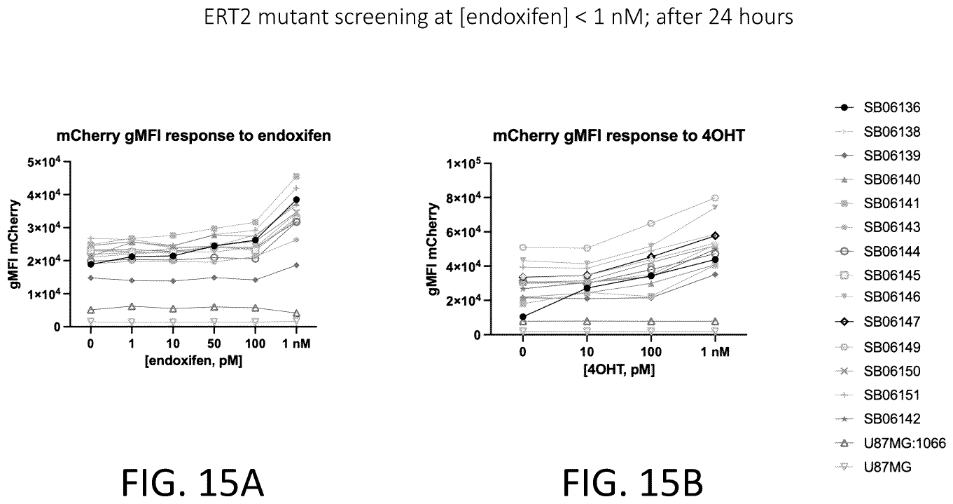

Screening of the ERT2 mutant library identified a subset of ERT2 variants that were sensitive to endoxifen at 1 nM. Among this subset, 15 variants (Table 19) were validated in U87MG cells for their ability to activate an mCherry reporter (SB01066; FIG. 14B) at a range of concentrations of 0 pM, 10 pM, 50 pM, 100 pM, and 1 nM of endoxifen (FIG. 15A) and 4-OHT (FIG. 15B). Expression of mCherry was quantified about 24 hours after treatment with endoxifen or 4-OHT. As demonstrated, all 15 variants were sensitive to endoxifen and 4-OHT at concentrations of about 1 nM or less, whereas negative controls U87MG and U87MG:1066 negative controls showed no sensitivity.

TABLE 19

| Construct | Description |
|---|---|
| SB06136 | ZF10-1_p65_ERT2_mutant 81 |
| SB06138 | ZF10-1_p65_ERT2_mutant 93 |
| SB06139 | ZF10-1_p65_ERT2_mutant 86 |
| SB06140 | ZF10-1_p65_ERT2_mutant 95 |
| SB06141 | ZF10-1_p65_ERT2_mutant 88 |
| SB06142 | ZF10-1_p65_ERT2_mutant 77 |
| SB06143 | ZF10-1_p65_ERT2_mutant 49 |
| SB06144 | ZF10-1_p65_ERT2_mutant 58 |
| SB06145 | ZF10-1_p65_ERT2_mutant 62 |
| SB06146 | ZF10-1_p65_ERT2_mutant 63 |
| SB06147 | ZF10-1_p65_ERT2_mutant 55 |
| SB06149 | ZF10-1_p65_ERT2_mutant 41 |
| SB06150 | ZF10-1_p65_ERT2_mutant 43 |
| SB06151 | ZF10-1_p65_ERT2_mutant 46 |
| SB06152 | ZF10-1_p65_ERT2_mutant 40 |

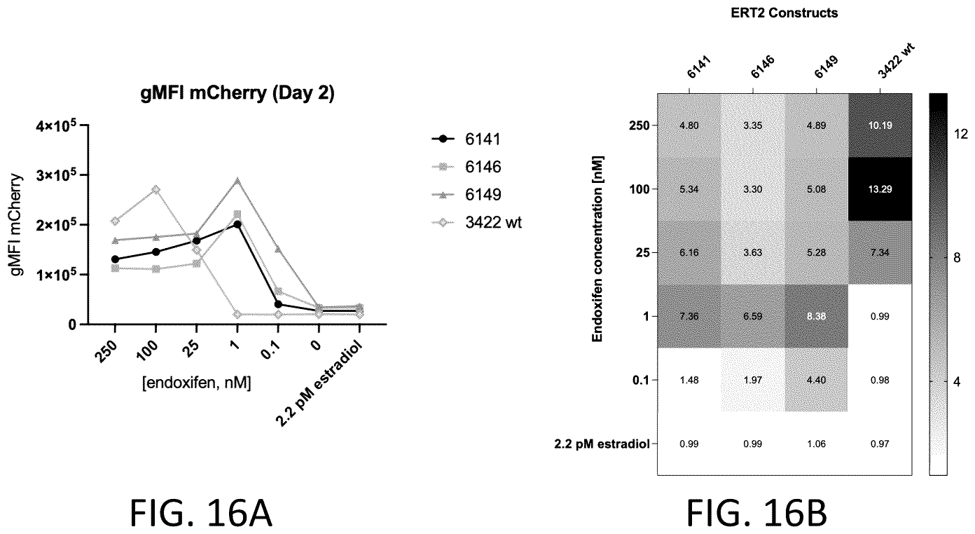

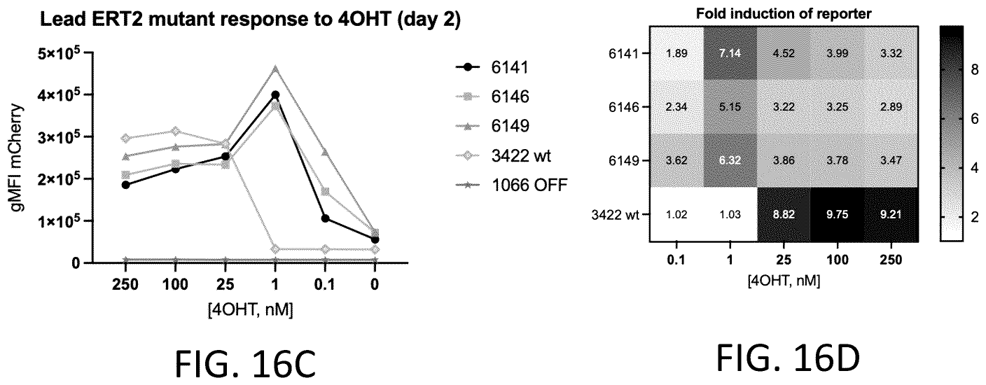

Select ERT2 variants (Table 20) from the previous screens were further tested for sensitivity to endoxifen and 4-OHT, compared to wild-type ERT2. Tests showed that activation of wild-type ERT2 begins at about 25 nM endoxifen and at about 25 nM 4-OHT, while three ERT2 variants tested activate mCherry expression at 1 nM and 0.1 nM endoxifen and at 1 nM and 0.1 nM 4-OHT (FIG. 16A-16D). Exemplary heat maps show fold activation of mCherry expression of constructs tested at various concentrations, including 2.2 pM estradiol (FIG. 16B). ERT2 is ER-alpha mutated to be insensitive to estradiol, the lead ERT2 variants were confirmed to continue to be insensitive to biologically observed concentrations of estradiol. Fold activation was calculated by gMFI mCherry levels of each test condition divided by gMFI mCherry levels of U87MG cells transduced with the reporter (SB01066).

TABLE 20

| Construct | Description |
|---|---|
| SB06141 | ZF10-1_p65_ERT2_mutant 88 |
| SB06146 | ZF10-1_p65_ERT2_mutant 63 |
| SB06149 | ZF10-1_p65_ERT2_mutant 41 |
| SB03422 | ZF10-1_p65_ERT2 wild-type |
| SB01066 | pMinYBTATA: mCherry reporter |

### Example 5: ERT2 Mutant Validation in NK Cells

### Materials and Methods

Validation ERT2 Mutants with mCherry Reporter in NK Cells

After 10 days of feeder cell activation, NK cells were co-transduced with ERT2 mutant virus and reporter SB01066 virus (Experimental Set-up 1). On Day 2 after transduction, transduced NK cells were treated with endoxifen or 4-OHT at a range of concentrations of 0 nM, 0.01 nM, 0.1 nM, 1 nM, and 10 nM. On Day 4, cells were checked for mCherry expression by flow cytometry.

Experimental Set-Up 1

Validation ERT2 Mutants with IL12 Reporter in NK Cells

After 10 days of feeder cell activation, NK cells were transduced with ERT2/IL12 vectors (Table 22; Experimental Set-up 2). On Day 2 after transduction, transduced NK cells were treated with endoxifen at a range of concentrations of 0 nM, 0.1 nM, 1 nM, 10 nM, 100 nM, and 1000 nM. On Day 4, cells were checked for IL-12 expression via Luminex.

Experimental Set-Up 2

### Results

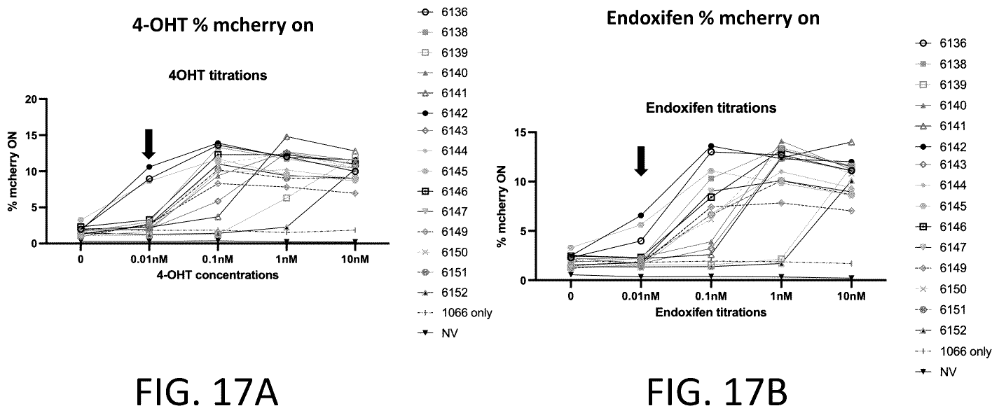

Select ERT2 variants (Table 21; FIG. 17A-17B) from the previous screens were further tested for sensitivity to endoxifen and 4-OHT in primary NK cells. Among the variants tested, SB 06142 (mutant 77; L354I/L391V/N413D/M421L/S463P/M517A/H524L), SB06136 (mutant 81; L384M/L391V/N413D/M421L/S463P/H524L), and SB06145 (mutant 62; L409V/N413D/Q414E/M421L/S463P/H524L) were sensitive to both endoxifen and 4-OHT at concentrations of 0.1 nM (FIG. 17A-17B).

TABLE 21

| Construct | Description |
|---|---|
| SB06136 | ZF10-1_p65_ERT2_mutant 81 |
| SB06138 | ZF10-1_p65_ERT2_mutant 93 |
| SB06139 | ZF10-1_p65_ERT2_mutant 86 |
| SB06140 | ZF10-1_p65_ERT2_mutant 95 |
| SB06141 | ZF10-1_p65_ERT2_mutant 88 |
| SB06142 | ZF10-1_p65_ERT2_mutant 77 |
| SB06143 | ZF10-1_p65_ERT2_mutant 49 |
| SB06144 | ZF10-1_p65_ERT2_mutant 58 |
| SB06145 | ZF10-1_p65_ERT2_mutant 62 |
| SB06146 | ZF10-1_p65_ERT2_mutant 63 |
| SB06147 | ZF10-1_p65_ERT2_mutant 55 |
| SB06149 | ZF10-1_p65_ERT2_mutant 41 |
| SB06150 | ZF10-1_p65_ERT2_mutant 43 |
| SB06151 | ZF10-1_p65_ERT2_mutant 46 |
| SB06152 | ZF10-1_p65_ERT2_mutant 40 |

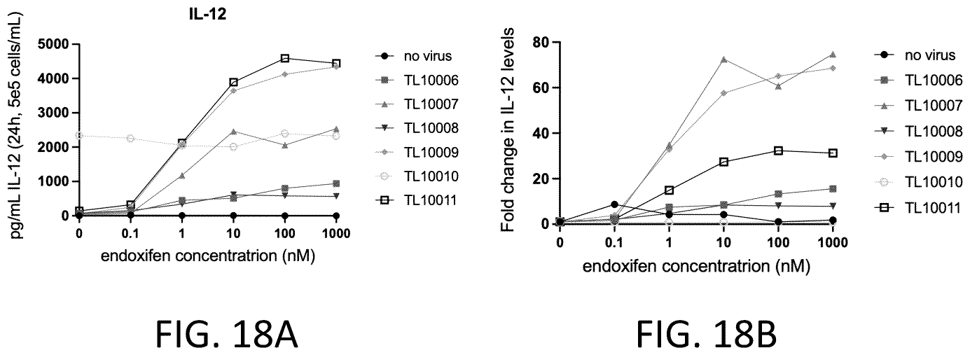

To demonstrate delivery of a therapeutic polypeptide, ERT2/IL-12 vectors were constructed as shown in Table 22, and tested for sensitivity to endoxifen in primary NK cells. Testing of ERT2/IL-12 vectors in NK cells showed that TL10009, with ERT2-L354I/L391V/N413D/S463P/H524L from SB06142 and crIL12 CD16 CS, shows best induction and fold change in IL-12 (FIG. 18A-18B).

TABLE 22

| | | Inducible | | | | | |
|---|---|---|---|---|---|---|---|
| | | IL-12 | | | | | |
| | | (driven by | | | ZF DNA | | |
| | | 4x ZF5BS | | Promoter: | Binding | | ERT2 |
| TL# | SB# | YBTATA) | Insulator | ZF | Domain | Activator | mutant |
| TL10006 | SB07123 | ZFN_YB- | A2 | SFFV: 5-7 | ZF5-7 | P65 | Mutant |
| | | TATA-sIL- | | | | | 81 |
| | | 12 1x | | | | | |
| | | AUSLDE | | | | | |
| TL10007 | SB07127 | ZFN_YB- | A2 | SV40: 5-7 | ZF5-7 | P65 | Mutant |
| | | TATA | | | | | 81 |
| | | IL12_CD16 | | | | | |
| | | TACE | | | | | |
| | | cleavage | | | | | |
| | | site_B7-1 | | | | | |
| | | TM | | | | | |
| TL10008 | SB07129 | ZFN_YB- | A2 | SFFV: 5-7 | ZF5-7 | P65 | Mutant |
| | | TATA-sIL- | | | | | 77 |
| | | 12 1x | | | | | |
| | | AUSLDE | | | | | |
| TL10009 | SB07133 | ZFN_YB- | A2 | SV40: 5-7 | ZF5-7 | P65 | Mutant |
| | | TATA | | | | | 77 |
| | | IL12_CD16 | | | | | |
| | | TACE | | | | | |
| | | cleavage | | | | | |
| | | site_B7-1 | | | | | |
| | | TM | | | | | |
| TL10010 | SB07135 | ZFN_YB- | A2 | SFFV: 5-7 | ZF5-7 | P65 | Mutant |
| | | TATA-sIL- | | | | | 62 |
| | | 12 1x | | | | | |
| | | AUSLDE | | | | | |
| TL10011 | SB07139 | ZFN_YB- | A2 | SV40: 5-7 | ZF5-7 | P65 | Mutant |
| | | TATA | | | | | 62 |
| | | IL12_CD16 | | | | | |
| | | TACE | | | | | |
| | | cleavage | | | | | |
| | | site_B7-1 | | | | | |
| | | TM | | | | | |

TABLE 17
Sequences

| | SEQ | |
|---|---|---|
| | ID | |
| Name | NO | Sequence |
| Estrogen | 1 | MTMTLHTKASGMALLHQIQGNELEPLNRPQLKIPLERPLGEVYL |
| Receptor | | DSSKPAVYNYPEGAAYEFNAAAAANAQVYGQTGLPYGPGSEAA |
| (Amino | | AFGSNGLGGFPPLNSVSPSPLMLLHPPPQLSPFLQPHGQQVPYYLE |
| Acid | | NEPSGYTVREAGPPAFYRPNSDNRRQGGRERLASTNDKGSMAM |
| Sequence) | | ESAKETRYCAVCNDYASGYHYGVWSCEGCKAFFKRSIQGHNDY |
| | | MCPATNQCTIDKNRRKSCQACRLRKCYEVGMMKGGIRKDRRGG |
| | | RMLKHKRQRDDGEGRGEVGSAGDMRAANLWPSPLMIKRSKKN |
| | | SLALSLTADQMVSALLDAEPPILYSEYDPTRPFSEASMMGLLTNL |
| | | ADRELVHMINWAKRVPGFVDLTLHDQVHLLECAWLEILMIGLV |
| | | WRSMEHPGKLLFAPNLLLDRNQGKCVEGMVEIFDMLLATSSRFR |
| | | MMNLQGEEFVCLKSIILLNSGVYTFLSSTLKSLEEKDHIHRVLDKI |
| | | TDTLIHLMAKAGLTLQQQHQRLAQLLLILSHIRHMSNKGMEHLY |
| | | SMKCKNVVPLYDLLLEMLDAHRLHAPTSRGGASVEETDQSHLA |
| | | TAGSTSSHSLQKYYITGEAEGFPATV |
| Example | 2 | SAGDMRAANLWPSPLMIKRSKKNSLALSLTADQMVSALLDAEPPILY |
| ERT2 | | SEYDPTRPFSEASMMGLLTNLADRELVHMINWAKRVPGFVDLTLHD |
| | | QVHLLECAWLEILMIGLVWRSMEHPVKLLFAPNLLLDRNQGKCVEG |
| | | MVEIFDMLLATSSRFRMMNLQGEEFVCLKSIILLNSGVYTFLSSTLKSL |
| | | EEKDHIHRVLDKITDTLIHLMAKAGLTLQQQHQRLAQLLLILSHIRHM |
| | | SNKGMEHLYSMKCKNVVPLYDLLLEAADAHRLHAPTSRGGASVEET |
| | | DQSHLATAGSTSSHSLQKYYITGEAEGFPATV |
| Example | 3 | SAGDMRAANLWPSPLMIKRSKKNSLALSLTADQMVSALLDAEPPILY |
| ERT2 | | SEYDPTRPFSEASMMGLLTNLADRELVHMINWAKRVPGFVDLTLHD |
| | | QVHLLECAWLEILMIGLVWRSMEHPVKLLFAPNLLLDRNQGKCVEG |
| | | MVEIFDMLLATSSRFRMMNLQGEEFVCLKSIILLNSGVYTFLSSTLKSL |
| | | EEKDHIHRVLDKITDTLIHLMAKAGLTLQQQHQRLAQLLLILSHIRHM |
| | | SNKGMEHLYSMKCKNVVPLYDLLLEAADAHRLHAPTSRGGASVEET |
| | | DQSHLATAGSTSSHSLQKYYITGEAEGFPATA |
| Peptide | 4 | GGGGSGGGGSGGGGSVDGF |
| Linker | | |
| Peptide | 5 | ASGGGGSAS |
| Linker | | |
| Zinc | 6 | SRPGERPFQCRICMRNFSRRHGLDRHTRTHTGEKPFQCRICMRNFSDH |
| Finger | | SSLKRHLRTHTGSQKPFQCRICMRNFSVRHNLTRHLRTHTGEKPFQCR |
| Protein | | ICMRNFSDHSNLSRHLKTHTGSQKPFQCRICMRNFSQRSSLVRHLRTH |
| Domain | | TGEKPFQCRICMRNFSESGHLKRHLRTHLRGS |
| ZF10-1 | | |
| minimal | 7 | AGAGGGTATATAATGGAAGCTCGACTTCCAG |
| promoter; | | |
| minP | | |
| NFKB | 8 | GGGAATTTCCGGGGACTTTCCGGGAATTTCCGGGGACTTTCCGGGA |
| response | | ATTTCC |
| element | | |
| protein | | |
| promoter; | | |
| 5x NFKB- | | |
| RE | | |
| CREB | 9 | CACCAGACAGTGACGTCAGCTGCCAGATCCCATGGCCGTCATACT |
| response | | GTGACGTCTTTCAGACACCCCATTGACGTCAATGGGAGAA |
| element | | |
| protein | | |
| promoter; | | |
| 4x CRE | | |
| NFAT | 10 | GGAGGAAAAACTGTTTCATACAGAAGGCGTGGAGGAAAAACTGTT |
| response | | TCATACAGAAGGCGTGGAGGAAAAACTGTTTCATACAGAAGGCGT |
| element | | |
| protein | | |
| promoter; | | |
| 3x NFAT | | |
| binding | | |
| sites | | |
| SRF | 11 | AGGATGTCCATATTAGGACATCTAGGATGTCCATATTAGGACATCT |
| response | | AGGATGTCCATATTAGGACATCTAGGATGTCCATATTAGGACATCT |
| element | | AGGATGTCCATATTAGGACATCT |
| protein | | |
| promoter; | | |
| 5x SRE | | |
| SRF | 12 | AGTATGTCCATATTAGGACATCTACCATGTCCATATTAGGACATCT |
| response | | ACTATGTCCATATTAGGACATCTTGTATGTCCATATTAGGACATCT |
| element | | AAAATGTCCATATTAGGACATCT |
| protein | | |
| promoter | | |
| 2; 5x SRF- | | |
| RE | | |
| AP1 | 13 | TGAGTCAGTGACTCAGTGAGTCAGTGACTCAGTGAGTCAGTGACTC |
| response | | AG |
| element | | |
| protein | | |
| promoter; | | |
| 6x AP1-RE | | |
| TCF-LEF | 14 | AGATCAAAGGGTTTAAGATCAAAGGGCTTAAGATCAAAGGGTATA |
| response | | AGATCAAAGGGCCTAAGATCAAAGGGACTAAGATCAAAGGGTTTA |
| element | | AGATCAAAGGGCTTAAGATCAAAGGGCCTA |
| promoter; | | |
| 8x TCF- | | |
| LEF-RE | | |
| SBEx4 | 15 | GTCTAGACGTCTAGACGTCTAGACGTCTAGAC |
| SMAD2/3- | 16 | CAGACACAGACACAGACACAGACA |
| 1 | | |
| CAGACA | | |
| x4 | | |
| STAT3 | 17 | GGATCCGGTACTCGAGATCTGCGATCTAAGTAAGCTTGGCATTCCG |
| binding | | GTACTGTTGGTAAAGCCAC |
| site | | |
| CMV | 18 | GTTGACATTGATTATTGACTAGTTATTAATAGTAATCAATTACGGG |
| | | GTCATTAGTTCATAGCCCATATATGGAGTTCCGCGTTACATAACTT |
| | | ACGGTAAATGGCCCGCCTGGCTGACCGCCCAACGACCCCCGCCCA |
| | | TTGACGTCAATAATGACGTATGTTCCCATAGTAACGCCAATAGGGA |
| | | CTTTCCATTGACGTCAATGGGTGGAGTATTTACGGTAAACTGCCCA |
| | | CTTGGCAGTACATCAAGTGTATCATATGCCAAGTACGCCCCCTATT |
| | | GACGTCAATGACGGTAAATGGCCCGCCTGGCATTATGCCCAGTAC |
| | | ATGACCTTATGGGACTTTCCTACTTGGCAGTACATCTACGTATTAG |
| | | TCATCGCTATTACCATGGTGATGCGGTTTTGGCAGTACATCAATGG |
| | | GCGTGGATAGCGGTTTGACTCACGGGGATTTCCAAGTCTCCACCCC |
| | | ATTGACGTCAATGGGAGTTTGTTTTGGCACCAAAATCAACGGGACT |
| | | TTCCAAAATGTCGTAACAACTCCGCCCCATTGACGCAAATGGGCG |
| | | GTAGGCGTGTACGGTGGGAGGTCTATATAAGCAGAGCTC |
| EF1a | 19 | GGCTCCGGTGCCCGTCAGTGGGCAGAGCGCACATCGCCCACAGTC |
| | | CCCGAGAAGTTGGGGGGAGGGGTCGGCAATTGAACCGGTGCCTAG |
| | | AGAAGGTGGCGCGGGGTAAACTGGGAAAGTGATGCCGTGTACTGG |
| | | CTCCGCCTTTTTCCCGAGGGTGGGGGAGAACCGTATATAAGTGCAG |
| | | TAGTCGCCGTGAACGTTCTTTTTCGCAACGGGTTTGCCGCCAGAAC |
| | | ACAGGTAAGTGCCGTGTGTGGTTCCCGCGGGCCTGGCCTCTTTACG |
| | | GGTTATGGCCCTTGCGTGCCTTGAATTACTTCCACCTGGCTGCAGT |
| | | ACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGT |
| | | TGAGGCCTGGCCTGGGCGCTGGGGCCGCCGCGTGCGAATCTGGTG |
| | | GCACCTTCGCGCCTGTCTCGCTGCTTTCGATAAGTCTCTAGCCATTT |
| | | AAAATTTTTGATGACCTGCTGCGACGCTTTTTTTCTGGCAAGATAG |
| | | TCTTGTAAATGCGGGCCAAGATCTGCACACTGGTATTTCGGTTTTT |
| | | GGGGCCGCGGGCGGCGACGGGGCCCGTGCGTCCCAGCGCACATGT |
| | | TCGGCGAGGCGGGGCCTGCGAGCGCGACCACCGAGAATCGGACGG |
| | | GGGTAGTCTCAAGCTGGCCGGCCTGCTCTGGTGCCTGTCCTCGCGC |
| | | CGCCGTGTATCGCCCCGCCCCGGGCGGCAAGGCTGGCCCGGTCGG |
| | | CACCAGTTGCGTGAGCGGAAAGATGGCCGCTTCCCGGTCCTGCTGC |
| | | AGGGAGCTCAAAATGGAGGACGCGGCGCTCGGGAGAGCGGGCGG |
| | | GTGAGTCACCCACACAAAGGAAAAGGGCCTTTCCGTCCTCAGCCG |
| | | TCGCTTCATGTGACTCCACGGAGTACCGGGCGCCGTCCAGGCACCT |
| | | CGATTAGTTCTCGAGCTTTTGGAGTACGTCGTCTTTAGGTTGGGGG |
| | | GAGGGGTTTTATGCGATGGAGTTTCCCCACACTGAGTGGGTGGAG |
| | | ACTGAAGTTAGGCCAGCTTGGCACTTGATGTAATTCTCCTTGGAAT |
| | | TTGCCCTTTTTGAGTTTGGATCTTGGTTCATTCTCAAGCCTCAGACA |
| | | GTGGTTCAAAGTTTTTTTCTTCCATTTCAGGTGTCGTGA |
| EFS | 20 | GGATCTGCGATCGCTCCGGTGCCCGTCAGTGGGCAGAGCGCACAT |
| | | CGCCCACAGTCCCCGAGAAGTTGGGGGGAGGGGTCGGCAATTGAA |
| | | CCGGTGCCTAGAGAAGGTGGCGCGGGGTAAACTGGGAAAGTGATG |
| | | TCGTGTACTGGCTCCGCCTTTTTCCCGAGGGTGGGGGAGAACCGTA |
| | | TATAAGTGCAGTAGTCGCCGTGAACGTTCTTTTTCGCAACGGGTTT |
| | | GCCGCCAGAACACAGCTGAAGCTTCGAGGGGCTCGCATCTCTCCTT |
| | | CACGCGCCCGCCGCCCTACCTGAGGCCGCCATCCACGCCGGTTGA |
| | | GTCGCGTTCTGCCGCCTCCCGCCTGTGGTGCCTCCTGAACTGCGTC |
| | | CGCCGTCTAGGTAAGTTTAAAGCTCAGGTCGAGACCGGGCCTTTGT |
| | | CCGGCGCTCCCTTGGAGCCTACCTAGACTCAGCCGGCTCTCCACGC |
| | | TTTGCCTGACCCTGCTTGCTCAACTCTACGTCTTTGTTTCGTTTTCT |
| | | GTTCTGCGCCGTTACAGATCCAAGCTGTGACCGGCGCCTAC |
| MND | 21 | TTTATTTAGTCTCCAGAAAAAGGGGGGAATGAAAGACCCCACCTG |
| | | TAGGTTTGGCAAGCTAGGATCAAGGTTAGGAACAGAGAGACAGCA |
| | | GAATATGGGCCAAACAGGATATCTGTGGTAAGCAGTTCCTGCCCC |
| | | GGCTCAGGGCCAAGAACAGTTGGAACAGCAGAATATGGGCCAAAC |
| | | AGGATATCTGTGGTAAGCAGTTCCTGCCCCGGCTCAGGGCCAAGA |
| | | ACAGATGGTCCCCAGATGCGGTCCCGCCCTCAGCAGTTTCTAGAGA |
| | | ACCATCAGATGTTTCCAGGGTGCCCCAAGGACCTGAAATGACCCT |
| | | GTGCCTTATTTGAACTAACCAATCAGTTCGCTTCTCGCTTCTGTTCG |
| | | CGCGCTTCTGCTCCCCGAGCTCAATAAAAGAGCCCA |
| PGK | 22 | GGGGTTGGGGTTGCGCCTTTTCCAAGGCAGCCCTGGGTTTGCGCAG |
| | | GGACGCGGCTGCTCTGGGCGTGGTTCCGGGAAACGCAGCGGCGCC |
| | | GACCCTGGGTCTCGCACATTCTTCACGTCCGTTCGCAGCGTCACCC |
| | | GGATCTTCGCCGCTACCCTTGTGGGCCCCCCGGCGACGCTTCCTGC |
| | | TCCGCCCCTAAGTCGGGAAGGTTCCTTGCGGTTCGCGGCGTGCCGG |
| | | ACGTGACAAACGGAAGCCGCACGTCTCACTAGTACCCTCGCAGAC |
| | | GGACAGCGCCAGGGAGCAATGGCAGCGCGCCGACCGCGATGGGCT |
| | | GTGGCCAATAGCGGCTGCTCAGCGGGGCGCGCCGAGAGCAGCGGC |
| | | CGGGAAGGGGCGGTGCGGGAGGCGGGGTGTGGGGCGGTAGTGTG |
| | | GGCCCTGTTCCTGCCCGCGCGGTGTTCCGCATTCTGCAAGCCTCCG |
| | | GAGCGCACGTCGGCAGTCGGCTCCCTCGTTGACCGAATCACCGAC |
| | | CTCTCTCCCCAG |
| SFFV | 23 | GTAACGCCATTTTGCAAGGCATGGAAAAATACCAAACCAAGAATA |
| | | GAGAAGTTCAGATCAAGGGCGGGTACATGAAAATAGCTAACGTTG |
| | | GGCCAAACAGGATATCTGCGGTGAGCAGTTTCGGCCCCGGCCCGG |
| | | GGCCAAGAACAGATGGTCACCGCAGTTTCGGCCCCGGCCCGAGGC |
| | | CAAGAACAGATGGTCCCCAGATATGGCCCAACCCTCAGCAGTTTCT |
| | | TAAGACCCATCAGATGTTTCCAGGCTCCCCCAAGGACCTGAAATG |
| | | ACCCTGCGCCTTATTTGAATTAACCAATCAGCCTGCTTCTCGCTTCT |
| | | GTTCGCGCGCTTCTGCTTCCCGAGCTCTATAAAAGAGCTCACAACC |
| | | CCTCACTCGGCGCGCCAGTCCTCCGACAGACTGAGTCGCCCGGG |
| SV40 | 24 | CTGTGGAATGTGTGTCAGTTAGGGTGTGGAAAGTCCCCAGGCTCCC |
| | | CAGCAGGCAGAAGTATGCAAAGCATGCATCTCAATTAGTCAGCAA |
| | | CCAGGTGTGGAAAGTCCCCAGGCTCCCCAGCAGGCAGAAGTATGC |
| | | AAAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCTGCCTCTGAGCTATTCCAGAAGTAGTGAGGAGGCTTTTTTGG |
| | | AGGCCTAGGCTTTTGCAAAAAGCT |
| UbC | 25 | GCGCCGGGTTTTGGCGCCTCCCGCGGGCGCCCCCCTCCTCACGGCG |
| | | AGCGCTGCCACGTCAGACGAAGGGCGCAGGAGCGTTCCTGATCCT |
| | | TCCGCCCGGACGCTCAGGACAGCGGCCCGCTGCTCATAAGACTCG |
| | | GCCTTAGAACCCCAGTATCAGCAGAAGGACATTTTAGGACGGGAC |
| | | TTGGGTGACTCTAGGGCACTGGTTTTCTTTCCAGAGAGCGGAACAG |
| | | GCGAGGAAAAGTAGTCCCTTCTCGGCGATTCTGCGGAGGGATCTC |
| | | CGTGGGGCGGTGAACGCCGATGATTATATAAGGACGCGCCGGGTG |
| | | TGGCACAGCTAGTTCCGTCGCAGCCGGGATTTGGGTCGCGGTTCTT |
| | | GTTTGTGGATCGCTGTGATCGTCACTTGGTGAGTTGCGGGCTGCTG |
| | | GGCTGGCCGGGGCTTTCGTGGCCGCCGGGCCGCTCGGTGGGACGG |
| | | AAGCGTGTGGAGAGACCGCCAAGGGCTGTAGTCTGGGTCCGCGAG |
| | | CAAGGTTGCCCTGAACTGGGGGTTGGGGGGAGCGCACAAAATGGC |
| | | GGCTGTTCCCGAGTCTTGAATGGAAGACGCTTGTAAGGCGGGCTGT |
| | | GAGGTCGTTGAAACAAGGTGGGGGGCATGGTGGGCGGCAAGAAC |
| | | CCAAGGTCTTGAGGCCTTCGCTAATGCGGGAAAGCTCTTATTCGGG |
| | | TGAGATGGGCTGGGGCACCATCTGGGGACCCTGACGTGAAGTTTG |
| | | TCACTGACTGGAGAACTCGGGTTTGTCGTCTGGTTGCGGGGGCGGC |
| | | AGTTATGCGGTGCCGTTGGGCAGTGCACCCGTACCTTTGGGAGCGC |
| | | GCGCCTCGTCGTGTCGTGACGTCACCCGTTCTGTTGGCTTATAATG |
| | | CAGGGTGGGGCCACCTGCCGGTAGGTGTGCGGTAGGCTTTTCTCCG |
| | | TCGCAGGACGCAGGGTTCGGGCCTAGGGTAGGCTCTCCTGAATCG |
| | | ACAGGCGCCGGACCTCTGGTGAGGGGAGGGATAAGTGAGGCGTCA |
| | | GTTTCTTTGGTCGGTTTTATGTACCTATCTTCTTAAGTAGCTGAAGC |
| | | TCCGGTTTTGAACTATGCGCTCGGGGTTGGCGAGTGTGTTTTGTGA |
| | | AGTTTTTTAGGCACCTTTTGAAATGTAATCATTTGGGTCAATATGT |
| | | AATTTTCAGTGTTAGACTAGTAAAGCTTCTGCAGGTCGACTCTAGA |
| | | AAATTGTCCGCTAAATTCTGGCCGTTTTTGGCTTTTTTGTTAGAC |
| hEF1aV1 | 26 | GGCTCCGGTGCCCGTCAGTGGGCAGAGCGCACATCGCCCACAGTC |
| | | CCCGAGAAGTTGGGGGGAGGGGTCGGCAATTGAACCGGTGCCTAG |
| | | AGAAGGTGGCGCGGGGTAAACTGGGAAAGTGATGTCGTGTACTGG |
| | | CTCCGCCTTTTTCCCGAGGGTGGGGGAGAACCGTATATAAGTGCAG |
| | | TAGTCGCCGTGAACGTTCTTTTTCGCAACGGGTTTGCCGCCAGAAC |
| | | ACAGGTAAGTGCCGTGTGTGGTTCCCGCGGGCCTGGCCTCTTTACG |
| | | GGTTATGGCCCTTGCGTGCCTTGAATTACTTCCACCTGGCTGCAGT |
| | | ACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGT |
| | | TGAGGCCTGGCCTGGGCGCTGGGGCCGCCGCGTGCGAATCTGGTG |
| | | GCACCTTCGCGCCTGTCTCGCTGCTTTCGATAAGTCTCTAGCCATTT |
| | | AAAATTTTTGATGACCTGCTGCGACGCTTTTTTTCTGGCAAGATAG |
| | | TCTTGTAAATGCGGGCCAAGATCTGCACACTGGTATTTCGGTTTTT |
| | | GGGGCCGCGGGCGGCGACGGGGCCCGTGCGTCCCAGCGCACATGT |
| | | TCGGCGAGGCGGGGCCTGCGAGCGCGGCCACCGAGAATCGGACGG |
| | | GGGTAGTCTCAAGCTGGCCGGCCTGCTCTGGTGCCTGGTCTCGCGC |
| | | CGCCGTGTATCGCCCCGCCCTGGGCGGCAAGGCTGGCCCGGTCGG |
| | | CACCAGTTGCGTGAGCGGAAAGATGGCCGCTTCCCGGCCCTGCTG |
| | | CAGGGAGCTCAAAATGGAGGACGCGGCGCTCGGGAGAGCGGGCG |
| | | GGTGAGTCACCCACACAAAGGAAAAGGGCCTTTCCGTCCTCAGCC |
| | | GTCGCTTCATGTGACTCCACGGAGTACCGGGCGCCGTCCAGGCACC |
| | | TCGATTAGTTCTCGAGCTTTTGGAGTACGTCGTCTTTAGGTTGGGG |
| | | GGAGGGGTTTTATGCGATGGAGTTTCCCCACACTGAGTGGGTGGA |
| | | GACTGAAGTTAGGCCAGCTTGGCACTTGATGTAATTCTCCTTGGAA |
| | | TTTGCCCTTTTTGAGTTTGGATCTTGGTTCATTCTCAAGCCTCAGAC |
| | | AGTGGTTCAAAGTTTTTTTCTTCCATTTCAGGTGTCGTGA |
| hCAGG | 27 | ACTAGTTATTAATAGTAATCAATTACGGGGTCATTAGTTCATAGCC |
| | | CATATATGGAGTTCCGCGTTACATAACTTACGGTAAATGGCCCGCC |
| | | TGGCTGACCGCCCAACGACCCCCGCCCATTGACGTCAATAATGAC |
| | | GTATGTTCCCATAGTAACGCCAATAGGGACTTTCCATTGACGTCAA |
| | | TGGGTGGAGTATTTACGGTAAACTGCCCACTTGGCAGTACATCAAG |
| | | TGTATCATATGCCAAGTACGCCCCCTATTGACGTCAATGACGGTAA |
| | | ATGGCCCGCCTGGCATTATGCCCAGTACATGACCTTATGGGACTTT |
| | | CCTACTTGGCAGTACATCTACGTATTAGTCATCGCTATTACCATGG |
| | | TCGAGGTGAGCCCCACGTTCTGCTTCACTCTCCCCATCTCCCCCCCC |
| | | TCCCCACCCCCAATTTTGTATTTATTTATTTTTTAATTATTTTGTGCA |
| | | GCGATGGGGGCGGGGGGGGGGGGGGGGCGCGCGCCAGGCGGGGC |
| | | GGGGCGGGGCGAGGGGCGGGGGGGGCGAGGCGGAGAGGTGCGG |
| | | CGGCAGCCAATCAGAGCGGCGCGCTCCGAAAGTTTCCTTTTATGGC |
| | | GAGGCGGCGGCGGCGGCGGCCCTATAAAAAGCGAAGCGCGCGGC |
| | | GGGCGGGGAGTCGCTGCGACGCTGCCTTCGCCCCGTGCCCCGCTCC |
| | | GCCGCCGCCTCGCGCCGCCCGCCCCGGCTCTGACTGACCGCGTTAC |
| | | TCCCACAGGTGAGCGGGCGGGACGGCCCTTCTCCTCCGGGCTGTA |
| | | ATTAGCGCTTGGTTTAATGACGGCTTGTTTCTTTTCTGTGGCTGCGT |
| | | GAAAGCCTTGAGGGGCTCCGGGAGGGCCCTTTGTGCGGGGGGAGC |
| | | GGCTCGGGGGGTGCGTGCGTGTGTGTGTGCGTGGGGAGCGCCGCG |
| | | TGCGGCTCCGCGCTGCCCGGCGGCTGTGAGCGCTGCGGGCGCGGC |
| | | GCGGGGCTTTGTGCGCTCCGCAGTGTGCGCGAGGGGAGCGCGGCC |
| | | GGGGGCGGTGCCCCGCGGTGCGGGGGGGGCTGCGAGGGGAACAA |
| | | AGGCTGCGTGCGGGGTGTGTGCGTGGGGGGGTGAGCAGGGGGTGT |
| | | GGGCGCGTCGGTCGGGCTGCAACCCCCCCTGCACCCCCCTCCCCGA |
| | | GTTGCTGAGCACGGCCCGGCTTCGGGTGCGGGGCTCCGTACGGGG |
| | | CGTGGCGCGGGGCTCGCCGTGCCGGGCGGGGGGTGGCGGCAGGTG |
| | | GGGGTGCCGGGCGGGGGGGGGCCGCCTCGGGCCGGGGAGGGCTC |
| | | GGGGGAGGGGCGCGGCGGCCCCCGGAGCGCCGGCGGCTGTCGAG |
| | | GCGCGGCGAGCCGCAGCCATTGCCTTTTATGGTAATCGTGCGAGA |
| | | GGGCGCAGGGACTTCCTTTGTCCCAAATCTGTGCGGAGCCGAAATC |
| | | TGGGAGGCGCCGCCGCACCCCCTCTAGCGGGCGCGGGGCGAAGCG |
| | | GTGCGGCGCCGGCAGGAAGGAAATGGGCGGGGAGGGCCTTCGTGC |
| | | GTCGCCGCGCCGCCGTCCCCTTCTCCCTCTCCAGCCTCGGGGCTGT |
| | | CCGCGGGGGGACGGCTGCCTTCGGGGGGGACGGGGCAGGGCGGG |
| | | GTTCGGCTTCTGGCGTGTGACCGGCGGCTCTAGAGCCTCTGCTAAC |
| | | CATGTTCATGCCTTCTTCTTTTTCCTACAGCTCCTGGGCAACGTGCT |
| | | GGTTATTGTGCTGTCTCATCATTTTGGCAAAGAATTC |
| hEF1aV2 | 28 | GGGCAGAGCGCACATCGCCCACAGTCCCCGAGAAGTTGGGGGGAG |
| | | GGGTCGGCAATTGAACCGGTGCCTAGAGAAGGTGGCGCGGGGTAA |
| | | ACTGGGAAAGTGATGTCGTGTACTGGCTCCGCCTTTTTCCCGAGGG |
| | | TGGGGGAGAACCGTATATAAGTGCAGTAGTCGCCGTGAACGTTCT |
| | | TTTTCGCAACGGGTTTGCCGCCAGAACACAG |
| hACTb | 29 | CCACTAGTTCCATGTCCTTATATGGACTCATCTTTGCCTATTGCGAC |
| | | ACACACTCAATGAACACCTACTACGCGCTGCAAAGAGCCCCGCAG |
| | | GCCTGAGGTGCCCCCACCTCACCACTCTTCCTATTTTTGTGTAAAA |
| | | ATCCAGCTTCTTGTCACCACCTCCAAGGAGGGGGAGGAGGAGGAA |
| | | GGCAGGTTCCTCTAGGCTGAGCCGAATGCCCCTCTGTGGTCCCACG |
| | | CCACTGATCGCTGCATGCCCACCACCTGGGTACACACAGTCTGTGA |
| | | TTCCCGGAGCAGAACGGACCCTGCCCACCCGGTCTTGTGTGCTACT |
| | | CAGTGGACAGACCCAAGGCAAGAAAGGGTGACAAGGACAGGGTC |
| | | TTCCCAGGCTGGCTTTGAGTTCCTAGCACCGCCCCGCCCCCAATCC |
| | | TCTGTGGCACATGGAGTCTTGGTCCCCAGAGTCCCCCAGCGGCCTC |
| | | CAGATGGTCTGGGAGGGCAGTTCAGCTGTGGCTGCGCATAGCAGA |
| | | CATACAACGGACGGTGGGCCCAGACCCAGGCTGTGTAGACCCAGC |
| | | CCCCCCGCCCCGCAGTGCCTAGGTCACCCACTAACGCCCCAGGCCT |
| | | GGTCTTGGCTGGGCGTGACTGTTACCCTCAAAAGCAGGCAGCTCCA |
| | | GGGTAAAAGGTGCCCTGCCCTGTAGAGCCCACCTTCCTTCCCAGGG |
| | | CTGCGGCTGGGTAGGTTTGTAGCCTTCATCACGGGCCACCTCCAGC |
| | | CACTGGACCGCTGGCCCCTGCCCTGTCCTGGGGAGTGTGGTCCTGC |
| | | GACTTCTAAGTGGCCGCAAGCCACCTGACTCCCCCAACACCACACT |
| | | CTACCTCTCAAGCCCAGGTCTCTCCCTAGTGACCCACCCAGCACAT |
| | | TTAGCTAGCTGAGCCCCACAGCCAGAGGTCCTCAGGCCCTGCTTTC |
| | | AGGGCAGTTGCTCTGAAGTCGGCAAGGGGGAGTGACTGCCTGGCC |
| | | ACTCCATGCCCTCCAAGAGCTCCTTCTGCAGGAGCGTACAGAACCC |
| | | AGGGCCCTGGCACCCGTGCAGACCCTGGCCCACCCCACCTGGGCG |
| | | CTCAGTGCCCAAGAGATGTCCACACCTAGGATGTCCCGCGGTGGG |
| | | TGGGGGGCCCGAGAGACGGGCAGGCCGGGGGCAGGCCTGGCCAT |
| | | GCGGGGCCGAACCGGGCACTGCCCAGCGTGGGGCGCGGGGGCCAC |
| | | GGCGCGCGCCCCCAGCCCCCGGGCCCAGCACCCCAAGGCGGCCAA |
| | | CGCCAAAACTCTCCCTCCTCCTCTTCCTCAATCTCGCTCTCGCTCTT |
| | | TTTTTTTTTCGCAAAAGGAGGGGAGAGGGGGTAAAAAAATGCTGC |
| | | ACTGTGCGGCGAAGCCGGTGAGTGAGCGGCGCGGGGCCAATCAGC |
| | | GTGCGCCGTTCCGAAAGTTGCCTTTTATGGCTCGAGCGGCCGCGGC |
| | | GGCGCCCTATAAAACCCAGCGGCGCGACGCGCCACCACCGCCGAG |
| | | ACCGCGTCCGCCCCGCGAGCACAGAGCCTCGCCTTTGCCGATCCGC |
| | | CGCCCGTCCACACCCGCCGCCAGGTAAGCCCGGCCAGCCGACCGG |
| | | GGCAGGCGGCTCACGGCCCGGCCGCAGGCGGCCGCGGCCCCTTCG |
| | | CCCGTGCAGAGCCGCCGTCTGGGCCGCAGCGGGGGGCGCATGGGG |
| | | GGGGAACCGGACCGCCGTGGGGGGCGCGGGAGAAGCCCCTGGGC |
| | | CTCCGGAGATGGGGGACACCCCACGCCAGTTCGGAGGCGCGAGGC |
| | | CGCGCTCGGGAGGCGCGCTCCGGGGGTGCCGCTCTCGGGGCGGGG |
| | | GCAACCGGCGGGGTCTTTGTCTGAGCCGGGCTCTTGCCAATGGGG |
| | | ATCGCAGGGTGGGCGCGGCGGAGCCCCCGCCAGGCCCGGTGGGGG |
| | | CTGGGGCGCCATTGCGCGTGCGCGCTGGTCCTTTGGGCGCTAACTG |
| | | CGTGCGCGCTGGGAATTGGCGCTAATTGCGCGTGCGCGCTGGGAC |
| | | TCAAGGCGCTAACTGCGCGTGCGTTCTGGGGCCCGGGGTGCCGCG |
| | | GCCTGGGCTGGGGCGAAGGCGGGCTCGGCCGGAAGGGGTGGGGTC |
| | | GCCGCGGCTCCCGGGCGCTTGCGCGCACTTCCTGCCCGAGCCGCTG |
| | | GCCGCCCGAGGGTGTGGCCGCTGCGTGCGCGCGCGCCGACCCGGC |
| | | GCTGTTTGAACCGGGCGGAGGCGGGGCTGGCGCCCGGTTGGGAGG |
| | | GGGTTGGGGCCTGGCTTCCTGCCGCGCGCCGCGGGGACGCCTCCG |
| | | ACCAGTGTTTGCCTTTTATGGTAATAACGCGGCCGGCCCGGCTTCC |
| | | TTTGTCCCCAATCTGGGCGCGCGCCGGCGCCCCCTGGCGGCCTAAG |
| | | GACTCGGCGCGCCGGAAGTGGCCAGGGCGGGGGCGACCTCGGCTC |
| | | ACAGCGCGCCCGGCTAT |
| heIF4A1 | 30 | GTTGATTTCCTTCATCCCTGGCACACGTCCAGGCAGTGTCGAATCC |
| | | ATCTCTGCTACAGGGGAAAACAAATAACATTTGAGTCCAGTGGAG |
| | | ACCGGGAGCAGAAGTAAAGGGAAGTGATAACCCCCAGAGCCCGG |
| | | AAGCCTCTGGAGGCTGAGACCTCGCCCCCCTTGCGTGATAGGGCCT |
| | | ACGGAGCCACATGACCAAGGCACTGTCGCCTCCGCACGTGTGAGA |
| | | GTGCAGGGCCCCAAGATGGCTGCCAGGCCTCGAGGCCTGACTCTT |
| | | CTATGTCACTTCCGTACCGGCGAGAAAGGCGGGCCCTCCAGCCAA |
| | | TGAGGCTGCGGGGCGGGCCTTCACCTTGATAGGCACTCGAGTTATC |
| | | CAATGGTGCCTGCGGGCCGGAGCGACTAGGAACTAACGTCATGCC |
| | | GAGTTGCTGAGCGCCGGCAGGCGGGGCCGGGGCGGCCAAACCAAT |
| | | GCGATGGCCGGGGCGGAGTCGGGCGCTCTATAAGTTGTCGATAGG |
| | | CGGGCACTCCGCCCTAGTTTCTAAGGACCATG |
| hGAPDH | 31 | AGTTCCCCAACTTTCCCGCCTCTCAGCCTTTGAAAGAAAGAAAGGG |
| | | GAGGGGGCAGGCCGCGTGCAGTCGCGAGCGGTGCTGGGCTCCGGC |
| | | TCCAATTCCCCATCTCAGTCGCTCCCAAAGTCCTTCTGTTTCATCCA |
| | | AGCGTGTAAGGGTCCCCGTCCTTGACTCCCTAGTGTCCTGCTGCCC |
| | | ACAGTCCAGTCCTGGGAACCAGCACCGATCACCTCCCATCGGGCC |
| | | AATCTCAGTCCCTTCCCCCCTACGTCGGGGCCCACACGCTCGGTGC |
| | | GTGCCCAGTTGAACCAGGCGGCTGCGGAAAAAAAAAAGCGGGGA |
| | | GAAAGTAGGGCCCGGCTACTAGCGGTTTTACGGGCGCACGTAGCT |
| | | CAGGCCTCAAGACCTTGGGCTGGGACTGGCTGAGCCTGGCGGGAG |
| | | GCGGGGTCCGAGTCACCGCCTGCCGCCGCGCCCCCGGTTTCTATAA |
| | | ATTGAGCCCGCAGCCTCCCGCTTCGCTCTCTGCTCCTCCTGTTCGAC |
| | | AGTCAGCCGCATCTTCTTTTGCGTCGCCAGGTGAAGACGGGCGGA |
| | | GAGAAACCCGGGAGGCTAGGGACGGCCTGAAGGCGGCAGGGGCG |
| | | GGCGCAGGCCGGATGTGTTCGCGCCGCTGCGGGGTGGGCCCGGGC |
| | | GGCCTCCGCATTGCAGGGGGGGGCGGAGGACGTGATGCGGCGCGG |
| | | GCTGGGCATGGAGGCCTGGTGGGGGAGGGGAGGGGAGGCGTGGG |
| | | TGTCGGCCGGGGCCACTAGGCGCTCACTGTTCTCTCCCTCCGCGCA |
| | | GCCGAGCCACATCGCTGAGACAC |
| hGRP78 | 32 | AGTGCGGTTACCAGCGGAAATGCCTCGGGGTCAGAAGTCGCAGGA |
| | | GAGATAGACAGCTGCTGAACCAATGGGACCAGCGGATGGGGCGG |
| | | ATGTTATCTACCATTGGTGAACGTTAGAAACGAATAGCAGCCAAT |
| | | GAATCAGCTGGGGGGGCGGAGCAGTGACGTTTATTGCGGAGGGGG |
| | | CCGCTTCGAATCGGCGGCGGCCAGCTTGGTGGCCTGGGCCAATGA |
| | | ACGGCCTCCAACGAGCAGGGCCTTCACCAATCGGCGGCCTCCACG |
| | | ACGGGGCTGGGGGAGGGTATATAAGCCGAGTAGGCGACGGTGAG |
| | | GTCGACGCCGGCCAAGACAGCACAGACAGATTGACCTATTGGGGT |
| | | GTTTCGCGAGTGTGAGAGGGAAGCGCCGCGGCCTGTATTTCTAGA |
| | | CCTGCCCTTCGCCTGGTTCGTGGCGCCTTGTGACCCCGGGCCCCTG |
| | | CCGCCTGCAAGTCGGAAATTGCGCTGTGCTCCTGTGCTACGGCCTG |
| | | TGGCTGGACTGCCTGCTGCTGCCCAACTGGCTGGCAC |
| hGRP94 | 33 | TAGTTTCATCACCACCGCCACCCCCCCGCCCCCCCGCCATCTGAAA |
| | | GGGTTCTAGGGGATTTGCAACCTCTCTCGTGTGTTTCTTCTTTCCGA |
| | | GAAGCGCCGCCACACGAGAAAGCTGGCCGCGAAAGTCGTGCTGGA |
| | | ATCACTTCCAACGAAACCCCAGGCATAGATGGGAAAGGGTGAAGA |
| | | ACACGTTGCCATGGCTACCGTTTCCCCGGTCACGGAATAAACGCTC |
| | | TCTAGGATCCGGAAGTAGTTCCGCCGCGACCTCTCTAAAAGGATG |
| | | GATGTGTTCTCTGCTTACATTCATTGGACGTTTTCCCTTAGAGGCCA |
| | | AGGCCGCCCAGGCAAAGGGGCGGTCCCACGCGTGAGGGGCCCGCG |
| | | GAGCCATTTGATTGGAGAAAAGCTGCAAACCCTGACCAATCGGAA |
| | | GGAGCCACGCTTCGGGCATCGGTCACCGCACCTGGACAGCTCCGA |
| | | TTGGTGGACTTCCGCCCCCCCTCACGAATCCTCATTGGGTGCCGTG |
| | | GGTGCGTGGTGCGGCGCGATTGGTGGGTTCATGTTTCCCGTCCCCC |
| | | GCCCGCGAGAAGTGGGGGTGAAAAGCGGCCCGACCTGCTTGGGGT |
| | | GTAGTGGGCGGACCGCGCGGCTGGAGGTGTGAGGATCCGAACCCA |
| | | GGGGTGGGGGGGGAGGCGGCTCCTGCGATCGAAGGGGACTTGAG |
| | | ACTCACCGGCCGCACGTC |
| hHSP70 | 34 | GGGCCGCCCACTCCCCCTTCCTCTCAGGGTCCCTGTCCCCTCCAGT |
| | | GAATCCCAGAAGACTCTGGAGAGTTCTGAGCAGGGGGCGGCACTC |
| | | TGGCCTCTGATTGGTCCAAGGAAGGCTGGGGGGCAGGACGGGAGG |
| | | CGAAAACCCTGGAATATTCCCGACCTGGCAGCCTCATCGAGCTCG |
| | | GTGATTGGCTCAGAAGGGAAAAGGCGGGTCTCCGTGACGACTTAT |
| | | AAAAGCCCAGGGGCAAGCGGTCCGGATAACGGCTAGCCTGAGGA |
| | | GCTGCTGCGACAGTCCACTACCTTTTTCGAGAGTGACTCCCGTTGT |
| | | CCCAAGGCTTCCCAGAGCGAACCTGTGCGGCTGCAGGCACCGGCG |
| | | CGTCGAGTTTCCGGCGTCCGGAAGGACCGAGCTCTTCTCGCGGATC |
| | | CAGTGTTCCGTTTCCAGCCCCCAATCTCAGAGCGGAGCCGACAGA |
| | | GAGCAGGGAACCC |
| hKINb | 35 | GCCCCACCCCCGTCCGCGTTACAACCGGGAGGCCCGCTGGGTCCTG |
| | | CACCGTCACCCTCCTCCCTGTGACCGCCCACCTGATACCCAAACAA |
| | | CTTTCTCGCCCCTCCAGTCCCCAGCTCGCCGAGCGCTTGCGGGGAG |
| | | CCACCCAGCCTCAGTTTCCCCAGCCCCGGGCGGGGCGAGGGGCGA |
| | | TGACGTCATGCCGGCGCGCGGCATTGTGGGGCGGGGCGAGGCGGG |
| | | GCGCCGGGGGGAGCAACACTGAGACGCCATTTTCGGCGGCGGGAG |
| | | CGGCGCAGGCGGCCGAGCGGGACTGGCTGGGTCGGCTGGGCTGCT |
| | | GGTGCGAGGAGCCGCGGGGCTGTGCTCGGCGGCCAAGGGGACAGC |
| | | GCGTGGGTGGCCGAGGATGCTGCGGGGCGGTAGCTCCGGCGCCCC |
| | | TCGCTGGTGACTGCTGCGCCGTGCCTCACACAGCCGAGGCGGGCTC |
| | | GGCGCACAGTCGCTGCTCCGCGCTCGCGCCCGGCGGCGCTCCAGG |
| | | TGCTGACAGCGCGAGAGAGCGCGGCCTCAGGAGCAACAC |
| hUBIb | 36 | TTCCAGAGCTTTCGAGGAAGGTTTCTTCAACTCAAATTCATCCGCC |
| | | TGATAATTTTCTTATATTTTCCTAAAGAAGGAAGAGAAGCGCATAG |
| | | AGGAGAAGGGAAATAATTTTTTAGGAGCCTTTCTTACGGCTATGAG |
| | | GAATTTGGGGCTCAGTTGAAAAGCCTAAACTGCCTCTCGGGAGGTT |
| | | GGGCGCGGCGAACTACTTTCAGCGGCGCACGGAGACGGCGTCTAC |
| | | GTGAGGGGTGATAAGTGACGCAACACTCGTTGCATAAATTTGCGC |
| | | TCCGCCAGCCCGGAGCATTTAGGGGCGGTTGGCTTTGTTGGGTGAG |
| | | CTTGTTTGTGTCCCTGTGGGTGGACGTGGTTGGTGATTGGCAGGAT |
| | | CCTGGTATCCGCTAACAGGTACTGGCCCACAGCCGTAAAGACCTG |
| | | CGGGGGCGTGAGAGGGGGGAATGGGTGAGGTCAAGCTGGAGGCT |
| | | TCTTGGGGTTGGGTGGGCCGCTGAGGGGAGGGGAGGGCGAGGTGA |
| | | CGCGACACCCGGCCTTTCTGGGAGAGTGGGCCTTGTTGACCTAAGG |
| | | GGGGCGAGGGCAGTTGGCACGCGCACGCGCCGACAGAAACTAAC |
| | | AGACATTAACCAACAGCGATTCCGTCGCGTTTACTTGGGAGGAAG |
| | | GCGGAAAAGAGGTAGTTTGTGTGGCTTCTGGAAACCCTAAATTTG |
| | | GAATCCCAGTATGAGAATGGTGTCCCTTCTTGTGTTTCAATGGGAT |
| | | TTTTACTTCGCGAGTCTTGTGGGTTTGGTTTTGTTTTCAGTTTGCCT |
| | | AACACCGTGCTTAGGTTTGAGGCAGATTGGAGTTCGGTCGGGGGA |
| | | GTTTGAATATCCGGAACAGTTAGTGGGGAAAGCTGTGGACGCTTG |
| | | GTAAGAGAGCGCTCTGGATTTTCCGCTGTTGACGTTGAAACCTTGA |
| | | ATGACGAATTTCGTATTAAGTGACTTAGCCTTGTAAAATTGAGGGG |
| | | AGGCTTGCGGAATATTAACGTATTTAAGGCATTTTGAAGGAATAGT |
| | | TGCTAATTTTGAAGAATATTAGGTGTAAAAGCAAGAAATACAATG |
| | | ATCCTGAGGTGACACGCTTATGTTTTACTTTTAAACTAGGTCACC |
| ZF binding | 37 | cgggtttcgtaacaatcgcatgaggattcgcaacgccttcGGCGTAGCCGATGTCGCGctccc |
| site | | gtctcagtaaaggtcGGCGTAGCCGATGTCGCGcaatcggactgccttcgtacGGCGTA |
| | | GCCGATGTCGCGcgtatcagtcgcctcggaacGGCGTAGCCGATGTCGCGcattc |
| | | gtaagaggctcactctcccttacacggagtggataACTAGTTCTAGAGGGTATATAATG |
| | | GGGGCCA |
| Exemplary | 38 | TCTGTTCCTGTTAATCAACCTCTGGATTACAAAATTTGTGAAAGAT |
| WPRE | | TGACTGATATTCTTAACTATGTTGCTCCTTTTACGCTGTGTGGATAT |
| | | GCTGCTTTAATGCCTCTGTATCATGCTATTGCTTCCCGTACGGCTTT |
| | | CGTTTTCTCCTCCTTGTATAAATCCTGGTTGCTGTCTCTTTATGAGG |
| | | AGTTGTGGCCCGTTGTCCGTCAACGTGGCGTGGTGTGCTCTGTGTT |
| | | TGCTGACGCAACCCCCACTGGCTGGGGCATTGCCACCACCTGTCAA |
| | | CTCCTTTCTGGGACTTTCGCTTTCCCCCTCCCGATCGCCACGGCAGA |
| | | ACTCATCGCCGCCTGCCTTGCCCGCTGCTGGACAGGGGCTAGGTTG |
| | | CTGGGCACTGATAATTCCGTGGTGTTGTCGGGGAAGCTGACGTCCT |
| | | TTCCATGGCTGCTCGCCTGTGTTGCCAACTGGATCCTGCGCGGGAC |
| | | GTCCTTCTGCTACGTCCCTTCGGCTCTCAATCCAGCGGACCTCCCTT |
| | | CCCGAGGCCTTCTGCCGGTTCTGCGGCCTCTCCCGCGTCTTCGCTTT |
| | | CGGCCTCCGACGAGTCGGATCTCCCTTTGGGCCGCCTCCCCGCCTG |
| | | TTTCGCCTCGGCGTCCGGTCCGTGTTGCTTGGTCGTCACCTGTGCA |
| | | GAATTGCGAACCATGGATTCCA |
| Exemplary | 39 | TCTGTTCCTGTTAATCAACCTCTGGATTACAAAATTTGTGAAAGAT |
| WPRE | | TGACTGATATTCTTAACTATGTTGCTCCTTTTACGCTGTGTGGATAT |
| | | GCTGCTTTAATGCCTCTGTATCATGCTATTGCTTCCCGTACGGCTTT |
| | | CGTTTTCTCCTCCTTGTATAAATCCTGGTTGCTGTCTCTTTATGAGG |
| | | AGTTGTGGCCCGTTGTCCGTCAACGTGGCGTGGTGTGCTCTGTGTT |
| | | TGCTGACGCAACCCCCACTGGCTGGGGCATTGCCACCACCTGTCAA |
| | | CTCCTTTCTGGGACTTTCGCTTTCCCCCTCCCGATCGCCACGGCAGA |
| | | ACTCATCGCCGCCTGCCTTGCCCGCTGCTGGACAGGGGCTAGGTTG |
| | | CTGGGCACTGATAATTCCGTGGTGTTGTCGGGGAAATCATCGTCCT |
| | | TTCCTTGGCTGCTCGCCTGTGTTGCCAACTGGATCCTGCGCGGGAC |
| | | GTCCTTCTGCTACGTCCCTTCGGCTCTCAATCCAGCGGACCTCCCTT |
| | | CCCGAGGCCTTCTGCCGGTTCTGCGGCCTCTCCCGCGTCTTCGCTTT |
| | | CGGCCTCCGACGAGTCGGATCTCCCTTTGGGCCGCCTCCCCGCCTG |
| | | TTTCGCCTCGGCGTCCGGTCCGTGTTGCTTGGTCGTCACCTGTGCA |
| | | GAATTGCGAACCATGGATTCCA |
| SB03422 | 40 | SAGDMRAANLWPSPLMIKRSKKNSLALSLTADQMVSALLDAEPPILY |
| Wild Type | | SEYDPTRPFSEASMMGLLTNLADRELVHMINWAKRVPGFVDLTLHD |
| | | QVHLLECAWLEILMIGLVWRSMEHPVKLLFAPNLLLDRNQGKCVEG |
| | | MVEIFDMLLATSSRFRMMNLQGEEFVCLKSIILLNSGVYTFLSSTLKSL |
| | | EEKDHIHRVLDKITDTLIHLMAKAGLTLQQQHQRLAQLLLILSHIRHM |
| | | SNKGMEHLYSMKCKNVVPLYDLLLEAADAHRLHAPTSRGGASVEET |
| | | DQSHLATAGSTSSHSLQKYYITGEAEGFPATA |
| SB06136 | 41 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.81 | | QVHLLECAWMEILMIGVVWRSMEHPVKLLFAPNLLLDRDQGKCVEG |
| L384M/ | | LVEIFDMLLATSSRFRMMNLQGEEFVCLKSIILLNSGVYTFLPSTLKSL |
| L391V/ | | EEKDHIHRVLDKITDTLIHLMAKAGLTLQQQHQRLAQLLLILSHIRHM |
| N413D/ | | SNKGMELLYSMKCKNVVPLYDLLLEAADAHRLHAPTSRGGASVEET |
| M421L/ | | DQSHLATAGSTSSHSLQKYYITGEAEGFPATA |
| S463P/ | | |
| H524L | | |
| SB06138 | 42 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.93 | | QVHLLECAWMEILMIGLVWRSMEHPVKLLFAPNLVLDRDQGKCVEG |
| L384M/ | | MVEIFDMLLATSSRFRMMNLQGEEFVCLKSIILLNSGVYTFLPSTLKSL |
| L409V/ | | EEKDHIHRVLDKITDTLIHLMAKAGLTLQQQHQRLAQLLLILSHIRHM |
| N413D/ | | SNKGMELLYSMKCKNVVPLYDLLLEAADAHRLHAPTSRGGASVEET |
| S463P/ | | DQSHLATAGSTSSHSLQKYYITGEAEGFPATA |
| H524L | | |
| SB06139 | 43 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.86 | | VHLLECAWMEILMIGVVWRSMEHPVKLLFAPNLLLDRNQGKCVEGM |
| L354I/ | | VEIFDMLLATSSRFRMMNLQGEEFVCLKSIILLNSGVYTFLPSTLKSLE |
| L384M/ | | EKDHIHRVLDKITDTLIHLMAKAGLTLQQQHQRLAQLLLILSHIRHMS |
| L391V/ | | NKGMEHLYSMKCKNVVPLYDLLLEAADAHRLHAPTSRGGASVEETD |
| S463P | | QSHLATAGSTSSHSLQKYYITGEAEGFPATA |
| SB06140 | 44 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.95 | | VHLLECAWMEILMIGLVWRSMEHPVKLLFAPNLVLDRDQGKCVEGL |
| L354I/ | | VEIFDMLLATSSRFRMMNLQGEEFVCLKSIILLNSGVYTFLPSTLKSLE |
| L384M/ | | EKDHIHRVLDKITDTLIHLMAKAGLTLQQQHQRLAQLLLILSHIRHMS |
| L409V/ | | NKGMEFLYSMKCKNVVPLYDLLLEAADAHRLHAPTSRGGASVEETD |
| N413D/ | | QSHLATAGSTSSHSLQKYYITGEAEGFPATA |
| M421L/ | | |
| S463P/ | | |
| H524F | | |
| SB06141 | 45 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.88 | | QVHLLECAWLEILMIGVVWRSMEHPVKLLFAPNLLLDRDEGKCVEG |
| L391V/ | | MVEIFDMLLATSSRFRMMNLQGEEFVCLKSIILLNSGVYTFLPSTLKSL |
| N413D/ | | EEKDHIHRVLDKITDTLIHLMAKAGLTLQQQHQRLAQLLLILSHIRHM |
| Q414E/ | | SNKGMEFLYSMKCKNVVPLYDLLLEAADAHRLHAPTSRGGASVEET |
| S463P/ | | DQSHLATAGSTSSHSLQKYYITGEAEGFPATA |
| H524F | | |
| SB06142 | 46 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.77 | | VHLLECAWLEILMIGVVWRSMEHPVKLLFAPNLLLDRDQGKCVEGL |
| L354I/ | | VEIFDMLLATSSRFRMMNLQGEEFVCLKSIILLNSGVYTFLPSTLKSLE |
| L391V/ | | EKDHIHRVLDKITDTLIHLMAKAGLTLQQQHQRLAQLLLILSHIRHAS |
| N413D/ | | NKGMELLYSMKCKNVVPLYDLLLEAADAHRLHAPTSRGGASVEETD |
| M421L/ | | QSHLATAGSTSSHSLQKYYITGEAEGFPATA |
| S463P/ | | |
| M517A/ | | |
| H524L | | |
| SB06143 | 47 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.49 | | QVHLLECAWLEILMIGVVWRSMEHPVKLLFAPNLLLDRNEGKCVEGL |
| L391V/ | | VEIFDMLLATSSRFRMMNLQGEEFVCLKSIILLNSGVYTFLPSTLKSLE |
| Q414E/ | | EKDHIHRVLDKITDTLIHLMAKAGLTLQQQHQRLAQLLLILSHIRHMS |
| M421L/ | | NKGMEFLYSMKCKNVVPLYDLLLEAADAHRLHAPTSRGGASVEETD |
| S463P/ | | QSHLATAGSTSSHSLQKYYITGEAEGFPATA |
| H524F | | |
| SB06144 | 48 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.58 | | VHLLECAWLEILMIGLVWRSMEHPVKLLFAPNLVLDRDQGKCVEGL |
| L354I/ | | VEIFDMLLATSSRFRMMNLQGEEFVCLKSIILLNSGVYTFLSSTLKSLE |
| L409V/ | | EKDHIHRVLDKITDTLIHLMAKAGLTLQQQHQRLAQLLLILSHIRHMS |
| N413D/ | | NKGMELLYSMKCKNVVPLYDLLLEAADAHRLHAPTSRGGASVEETD |
| M421L/ | | QSHLATAGSTSSHSLQKYYITGEAEGFPATA |
| H524L | | |
| SB06145 | 49 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.62 | | QVHLLECAWLEILMIGLVWRSMEHPVKLLFAPNLVLDRDEGKCVEGL |
| L409V/ | | VEIFDMLLATSSRFRMMNLQGEEFVCLKSIILLNSGVYTFLPSTLKSLE |
| N413D/ | | EKDHIHRVLDKITDTLIHLMAKAGLTLQQQHQRLAQLLLILSHIRHMS |
| Q414E/ | | NKGMELLYSMKCKNVVPLYDLLLEAADAHRLHAPTSRGGASVEETD |
| M421L/ | | QSHLATAGSTSSHSLQKYYITGEAEGFPATA |
| S463P/ | | |
| H524L | | |
| SB06146 | 50 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.63 | | VHLLECAWLEILMIGVVWRSMEHPVKLLFAPNLLLDRDEGKCVEGLV |
| L354I/ | | EIFDMLLATSSRFRMMNLQGEEFVCLKSIILLNSGVYTFLSSTLKSLEE |
| L391V/ | | KDHIHRVLDKITDTLIHLMAKAGLTLQQQHQRLAQLLLILSHIRHASN |
| N413D/ | | KGMEFLYSMKCKNVVPLYDLLLEAADAHRLHAPTSRGGASVEETDQ |
| Q414E/ | | SHLATAGSTSSHSLQKYYITGEAEGFPATA |
| M421L/ | | |
| M517A/ | | |
| H524F | | |
| SB06147 | 51 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.55 | | VHLLECAWLEILMIGLVWRSMEHPVKLLFAPNLVLDRNQGKCVEGL |
| L354I/ | | VEIFDMLLATSSRFRMMNLQGEEFVCLKSIILLNSGVYTFLPSTLKSLE |
| L409V/ | | EKDHIHRVLDKITDTLIHLMAKAGLTLQQQHQRLAQLLLILSHIRHMS |
| M421L/ | | NKGMELLYSMKCKNVVPLYDLLLEAADAHRLHAPTSRGGASVEETD |
| S463P/ | | QSHLATAGSTSSHSLQKYYITGEAEGFPATA |
| H524L | | |
| SB06149 | 52 | SAGDMRAANLWPSPLMIKRSKKNSLALSLTADQMVSALLDAEPPILY |
| rld.ERT2. | | SEYDPTRPFSEASMMGLLTNLADREIVHMINWAKRVPGFVDLTLHDQ |
| mutant.41 | | VHLLECAWLEILMIGVVWRSMEHPVKLLFAPNLVLDRDEGKCVEGM |
| L354I/ | | VEIFDMLLATSSRFRMMNLQGEEFVCLKSIILLNSGVYTFLSSTLKSLE |
| L391V/ | | EKDHIHRVLDKITDTLIHLMAKAGLTLQQQHQRLAQLLLILSHIRHMS |
| L409V/ | | NKGMELLYSMKCKNVVPLYDLLLEAADAHRLHAPTSRGGASVEETD |
| N413D/ | | QSHLATAGSTSSHSLQKYYITGEAEGFPATA |
| Q414E/ | | |
| H524L | | |
| SB06150 | 53 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.43 | | QVHLLECAWLEILMIGLVWRSMEHPVKLLFAPNLLLDRNEGKCVEG |
| Q414E/ | | MVEIFDMLLATSSRFRMMNLQGEEFVCLKSIILLNSGVYTFLPSTLKSL |
| S463P/ | | EEKDHIHRVLDKITDTLIHLMAKAGLTLQQQHQRLAQLLLILSHIRHM |
| H524L | | SNKGMELLYSMKCKNVVPLYDLLLEAADAHRLHAPTSRGGASVEET |
| | | DQSHLATAGSTSSHSLQKYYITGEAEGFPATA |
| SB06151 | 54 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.46 | | QVHLLECAWMEILMIGVVWRSMEHPVKLLFAPNLVLDRDQGKCVEG |
| L384M/ | | LVEIFDMLLATSSRFRMMNLQGEEFVCLKSIILLNSGVYTFLPSTLKSL |
| L391V/ | | EEKDHIHRVLDKITDTLIHLMAKAGLTLQQQHQRLAQLLLILSHIRHA |
| L409V/ | | SNKGMEFLYSMKCKNVVPLYDLLLEAADAHRLHAPTSRGGASVEET |
| N413D/ | | DQSHLATAGSTSSHSLQKYYITGEAEGFPATA |
| M421L/ | | |
| S463P/ | | |
| M517A/ | | |
| H524F | | |
| SB06152 | 55 | SAGDMRAANLWPSPLMIKRSKKNSLALSLTADQMVSALLDAEPPILY |
| rld.ERT2. | | SEYDPTRPFSEASMMGLLTNLADRELVHMINWAKRVPGFVDLTLHD |
| mutant.40 | | QVHLLECAWMEILMIGVVWRSMEHPVKLLFAPNLLLDRDQGKCVEG |
| L384M/ | | MVEIFDMLLATSSRFRMMNLQGEEFVCLKSIILLNSGVYTFLSSTLKSL |
| L391V/ | | EEKDHIHRVLDKITDTLIHLMAKAGLTLQQQHQRLAQLLLILSHIRHM |
| N413D/ | | SNKGMEFLYSMKCKNVVPLYDLLLEAADAHRLHAPTSRGGASVEET |
| H524F | | DQSHLATAGSTSSHSLQKYYITGEAEGFPATA |
| 4X ZF5 | 56 | cgggtttcgtaacaatcgcatgaggattcgcaacgcctttGAAGCAGTCGACGCCGAAgtccc |
| BD + YB- | | gtctcagtaaaggttGAAGCAGTCGACGCCGAAgaatcggactgccttcgtatGAAGCA |
| TATA min | | GTCGACGCCGAAggtatcagtcgcctcggaatGAAGCAGTCGACGCCGAAgattc |
| (Syn | | gtaagaggctcactctcccttacacggagtggataACTAGTTCTAGAGGGTATATAATG |
| promoter) | | GGGGCCA |
| IL-12 | 57 | ATGTGCCATCAGCAACTCGTCATCTCCTGGTTCTCCCTTGTGTTCCT |
| | | CGCTTCCCCTCTGGTCGCCATTTGGGAACTGAAGAAGGACGTCTAC |
| | | GTGGTCGAGCTGGATTGGTACCCGGACGCCCCTGGAGAAATGGTC |
| | | GTGCTGACTTGCGATACGCCAGAAGAGGACGGCATAACCTGGACC |
| | | CTGGATCAGAGCTCCGAGGTGCTCGGAAGCGGAAAGACCCTGACC |
| | | ATTCAAGTCAAGGAGTTCGGCGACGCGGGCCAGTACACTTGCCAC |
| | | AAGGGTGGCGAAGTGCTGTCCCACTCCCTGCTGCTGCTGCACAAG |
| | | AAAGAGGATGGAATCTGGTCCACTGACATCCTCAAGGACCAAAAA |
| | | GAACCGAAGAACAAGACCTTCCTCCGCTGCGAAGCCAAGAACTAC |
| | | AGCGGTCGGTTCACCTGTTGGTGGCTGACGACAATCTCCACCGACC |
| | | TGACTTTCTCCGTGAAGTCGTCACGGGGATCAAGCGATCCTCAGGG |
| | | CGTGACCTGTGGAGCCGCCACTCTGTCCGCCGAGAGAGTCAGGGG |
| | | AGACAACAAGGAATATGAGTACTCCGTGGAATGCCAGGAGGACAG |
| | | CGCCTGCCCTGCCGCGGAAGAGTCCCTGCCTATCGAGGTCATGGTC |
| | | GATGCCGTGCATAAGCTGAAATACGAGAACTACACTTCCTCCTTCT |
| | | TTATCCGCGACATCATCAAGCCTGACCCCCCCAAGAACTTGCAGCT |
| | | GAAGCCACTCAAGAACTCCCGCCAAGTGGAAGTGTCTTGGGAATA |
| | | TCCAGACACTTGGAGCACCCCGCACTCATACTTCTCGCTCACTTTC |
| | | TGTGTGCAAGTGCAGGGAAAGTCCAAACGGGAGAAGAAAGACCG |
| | | GGTGTTCACCGACAAAACCTCCGCCACTGTGATTTGTCGGAAGAAC |
| | | GCGTCAATCAGCGTCCGGGCGCAGGATAGATACTACTCGTCCTCCT |
| | | GGAGCGAATGGGCCAGCGTGCCTTGTTCCGGTGGCGGATCAGGCG |
| | | GAGGTTCAGGAGGAGGCTCCGGAGGAGGTTCCCGGAACCTCCCTG |
| | | TGGCAACCCCCGACCCTGGAATGTTCCCGTGCCTACACCACTCCCA |
| | | AAACCTCCTGAGGGCTGTGTCGAACATGTTGCAGAAGGCCCGCCA |
| | | GACCCTTGAGTTCTACCCCTGCACCTCGGAAGAAATTGATCACGAG |
| | | GACATCACCAAGGACAAGACCTCGACCGTGGAAGCCTGCCTGCCG |
| | | CTGGAACTGACCAAGAACGAATCGTGTCTGAACTCCCGCGAGACA |
| | | AGCTTTATCACTAACGGCAGCTGCCTGGCGTCGAGAAAGACCTCAT |
| | | TCATGATGGCGCTCTGTCTTTCCTCGATCTACGAAGATCTGAAGAT |
| | | GTATCAGGTCGAGTTCAAGACCATGAACGCCAAGCTGCTCATGGA |
| | | CCCGAAGCGGCAGATCTTCCTGGACCAGAATATGCTCGCCGTGATT |
| | | GATGAACTGATGCAGGCCCTGAATTTCAACTCCGAGACTGTGCCTC |
| | | AAAAGTCCAGCCTGGAAGAACCGGACTTCTACAAGACCAAGATCA |
| | | AGCTGTGCATCCTGTTGCACGCTTTCCGCATTCGAGCCGTGACCAT |
| | | TGACCGCGTGATGTCCTACCTGAACGCCAGT |
| IL-12 | 58 | MCHQQLVISWFSLVFLASPLVAIWELKKDVYVVELDWYPDAPGEMV |
| | | VLTCDTPEEDGITWTLDQSSEVLGSGKTLTIQVKEFGDAGQYTCHKG |
| | | GEVLSHSLLLLHKKEDGIWSTDILKDQKEPKNKTFLRCEAKNYSGRFT |
| | | CWWLTTISTDLTFSVKSSRGSSDPQGVTCGAATLSAERVRGDNKEYE |
| | | YSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKRE |
| | | KKDRVFTDKTSATVICRKNASISVRAQDRYYSSSWSEWASVPCSGGG |
| | | SGGGSGGGSGGGSRNLPVATPDPGMFPCLHHSQNLLRAVSNMLQKA |
| | | RQTLEFYPCTSEEIDHEDITKDKTSTVEACLPLELTKNESCLNSRETSFI |
| | | TNGSCLASRKTSFMMALCLSSIYEDLKMYQVEFKTMNAKLLMDPKR |
| | | QIFLDQNMLAVIDELMQALNFNSETVPQKSSLEEPDFYKTKIKLCILLH |
| | | AFRIRAVTIDRVMSYLNAS |
| AU/SLDE | 59 | ATTTATTTATTTATTTATTTAacatcggttccCTGTTTAATATTTAAACAG |
| (destablizat | | |
| i-on | | |
| domain) | | |
| A2 | 60 | AGAGCGAGATTCCGTCTCAAAGAAAAAAAAAGTAATGAAATGAAT |
| (insulator) | | AAAATGAGTCCTAGAGCCAGTAAATGTCGTAAATGTCTCAGCTAG |
| | | TCAGGTAGTAAAAGGTCTCAACTAGGCAGTGGCAGAGCAGGATTC |
| | | AAATTCAGGGCTGTTGTGATGCCTCCGCAGACTCTGAGCGCCACCT |
| | | GGTGGTAATTTGTCTGTGCCTCTTCTGACGTGGAAGAACAGCAACT |
| | | AACACACTAACACGGCATTTACTATGGGCCAGCCATTGT |
| ZF5-7 | 61 | ATGTCTAGACCTGGCGAGAGGCCCTTCCAGTGCCGGATCTGCATGC |
| DBD (ZF | | GGAACTTCAGCAACATGAGCAACCTGACCAGACACACCCGGACAC |
| DNA BD) | | ACACAGGCGAGAAGCCTTTTCAGTGCAGAATCTGTATGCGCAATTT |
| | | CTCCGACAGAAGCGTGCTGCGGAGACACCTGAGAACCCACACCGG |
| | | CAGCCAGAAACCATTCCAGTGTCGCATCTGTATGAGAAACTTTAGC |
| | | GACCCCTCCAATCTGGCCCGGCACACCAGAACACATACCGGGGAA |
| | | AAACCCTTTCAGTGTAGGATATGCATGAGGAATTTTTCCGACCGGT |
| | | CCAGCCTGAGGCGGCACCTGAGGACACATACTGGCTCCCAAAAGC |
| | | CGTTCCAATGTCGGATATGTATGCGCAACTTTAGCCAGAGCGGCAC |
| | | CCTGCACAGACACACAAGAACCCATACTGGCGAGAAACCTTTCCA |
| | | ATGTAGAATCTGCATGCGAAATTTTTCCCAGCGGCCTAATCTGACC |
| | | AGGCATCTGAGGACCCACCTGAGAGGATCT |
| ZF5-7 | 62 | MSRPGERPFQCRICMRNFSNMSNLTRHTRTHTGEKPFQCRICMRNFSD |
| DBD (ZF | | RSVLRRHLRTHTGSQKPFQCRICMRNFSDPSNLARHTRTHTGEKPFQC |
| DNA BD) | | RICMRNFSDRSSLRRHLRTHTGSQKPFQCRICMRNFSQSGTLHRHTRT |
| | | HTGEKPFQCRICMRNFSQRPNLTRHLRTHLRGS |
| P65 | 63 | GATGAGTTTCCCACCATGGTGTTTCCTTCTGGGCAGATCAGCCAGG |
| (trans- | | CCTCGGCCTTGGCCCCGGCCCCTCCCCAAGTCCTGCCCCAGGCTCC |
| criptional | | AGCCCCTGCCCCTGCTCCAGCCATGGTATCAGCTCTGGCCCAGGCC |
| activator) | | CCAGCCCCTGTCCCAGTCCTAGCCCCAGGCCCTCCTCAGGCTGTGG |
| | | CCCCACCTGCCCCCAAGCCCACCCAGGCTGGGGAAGGAACGCTGT |
| | | CAGAGGCCCTGCTGCAGCTGCAGTTTGATGATGAAGACCTGGGGG |
| | | CCTTGCTTGGCAACAGCACAGACCCAGCTGTGTTCACAGACCTGGC |
| | | ATCCGTCGACAACTCCGAGTTTCAGCAGCTGCTGAACCAGGGCAT |
| | | ACCTGTGGCCCCCCACACAACTGAGCCCATGCTGATGGAGTACCCT |
| | | GAGGCTATAACTCGCCTAGTGACAGGGGCCCAGAGGCCCCCCGAC |
| | | CCAGCTCCTGCTCCACTGGGGGCCCCGGGGCTCCCCAATGGCCTCC |
| | | TTTCAGGAGATGAAGACTTCTCCTCCATTGCGGACATGGACTTCTC |
| | | AGCCCTGCTGAGTCAGATCAGCTCC |
| P65 | 64 | DEFPTMVFPSGQISQASALAPAPPQVLPQAPAPAPAPAMVSALAQAPA |
| (trans- | | PVPVLAPGPPQAVAPPAPKPTQAGEGTLSEALLQLQFDDEDLGALLGN |
| criptional | | STDPAVFTDLASVDNSEFQQLLNQGIPVAPHTTEPMLMEYPEAITRL V |
| activator) | | TGAQRPPDPAPAPLGAPGLPNGLLSGDEDFSSIADMDFSALLSQISS |
| AA | | |
| Mutant 81 | 65 | gctggagacatgagagctgccaacctttggccaagcccgctcatgatcaaacgctctaagaaga |
| (ERT | | acagcctggccttgtccctgacggccgaccagatggtcagtgccttgttggatgctgagccccc |
| mutant) | | catactctattccgagtatgatcctaccagacccttcagtgaagcttcgatgatgggcttactg |
| | | accaacctggcagacagggagCTGGTTCACATGATCAACTGGGCGAAGAGGGTGCCAGGCTTTG |
| | | TGGATTTGACCCTCCATGATCAGGTCCACCTTCTAGAATGTGCCTGGATGGAG |
| | | ATCCTGATGATTGGTGTGGTCTGGCGCTCCATGGAGCACCCAGTGA |
| | | AGCTACTGTTTGCTCCTAACTTGCTCTTGGACAGGGACCAGGGAAA |
| | | ATGTGTAGAGGGCCTGGTGGAGATCTTCGACATGCTGCTGGCTACA |
| | | TCATCTCGGTTCCGCATGATGAATCTGCAGGGAGAGGAGTTTGTGT |
| | | GCCTCAAATCTATTATTTTGCTTAATTCTGGAGTGTACACATTTCTG |
| | | CCCAGCACCCTGAAGTCTCTGGAAGAGAAGGACCATATCCACCGA |
| | | GTCCTGGACAAGATCACAGACACTTTGATCCACCTGATGGCCAAG |
| | | GCAGGCCTGACCCTGCAGCAGCAGCACCAGCGGCTGGCCCAGCTC |
| | | CTCCTCATCCTCTCCCACATCAGGCACATGAGTAACAAAGGCATGG |
| | | AGCTGctgtacagcatgaagtgcaagaacgtggtgcccctctatGaCctgctgctggaggcggc |
| | | ggacgcccaccgcctacatgcgcccactagccgtggaggggcatccgtggaggagacggaccaa |
| | | agccacttggccactgcgggctctacttcatcgcattccttgcaaaagtattacatcacggggg |
| | | aggcagagggtttccctgccaca |
| Mutant 81 | 66 | AGDMRAANLWPSPLMIKRSKKNSLALSLTADQMVSALLDAEPPILYS |
| (ERT | | EYDPTRPFSEASMMGLLTNLADRELVHMINWAKRVPGFVDLTLHDQ |
| mutant) | | VHLLECAWMEILMIGVVWRSMEHPVKLLFAPNLLLDRDQGKCVEGL |
| | | VEIFDMLLATSSRFRMMNLQGEEFVCLKSIILLNSGVYTFLPSTLKSLE |
| | | EKDHIHRVLDKITDTLIHLMAKAGLTLQQQHQRLAQLLLILSHIRHMS |
| | | NKGMELLYSMKCKNVVPLYDLLLEAADAHRLHAPTSRGGASVEETD |
| | | QSHLATAGSTSSHSLQKYYITGEAEGFPAT |
| SB07123 | 67 | aagcttgaattcgagcttgcatgcctgcaggtcgttacataacttacggtaaatggcccgcct |
| vector | | ggctgaccgcccaacgacccccgcccattgacgtcaataatgacgtatgttcccatagtaacgc |
| | | caatagggactttccattgacgtcaatgggtggagtatttacggtaaactgcccacttggcagt |
| | | acatcaagtgtatcatatgccaagtacgccccctattgacgtcaatgacggtaaatggcccgcc |
| | | tggcattatgcccagtacatgaccttatgggactttcctacttggcagtacatctacgtattag |
| | | tcatcgctattaccatggtgatgcggttttggcagtacatcaatgggcgtggatagcggtttga |
| | | ctcacggggatttccaagtctccaccccattgacgtcaatgggagtttgttttggcaccaaaatc |
| | | aacgggactttccaaaatgtcgtaacaactccgccccattgacgcaaatgggcggtaggcgtgt |
| | | acggtgggaggtctatataagcagagctcaataaaagagcccacaacccctcactcggcgcgcc |
| | | agtcctccgattgactgagtcgcccgggtacccgtgtatccaataaaccctcttgcagttgcat |
| | | ccgacttgtggtctcgctgttccttgggagggtctcctctgagtgattgactacccgtcagcgg |
| | | gggtctttcatttgggggctcgtccgagatcgggagacccctgcccagggaccaccgacccacc |
| | | accgggaggtaagctggccagcaacttatctgtgtctgtccgattgtctagtgtctatgactga |
| | | ttttatgcgcctgcgtcggtactagttagctaactagctctgtatctggcggacccgtggtgga |
| | | actgacgagttcggaacacccggccgcaaccctgggagacgtcccagggacttcgggggccgtt |
| | | tttgtggcccgacctgagtcctaaaatcccgatcgtttaggactctttggtgcaccccccttag |
| | | aggagggatatgtggttctggtaggagacgagaacctaaaacagttcccgcctccgtctgaatt |
| | | tttgctttcggtttgggaccgaagccgcgccgcgcgtcttgtctgctgcagcatcgttctgtgt |
| | | tgtctctgtctgactgtgtttctgtatttgtctgaaaatatgggccccccctcgagtccccagc |
| | | atgcctgctattctcttcccaatcctcccccttgctgtcctgccccaccccaccccccagaata |
| | | gaatgacacctactcagacaatgcgatgcaatttcctcattttattaggaaaggacagtgggag |
| | | tggcaccttccagggtcaaggaaggcacgggggaggggcaaacaacagatggctggcaactaga |
| | | aggcacagttacttaCTGTTTAAATATTAAACAGggaaccgatgtTAAATAAATAAATAAATA |
| | | AATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAGG |
| | | CTTCCACGGTCGAGGTCTTGTCCTTGGTGATGTCCTCGTGATCAATT |
| | | TCTTCCGAGGTGCAGGGGTAGAACTCAAGGGTCTGGCGGGCCTTCT |
| | | GCAACATGTTCGACACAGCCCTCAGGAGGTTTTGGGAGTGGTGTA |
| | | GGCACGGGAACATTCCAGGGTCGGGGGTTGCCACAGGGAGGTTCC |
| | | GGGAACCTCCTCCGGAGCCTCCTCCTGAACCTCCGCCTGATCCGCC |
| | | ACCGGAACAAGGCACGCTGGCCCATTCGCTCCAGGAGGACGAGTA |
| | | GTATCTATCCTGCGCCCGGACGCTGATTGACGCGTTCTTCCGACAA |
| | | ATCACAGTGGCGGAGGTTTTGTCGGTGAACACCCGGTCTTTCTTCT |
| | | CCCGTTTGGACTTTCCCTGCACTTGCACACAGAAAGTGAGCGAGAA |
| | | GTATGAGTGCGGGGTGCTCCAAGTGTCTGGATATTCCCAAGACACT |
| | | TCCACTTGGCGGGAGTTCTTGAGTGGCTTCAGCTGCAAGTTCTTGG |
| | | GGGGGTCAGGCTTGATGATGTCGCGGATAAAGAAGGAGGAAGTGT |
| | | AGTTCTCGTATTTCAGCTTATGCACGGCATCGACCATGACCTCGAT |
| | | AGGCAGGGACTCTTCCGCGGCAGGGCAGGCGCTGTCCTCCTGGCA |
| | | TTCCACGGAGTACTCATATTCCTTGTTGTCTCCCCTGACTCTCTCGG |
| | | CGGACAGAGTGGCGGCTCCACAGGTCACGCCCTGAGGATCGCTTG |
| | | ATCCCCGTGACGACTTCACGGAGAAAGTCAGGTCGGTGGAGATTG |
| | | TCGTCAGCCACCAACAGGTGAACCGACCGCTGTAGTTCTTGGCTTC |
| | | GCAGCGGAGGAAGGTCTTGTTCTTCGGTTCTTTTTGGTCCTTGAGG |
| | | ATGTCAGTGGACCAGATTCCATCCTCTTTCTTGTGCAGCAGCAGCA |
| | | GGGAGTGGGACAGCACTTCGCCACCCTTGTGGCAAGTGTACTGGC |
| | | CCGCGTCGCCGAACTCCTTGACTTGAATGGTCAGGGTCTTTCCGCT |
| | | TCCGAGCACCTCGGAGCTCTGATCCAGGGTCCAGGTTATGCCGTCC |
| | | TCTTCTGGCGTATCGCAAGTCAGCACGACCATTTCTCCAGGGGCGT |
| | | CCGGGTACCAATCCAGCTCGACCACGTAGACGTCCTTCTTCAGTTC |
| | | CCAAATGGCGACCAGAGGGGAAGCGAGGAACACAAGGGAGAACC |
| | | AGGAGATGACGAGTTGCTGATGGCACATCATGGTGGCGACACCGG |
| | | TACGCGTTGGCCCCCATTATATACCCTCTAGAACTAGTtatccactccgtgt |
| | | aagggagagtgagcctcttacgaatcTTCGGCGTCGACTGCTTCattccgaggcgactgatac |
| | | cTTCGGCGTCGACTGCTTCatacgaaggcagtccgattcTTCGGCGTCGACTGC |
| | | TTCaacctttactgagacgggacTTCGGCGTCGACTGCTTCaaaggcgttgcgaatcctcat |
| | | gcgattgttacgaaacccgTTAATTAAAGAGCGAGATTCCGTCTCAAAGAAA |
| | | AAAAAAGTAATGAAATGAATAAAATGAGTCCTAGAGCCAGTAAAT |
| | | GTCGTAAATGTCTCAGCTAGTCAGGTAGTAAAAGGTCTCAACTAG |
| | | GCAGTGGCAGAGCAGGATTCAAATTCAGGGCTGTTGTGATGCCTC |
| | | CGCAGACTCTGAGCGCCACCTGGTGGTAATTTGTCTGTGCCTCTTC |
| | | TGACGTGGAAGAACAGCAACTAACACACTAACACGGCATTTACTA |
| | | TGGGCCAGCCATTGTCCATCTAGATGGccgataaaataaaagattttatttagtctccag |
| | | aaaaaggggggaatgaaagaccccacctgtaggtttggcaagctagctgcagtaacgccattt |
| | | tgcaaggcatggaaaaataccaaaccaagaatagagaagttcagatcaagggcgggtacatgaa |
| | | aatagctaacgttgggccaaacaggatatctgcggtgagcagtttcggccccggcccggggcca |
| | | agaacagatggtcaccgcagtttcggccccggcccgaggccaagaacagatggtccccagata |
| | | tggcccaaccctcagcagtttcttaagacccatcagatgtttccaggctcccccaaggacctg |
| | | aaatgaccctgcgccttatttgaattaaccaatcagcctgcttctcgcttctgttcgcgcgct |
| | | tctgcttcccgagctctataaaagagctcacaacccctcactcggcgcgccagtcctccgac |
| | | agactgagtcgcccgggGGATCCGCCACCATGTCTAGACCTGGCGAGAGG |
| | | CCCTTCCAGTGCCGGATCTGCATGCGGAACTTCAGCAACATGAGCA |
| | | ACCTGACCAGACACACCCGGACACACACAGGCGAGAAGCCTTTTC |
| | | AGTGCAGAATCTGTATGCGCAATTTCTCCGACAGAAGCGTGCTGCG |
| | | GAGACACCTGAGAACCCACACCGGCAGCCAGAAACCATTCCAGTG |
| | | TCGCATCTGTATGAGAAACTTTAGCGACCCCTCCAATCTGGCCCGG |
| | | CACACCAGAACACATACCGGGGAAAAACCCTTTCAGTGTAGGATA |
| | | TGCATGAGGAATTTTTCCGACCGGTCCAGCCTGAGGCGGCACCTGA |
| | | GGACACATACTGGCTCCCAAAAGCCGTTCCAATGTCGGATATGTAT |
| | | GCGCAACTTTAGCCAGAGCGGCACCCTGCACAGACACACAAGAAC |
| | | CCATACTGGCGAGAAACCTTTCCAATGTAGAATCTGCATGCGAAAT |
| | | TTTTCCCAGCGGCCTAATCTGACCAGGCATCTGAGGACCCACCTGA |
| | | GAGGATCTaCCTGCAGGGATGAGTTTCCCACCATGGTGTTTCCTTCT |
| | | GGGCAGATCAGCCAGGCCTCGGCCTTGGCCCCGGCCCCTCCCCAA |
| | | GTCCTGCCCCAGGCTCCAGCCCCTGCCCCTGCTCCAGCCATGGTAT |
| | | CAGCTCTGGCCCAGGCCCCAGCCCCTGTCCCAGTCCTAGCCCCAGG |
| | | CCCTCCTCAGGCTGTGGCCCCACCTGCCCCCAAGCCCACCCAGGCT |
| | | GGGGAAGGAACGCTGTCAGAGGCCCTGCTGCAGCTGCAGTTTGAT |
| | | GATGAAGACCTGGGGGCCTTGCTTGGCAACAGCACAGACCCAGCT |
| | | GTGTTCACAGACCTGGCATCCGTCGACAACTCCGAGTTTCAGCAGC |
| | | TGCTGAACCAGGGCATACCTGTGGCCCCCCACACAACTGAGCCCA |
| | | TGCTGATGGAGTACCCTGAGGCTATAACTCGCCTAGTGACAGGGG |
| | | CCCAGAGGCCCCCCGACCCAGCTCCTGCTCCACTGGGGGCCCCGG |
| | | GGCTCCCCAATGGCCTCCTTTCAGGAGATGAAGACTTCTCCTCCAT |
| | | TGCGGACATGGACTTCTCAGCCCTGCTGAGTCAGATCAGCTCCcaatt |
| | | gtgcgtacgcggatcctctgctggagacatgagagctgccaacctttggccaagcccgctcat |
| | | gatcaaacgctctaagaagaacagcctggccttgtccctgacggccgaccagatggtcagtgc |
| | | cttgttggatgctgagccccccatactctattccgagtatgatcctaccagacccttcagtg |
| | | aagcttcgatgatgggcttactgaccaacctggcagacagggagCTGGTTCACATGATCAAC |
| | | TGGGCGAAGAGGGTGCCAGGCTTTGTGGATTTGACCCTCCATGATCAGGTCCACCTTCTAGAA |
| | | TGTGCCTGGATGGAGATCCTGATGATTGGTGTGGTCTGGCGCTCCATGGA |
| | | GCACCCAGTGAAGCTACTGTTTGCTCCTAACTTGCTCTTGGACAGG |
| | | GACCAGGGAAAATGTGTAGAGGGCCTGGTGGAGATCTTCGACATG |
| | | CTGCTGGCTACATCATCTCGGTTCCGCATGATGAATCTGCAGGGAG |
| | | AGGAGTTTGTGTGCCTCAAATCTATTATTTTGCTTAATTCTGGAGTG |
| | | TACACATTTCTGCCCAGCACCCTGAAGTCTCTGGAAGAGAAGGAC |
| | | CATATCCACCGAGTCCTGGACAAGATCACAGACACTTTGATCCACC |
| | | TGATGGCCAAGGCAGGCCTGACCCTGCAGCAGCAGCACCAGCGGC |
| | | TGGCCCAGCTCCTCCTCATCCTCTCCCACATCAGGCACATGAGTAA |
| | | CAAAGGCATGGAGCTGctgtacagcatgaagtgcaagaacgtggtgcccctctatGaCctgct |
| | | gctggaggcggcggacgcccaccgcctacatgcgcccactagccgtggaggggcatccgtgga |
| | | ggagacggaccaaagccacttggccactggggctctacttcatcgcattccttgcaaaagtat |
| | | tacatcacgggggaggcagagggtttccctgccacaTaAGTCGACAATCAACCTCtggattaca |
| | | aaatttgtgaaagattgactggtattcttaactatgttgctccttttacgctatgtggatacg |
| | | ctgctttaatgcctttgtatcatgctattgcttcccgtatggctttcattttctcctccttgt |
| | | ataaatcctggttgctgtctctttatgaggagttgtggcccgttgtcaggcaacgtggcgtggt |
| | | gtgcactgtgtttgctgacgcaacccccactggttggggcattgccaccacctgtcagctcct |
| | | ttccgggactttcgctttccccctccctattgccacggcggaactcatcgccgcctgccttgcc |
| | | cgctgctggacaggggctcggctgttgggcactgacaattccgtggtgttgtcggggaaatcat |
| | | cgtcctttccttggctgctcgcctgtgttgccacctggattctgcgcgggacgtccttctgcta |
| | | cgtcccttcggccctcaatccagcggaccttccttcccgcggcctgctgccggctctgcggcct |
| | | cttccgcgtctacgccttcgccctcagacgagtcggatctccctttgggccgcctccccgcga |
| | | tatcagtggtccaggctctagttttgactcaacaatatcaccagctgaagcctatagagtacga |
| | | gccatagataaaataaaagattttatttagtctccagaaaaaggggggaatgaaagaccccac |
| | | ctgtaggtttggcaagctagcaataaaagagcccacaacccctcactcggggcgccagtcctcc |
| | | gattgactgagtcgcccggccgcttcgagcagacatgataagatacattgatgagtttggacaa |
| | | accacaactagaatgcagtgaaaaaaatgctttatttgtgaaatttgtgatgctattgctttat |
| | | ttgtaaccattataagctgcaataaacaagttaacaacaacaattgcattcattttatgtttca |
| | | ggttcagggggagatgtgggaggttttttaaagcaagtaaaacctctacaaatgtggtaaaatc |
| | | gataaggatcgggtacccgtgtatccaataaaccctcttgcagttgcatccgacttgtggtct |
| | | cgctgttccttgggagggtctcctctgagtgattgactacccgtcagcgggggtctttcacaca |
| | | tgcagcatgtatcaaaattaatttggttttttttcttaagctgtgccttctagttgccagccat |
| | | ctgttgtttgcccctcccccgtgccttccttgaccctggaaggtgccactcccactgtcctttc |
| | | ctaataaaatgaggaaattgcatcgcattgtctgagtaggtgtcattctattctggggggtggggggggca |
| | | ggacagcaagggggaggattgggaagacaatagcaggcatgctggggatgcggtgggctctatggagatccc |
| | | gcggtacctcgcgaatgcatctagatccaatggcctttttggcccagacatgataagatacattgatgagtt |
| | | tggacaaaccacaactagaatgcagtgaaaaaaatgctttatttgtgaaatttgtgatgctattgctttatt |
| | | tgtaaccattataagctgcaataaacaagttgcggccgcttagccctcccac |
| | | acataaccagagggcagcaattcacgaatcccaactgccgtcggctgtccatcactgtccttcactatggctt |
| | | tgatcccaggatgcagatcgagaagcacctgtcggcaccgtccgcaggggctcaagatgcccctgttctcatt |
| | | tccgatcgcgacgatacaagtcaggttgccagctgccgcagcagcagcagtgcccagcaccacgagttctgca |
| | | caaggtcccccagtaaaatgatatacattgacaccagtgaagatgcggccgtcgctagagagagctgcgctg |
| | | gcgacgctgtagtcttcagagatggggatgctgttgattgtagccgttgctctttcaatgagggtggattctt |
| | | cttgagacaaaggcttggccatgcggccgccgctcggtgttcgaggccacacgcgtcaccttaatatgcgaag |
| | | tggacctcggaccgcgccgccccgactgcatctgcgtgttcgaattcgccaatgacaagacgctgggcggggt |
| | | ttgtgtcatcatagaactaaagacatgcaaatatatttcttccggggggtaccggcctttttggccATTGGat |
| | | cggatctggccaaaaaggcccttaagtatttacattaaatggccatagtacttaaagttacattggcttcctt |
| | | gaaataaacatggagtattcagaatgtgtcataaatatttctaattttaagatagtatctccattggctttct |
| | | actttttcttttatttttttttgtcctctgtcttccatttgttgttgttgttgtttgtttgtttgtttgttgg |
| | | ttggttggttaatttttttttaaagatcctacactatagttcaagctagactattagctactctgtaacccag |
| | | ggtgaccttgaagtcatgggtagcctgctgttttagccttcccacatctaagattacaggtatgagctatcat |
| | | ttttggtatattgattgattgattgattgatgtgtgtgtgtgtgattgtgtttgtgtgtgtgaTtgtgTaTat |
| | | gtgtgtatggTtgtgtgtgaTtgtgtgtatgtatgTTtgtgtgtgaTtgTgtgtgtgtgaTtgtgcatgtgtg |
| | | tgtgtgtgaTtgtgtTtatgtgtatgaTtgtgtgtgtgtgtgtgtgtgtgtgtgtgtgtgtgtgtgtgtgtgt |
| | | gttgtgTaTaTatatttatggtagtgagagGcaacgctccggctcaggtgtcaggttggtttttgagacagag |
| | | tctttcacttagcttggaattcactggccgtcgttttacaacgtcgtgactgggaaaaccctggcgttaccca |
| | | acttaatcgccttgcagcacatccccctttcgccagctggcgtaatagcgaagaggcccgcaccgatcgccct |
| | | tcccaacagttgcgcagcctgaatggcgaatggcgcctgatgcggtattttctccttacgcatctgtgcggtat |
| | | ttcacaccgcatatggtgcactctcagtacaatctgctctgatgccgcatagttaagccagccccgacacccg |
| | | ccaacacccgctgacgcgccctgacgggcttgtctgctcccggcatccgcttacagacaagctgtgaccgtct |
| | | ccgggagctgcatgtgtcagaggttttcaccgtcatcaccgaaacgcgcgagacgaaagggcctcgtgatacg |
| | | cctatttttataggttaatgtcatgataataatggtttcttagacgtcaggtggcacttttcggggaaatgtg |
| | | cgcggaacccctatttgtttatttttctaaatacattcaaatatgtatccgctcatgagacaataaccctgat |
| | | aaatgcttcaataatattgaaaaaggaagagtatgagtattcaacatttccgtgtcgcccttattcccttttt |
| | | tgcggcattttgccttcctgtttttgctcacccagaaacgctggtgaaagtaaaagatgctgaagatcagttg |
| | | ggtgcacgagtgggttacatcgaactggatctcaacagcggtaagatccttgagagttttcgccccgaagaac |
| | | gttttccaatgatgagcacttttaaagttctgctatgtggcgcggtattatcccgtattgacgccgggcaag |
| | | agcaactcggtcgccgcatacactattctcagaatgacttggttgagtactcaccagtcacagaaaagcatct |
| | | tacggatggcatgacagtaagagaattatgcagtgctgccataaccatgagtgataacactgcggccaactta |
| | | cttctgacaacgatcggaggaccgaaggagctaaccgcttttttgcacaacatgggggatcatgtaactcgc |
| | | cttgatcgttgggaaccggagctgaatgaagccataccaaacgacgagcgtgacaccacgatgcctgtagca |
| | | atggcaacaacgttgcgcaaactattaactggcgaactacttactctagcttcccggcaacaattaatagact |
| | | ggatggaggggataaagttgcaggaccacttctgcgctcggcccttccggctggctggtttattgctgataaa |
| | | tctggagccggtgagcgtgggtctcgcggtatcattgcagcactggggccagatggtaagccctcccgtatcg |
| | | tagttatctacacgacggggagtcaggcaactatggatgaacgaaatagacagatcgctgagataggtgcctc |
| | | actgattaagcattggtaactgtcagaccaagtttactcatatatactttagattgatttaaaacttcatttt |
| | | taatttaaaaggatctaggtgaagatcctttttgataatctcatgaccaaaatcccttaacgtgagttttcgt |
| | | tccactgagcgtcagaccccgtagaaaagatcaaaggatcttcttgagatcctttttttctgcgcgtaatctg |
| | | ctgcttgcaaacaaaaaaaccaccgctaccagcggtggtttgtttgccggatcaagagctaccaactcttttt |
| | | ccgaaggtaactggcttcagcagagcgcagataccaaatactgtccttctagtgtagccgtagttaggccacc |
| | | acttcaagaactctgtagcaccgcctacatacctcgctctgctaatcctgttaccagtggctgctgccagtgg |
| | | cgataagtcgtgtcttaccgggttggactcaagacgatagttaccggataaggcgcagcggtcgggctgaacg |
| | | gggggttcgtgcacacagcccagcttggagcgaacgacctacaccgaactgagatacctacagcgtgagctat |
| | | gagaaagcgccacgcttcccgaagggagaaaggcggacaggtatccggtaagcggcagggtcggaacaggag |
| | | agcgcacgagggagcttccagggggaaacgcctggtatctttatagtcctgtcgggtttcgccacctctgact |
| | | tgagcgtcgatttttgtgatgctcgtcaggggggcggagcctatggaaaaacgccagcaacgcggccttttt |
| | | acggttcctggccttttgctggccttttgctcacatgttctttcctgcgttatcccctgattctgtggataac |
| | | cgtattaccgcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgcagctggca |
| | | cgacaggtttcccgactggaaagcgggcagtgagcgcaacgcaattaatgtgagttagctcactcattaggca |
| | | ccccaggctttacactttatgcttccggctcgtatgttgtgtggaattgtgagcggataacaatttcacacag |
| | | gaaacagctatgaccatgattacgcc |
| Mutant 77 | 68 | gctggagacatgagagctgccaacctttggccaagcccgctcatgatcaaacgctctaagaagaacagcctg |
| (ERT | | gccttgtccctgacggccgaccagatggtcagtgccttgttggatgctgagccccccatactctattccgag |
| mutant) | | tatgatcctaccagacccttcagtgaagcttcgatgatgggcttactgaccaacctggcagacagggagATCG |
| | | TTCACATGATCAACTGGGCGAAGAGGGTGCCAGGCTTTGTGGATTT |
| | | GACCCTCCATGATCAGGTCCACCTTCTAGAATGTGCCTGGCTAGAG |
| | | ATCCTGATGATTGGTGTGGTCTGGCGCTCCATGGAGCACCCAGTGA |
| | | AGCTACTGTTTGCTCCTAACTTGCTCTTGGACAGGGACCAGGGAAA |
| | | ATGTGTAGAGGGCCTGGTGGAGATCTTCGACATGCTGCTGGCTACA |
| | | TCATCTCGGTTCCGCATGATGAATCTGCAGGGAGAGGAGTTTGTGT |
| | | GCCTCAAATCTATTATTTTGCTTAATTCTGGAGTGTACACATTTCTG |
| | | CCCAGCACCCTGAAGTCTCTGGAAGAGAAGGACCATATCCACCGA |
| | | GTCCTGGACAAGATCACAGACACTTTGATCCACCTGATGGCCAAG |
| | | GCAGGCCTGACCCTGCAGCAGCAGCACCAGCGGCTGGCCCAGCTC |
| | | CTCCTCATCCTCTCCCACATCAGGCACGCCAGTAACAAAGGCATGG |
| | | AGCTGctgtacagcatgaagtgcaagaacgtggtgcccctctatGaCctgctgctggaggcggcggac |
| | | gcccaccgcctacatgcgcccactagccgtggaggggcatccgtggaggagacggaccaaagccacttgg |
| | | ccactgcgggctctacttcatcgcattccttgcaaaagtattacatcacgggggaggcagagggtttccctgc |
| | | caca |
| Mutant 77 | 69 | AGDMRAANLWPSPLMIKRSKKNSLALSLTADQMVSALLDAEPPILYS |
| (ERT | | EYDPTRPFSEASMMGLLTNLADREIVHMINWAKRVPGFVDLTLHDQV |
| mutant) | | HLLECAWLEILMIGVVWRSMEHPVKLLFAPNLLLDRDQGKCVEGLVE |
| | | IFDMLLATSSRFRMMNLQGEEFVCLKSIILLNSGVYTFLPSTLKSLEEK |
| | | DHIHRVLDKITDTLIHLMAKAGLTLQQQHQRLAQLLLILSHIRHASNK |
| | | GMELLYSMKCKNVVPLYDLLLEAADAHRLHAPTSRGGASVEETDQS |
| | | HLATAGSTSSHSLQKYYITGEAEGFPAT |
| SB07129 | 70 | aagcttgaattcgagcttgcatgcctgcaggtcgttacataacttacggtaaatggcccgcctggctgaccgc |
| vector | | ccaacgacccccgcccattgacgtcaataatgacgtatgttcccatagtaacgccaatagggactttccattg |
| | | acgtcaatgggtggagtatttacggtaaactgcccacttggcagtacatcaagtgtatcatatgccaagtacg |
| | | ccccctattgacgtcaatgacggtaaatggcccgcctggcattatgcccagtacatgaccttatgggactttc |
| | | ctacttggcagtacatctacgtattagtcatcgctattaccatggtgatgcggttttggcagtacatcaatgg |
| | | gcgtggatagcggtttgactcacggggatttccaagtctccaccccattgacgtcaatgggagtttgttttg |
| | | gcaccaaaatcaacgggactttccaaaatgtcgtaacaactccgccccattgacgcaaatgggcggtaggcgt |
| | | gtacggtgggaggtctatataagcagagctcaataaaagagcccacaacccctcactcggcgcgccagtcct |
| | | ccgattgactgagtcgcccgggtacccgtgtatccaataaaccctcttgcagttgcatccgacttgtggtct |
| | | cgctgttccttgggagggtctcctctgagtgattgactacccgtcagcgggggtctttcatttgggggctcg |
| | | tccgagatcgggagacccctgcccagggaccaccgacccaccaccgggaggtaagctggccagcaacttatct |
| | | gtgtctgtccgattgtctagtgtctatgactgattttatgcgcctgcgtcggtactagttagctaactagct |
| | | ctgtatctggcggacccgtggtggaactgacgagttcggaacacccggccgcaaccctgggagacgtcccagg |
| | | gacttcgggggccgtttttgtggcccgacctgagtcctaaaatcccgatcgtttaggactctttggtgcacc |
| | | ccccttagaggagggatatgtggttctggtaggagacgagaacctaaaacagttcccgcctccgtctgaattt |
| | | ttgctttcggtttgggaccgaagccgcgccgcgcgtcttgtctgctgcagcatcgttctgtgttgtctctgtc |
| | | tgactgtgtttctgtatttgtctgaaaatatgggccccccctcgagtccccagcatgcctgctattctcttcc |
| | | caatcctcccccttgctgtcctgccccaccccaccccccagaatagaatgacacctactcagacaatgcgatg |
| | | caatttcctcattttattaggaaaggacagtgggagtggcaccttccagggtcaaggaaggcacgggggaggg |
| | | gcaaacaacagatggctggcaactagaaggcacagttacttaCTGTTTAAATATTAAACAGggaaccgatgtT |
| | | AAATAAATAAATAAATAAATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAGG |
| | | CTTCCACGGTCGAGGTCTTGTCCTTGGTGATGTCCTCGTGATCAATT |
| | | TCTTCCGAGGTGCAGGGGTAGAACTCAAGGGTCTGGCGGGCCTTCT |
| | | GCAACATGTTCGACACAGCCCTCAGGAGGTTTTGGGAGTGGTGTA |
| | | GGCACGGGAACATTCCAGGGTCGGGGGTTGCCACAGGGAGGTTCC |
| | | GGGAACCTCCTCCGGAGCCTCCTCCTGAACCTCCGCCTGATCCGCC |
| | | ACCGGAACAAGGCACGCTGGCCCATTCGCTCCAGGAGGACGAGTA |
| | | GTATCTATCCTGCGCCCGGACGCTGATTGACGCGTTCTTCCGACAA |
| | | ATCACAGTGGCGGAGGTTTTGTCGGTGAACACCCGGTCTTTCTTCT |
| | | CCCGTTTGGACTTTCCCTGCACTTGCACACAGAAAGTGAGCGAGAA |
| | | GTATGAGTGCGGGGTGCTCCAAGTGTCTGGATATTCCCAAGACACT |
| | | TCCACTTGGCGGGAGTTCTTGAGTGGCTTCAGCTGCAAGTTCTTGG |
| | | GGGGGTCAGGCTTGATGATGTCGCGGATAAAGAAGGAGGAAGTGT |
| | | AGTTCTCGTATTTCAGCTTATGCACGGCATCGACCATGACCTCGAT |
| | | AGGCAGGGACTCTTCCGCGGCAGGGCAGGCGCTGTCCTCCTGGCA |
| | | TTCCACGGAGTACTCATATTCCTTGTTGTCTCCCCTGACTCTCTCGG |
| | | CGGACAGAGTGGCGGCTCCACAGGTCACGCCCTGAGGATCGCTTG |
| | | ATCCCCGTGACGACTTCACGGAGAAAGTCAGGTCGGTGGAGATTG |
| | | TCGTCAGCCACCAACAGGTGAACCGACCGCTGTAGTTCTTGGCTTC |
| | | GCAGCGGAGGAAGGTCTTGTTCTTCGGTTCTTTTTGGTCCTTGAGG |
| | | ATGTCAGTGGACCAGATTCCATCCTCTTTCTTGTGCAGCAGCAGCA |
| | | GGGAGTGGGACAGCACTTCGCCACCCTTGTGGCAAGTGTACTGGC |
| | | CCGCGTCGCCGAACTCCTTGACTTGAATGGTCAGGGTCTTTCCGCT |
| | | TCCGAGCACCTCGGAGCTCTGATCCAGGGTCCAGGTTATGCCGTCC |
| | | TCTTCTGGCGTATCGCAAGTCAGCACGACCATTTCTCCAGGGGCGT |
| | | CCGGGTACCAATCCAGCTCGACCACGTAGACGTCCTTCTTCAGTTC |
| | | CCAAATGGCGACCAGAGGGGAAGCGAGGAACACAAGGGAGAACC |
| | | AGGAGATGACGAGTTGCTGATGGCACATCATGGTGGCGACACCGG |
| | | TACGCGTTGGCCCCCATTATATACCCTCTAGAACTAGTtatccactccgtgt |
| | | aagggagagtgagcctcttacgaatcTTCGGCGTCGACTGCTTCattccgaggcgactgatac |
| | | cTTCGGCGTCGACTGCTTCatacgaaggcagtccgattcTTCGGCGTCGACTGC |
| | | TTCaacctttactgagacgggacTTCGGCGTCGACTGCTTCaaaggcgttgcgaatcctcat |
| | | gcgattgttacgaaacccgTTAATTAAAGAGCGAGATTCCGTCTCAAAGAAA |
| | | AAAAAAGTAATGAAATGAATAAAATGAGTCCTAGAGCCAGTAAAT |
| | | GTCGTAAATGTCTCAGCTAGTCAGGTAGTAAAAGGTCTCAACTAG |
| | | GCAGTGGCAGAGCAGGATTCAAATTCAGGGCTGTTGTGATGCCTC |
| | | CGCAGACTCTGAGCGCCACCTGGTGGTAATTTGTCTGTGCCTCTTC |
| | | TGACGTGGAAGAACAGCAACTAACACACTAACACGGCATTTACTA |
| | | TGGGCCAGCCATTGTCCATCTAGATGGccgataaaataaaagattttatttagtctccag |
| | | aaaaaggggggaatgaaagaccccacctgtaggtttggcaagctagctgcagtaacgccattttgcaaggcat |
| | | ggaaaaataccaaaccaagaatagagaagttcagatcaaggggggtacatgaaaatagctaacgttgggcc |
| | | aaacaggatatctgcggtgagcagtttcggccccggcccggggccaagaacagatggtcaccgcagtttcgg |
| | | ccccggcccgaggccaagaacagatggtccccagatatggcccaaccctcagcagtttcttaagacccatca |
| | | gatgtttccaggctcccccaaggacctgaaatgaccctgcgccttatttgaattaaccaatcagcctgcttct |
| | | cgcttctgttcgcgcgcttctgcttcccgagctctataaaagagctcacaacccctcactcggcgcgccagtc |
| | | ctccgacagactgagtcgcccgggGGATCCGCCACCATGTCTAGACCTGGCGAGAGG |
| | | CCCTTCCAGTGCCGGATCTGCATGCGGAACTTCAGCAACATGAGCA |
| | | ACCTGACCAGACACACCCGGACACACACAGGCGAGAAGCCTTTTC |
| | | AGTGCAGAATCTGTATGCGCAATTTCTCCGACAGAAGCGTGCTGCG |
| | | GAGACACCTGAGAACCCACACCGGCAGCCAGAAACCATTCCAGTG |
| | | TCGCATCTGTATGAGAAACTTTAGCGACCCCTCCAATCTGGCCCGG |
| | | CACACCAGAACACATACCGGGGAAAAACCCTTTCAGTGTAGGATA |
| | | TGCATGAGGAATTTTTCCGACCGGTCCAGCCTGAGGCGGCACCTGA |
| | | GGACACATACTGGCTCCCAAAAGCCGTTCCAATGTCGGATATGTAT |
| | | GCGCAACTTTAGCCAGAGCGGCACCCTGCACAGACACACAAGAAC |
| | | CCATACTGGCGAGAAACCTTTCCAATGTAGAATCTGCATGCGAAAT |
| | | TTTTCCCAGCGGCCTAATCTGACCAGGCATCTGAGGACCCACCTGA |
| | | GAGGATCTaCCTGCAGGGATGAGTTTCCCACCATGGTGTTTCCTTCT |
| | | GGGCAGATCAGCCAGGCCTCGGCCTTGGCCCCGGCCCCTCCCCAA |
| | | GTCCTGCCCCAGGCTCCAGCCCCTGCCCCTGCTCCAGCCATGGTAT |
| | | CAGCTCTGGCCCAGGCCCCAGCCCCTGTCCCAGTCCTAGCCCCAGG |
| | | CCCTCCTCAGGCTGTGGCCCCACCTGCCCCCAAGCCCACCCAGGCT |
| | | GGGGAAGGAACGCTGTCAGAGGCCCTGCTGCAGCTGCAGTTTGAT |
| | | GATGAAGACCTGGGGGCCTTGCTTGGCAACAGCACAGACCCAGCT |
| | | GTGTTCACAGACCTGGCATCCGTCGACAACTCCGAGTTTCAGCAGC |
| | | TGCTGAACCAGGGCATACCTGTGGCCCCCCACACAACTGAGCCCA |
| | | TGCTGATGGAGTACCCTGAGGCTATAACTCGCCTAGTGACAGGGG |
| | | CCCAGAGGCCCCCCGACCCAGCTCCTGCTCCACTGGGGGCCCCGG |
| | | GGCTCCCCAATGGCCTCCTTTCAGGAGATGAAGACTTCTCCTCCAT |
| | | TGCGGACATGGACTTCTCAGCCCTGCTGAGTCAGATCAGCTCCcaatt |
| | | gtgcgtacgcggatcctctgctggagacatgagagctgccaacctttggccaagcccgctcatgatcaaacgc |
| | | tctaagaagaacagcctggccttgtccctgacggccgaccagatggtcagtgccttgttggatgctgagccccc |
| | | catactctattccgagtatgatcctaccagacccttcagtgaagcttcgatgatgggcttactgaccaacctg |
| | | gcagacagggagATCGTTCACATGATCAACTGGGCGAAGAGGGTGCCAGGC |
| | | TTTGTGGATTTGACCCTCCATGATCAGGTCCACCTTCTAGAATGTG |
| | | CCTGGCTAGAGATCCTGATGATTGGTGTGGTCTGGCGCTCCATGGA |
| | | GCACCCAGTGAAGCTACTGTTTGCTCCTAACTTGCTCTTGGACAGG |
| | | GACCAGGGAAAATGTGTAGAGGGCCTGGTGGAGATCTTCGACATG |
| | | CTGCTGGCTACATCATCTCGGTTCCGCATGATGAATCTGCAGGGAG |
| | | AGGAGTTTGTGTGCCTCAAATCTATTATTTTGCTTAATTCTGGAGTG |
| | | TACACATTTCTGCCCAGCACCCTGAAGTCTCTGGAAGAGAAGGAC |
| | | CATATCCACCGAGTCCTGGACAAGATCACAGACACTTTGATCCACC |
| | | TGATGGCCAAGGCAGGCCTGACCCTGCAGCAGCAGCACCAGCGGC |
| | | TGGCCCAGCTCCTCCTCATCCTCTCCCACATCAGGCACGCCAGTAA |
| | | CAAAGGCATGGAGCTGctgtacagcatgaagtgcaagaacgtggtgcccctctatGaCctgct |
| | | gctggaggcggggacgcccaccgcctacatgcgcccactagccgtggaggggcatccgtggaggagac |
| | | ggaccaaagccacttggccactgcgggctctacttcatcgcattccttgcaaaagtattacatcacgggggag |
| | | gcagagggtttccctgccacaTaAGTCGACAATCAACCTCtggattacaaaatttgtgaaagatt |
| | | gactggtattcttaactatgttgctccttttacgctatgtggatacgctgctttaatgcctttgtatcatgcta |
| | | ttgcttcccgtatggctttcattttctcctccttgtataaatcctggttgctgtctctttatgaggagttgtg |
| | | gcccgttgtcaggcaacgtggcgtggtgtgcactgtgtttgctgacgcaacccccactggttggggcattgcc |
| | | accacctgtcagctcctttccgggactttcgctttccccctccctattgccacggcggaactcatcgccgcct |
| | | gccttgcccgctgctggacaggggctcggctgttgggcactgacaattccgtggtgttgtcggggaaatcatc |
| | | gtcctttccttggctgctcgcctgtgttgccacctggattctgcgcgggacgtccttctgctacgtcccttcg |
| | | gccctcaatccagcggaccttccttcccgcggcctgctgccggctctgcggcctcttccgcgtctacgcctt |
| | | cgccctcagacgagtcggatctccctttgggccgcctccccgcgatatcagtggtccaggctctagttttga |
| | | ctcaacaatatcaccagctgaagcctatagagtacgagccatagataaaataaaagattttatttagtctcc |
| | | agaaaaaggggggaatgaaagaccccacctgtaggtttggcaagctagcaataaaagagcccacaacccctca |
| | | ctcggggcgccagtcctccgattgactgagtcgcccggccgcttcgagcagacatgataagatacattgatg |
| | | agtttggacaaaccacaactagaatgcagtgaaaaaaatgctttatttgtgaaatttgtgatgctattgctt |
| | | tatttgtaaccattataagctgcaataaacaagttaacaacaacaattgcattcattttatgtttcaggttca |
| | | gggggagatgtgggaggttttttaaagcaagtaaaacctctacaaatgtggtaaaatcgataaggatcgggta |
| | | cccgtgtatccaataaaccctcttgcagttgcatccgacttgtggtctcgctgttccttgggagggtctcctc |
| | | tgagtgattgactacccgtcagcgggggtctttcacacatgcagcatgtatcaaaattaatttggtttttttt |
| | | cttaagctgtgccttctagttgccagccatctgttgtttgcccctcccccgtgccttccttgaccctggaagg |
| | | tgccactcccactgtcctttcctaataaaatgaggaaattgcatcgcattgtctgagtaggtgtcattctatt |
| | | ctggggggtggggggggcaggacagcaagggggaggattgggaagacaatagcaggcatgctggggatgcggt |
| | | gggctctatggagatcccgcggtacctcgcgaatgcatctagatccaatggcctttttggcccagacatgat |
| | | aagatacattgatgagtttggacaaaccacaactagaatgcagtgaaaaaaatgctttatttgtgaaatttgt |
| | | gatgctattgctttatttgtaaccattataagctgcaataaacaagttgcggccgcttagccctcccacaca |
| | | taaccagagggcagcaattcacgaatcccaactgccgtcggctgtccatcactgtccttcactatggctttga |
| | | tcccaggatgcagatcgagaagcacctgtcggcaccgtccgcaggggctcaagatgcccctgttctcatttcc |
| | | gatcgcgacgatacaagtcaggttgccagctgccgcagcagcagcagtgcccagcaccacgagttctgcacaa |
| | | ggtcccccagtaaaatgatatacattgacaccagtgaagatgcggccgtcgctagagagagctgcgctggcg |
| | | acgctgtagtcttcagagatggggatgctgttgattgtagccgttgctctttcaatgagggtggattcttctt |
| | | gagacaaaggcttggccatgcggccgccgctcggtgttcgaggccacacgcgtcaccttaatatgcgaagtgg |
| | | acctcggaccgcgccgccccgactgcatctgcgtgttcgaattcgccaatgacaagacgctgggcggggtttg |
| | | tgtcatcatagaactaaagacatgcaaatatatttcttccggggggtaccggcctttttggccATTGGatcg |
| | | gatctggccaaaaaggcccttaagtatttacattaaatggccatagtacttaaagttacattggcttccttga |
| | | aataaacatggagtattcagaatgtgtcataaatatttctaattttaagatagtatctccattggctttcta |
| | | ctttttcttttatttttttttgtcctctgtcttccatttgttgttgttgttgtttgtttgtttgtttgttgg |
| | | ttggttggttaatttttttttaaagatcctacactatagttcaagctagactattagctactctgtaacccag |
| | | ggtgaccttgaagtcatgggtagcctgctgttttagccttcccacatctaagattacaggtatgagctatcat |
| | | ttttggtatattgattgattgattgattgatgtgtgtgtgtgtgattgtgtttgtgtgtgtgaTtgtgTaTa |
| | | tgtgtgtatggTtgtgtgtgaTtgtgtgtatgtatgTTtgtgtgtgaTtg |
| | | TgtgtgtgtgaTtgtgcatgtgtgtgtgtgtgaTtgtgtTtatgtgtatgaTtgtgtgtgtgtgtgtgtgtgt |
| | | gtgtgtgtgtgtgtgtgtgtgtgtgttgtgTaTaTatatttatggtagtgagagGcaacgctccggctcaggt |
| | | gtcaggttggtttttgagacagagtctttcacttagcttggaattcactggccgtcgttttacaacgtcgtg |
| | | actgggaaaaccctggcgttacccaacttaatcgccttgcagcacatccccctttcgccagctgggtaatag |
| | | cgaagaggcccgcaccgatcgcccttcccaacagttgcgcagcctgaatggcgaatggcgcctgatgcggta |
| | | ttttctccttacgcatctgtgcggtatttcacaccgcatatggtgcactctcagtacaatctgctctgatgcc |
| | | gcatagttaagccagccccgacacccgccaacacccgctgacgcgccctgacgggcttgtctgctcccggcat |
| | | ccgcttacagacaagctgtgaccgtctccgggagctgcatgtgtcagaggttttcaccgtcatcaccgaaac |
| | | gcgcgagacgaaagggcctcgtgatacgcctatttttataggttaatgtcatgataataatggtttcttaga |
| | | cgtcaggtggcacttttcggggaaatgtgcgcggaacccctatttgtttatttttctaaatacattcaaata |
| | | tgtatccgctcatgagacaataaccctgataaatgcttcaataatattgaaaaaggaagagtatgagtattca |
| | | acatttccgtgtcgcccttattcccttttttgcggcattttgccttcctgtttttgctcacccagaaacgctg |
| | | gtgaaagtaaaagatgctgaagatcagttgggtgcacgagtgggttacatcgaactggatctcaacagcggta |
| | | agatccttgagagttttcgccccgaagaacgttttccaatgatgagcacttttaaagttctgctatgtggcgc |
| | | ggtattatcccgtattgacgccgggcaagagcaactcggtcgccgcatacactattctcagaatgacttggt |
| | | tgagtactcaccagtcacagaaaagcatcttacggatggcatgacagtaagagaattatgcagtgctgccata |
| | | accatgagtgataacactgcggccaacttacttctgacaacgatcggaggaccgaaggagctaaccgcttttt |
| | | tgcacaacatgggggatcatgtaactcgccttgatcgttgggaaccggagctgaatgaagccataccaaacg |
| | | acgagcgtgacaccacgatgcctgtagcaatggcaacaacgttgcgcaaactattaactggcgaactactta |
| | | ctctagcttcccggcaacaattaatagactggatggaggcggataaagttgcaggaccacttctgcgctcgg |
| | | cccttccggctggctggtttattgctgataaatctggagccggtgagcgtgggtctcgcggtatcattgcagc |
| | | actggggccagatggtaagccccccgtatcgtagttatctacacgacggggagtcaggcaactatggatgaa |
| | | cgaaatagacagatcgctgagataggtgcctcactgattaagcattggtaactgtcagaccaagtttactca |
| | | tatatactttagattgatttaaaacttcatttttaatttaaaaggatctaggtgaagatcctttttgataat |
| | | ctcatgaccaaaatcccttaacgtgagttttcgttccactgagcgtcagaccccgtagaaaagatcaaaggat |
| | | cttcttgagatcctttttttctgcgcgtaatctgctgcttgcaaacaaaaaaaccaccgctaccagcggtggt |
| | | ttgtttgccggatcaagagctaccaactctttttccgaaggtaactggcttcagcagagcgcagataccaaat |
| | | actgtccttctagtgtagccgtagttaggccaccacttcaagaactctgtagcaccgcctacatacctcgct |
| | | ctgctaatcctgttaccagtggctgctgccagtggcgataagtcgtgtcttaccgggttggactcaagacgat |
| | | agttaccggataaggcgcagcggtcgggctgaacggggggttcgtgcacacagcccagcttggagcgaacgac |
| | | ctacaccgaactgagatacctacagcgtgagctatgagaaagcgccacgcttcccgaagggagaaaggcggac |
| | | aggtatccggtaagcggcagggtcggaacaggagagcgcacgagggagcttccagggggaaacgcctggtatc |
| | | tttatagtcctgtcgggtttcgccacctctgacttgagcgtcgatttttgtgatgctcgtcaggggggggagc |
| | | ctatggaaaaacgccagcaacgcggcctttttacggttcctggccttttgctggccttttgctcacatgttct |
| | | ttcctgcgttatcccctgattctgtggataaccgtattaccgcctttgagtgagctgataccgctcgccgcag |
| | | ccgaacgaccgagcgcagcgagtcagtgagcgaggaagcggaagagcgcccaatacgcaaaccgcctctcccc |
| | | gcgcgttggccgattcattaatgcagctggcacgacaggtttcccgactggaaagcgggcagtgagcgcaacg |
| | | caattaatgtgagttagctcactcattaggcaccccaggctttacactttatgcttccggctcgtatgttgtg |
| | | tggaattgtgagcggataacaatttcacacaggaaacagctatgaccatgattacgcc |
| Mutant 62 | 71 | gctggagacatgagagctgccaacctttggccaagcccgctcatgatcaaacgctctaagaagaacagcctg |
| (ERT | | gccttgtccctgacggccgaccagatggtcagtgccttgttggatgctgagccccccatactctattccgagtat |
| mutant) | | gatcctaccagacccttcagtgaagcttcgatgatgggcttactgaccaacctggcagacagggagCTGG |
| | | TTCACATGATCAACTGGGCGAAGAGGGTGCCAGGCTTTGTGGATTT |
| | | GACCCTCCATGATCAGGTCCACCTTCTAGAATGTGCCTGGCTAGAG |
| | | ATCCTGATGATTGGTCTCGTCTGGCGCTCCATGGAGCACCCAGTGA |
| | | AGCTACTGTTTGCTCCTAACTTGGTGTTGGACAGGGACGAGGGAA |
| | | AATGTGTAGAGGGCCTGGTGGAGATCTTCGACATGCTGCTGGCTAC |
| | | ATCATCTCGGTTCCGCATGATGAATCTGCAGGGAGAGGAGTTTGTG |
| | | TGCCTCAAATCTATTATTTTGCTTAATTCTGGAGTGTACACATTTCT |
| | | GCCCAGCACCCTGAAGTCTCTGGAAGAGAAGGACCATATCCACCG |
| | | AGTCCTGGACAAGATCACAGACACTTTGATCCACCTGATGGCCAA |
| | | GGCAGGCCTGACCCTGCAGCAGCAGCACCAGCGGCTGGCCCAGCT |
| | | CCTCCTCATCCTCTCCCACATCAGGCACATGAGTAACAAAGGCATG |
| | | GAGCTGctgtacagcatgaagtgcaagaacgtggtgcccctctatGaCctgctgctggaggcggcgga |
| | | cgcccaccgcctacatgcgcccactagccgtggaggggcatccgtggaggagacggaccaaagccacttg |
| | | gccactgcgggctctacttcatcgcattccttgcaaaagtattacatcacgggggaggcagagggtttccctgc |
| | | caca |
| Mutant 62 | 72 | AGDMRAANLWPSPLMIKRSKKNSLALSLTADQMVSALLDAEPPILYS |
| (ERT | | EYDPTRPFSEASMMGLLTNLADRELVHMINWAKRVPGFVDLTLHDQ |
| mutant) | | VHLLECAWLEILMIGLVWRSMEHPVKLLFAPNLVLDRDEGKCVEGLV |
| | | EIFDMLLATSSRFRMMNLQGEEFVCLKSIILLNSGVYTFLPSTLKSLEE |
| | | KDHIHRVLDKITDTLIHLMAKAGLTLQQQHQRLAQLLLILSHIRHMSN |
| | | KGMELLYSMKCKNVVPLYDLLLEAADAHRLHAPTSRGGASVEETDQ |
| | | SHLATAGSTSSHSLQKYYITGEAEGFPAT |
| SB07135 | 73 | aagcttgaattcgagcttgcatgcctgcaggtcgttacataacttacggtaaatggcccgcctggctgaccgc |
| vector | | ccaacgacccccgcccattgacgtcaataatgacgtatgttcccatagtaacgccaatagggactttccattg |
| | | acgtcaatgggtggagtatttacggtaaactgcccacttggcagtacatcaagtgtatcatatgccaagtacg |
| | | ccccctattgacgtcaatgacggtaaatggcccgcctggcattatgcccagtacatgaccttatgggactttc |
| | | ctacttggcagtacatctacgtattagtcatcgctattaccatggtgatgcggttttggcagtacatcaatgg |
| | | gcgtggatagcggtttgactcacggggatttccaagtctccaccccattgacgtcaatgggagtttgttttgg |
| | | caccaaaatcaacgggactttccaaaatgtcgtaacaactccgccccattgacgcaaatgggcggtaggcgtg |
| | | tacggtgggaggtctatataagcagagctcaataaaagagcccacaacccctcactcggcgcgccagtcctc |
| | | cgattgactgagtcgcccgggtacccgtgtatccaataaaccctcttgcagttgcatccgacttgtggtctc |
| | | cgctgttccttgggagggtctctctgagtgattgactacccgtcagcgggggtctttcatttgggggctcgtc |
| | | cgagatcgggagacccctgcccagggaccaccgacccaccaccgggaggtaagctggccagcaacttatctgt |
| | | gtctgtccgattgtctagtgtctatgactgattttatgcgcctgcgtcggtactagttagctaactagctct |
| | | gtatctggcggacccgtggtggaactgacgagttcggaacacccggccgcaaccctgggagacgtcccaggga |
| | | cttcgggggccgtttttgtggcccgacctgagtcctaaaatcccgatcgtttaggactctttggtgcaccccc |
| | | cttagaggagggatatgtggttctggtaggagacgagaacctaaaacagttcccgcctccgtctgaattttt |
| | | gctttcggtttgggaccgaagccgcgccgcgcgtcttgtctgctgcagcatcgttctgtgttgtctctgtct |
| | | gactgtgtttctgtatttgtctgaaaatatgggccccccctcgagtccccagcatgcctgctattctcttcc |
| | | caatcctcccccttgctgtcctgccccaccccaccccccagaatagaatgacacctactcagacaatgcgatg |
| | | caatttcctcattttattaggaaaggacagtgggagtggcaccttccagggtcaaggaaggcacgggggaggg |
| | | gcaaacaacagatggctggcaactagaaggcacagttacttaCTGTTTAAATATTAAACAGggaaccgatgtT |
| | | AAATAAATAAATAAATAAATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAGG |
| | | CTTCCACGGTCGAGGTCTTGTCCTTGGTGATGTCCTCGTGATCAATT |
| | | TCTTCCGAGGTGCAGGGGTAGAACTCAAGGGTCTGGCGGGCCTTCT |
| | | GCAACATGTTCGACACAGCCCTCAGGAGGTTTTGGGAGTGGTGTA |
| | | GGCACGGGAACATTCCAGGGTCGGGGGTTGCCACAGGGAGGTTCC |
| | | GGGAACCTCCTCCGGAGCCTCCTCCTGAACCTCCGCCTGATCCGCC |
| | | ACCGGAACAAGGCACGCTGGCCCATTCGCTCCAGGAGGACGAGTA |
| | | GTATCTATCCTGCGCCCGGACGCTGATTGACGCGTTCTTCCGACAA |
| | | ATCACAGTGGCGGAGGTTTTGTCGGTGAACACCCGGTCTTTCTTCT |
| | | CCCGTTTGGACTTTCCCTGCACTTGCACACAGAAAGTGAGCGAGAA |
| | | GTATGAGTGCGGGGTGCTCCAAGTGTCTGGATATTCCCAAGACACT |
| | | TCCACTTGGCGGGAGTTCTTGAGTGGCTTCAGCTGCAAGTTCTTGG |
| | | GGGGGTCAGGCTTGATGATGTCGCGGATAAAGAAGGAGGAAGTGT |
| | | AGTTCTCGTATTTCAGCTTATGCACGGCATCGACCATGACCTCGAT |
| | | AGGCAGGGACTCTTCCGCGGCAGGGCAGGCGCTGTCCTCCTGGCA |
| | | TTCCACGGAGTACTCATATTCCTTGTTGTCTCCCCTGACTCTCTCGG |
| | | CGGACAGAGTGGCGGCTCCACAGGTCACGCCCTGAGGATCGCTTG |
| | | ATCCCCGTGACGACTTCACGGAGAAAGTCAGGTCGGTGGAGATTG |
| | | TCGTCAGCCACCAACAGGTGAACCGACCGCTGTAGTTCTTGGCTTC |
| | | GCAGCGGAGGAAGGTCTTGTTCTTCGGTTCTTTTTGGTCCTTGAGG |
| | | ATGTCAGTGGACCAGATTCCATCCTCTTTCTTGTGCAGCAGCAGCA |
| | | GGGAGTGGGACAGCACTTCGCCACCCTTGTGGCAAGTGTACTGGC |
| | | CCGCGTCGCCGAACTCCTTGACTTGAATGGTCAGGGTCTTTCCGCT |
| | | TCCGAGCACCTCGGAGCTCTGATCCAGGGTCCAGGTTATGCCGTCC |
| | | TCTTCTGGCGTATCGCAAGTCAGCACGACCATTTCTCCAGGGGCGT |
| | | CCGGGTACCAATCCAGCTCGACCACGTAGACGTCCTTCTTCAGTTC |
| | | CCAAATGGCGACCAGAGGGGAAGCGAGGAACACAAGGGAGAACC |
| | | AGGAGATGACGAGTTGCTGATGGCACATCATGGTGGCGACACCGG |
| | | TACGCGTTGGCCCCCATTATATACCCTCTAGAACTAGTtatccactccgtgt |
| | | aagggagagtgagcctcttacgaatcTTCGGCGTCGACTGCTTCattccgaggcgactgatac |
| | | cTTCGGCGTCGACTGCTTCatacgaaggcagtccgattcTTCGGCGTCGACTGC |
| | | TTCaacctttactgagacgggacTTCGGCGTCGACTGCTTCaaaggcgttgcgaatcctcat |
| | | gcgattgttacgaaacccgTTAATTAAAGAGCGAGATTCCGTCTCAAAGAAA |
| | | AAAAAAGTAATGAAATGAATAAAATGAGTCCTAGAGCCAGTAAAT |
| | | GTCGTAAATGTCTCAGCTAGTCAGGTAGTAAAAGGTCTCAACTAG |
| | | GCAGTGGCAGAGCAGGATTCAAATTCAGGGCTGTTGTGATGCCTC |
| | | CGCAGACTCTGAGCGCCACCTGGTGGTAATTTGTCTGTGCCTCTTC |
| | | TGACGTGGAAGAACAGCAACTAACACACTAACACGGCATTTACTA |
| | | TGGGCCAGCCATTGTCCATCTAGATGGccgataaaataaaagattttatttagtctccag |
| | | aaaaaggggggaatgaaagaccccacctgtaggtttggcaagctagctgcagtaacgccattttgcaaggcat |
| | | ggaaaaataccaaaccaagaatagagaagttcagatcaagggcgggtacatgaaaatagctaacgttgggcc |
| | | aaacaggatatctgcggtgagcagtttcggccccggcccggggccaagaacagatggtcaccgcagtttcgg |
| | | ccccggcccgaggccaagaacagatggtccccagatatggcccaaccctcagcagtttcttaagacccatca |
| | | gatgtttccaggctcccccaaggacctgaaatgaccctgcgccttatttgaattaaccaatcagcctgcttct |
| | | cgcttctgttcgcgcgcttctgcttcccgagctctataaaagagctcacaacccctcactcggcgcgccagtc |
| | | ctccgacagactgagtcgcccgggGGATCCGCCACCATGTCTAGACCTGGCGAGAGG |
| | | CCCTTCCAGTGCCGGATCTGCATGCGGAACTTCAGCAACATGAGCA |
| | | ACCTGACCAGACACACCCGGACACACACAGGCGAGAAGCCTTTTC |
| | | AGTGCAGAATCTGTATGCGCAATTTCTCCGACAGAAGCGTGCTGCG |
| | | GAGACACCTGAGAACCCACACCGGCAGCCAGAAACCATTCCAGTG |
| | | TCGCATCTGTATGAGAAACTTTAGCGACCCCTCCAATCTGGCCCGG |
| | | CACACCAGAACACATACCGGGGAAAAACCCTTTCAGTGTAGGATA |
| | | TGCATGAGGAATTTTTCCGACCGGTCCAGCCTGAGGCGGCACCTGA |
| | | GGACACATACTGGCTCCCAAAAGCCGTTCCAATGTCGGATATGTAT |
| | | GCGCAACTTTAGCCAGAGCGGCACCCTGCACAGACACACAAGAAC |
| | | CCATACTGGCGAGAAACCTTTCCAATGTAGAATCTGCATGCGAAAT |
| | | TTTTCCCAGCGGCCTAATCTGACCAGGCATCTGAGGACCCACCTGA |
| | | GAGGATCTaCCTGCAGGGATGAGTTTCCCACCATGGTGTTTCCTTCT |
| | | GGGCAGATCAGCCAGGCCTCGGCCTTGGCCCCGGCCCCTCCCCAA |
| | | GTCCTGCCCCAGGCTCCAGCCCCTGCCCCTGCTCCAGCCATGGTAT |
| | | CAGCTCTGGCCCAGGCCCCAGCCCCTGTCCCAGTCCTAGCCCCAGG |
| | | CCCTCCTCAGGCTGTGGCCCCACCTGCCCCCAAGCCCACCCAGGCT |
| | | GGGGAAGGAACGCTGTCAGAGGCCCTGCTGCAGCTGCAGTTTGAT |
| | | GATGAAGACCTGGGGGCCTTGCTTGGCAACAGCACAGACCCAGCT |
| | | GTGTTCACAGACCTGGCATCCGTCGACAACTCCGAGTTTCAGCAGC |
| | | TGCTGAACCAGGGCATACCTGTGGCCCCCCACACAACTGAGCCCA |
| | | TGCTGATGGAGTACCCTGAGGCTATAACTCGCCTAGTGACAGGGG |
| | | CCCAGAGGCCCCCCGACCCAGCTCCTGCTCCACTGGGGGCCCCGG |
| | | GGCTCCCCAATGGCCTCCTTTCAGGAGATGAAGACTTCTCCTCCAT |
| | | TGCGGACATGGACTTCTCAGCCCTGCTGAGTCAGATCAGCTCCcaatt |
| | | gtgcgtacgcggatcctctgctggagacatgagagctgccaacctttggccaagcccgctcatgatcaaacgc |
| | | tctaagaagaacagcctggccttgtccctgacggccgaccagatggtcagtgccttgttggatgctgagcccc |
| | | ccatactctattccgagtatgatcctaccagacccttcagtgaagcttcgatgatgggcttactgaccaacc |
| | | tggcagacagggagCTGGTTCACATGATCAACTGGGCGAAGAGGGTGCCAGGC |
| | | TTTGTGGATTTGACCCTCCATGATCAGGTCCACCTTCTAGAATGTG |
| | | CCTGGCTAGAGATCCTGATGATTGGTCTCGTCTGGCGCTCCATGGA |
| | | GCACCCAGTGAAGCTACTGTTTGCTCCTAACTTGGTGTTGGACAGG |
| | | GACGAGGGAAAATGTGTAGAGGGCCTGGTGGAGATCTTCGACATG |
| | | CTGCTGGCTACATCATCTCGGTTCCGCATGATGAATCTGCAGGGAG |
| | | AGGAGTTTGTGTGCCTCAAATCTATTATTTTGCTTAATTCTGGAGTG |
| | | TACACATTTCTGCCCAGCACCCTGAAGTCTCTGGAAGAGAAGGAC |
| | | CATATCCACCGAGTCCTGGACAAGATCACAGACACTTTGATCCACC |
| | | TGATGGCCAAGGCAGGCCTGACCCTGCAGCAGCAGCACCAGCGGC |
| | | TGGCCCAGCTCCTCCTCATCCTCTCCCACATCAGGCACATGAGTAA |
| | | CAAAGGCATGGAGCTGctgtacagcatgaagtgcaagaacgtggtgcccctctatGaCctgct |
| | | gctggaggcggcggacgcccaccgcctacatgcgcccactagccgtggaggggcatccgtggaggagac |
| | | ggaccaaagccacttggccactgcgggctctacttcatcgcattccttgcaaaagtattacatcacgggggagg |
| | | cagagggtttccctgccacaTaAGTCGACAATCAACCTCtggattacaaaatttgtgaaagatt |
| | | gactggtattcttaactatgttgctccttttacgctatgtggatacgctgctttaatgcctttgtatcatgct |
| | | attgcttcccgtatggctttcattttctcctccttgtataaatcctggttgctgtctctttatgaggagttg |
| | | tggcccgttgtcaggcaacgtggcgtggtgtgcactgtgtttgctgacgcaacccccactggttggggcatt |
| | | gccaccacctgtcagctcctttccgggactttcgctttccccctccctattgccacggcggaactcatcgcc |
| | | gcctgccttgcccgctgctggacaggggctcggctgttgggcactgacaattccgtggtgttgtcggggaaat |
| | | catcgtcctttccttggctgctcgcctgtgttgccacctggattctgcgcgggacgtccttctgctacgtccc |
| | | ttcggccctcaatccagcggaccttccttcccgcggcctgctgccggctctgcggcctcttccgcgtctacgc |
| | | cttcgccctcagacgagtcggatctccctttgggccgcctccccgcgatatcagtggtccaggctctagtttt |
| | | gactcaacaatatcaccagctgaagcctatagagtacgagccatagataaaataaaagattttatttagtctc |
| | | cagaaaaaggggggaatgaaagaccccacctgtaggtttggcaagctagcaataaaagagcccacaacccctc |
| | | actcggggcgccagtcctccgattgactgagtcgcccggccgcttcgagcagacatgataagatacattgat |
| | | gagtttggacaaaccacaactagaatgcagtgaaaaaaatgctttatttgtgaaatttgtgatgctattgct |
| | | ttatttgtaaccattataagctgcaataaacaagttaacaacaacaattgcattcattttatgtttcaggtt |
| | | cagggggagatgtgggaggttttttaaagcaagtaaaacctctacaaatgtggtaaaatcgataaggatcgg |
| | | gtacccgtgtatccaataaaccctcttgcagttgcatccgacttgtggtctcgctgttccttgggagggtct |
| | | cctctgagtgattgactacccgtcagcgggggtctttcacacatgcagcatgtatcaaaattaatttggttt |
| | | tttttcttaagctgtgccttctagttgccagccatctgttgtttgcccctcccccgtgccttccttgaccct |
| | | ggaaggtgccactcccactgtcctttcctaataaaatgaggaaattgcatcgcattgtctgagtagg |
| | | tgtcattctattctggggggtggggggggcaggacagcaagggggaggattgggaagacaatagcaggca |
| | | tgctggggatggggggctctatggagatcccgcggtacctcgcgaatgcatctagatccaatggcctttttgg |
| | | cccagacatgataagatacattgatgagtttggacaaaccacaactagaatgcagtgaaaaaaatgctttatt |
| | | tgtgaaatttgtgatgctattgctttatttgtaaccattataagctgcaataaacaagttgcggccgcttagc |
| | | cctcccacacataaccagagggcagcaattcacgaatcccaactgccgtcggctgtccatcactgtccttcac |
| | | tatggctttgatcccaggatgcagatcgagaagcacctgtcggcaccgtccgcaggggctcaagatgcccctg |
| | | ttctcatttccgatcgcgacgatacaagtcaggttgccagctgccgcagcagcagcagtgcccagcaccacga |
| | | gttctgcacaaggtcccccagtaaaatgatatacattgacaccagtgaagatgcggccgtcgctagagagagc |
| | | tgcgctggcgacgctgtagtcttcagagatggggatgctgttgattgtagccgttgctctttcaatgagggt |
| | | ggattcttcttgagacaaaggcttggccatgcggccgccgctcggtgttcgaggccacacgcgtcaccttaa |
| | | tatgcgaagtggacctcggaccgcgccgccccgactgcatctgcgtgttcgaattcgccaatgacaagacgct |
| | | gggggggtttgtgtcatcatagaactaaagacatgcaaatatatttcttccggggggtaccggcctttttgg |
| | | ccATTGGatcggatctggccaaaaaggcccttaagtatttacattaaatggccatagtacttaaagttacatt |
| | | ggcttccttgaaataaacatggagtattcagaatgtgtcataaatatttctaattttaagatagtatctccat |
| | | tggctttctactttttcttttatttttttttgtcctctgtcttccatttgttgttgttgttgtttgtttgttt |
| | | gtttgttggttggttggttaatttttttttaaagatcctacactatagttcaagctagactattagctactct |
| | | gtaacccagggtgaccttgaagtcatgggtagcctgctgttttagccttcccacatctaagattacaggtatg |
| | | agctatcatttttggtatattgattgattgattgattgatgtgtgtgtgtgtgattgtgtttgtgtgtgtgaT |
| | | tgtgTaTatgtgtgtatggTtgtgtgtgaTtgtgtgtatgtatgTTtgtgtgtgaTtgTgtgtgtgtgaTtg |
| | | tgcatgtgtgtgtgtgtgaTtgtgtTtatgtgtatgaTtgtgtgtgtgtgtgtgtgtgtgtgtgtgtgtgtgt |
| | | gtgtgtgtgtgttgtgTaTaTatatttatggtagtgagagGcaacgctccggctcaggtgtcaggttggtttt |
| | | tgagacagagtctttcacttagcttggaattcactggccgtcgttttacaacgtcgtgactgggaaaaccctg |
| | | gcgttacccaacttaatcgccttgcagcacatccccctttcgccagctggcgtaatagcgaagaggcccgcac |
| | | cgatcgcccttcccaacagttgcgcagcctgaatggcgaatggcgcctgatgcggtattttctccttacgcat |
| | | ctgtgcggtatttcacaccgcatatggtgcactctcagtacaatctgctctgatgccgcatagttaagccagc |
| | | cccgacacccgccaacacccgctgacgcgccctgacgggcttgtctgctcccggcatccgcttacagacaagc |
| | | tgtgaccgtctccgggagctgcatgtgtcagaggttttcaccgtcatcaccgaaacgcgcgagacgaaagggc |
| | | ctcgtgatacgcctatttttataggttaatgtcatgataataatggtttcttagacgtcaggtggcacttttc |
| | | ggggaaatgtgcgcggaacccctatttgtttatttttctaaatacattcaaatatgtatccgctcatgagaca |
| | | ataaccctgataaatgcttcaataatattgaaaaaggaagagtatgagtattcaacatttccgtgtcgccctt |
| | | attcccttttttgcggcattttgccttcctgtttttgctcacccagaaacgctggtgaaagtaaaagatgct |
| | | gaagatcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgtattg |
| | | acgccgggcaagagcaactcggtcgccgcatacactattctcagaatgacttggttgagtactcaccagtcac |
| | | agaaaagcatcttacggatggcatgacagtaagagaattatgcagtgctgccataaccatgagtgataacac |
| | | tgcggccaacttacttctgacaacgatcggaggaccgaaggagctaaccgcttttttgcacaacatgggggat |
| | | catgtaactcgccttgatcgttgggaaccggagctgaatgaagccataccaaacgacgagcgtgacaccacga |
| | | tgcctgtagcaatggcaacaacgttgcgcaaactattaactggcgaactacttactctagcttcccggcaac |
| | | aattaatagactggatggaggcggataaagttgcaggaccacttctgcgctcggcccttccggctggctggtt |
| | | tattgctgataaatctggagccggtgagcgtgggtctcgcggtatcattgcagcactggggccagatggtaag |
| | | ccctcccgtatcgtagttatctacacgacggggagtcaggcaactatggatgaacgaaatagacagatcgctg |
| | | agataggtgcctcactgattaagcattggtaactgtcagaccaagtttactcatatatactttagattgattt |
| | | aaaacttcatttttaatttaaaaggatctaggtgaagatcctttttgataatctcatgaccaaaatcccttaa |
| | | cgtgagttttcgttccactgagcgtcagaccccgtagaaaagatcaaaggatcttcttgagatcctttttttc |
| | | tgcgcgtaatctgctgcttgcaaacaaaaaaaccaccgctaccagcggtggtttgtttgccggatcaagagct |
| | | accaactctttttccgaaggtaactggcttcagcagagcgcagataccaaatactgtccttctagtgtagccg |
| | | tagttaggccaccacttcaagaactctgtagcaccgcctacatacctcgctctgctaatcctgttaccagtgg |
| | | ctgctgccagtggcgataagtcgtgtcttaccgggttggactcaagacgatagttaccggataaggcgcagcg |
| | | gtcgggctgaacggggggttcgtgcacacagcccagcttggagcgaacgacctacaccgaactgagatacct |
| | | acagcgtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggtttc |
| | | gccacctctgacttgagcgtcgatttttgtgatgctcgtcaggggggcggagcctatggaaaaacgccagcaa |
| | | cgcggcctttttacggttcctggccttttgctggccttttgctcacatgttctttcctgcgttatcccctgat |
| | | tctgtggataaccgtattaccgcctttgagtgagctgataccgctcgccgcagccgaacgaccgagcgcagc |
| | | gagtcagtgagcgaggaagcggaagagcgcccaatacgcaaaccgcctctccccgcgcgttggccgattcat |
| | | taatgcagctggcacgacaggtttcccgactggaaagcgggcagtgagcgcaacgcaattaatgtgagttagc |
| | | tcactcattaggcaccccaggctttacactttatgcttccggctcgtatgttgtgtggaattgtgagcggata |
| | | acaatttcacacaggaaacagctatgaccatgattacgcc |
| LR1 split | 74 | AGCGGCGGAGGTGGTAGCGGAGGCGGAGGATCTGGAATTACACA |
| N term | | GGGACTCGCCGTGTCTACAATCTCCAGCTTCTTTGGTGGCGGTAGT |
| linker + | | GGCGGCGGTGGCAGTGGCGGTGGATCTCTTCAA |
| CD16Tace | | |
| (cleavage | | |
| site) | | |
| LR1 split | 75 | SGGGGSGGGGSGITQGLAVSTISSFFGGGSGGGGSGGGSLQ |
| N term | | |
| linker + | | |
| CD16Tace | | |
| (cleavage | | |
| site) | | |
| B7-1 (TM | 76 | CTGCTGCCAAGCTGGGCCATCACACTGATCTCCGTGAACGGCATCT |
| domain) | | TCGTGATCTGTTGCCTGACCTACTGCTTCGCCCCTCGGTGCAGAGA |
| | | GCGGAGAAGAAACGAACGGCTGCGGAGAGAATCTGTGCGGCCTGT |
| | | G |
| B7-1 (TM | 77 | LLPSWAITLISVNGIFVICCLTYCFAPRCRERRRNERLRRESVRPV |
| domain) | | |
| SV40 | 78 | GTGTGTCAGTTAGGGTGTGGAAAGTCCCCAGGCTCCCCAGCAGGC |
| promoter | | AGAAGTATGCAAAGCATGCATCTCAATTAGTCAGCAACCAGGTGT |
| | | GGAAAGTCCCCAGGCTCCCCAGCAGGCAGAAGTATGCAAAGCATG |
| | | CATCTCAATTAGTCAGCAACCATAGTCCCGCCCCTAACTCCGCCCA |
| | | TCCCGCCCCTAACTCCGCCCAGTTCCGCCCATTCTCCGCCCCATGG |
| | | CTGACTAATTTTTTTTATTTATGCAGAGGCCGAGGCCGCCTCTGCCT |
| | | CTGAGCTATTCCAGAAGTAGTGAGGAGGCTTTTTTGGAGGCCTAGG |
| | | CTTTTGCAAA |
| SB07127 | 79 | aagcttgaattcgagcttgcatgcctgcaggtcgttacataacttacggtaaatggcccgcctggctgaccgc |
| vector | | ccaacgacccccgcccattgacgtcaataatgacgtatgttcccatagtaacgccaatagggactttccattg |
| | | acgtcaatgggtggagtatttacggtaaactgcccacttggcagtacatcaagtgtatcatatgccaagtacg |
| | | ccccctattgacgtcaatgacggtaaatggcccgcctggcattatgcccagtacatgaccttatgggacttt |
| | | cctacttggcagtacatctacgtattagtcatcgctattaccatggtgatgcggttttggcagtacatcaat |
| | | gggcgtggatagcggtttgactcacggggatttccaagtctccaccccattgacgtcaatgggagtttgttt |
| | | tggcaccaaaatcaacgggactttccaaaatgtcgtaacaactccgccccattgacgcaaatgggcggtaggc |
| | | gtgtacgggggaggtctatataagcagagctcaataaaagagcccacaacccctcactcggcgcgccagtcct |
| | | ccgattgactgagtcgcccgggtacccgtgtatccaataaaccctcttgcagttgcatccgacttgtggtctc |
| | | gctgttccttgggagggtctcctctgagtgattgactacccgtcaggggggtctttcatttgggggctcgtcc |
| | | cgagatcgggagacccctgccagggaccaccgacccaccaccgggaggtaagctggccagcaacttatctgtg |
| | | tctgtccgattgtctagtgtctatgactgattttatgcgcctgcgtcggtactagttagctaactagctctgt |
| | | atctggcggacccgtggtggaactgacgagttcggaacacccggccgcaaccctgggagacgtcccagggact |
| | | tcgggggccgtttttgtggcccgacctgagtcctaaaatcccgatcgtttaggactctttggtgcacccccct |
| | | tagaggagggatatgtggttctggtaggagacgagaacctaaaacagttcccgcctccgtctgaatttttgct |
| | | ttcggtttgggaccgaagccgcgccgcgcgtcttgtctgctgcagcatcgttctgtgttgtctctgtctgact |
| | | gtgtttctgtatttgtctgaaaatatgggccccccctcgagtccccagcatgcctgctattctcttcccaat |
| | | cctcccccttgctgtcctgccccaccccaccccccagaatagaatgacacctactcagacaatgcgatgcaa |
| | | tttcctcattttattaggaaaggacagtgggagtggcaccttccagggtcaaggaaggcacgggggaggggc |
| | | aaacaacagatggctggcaactagaaggcacagttacttaCACAGGCCGCACAGATTCTCTCCGCAGCCGT |
| | | TCGTTTCTTCTCCGCTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGGG |
| | | TTCTTCCAGGCTAGACTTCTGGGGCACTGTCTCGCTGTTGAAGTTC |
| | | AGGGCCTGCATCAGCTCGTCGATCACGGCCAGCATATTCTGGTCCA |
| | | GGAAGATCTGCCGCTTGGGGTCCATCAGCAGCTTGGCGTTCATGGT |
| | | CTTGAATTCCACCTGGTACATCTTCAGGTCCTCGTAGATGCTGCTC |
| | | AGGCACAGGGCCATCATGAAGGAGGTCTTTCTGCTGGCCAGGCAA |
| | | GAGCCGTTGGTGATGAAGCTGGTTTCCCGGCTGTTCAGGCAGCTCT |
| | | CGTTCTTGGTCAGTTCCAGAGGCAGGCAGGCTTCCACGGTGCTGGT |
| | | CTTATCCTTGGTGATGTCCTCGTGGTCGATTTCCTCGCTGGTGCAGG |
| | | GGTAGAATTCCAGGGTCTGTCTGGCCTTCTGCAGCATGTTGGACAC |
| | | GGCTCTCAGCAGGTTCTGGCTGTGGTGCAGACAAGGGAACATGCC |
| | | AGGATCAGGAGTGGCCACAGGCAGGTTTCTAGATCCGCCGCCAGA |
| | | TCCACCACCTGATCCGCCACCGCTTCCTCCGCCAGAACATGGCACG |
| | | CTGGCCCATTCGCTCCAAGAGCTGCTGTAGTACCGGTCCTGGGCTC |
| | | TGACGCTGATGCTGGCGTTCTTTCTGCAGATCACGGTGGCGCTGGT |
| | | CTTGTCGGTGAACACCCGGTCCTTTTTCTCGCGCTTGGACTTGCCCT |
| | | GCACTTGCACGCAAAAGGTCAGGCTGAAGTAGCTGTGGGGTGTAG |
| | | ACCAGGTGTCGGGGTACTCCCAGGACACTTCCACCTGTCTGCTGTT |
| | | CTTCAGAGGCTTCAGCTGCAGGTTCTTTGGAGGATCGGGCTTGATG |
| | | ATGTCCCGGATGAAAAAGCTGGAGGTGTAGTTCTCGTACTTCAGCT |
| | | TGTGCACGGCGTCCACCATCACTTCGATAGGCAGAGACTCTTCGGC |
| | | GGCTGGACAGGCGCTGTCCTCTTGGCATTCCACGCTGTACTCGTAT |
| | | TCTTTGTTGTCGCCCCGCACTCTTTCGGCAGACAGTGTAGCGGCGC |
| | | CACATGTAACGCCCTGAGGATCACTGCTGCCTCTGCTGGACTTCAC |
| | | GCTGAAGGTCAGGTCGGTGCTGATGGTGGTCAGCCACCAACATGT |
| | | GAACCGGCCGCTGTAGTTCTTGGCCTCGCATCTCAGGAAGGTCTTG |
| | | TTCTTGGGCTCTTTCTGGTCCTTCAGGATGTCGGTGCTCCAAATGCC |
| | | ATCCTCTTTCTTGTGGAGCAGCAGCAGGCTGTGGCTCAGCACTTCT |
| | | CCGCCTTTGTGACAGGTGTACTGGCCGGCGTCGCCAAACTCTTTCA |
| | | CTTGGATGGTCAGGGTCTTGCCGCTGCCGAGCACCTCGCTAGACTG |
| | | ATCCAGTGTCCAGGTGATGCCGTCCTCTTCAGGGGTATCGCAGGTC |
| | | AGCACCACCATCTCGCCAGGAGCATCGGGATACCAGTCCAGTTCC |
| | | ACCACGTACACGTCTTTCTTCAGCTCCCAGATGGCCACCAGAGGAG |
| | | AGGCCAGGAACACCAGGCTGAACCAGCTGATGACCAGCTGCTGGT |
| | | GACACATCATGGTGGCGACACCGGTACGCGTTGGCCCCCATTATAT |
| | | ACCCTCTAGAACTAGTtatccactccgtgtaagggagagtgagcctcttacgaatcTTCGG |
| | | CGTCGACTGCTTCattccgaggcgactgataccTTCGGCGTCGACTGCTTCatacg |
| | | aaggcagtccgattcTTCGGCGTCGACTGCTTCaacctttactgagacgggacTTCGGC |
| | | GTCGACTGCTTCaaaggcgttgcgaatcctcatgcgattgttacgaaacccgTTAATTAAA |
| | | GAGCGAGATTCCGTCTCAAAGAAAAAAAAAGTAATGAAATGAATA |
| | | AAATGAGTCCTAGAGCCAGTAAATGTCGTAAATGTCTCAGCTAGTC |
| | | AGGTAGTAAAAGGTCTCAACTAGGCAGTGGCAGAGCAGGATTCAA |
| | | ATTCAGGGCTGTTGTGATGCCTCCGCAGACTCTGAGCGCCACCTGG |
| | | TGGTAATTTGTCTGTGCCTCTTCTGACGTGGAAGAACAGCAACTAA |
| | | CACACTAACACGGCATTTACTATGGGCCAGCCATTGTCCATCTAGA |
| | | TGGccgataaaataaaagattttatttagtctccagaaaaaggggggaatgaaagaccccacctgtaggtttg |
| | | gcaagctagctgcaGTGTGTCAGTTAGGGTGTGGAAAGTCCCCAGGCTCCC |
| | | CAGCAGGCAGAAGTATGCAAAGCATGCATCTCAATTAGTCAGCAA |
| | | CCAGGTGTGGAAAGTCCCCAGGCTCCCCAGCAGGCAGAAGTATGC |
| | | AAAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCTGCCTCTGAGCTATTCCAGAAGTAGTGAGGAGGCTTTTTTGG |
| | | AGGCCTAGGCTTTTGCAAAGGATCCGCCACCATGTCTAGACCTGGC |
| | | GAGAGGCCCTTCCAGTGCCGGATCTGCATGCGGAACTTCAGCAAC |
| | | ATGAGCAACCTGACCAGACACACCCGGACACACACAGGCGAGAA |
| | | GCCTTTTCAGTGCAGAATCTGTATGCGCAATTTCTCCGACAGAAGC |
| | | GTGCTGCGGAGACACCTGAGAACCCACACCGGCAGCCAGAAACCA |
| | | TTCCAGTGTCGCATCTGTATGAGAAACTTTAGCGACCCCTCCAATC |
| | | TGGCCCGGCACACCAGAACACATACCGGGGAAAAACCCTTTCAGT |
| | | GTAGGATATGCATGAGGAATTTTTCCGACCGGTCCAGCCTGAGGC |
| | | GGCACCTGAGGACACATACTGGCTCCCAAAAGCCGTTCCAATGTC |
| | | GGATATGTATGCGCAACTTTAGCCAGAGCGGCACCCTGCACAGAC |
| | | ACACAAGAACCCATACTGGCGAGAAACCTTTCCAATGTAGAATCT |
| | | GCATGCGAAATTTTTCCCAGCGGCCTAATCTGACCAGGCATCTGAG |
| | | GACCCACCTGAGAGGATCTaCCTGCAGGGATGAGTTTCCCACCATG |
| | | GTGTTTCCTTCTGGGCAGATCAGCCAGGCCTCGGCCTTGGCCCCGG |
| | | CCCCTCCCCAAGTCCTGCCCCAGGCTCCAGCCCCTGCCCCTGCTCC |
| | | AGCCATGGTATCAGCTCTGGCCCAGGCCCCAGCCCCTGTCCCAGTC |
| | | CTAGCCCCAGGCCCTCCTCAGGCTGTGGCCCCACCTGCCCCCAAGC |
| | | CCACCCAGGCTGGGGAAGGAACGCTGTCAGAGGCCCTGCTGCAGC |
| | | TGCAGTTTGATGATGAAGACCTGGGGGCCTTGCTTGGCAACAGCA |
| | | CAGACCCAGCTGTGTTCACAGACCTGGCATCCGTCGACAACTCCGA |
| | | GTTTCAGCAGCTGCTGAACCAGGGCATACCTGTGGCCCCCCACACA |
| | | ACTGAGCCCATGCTGATGGAGTACCCTGAGGCTATAACTCGCCTAG |
| | | TGACAGGGGCCCAGAGGCCCCCCGACCCAGCTCCTGCTCCACTGG |
| | | GGGCCCCGGGGCTCCCCAATGGCCTCCTTTCAGGAGATGAAGACTT |
| | | CTCCTCCATTGCGGACATGGACTTCTCAGCCCTGCTGAGTCAGATC |
| | | AGCTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgccttgttg |
| | | gatgctgagccccccatactctattccgagtatgatcctaccagacccttcagtgaagcttcgatgatgggct |
| | | tactgaccaacctggcagacagggagCTGGTTCACATGATCAACTGGGCGAAGAGG |
| | | GTGCCAGGCTTTGTGGATTTGACCCTCCATGATCAGGTCCACCTTC |
| | | TAGAATGTGCCTGGATGGAGATCCTGATGATTGGTGTGGTCTGGCG |
| | | CTCCATGGAGCACCCAGTGAAGCTACTGTTTGCTCCTAACTTGCTC |
| | | TTGGACAGGGACCAGGGAAAATGTGTAGAGGGCCTGGTGGAGATC |
| | | TTCGACATGCTGCTGGCTACATCATCTCGGTTCCGCATGATGAATC |
| | | TGCAGGGAGAGGAGTTTGTGTGCCTCAAATCTATTATTTTGCTTAA |
| | | TTCTGGAGTGTACACATTTCTGCCCAGCACCCTGAAGTCTCTGGAA |
| | | GAGAAGGACCATATCCACCGAGTCCTGGACAAGATCACAGACACT |
| | | TTGATCCACCTGATGGCCAAGGCAGGCCTGACCCTGCAGCAGCAG |
| | | CACCAGCGGCTGGCCCAGCTCCTCCTCATCCTCTCCCACATCAGGC |
| | | ACATGAGTAACAAAGGCATGGAGCTGctgtacagcatgaagtgcaagaacgtggtg |
| | | cccctctatGaCctgctgctggaggcggcggacgcccaccgcctacatgcgcccactagccgtggagggg |
| | | catccgtggaggagacggaccaaagccacttggccactgcgggctctacttcatcgcattccttgcaaaagtat |
| | | tacatcacgggggaggcagagggtttccctgccacaTaAGTCGACAATCAACCTCtggatta |
| | | caaaatttgtgaaagattgactggtattcttaactatgttgctccttttacgctatgtggatacgctgcttta |
| | | atgcctttgtatcatgctattgcttcccgtatggctttcattttctcctccttgtataaatcctggttgctgt |
| | | ctctttatgaggagttgtggcccgttgtcaggcaacgtggcgtggtgtgcactgtgtttgctgacgcaaccccc |
| | | actggttggggcattgccaccacctgtcagctcctttccgggactttcgctttccccctccctattgccacgg |
| | | cggaactcatcgccgcctgccttgcccgctgctggacaggggctcggctgttgggcactgacaattccgtggt |
| | | gttgtcggggaaatcatcgtcctttccttggctgctcgcctgtgttgccacctggattctgcgcgggacgtcc |
| | | ttctgctacgtcccttcggccctcaatccagcggaccttccttcccgcggcctgctgccggctctgcggcctc |
| | | ttccgcgtctacgccttcgccctcagacgagtcggatctccctttgggccgcctccccgcgatatcagtggt |
| | | ccaggctctagttttgactcaacaatatcaccagctgaagcctatagagtacgagccatagataaaataaaa |
| | | gattttatttagtctccagaaaaaggggggaatgaaagaccccacctgtaggtttggcaagctagcaataaaa |
| | | gagcccacaacccctcactcggggcgccagtcctccgattgactgagtcgcccggccgcttcgagcagacat |
| | | gataagatacattgatgagtttggacaaaccacaactagaatgcagtgaaaaaaatgctttatttgtgaaatt |
| | | tgtgatgctattgctttatttgtaaccattataagctgcaataaacaagttaacaacaacaattgcattcat |
| | | tttatgtttcaggttcagggggagatgtgggaggttttttaaagcaagtaaaacctctacaaatgtggtaaa |
| | | atcgataaggatcgggtacccgtgtatccaataaaccctcttgcagttgcatccgacttgtggtctcgctgt |
| | | tccttgggagggtctcctctgagtgattgactacccgtcagcgggggtctttcacacatgcagcatgtatca |
| | | aaattaatttggttttttttcttaagctgtgccttctagttgccagccatctgttgtttgcccctcccccgtg |
| | | ccttccttgaccctggaaggtgccactcccactgtcctttcctaataaaatgaggaaattgcatcgcattgt |
| | | ctgagtaggtgtcattctattctggggggtggggggggcaggacagcaagggggaggattgggaagacaata |
| | | gcaggcatgctggggatgcggtgggctctatggagatcccgcggtacctcgcgaatgcatctagatccaatg |
| | | gcctttttggcccagacatgataagatacattgatgagtttggacaaaccacaactagaatgcagtgaaaaaa |
| | | atgctttatttgtgaaatttgtgatgctattgctttatttgtaaccattataagctgcaataaacaagttgcg |
| | | gccgcttagccctcccacacataaccagagggcagcaattcacgaatcccaactgccgtcggctgtccatcac |
| | | tgtccttcactatggctttgatcccaggatgcagatcgagaagcacctgtcggcaccgtccgcaggggctcaag |
| | | atgcccctgttctcatttccgatcgcgacgatacaagtcaggttgccagctgccgcagcagcagcagtgccca |
| | | gcaccacgagttctgcacaaggtcccccagtaaaatgatatacattgacaccagtgaagatgcggccgtcgct |
| | | agagagagctgcgctggcgacgctgtagtcttcagagatggggatgctgttgattgtagccgttgctctttca |
| | | atgagggtggattcttcttgagacaaaggcttggccatgcggccgccgctcggtgttcgaggccacacgcgtc |
| | | accttaatatgcgaagtggacctcggaccgcgccgccccgactgcatctgcgtgttcgaattcgccaatgaca |
| | | agacgctgggcggggtttgtgtcatcatagaactaaagacatgcaaatatatttcttccggggggtaccggcct |
| | | ttttggccATTGGatcggatctggccaaaaaggcccttaagtatttacattaaatggccatagtacttaaag |
| | | ttacattggcttccttgaaataaacatggagtattcagaatgtgtcataaatatttctaattttaagatagta |
| | | tctccattggctttctactttttcttttatttttttttgtcctctgtcttccatttgttgttgttgttgtttg |
| | | tttgtttgtttgttggttggttggttaatttttttttaaagatcctacactatagttcaagctagactattag |
| | | ctactctgtaacccagggtgaccttgaagtcatgggtagcctgctgttttagccttcccacatctaagattac |
| | | aggtatgagctatcatttttggtatattgattgattgattgattgatgtgtgtgtgtgtgattgtgtttgtg |
| | | tgtgtgaTtgtgTaTatgtgtgtatggTtgtgtgtgaTtgtgtgtatgtatgTTtgtgtgtgaTtgTgtgtg |
| | | tgtgaTtgtgcatgtgtgtgtgtgtgaTtgtgtTtatgtgtatgaTtgtgtgtgtgtgtgtgtgtgtgtgtg |
| | | tgtgtgtgtgtgtgtgtgtgttgtgTaTaTatatttatggtagtgagagGcaacgctccggctcaggtgtcag |
| | | gttggtttttgagacagagtctttcacttagcttggaattcactggccgtcgttttacaacgtcgtgactggg |
| | | aaaaccctggcgttacccaacttaatcgccttgcagcacatccccctttcgccagctggcgtaatagcgaaga |
| | | cggcccgcaccgatcgcccttcccaacagttgcgcagcctgaatggcgaatggcgcctgatgggtattttctc |
| | | cttacgcatctgtgcggtatttcacaccgcatatggtgcactctcagtacaatctgctctgatgccgcatagt |
| | | taagccagccccgacacccgccaacacccgctgacgcgccctgacgggcttgtctgctcccggca |
| | | tccgcttacagacaagctgtgaccgtctccgggagctgcatgtgtcagaggttttcaccgtcatcaccgaaac |
| | | gcgcgagacgaaagggcctcgtgatacgcctatttttataggttaatgtcatgataataatggtttcttagac |
| | | gtcaggtggcacttttcggggaaatgtgcgcggaacccctatttgtttatttttctaaatacattcaaatatg |
| | | tatccgctcatgagacaataaccctgataaatgcttcaataatattgaaaaaggaagagtatgagtattcaa |
| | | catttccgtgtcgcccttattcccttttttgcggcattttgccttcctgtttttgctcacccagaaacgctgg |
| | | tgaaagtaaaagatgctgaagatcagttgggtgcacgagtgggttacatcgaactggatctcaacagcggtaa |
| | | gatccttgagagttttcgccccgaagaacgttttccaatgatgagcacttttaaagttctgctatgtggcgc |
| | | ggtattatcccgtattgacgccgggcaagagcaactcggtcgccgcatacactattctcagaatgacttggt |
| | | tgagtactcaccagtcacagaaaagcatcttacggatggcatgacagtaagagaattatgcagtgctgccat |
| | | aaccatgagtgataacactgcggccaacttacttctgacaacgatcggaggaccgaaggagctaaccgctttt |
| | | ttgcacaacatgggggatcatgtaactcgccttgatcgttgggaaccggagctgaatgaagccataccaaacg |
| | | acgagcgtgacaccacgatgcctgtagcaatggcaacaacgttgcgcaaactattaactggcgaactacttac |
| | | tctagcttcccggcaacaattaatagactggatggaggcggataaagttgcaggaccacttctgcgctcggcc |
| | | cttccggctggctggtttattgctgataaatctggagccggtgagcgtgggtctcgcggtatcattgcagca |
| | | ctggggccagatggtaagccctcccgtatcgtagttatctacacgacggggagtcaggcaactatggatgaac |
| | | gaaatagacagatcgctgagataggtgcctcactgattaagcattggtaactgtcagaccaagtttactcata |
| | | tatactttagattgatttaaaacttcatttttaatttaaaaggatctaggtgaagatcctttttgataatctc |
| | | atgaccaaaatcccttaacgtgagttttcgttccactgagcgtcagaccccgtagaaaagatcaaaggatctt |
| | | cttgagatcctttttttctgcgcgtaatctgctgcttgcaaacaaaaaaaccaccgctaccagcggtggttt |
| | | gtttgccggatcaagagctaccaactctttttccgaaggtaactggcttcagcagag |
| | | cgcagataccaaatactgtccttctagtgtagccgtagttaggccaccacttcaagaactctgtagcaccgcct |
| | | acatacctcgctctgctaatcctgttaccagtggctgctgccagtggcgataagtcgtgtcttaccgggttgg |
| | | actcaagacgatagttaccggataaggcgcagcggtcgggctgaacggggggttcgtgcacacagcccagctt |
| | | ggagcgaacgacctacaccgaactgagatacctacagcgtgagctatgagaaagcgccacgcttcccgaaggg |
| SB07133 | 80 | agaaaggcggacaggtatccggtaagcggcagggtcggaacaggagagcgcacgagggagcttccaggg |
| vector | | ggaaacgcctggtatctttatagtcctgtcgggtttcgccacctctgacttgagcgtcgatttttgtgatgct |
| | | cgtcaggggggcggagcctatggaaaaacgccagcaacgcggcctttttacggttcctggccttttgctggcc |
| | | ttttgctcacatgttctttcctgcgttatcccctgattctgtggataaccgtattaccgcctttgagtgagc |
| | | tgataccgctcgccgcagccgaacgaccgagcgcagcgagtcagtgagcgaggaagcggaagagcgcccaata |
| | | cgcaaaccgcctctccccgcgcgttggccgattcattaatgcagctggcacgacaggtttcccgactggaaag |
| | | cgggcagtgagcgcaacgcaattaatgtgagttagctcactcattaggcaccccaggctttacactttatgct |
| | | tccggctcgtatgttgtgtggaattgtgagcggataacaatttcacacaggaaacagctatgaccatgattac |
| | | gccaagcttgaattcgagcttgcatgcctgcaggtgttacataacttacggtaaatggcccgcctggctgacc |
| | | gcccaacgacccccgcccattgacgtcaataatgacgtatgttcccatagtaacgccaatagggactttccat |
| | | tgacgtcaatgggtggagtatttacggtaaactgcccacttggcagtacatcaagtgtatcatatgccaagta |
| | | cgccccctattgacgtcaatgacggtaaatggcccgcctggcattatgcccagtacatgaccttatgggactt |
| | | tcctacttggcagtacatctacgtattagtcatcgctattaccatggtgatgcggttttggcagtacatcaat |
| | | gggcgtggatagcggtttgactcacggggatttccaagtctccaccccattgacgtcaatgggagtttgtttt |
| | | ggcaccaaaatcaacgggactttccaaaatgtcgtaacaactccgccccattgacgcaaatgggcggtaggcg |
| | | tgtacggtgggaggtctatataagcagagctcaataaaagagcccacaacccctcactcggcgcgccagtcct |
| | | ccgattgactgagtcgcccgggtacccgtgtatccaataaaccctcttgcagttgcatccgacttgtggtctc |
| | | gctgttccttgggagggtctcctctgagtgattgactacccgtcagcgggggtctttcatttgggggctcgtc |
| | | cgagatcgggagacccctgcccagggaccaccgacccaccaccgggaggtaagctggccagcaacttatctg |
| | | tgtctgtccgattgtctagtgtctatgactgattttatgcgcctgcgtcggtactagttagctaactagctct |
| | | gtatctggcggacccgtggtggaactgacgagttcggaacacccggccgcaaccctgggagacgtcccaggga |
| | | cttcgggggccgtttttgtggcccgacctgagtcctaaaatcccgatcgtttaggactctttggtgcacccc |
| | | ccttagaggagggatatgtggttctggtaggagacgagaacctaaaacagttcccgcctccgtctgaattttt |
| | | gctttcggtttgggaccgaagccgcgccgcgcgtcttgtctgctgcagcatcgttctgtgttgtctctgtctg |
| | | actgtgtttctgtatttgtctgaaaatatgggccccccctcgagtccccagcatgcctgctattctcttccca |
| | | atcctcccccttgctgtcctgccccaccccaccccccagaatagaatgacacctactcagacaatgcgatgcaa |
| | | tttcctcattttattaggaaaggacagtgggagtggcaccttccagggtcaaggaaggcacgggggaggggca |
| | | aacaacagatggctggcaactagaaggcacagttacttaCACAGGCCGCACAGATTCTCTCCGCAGCCGTTC |
| | | GTTTCTTCTCCGCTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGGG |
| | | TTCTTCCAGGCTAGACTTCTGGGGCACTGTCTCGCTGTTGAAGTTC |
| | | AGGGCCTGCATCAGCTCGTCGATCACGGCCAGCATATTCTGGTCCA |
| | | GGAAGATCTGCCGCTTGGGGTCCATCAGCAGCTTGGCGTTCATGGT |
| | | CTTGAATTCCACCTGGTACATCTTCAGGTCCTCGTAGATGCTGCTC |
| | | AGGCACAGGGCCATCATGAAGGAGGTCTTTCTGCTGGCCAGGCAA |
| | | GAGCCGTTGGTGATGAAGCTGGTTTCCCGGCTGTTCAGGCAGCTCT |
| | | CGTTCTTGGTCAGTTCCAGAGGCAGGCAGGCTTCCACGGTGCTGGT |
| | | CTTATCCTTGGTGATGTCCTCGTGGTCGATTTCCTCGCTGGTGCAGG |
| | | GGTAGAATTCCAGGGTCTGTCTGGCCTTCTGCAGCATGTTGGACAC |
| | | GGCTCTCAGCAGGTTCTGGCTGTGGTGCAGACAAGGGAACATGCC |
| | | AGGATCAGGAGTGGCCACAGGCAGGTTTCTAGATCCGCCGCCAGA |
| | | TCCACCACCTGATCCGCCACCGCTTCCTCCGCCAGAACATGGCACG |
| | | CTGGCCCATTCGCTCCAAGAGCTGCTGTAGTACCGGTCCTGGGCTC |
| | | TGACGCTGATGCTGGCGTTCTTTCTGCAGATCACGGTGGCGCTGGT |
| | | CTTGTCGGTGAACACCCGGTCCTTTTTCTCGCGCTTGGACTTGCCCT |
| | | GCACTTGCACGCAAAAGGTCAGGCTGAAGTAGCTGTGGGGTGTAG |
| | | ACCAGGTGTCGGGGTACTCCCAGGACACTTCCACCTGTCTGCTGTT |
| | | CTTCAGAGGCTTCAGCTGCAGGTTCTTTGGAGGATCGGGCTTGATG |
| | | ATGTCCCGGATGAAAAAGCTGGAGGTGTAGTTCTCGTACTTCAGCT |
| | | TGTGCACGGCGTCCACCATCACTTCGATAGGCAGAGACTCTTCGGC |
| | | GGCTGGACAGGCGCTGTCCTCTTGGCATTCCACGCTGTACTCGTAT |
| | | TCTTTGTTGTCGCCCCGCACTCTTTCGGCAGACAGTGTAGCGGCGC |
| | | CACATGTAACGCCCTGAGGATCACTGCTGCCTCTGCTGGACTTCAC |
| | | GCTGAAGGTCAGGTCGGTGCTGATGGTGGTCAGCCACCAACATGT |
| | | GAACCGGCCGCTGTAGTTCTTGGCCTCGCATCTCAGGAAGGTCTTG |
| | | TTCTTGGGCTCTTTCTGGTCCTTCAGGATGTCGGTGCTCCAAATGCC |
| | | ATCCTCTTTCTTGTGGAGCAGCAGCAGGCTGTGGCTCAGCACTTCT |
| | | CCGCCTTTGTGACAGGTGTACTGGCCGGCGTCGCCAAACTCTTTCA |
| | | CTTGGATGGTCAGGGTCTTGCCGCTGCCGAGCACCTCGCTAGACTG |
| | | ATCCAGTGTCCAGGTGATGCCGTCCTCTTCAGGGGTATCGCAGGTC |
| | | AGCACCACCATCTCGCCAGGAGCATCGGGATACCAGTCCAGTTCC |
| | | ACCACGTACACGTCTTTCTTCAGCTCCCAGATGGCCACCAGAGGAG |
| | | AGGCCAGGAACACCAGGCTGAACCAGCTGATGACCAGCTGCTGGT |
| | | GACACATCATGGTGGCGACACCGGTACGCGTTGGCCCCCATTATAT |
| | | ACCCTCTAGAACTAGTtatccactccgtgtaagggagagtgagcctcttacgaatcTTCGG |
| | | CGTCGACTGCTTCattccgaggcgactgataccTTCGGCGTCGACTGCTTCatacg |
| | | aaggcagtccgattcTTCGGCGTCGACTGCTTCaacctttactgagacgggacTTCGGC |
| | | GTCGACTGCTTCaaaggcgttgcgaatcctcatgcgattgttacgaaacccgTTAATTAAA |
| | | GAGCGAGATTCCGTCTCAAAGAAAAAAAAAGTAATGAAATGAATA |
| | | AAATGAGTCCTAGAGCCAGTAAATGTCGTAAATGTCTCAGCTAGTC |
| | | AGGTAGTAAAAGGTCTCAACTAGGCAGTGGCAGAGCAGGATTCAA |
| | | ATTCAGGGCTGTTGTGATGCCTCCGCAGACTCTGAGCGCCACCTGG |
| | | TGGTAATTTGTCTGTGCCTCTTCTGACGTGGAAGAACAGCAACTAA |
| | | CACACTAACACGGCATTTACTATGGGCCAGCCATTGTCCATCTAGA |
| | | TGGccgataaaataaaagattttatttagtctccagaaaaaggggggaatgaaagaccccacctgtaggtttg |
| | | gcaagctagctgcaGTGTGTCAGTTAGGGTGTGGAAAGTCCCCAGGCTCCC |
| | | CAGCAGGCAGAAGTATGCAAAGCATGCATCTCAATTAGTCAGCAA |
| | | CCAGGTGTGGAAAGTCCCCAGGCTCCCCAGCAGGCAGAAGTATGC |
| | | AAAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCTGCCTCTGAGCTATTCCAGAAGTAGTGAGGAGGCTTTTTTGG |
| | | AGGCCTAGGCTTTTGCAAAGGATCCGCCACCATGTCTAGACCTGGC |
| | | GAGAGGCCCTTCCAGTGCCGGATCTGCATGCGGAACTTCAGCAAC |
| | | ATGAGCAACCTGACCAGACACACCCGGACACACACAGGCGAGAA |
| | | GCCTTTTCAGTGCAGAATCTGTATGCGCAATTTCTCCGACAGAAGC |
| | | GTGCTGCGGAGACACCTGAGAACCCACACCGGCAGCCAGAAACCA |
| | | TTCCAGTGTCGCATCTGTATGAGAAACTTTAGCGACCCCTCCAATC |
| | | TGGCCCGGCACACCAGAACACATACCGGGGAAAAACCCTTTCAGT |
| | | GTAGGATATGCATGAGGAATTTTTCCGACCGGTCCAGCCTGAGGC |
| | | GGCACCTGAGGACACATACTGGCTCCCAAAAGCCGTTCCAATGTC |
| | | GGATATGTATGCGCAACTTTAGCCAGAGCGGCACCCTGCACAGAC |
| | | ACACAAGAACCCATACTGGCGAGAAACCTTTCCAATGTAGAATCT |
| | | GCATGCGAAATTTTTCCCAGCGGCCTAATCTGACCAGGCATCTGAG |
| | | GACCCACCTGAGAGGATCTaCCTGCAGGGATGAGTTTCCCACCATG |
| | | GTGTTTCCTTCTGGGCAGATCAGCCAGGCCTCGGCCTTGGCCCCGG |
| | | CCCCTCCCCAAGTCCTGCCCCAGGCTCCAGCCCCTGCCCCTGCTCC |
| | | AGCCATGGTATCAGCTCTGGCCCAGGCCCCAGCCCCTGTCCCAGTC |
| | | CTAGCCCCAGGCCCTCCTCAGGCTGTGGCCCCACCTGCCCCCAAGC |
| | | CCACCCAGGCTGGGGAAGGAACGCTGTCAGAGGCCCTGCTGCAGC |
| | | TGCAGTTTGATGATGAAGACCTGGGGGCCTTGCTTGGCAACAGCA |
| | | CAGACCCAGCTGTGTTCACAGACCTGGCATCCGTCGACAACTCCGA |
| | | GTTTCAGCAGCTGCTGAACCAGGGCATACCTGTGGCCCCCCACACA |
| | | ACTGAGCCCATGCTGATGGAGTACCCTGAGGCTATAACTCGCCTAG |
| | | TGACAGGGGCCCAGAGGCCCCCCGACCCAGCTCCTGCTCCACTGG |
| | | GGGCCCCGGGGCTCCCCAATGGCCTCCTTTCAGGAGATGAAGACTT |
| | | CTCCTCCATTGCGGACATGGACTTCTCAGCCCTGCTGAGTCAGATC |
| | | AGCTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgccttgtt |
| | | ggatgctgagccccccatactctattccgagtatgatcctaccagacccttcagtgaagcttcgatgatggg |
| | | cttactgaccaacctggcagacagggagATCGTTCACATGATCAACTGGGCGAAGAGG |
| | | GTGCCAGGCTTTGTGGATTTGACCCTCCATGATCAGGTCCACCTTC |
| | | TAGAATGTGCCTGGCTAGAGATCCTGATGATTGGTGTGGTCTGGCG |
| | | CTCCATGGAGCACCCAGTGAAGCTACTGTTTGCTCCTAACTTGCTC |
| | | TTGGACAGGGACCAGGGAAAATGTGTAGAGGGCCTGGTGGAGATC |
| | | TTCGACATGCTGCTGGCTACATCATCTCGGTTCCGCATGATGAATC |
| | | TGCAGGGAGAGGAGTTTGTGTGCCTCAAATCTATTATTTTGCTTAA |
| | | TTCTGGAGTGTACACATTTCTGCCCAGCACCCTGAAGTCTCTGGAA |
| | | GAGAAGGACCATATCCACCGAGTCCTGGACAAGATCACAGACACT |
| | | TTGATCCACCTGATGGCCAAGGCAGGCCTGACCCTGCAGCAGCAG |
| | | CACCAGCGGCTGGCCCAGCTCCTCCTCATCCTCTCCCACATCAGGC |
| | | ACGCCAGTAACAAAGGCATGGAGCTGctgtacagcatgaagtgcaagaacgtggtg |
| | | cccctctatGaCctgctgctggaggcggcggacgcccaccgcctacatgcgcccactagccgtggagggg |
| | | catccgtggaggagacggaccaaagccacttggccactgcgggctctacttcatcgcattccttgcaaaagtat |
| | | tacatcacgggggaggcagagggtttccctgccacaTaAGTCGACAATCAACCTCtggatta |
| | | caaaatttgtgaaagattgactggtattcttaactatgttgctccttttacgctatgtggatacgctgcttt |
| | | aatgcctttgtatcatgctattgcttcccgtatggctttcattttctcctccttgtataaatcctggttgct |
| | | gtctctttatgaggagttgtggcccgttgtcaggcaacgtggcgtggtgtgcactgtgtttgctgacgcaacc |
| | | cccactggttggggcattgccaccacctgtcagctcctttccgggactttcgctttccccctccctattgcca |
| | | cggcggaactcatcgccgcctgccttgcccgctgctggacaggggctcggctgttgggcactgacaattccg |
| | | tggtgttgtcggggaaatcatcgtcctttccttggctgctcgcctgtgttgccacctggattctgcgcgggac |
| | | gtccttctgctacgtcccttcggccctcaatccagcggaccttccttcccgcggcctgctgccggctctgcgg |
| | | cctcttccgcgtctacgccttcgccctcagacgagtcggatctccctttgggccgcctccccgcgatatcag |
| | | tggtccaggctctagttttgactcaacaatatcaccagctgaagcctatagagtacgagccatagataaaata |
| | | aaagattttatttagtctccagaaaaaggggggaatgaaagaccccacctgtaggtttggcaagctagcaata |
| | | aaagagcccacaacccctcactcggggcgccagtcctccgattgactgagtcgcccggccgcttcgagcagac |
| | | atgataagatacattgatgagtttggacaaaccacaactagaatgcagtgaaaaaaatgctttatttgtgaaa |
| | | tttgtgatgctattgctttatttgtaaccattataagctgcaataaacaagttaacaacaacaattgcattca |
| | | ttttatgtttcaggttcagggggagatgtgggaggttttttaaagcaagtaaaacctctacaaatgtggtaaa |
| | | atcgataaggatcgggtacccgtgtatccaataaaccctcttgcagttgcatccgacttgtggtctcgctgtt |
| | | ccttgggagggtctcctctgagtgattgactacccgtcagcgggggtctttcacacatgcagcatgtatcaaa |
| | | attaatttggttttttttcttaagctgtgccttctagttgccagccatctgttgtttgcccctcccccgtgcc |
| | | ttccttgaccctggaaggtgccactcccactgtcctttcctaataaaatgaggaaattgcat |
| | | cgcattgtctgagtaggtgtcattctattctggggggtggggggggcaggacagcaagggggaggattggg |
| | | aagacaatagcaggcatgctggggatgcggtgggctctatggagatcccgcggtacctcgcgaatgcatcta |
| | | gtgatccaatggcctttttggcccagacatgataagatacattgatgagtttggacaaaccacaactagaatg |
| | | cagaaaaaaatgctttatttgtgaaatttgtgatgctattgctttatttgtaaccattataagctgcaataaac |
| | | aagttgcggccgcttagccctcccacacataaccagagggcagcaattcacgaatcccaactgccgtcggctgt |
| | | ccatcactgtccttcactatggctttgatcccaggatgcagatcgagaagcacctgtcggcaccgtccgcagg |
| | | ggctcaagatgcccctgttctcatttccgatcgcgacgatacaagtcaggttgccagctgccgcagcagcagc |
| | | agtgcccagcaccacgagttctgcacaaggtcccccagtaaaatgatatacattgacaccagtgaagatgcggc |
| | | cgtcgctagagagagctgcgctggcgacgctgtagtcttcagagatggggatgctgttgattgtagccgttgc |
| | | tctttcaatgagggtggattcttcttgagacaaaggcttggccatgcggccgccgctcggtgttcgaggccac |
| | | acgcgtcaccttaatatgcgaagtggacctcggaccgcgccgccccgactgcatctgcgtgttcgaattcgcc |
| | | aatgacaagacgctgggcggggtttgtgtcatcatagaactaaagacatgcaaatatatttcttccggggggt |
| | | accggcctttttggccATTGGatcggatctggccaaaaaggcccttaagtatttacattaaatggccatagt |
| | | acttaaagttacattggcttccttgaaataaacatggagtattcagaatgtgtcataaatatttctaatttt |
| | | aagatagtatctccattggctttctactttttcttttatttttttttgtcctctgtcttccatttgttgttgt |
| | | tgttgtttgtttgtttgtttgttggttggttggttaatttttttttaaagatcctacactatagttcaagcta |
| | | gactattagctactctgtaacccagggtgaccttgaagtcatgggtagcctgctgttttagccttcccacatc |
| | | taagattacaggtatgagctatcatttttggtatattgattgattgattgattgatgtgtgtgtgtgtgattg |
| | | tgtttgtgtgtgtgaTtgtgTaTatgtgtgtatggTtgtgtgtgaTtgtgtgtatgtatgTTtgtgtgtgaTt |
| | | gTgtgtgtgtgaTtgtgcatgtgtgtgtgtgtgaTtgtgtTtatgtgtatgaTtgtgtgtgtgtgtgtgtgt |
| | | gtgtgtgtgtgtgtgtgtgtgtgtgtgttgtgTaTaTatatttatggtagtgagagGcaacgctccggctcag |
| | | gtgtcaggttggtttttgagacagagtctttcacttagcttggaattcactggccgtcgttttacaacgtcgt |
| | | gactgggaaaaccctggcgttacccaacttaatcgccttgcagcacatccccctttcgccagctggcgtaata |
| | | gcgaagaggcccgcaccgatcgcccttcccaacagttgcgcagcctgaatggcgaatggcgcctgatgcggta |
| | | ttttctccttacgcatctgtgcggtatttcacaccgcatatggtgcactctcagtacaatctgctctgatgcc |
| | | gcatagttaagccagccccgacacccgccaacacccgctgacgcgccctgacgggcttgtctgctcccggcat |
| | | ccgcttacagacaagctgtgaccgtctccgggagctgcatgtgtcagaggttttcaccgtcatcaccgaaacg |
| | | cgcgagacgaaagggcctcgtgatacgcctatttttataggttaatgtcatgataataatggtttcttagacg |
| | | tcaggtggcacttttcggggaaatgtgcgcggaacccctatttgtttatttttctaaatacattcaaatatg |
| | | tatccgctcatgagacaataaccctgataaatgcttcaataatattgaaaaaggaagagtatgagtattcaac |
| | | atttccgtgtcgcc |
| SB07139 | 81 | cttattcccttttttgcggcattttgccttcctgtttttgctcacccagaaacgctggtgaaagtaaaagat |
| vector | | gctgaagatcagttgggtgcacgagtgggttacatcgaactggatctcaacagcggtaagatccttgagagtt |
| | | ttcgccccgaagaacgttttccaatgatgagcacttttaaagttctgctatgtggcgcggtattatcccgtat |
| | | tgacgccgggcaagagcaactcggtcgccgcatacactattctcagaatgacttggttgagtactcaccagtc |
| | | acagaaaagcatcttacggatggcatgacagtaagagaattatgcagtgctgccataaccatgagtgataaca |
| | | ctgcggccaacttacttctgacaacgatcggaggaccgaaggagctaaccgcttttttgcacaacatggggga |
| | | tcatgtaactcgccttgatcgttgggaaccggagctgaatgaagccataccaaacgacgagcgtgacaccacg |
| | | atgcctgtagcaatggcaacaacgttgcgcaaactattaactggcgaactacttactctagcttcccggcaac |
| | | aattaatagactggatggaggcggataaagttgcaggaccacttctgcgctcggcccttccggctggctggtt |
| | | tattgctgataaatctggagccggtgagcgtgggtctcgcggtatcattgcagcactggggccagatggtaag |
| | | ccctcccgtatcgtagttatctacacgacggggagtcaggcaactatggatgaacgaaatagacagatcgctg |
| | | agataggtgcctcactgattaagcattggtaactgtcagaccaagtttactcatatatactttagattgattt |
| | | aaaacttcatttttaatttaaaaggatctaggtgaagatcctttttgataatctcatgaccaaaatcccttaa |
| | | cgtgagttttcgttccactgagcgtcagaccccgtagaaaagatcaaaggatcttcttgagatccttttttt |
| | | ctgcgcgtaatctgctgcttgcaaacaaaaaaaccaccgctaccagcggtggtttgtttgccggatcaagagc |
| | | taccaactctttttccgaaggtaactggcttcagcagagcgcagataccaaatactgtccttctagtgtagcc |
| | | gtagttaggccaccacttcaagaactctgtagcaccgccta |
| | | catacctcgctctgctaatcctgttaccagtggctgctgccagtggcgataagtcgtgtcttaccgggttgga |
| | | ctcaagacgatagttaccggataaggcgcagcggtcgggctgaacggggggttcgtgcacacagcccagcttgg |
| | | agcgaacgacctacaccgaactgagatacctacagcgtgagctatgagaaagcgccacgcttcccgaaggg |
| | | agaaaggcggacaggtatccggtaagcggcagggtcggaacaggagagcgcacgagggagcttccaggg |
| | | ggaaacgcctggtatctttatagtcctgtcgggtttcgccacctctgacttgagcgtcgatttttgtgatgct |
| | | cgtcaggggggcggagcctatggaaaaacgccagcaacgcggcctttttacggttcctggccttttgctggcc |
| | | ttttgctcacatgttctttcctgcgttatcccctgattctgtggataaccgtattaccgcctttgagtgagct |
| | | gataccgctcgccgcagccgaacgaccgagcgcagcgagtcagtgagcgaggaagcggaagagcgcccaatac |
| | | gcaaaccgcctctccccgcgcgttggccgattcattaatgcagctggcacgacaggtttcccgactggaaagc |
| | | gggcagtgagcgcaacgcaattaatgtgagttagctcactcattaggcaccccaggctttacactttatgctt |
| | | ccggctcgtatgttgtgtggaattgtgagcggataacaatttcacacaggaaacagctatgaccatgattacg |
| | | ccaagcttgaattcgagcttgcatgcctgcaggtcgttacataacttacggtaaatggcccgcctggctgacc |
| | | gcccaacgacccccgcccattgacgtcaataatgacgtatgttcccatagtaacgccaatagggactttccat |
| | | tgacgtcaatgggtggagtatttacggtaaactgcccacttggcagtacatcaagtgtatcatatgccaagta |
| | | cgccccctattgacgtcaatgacggtaaatggcccgcctggcattatgcccagtacatgaccttatgggactt |
| | | tcctacttggcagtacatctacgtattagtcatcgctattaccatggtgatgcggttttggcagtacatcaat |
| | | gggcgtggatagcggtttgactcacggggatttccaagtctccaccccattgacgtcaatgggagtttgtttt |
| | | ggcaccaaaatcaacgggactttccaaaatgtcgtaacaactccgccccattgacgcaaatgggcggtaggcg |
| | | tgtacggtgggaggtctatataagcagagctcaataaaagagcccacaacccctcactcggcgcgccagtcct |
| | | ccgattgactgagtcgcccgggtacccgtgtatccaataaaccctcttgcagttgcatccgacttgtggtctc |
| | | gctgttccttgggagggtctcctctgagtgattgactacccgtcagcgggggtctttcatttgggggctcgtc |
| | | cgagatcgggagacccctgcccagggaccaccgacccaccaccgggaggtaagctggccagcaacttatctgt |
| | | gtctgtccgattgtctagtgtctatgactgattttatgcgcctgcgtcggtactagttagctaactagctct |
| | | gtatctggcggacccgtggtggaactgacgagttcggaacacccggccgcaaccctgggagacgtcccaggg |
| | | acttcgggggccgtttttgtggcccgacctgagtcctaaaatcccgatcgtttaggactctttggtgcacccc |
| | | ccttagaggagggatatgtggttctggtaggagacgagaacctaaaacagttcccgcctccgtctgaatttt |
| | | tgctttcggtttgggaccgaagccgcgccgcgcgtcttgtctgctgcagcatcgttctgtgttgtctctgtc |
| | | tgactgtgtttctgtatttgtctgaaaatatgggccccccctcgagtccccagcatgcctgctattctcttcc |
| | | caatcctcccccttgctgtcctgccccaccccaccccccagaatagaatgacacctactcagacaatgcgatg |
| | | caatttcctcattttattaggaaaggacagtgggagtggcaccttccagggtcaaggaaggcacgggggaggg |
| | | gcaaacaacagatggctggcaactagaaggcacagttacttaCACAGGCCGCACAGATTCTCTCCGCAGCCGT |
| | | TCGTTTCTTCTCCGCTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGGG |
| | | TTCTTCCAGGCTAGACTTCTGGGGCACTGTCTCGCTGTTGAAGTTC |
| | | AGGGCCTGCATCAGCTCGTCGATCACGGCCAGCATATTCTGGTCCA |
| | | GGAAGATCTGCCGCTTGGGGTCCATCAGCAGCTTGGCGTTCATGGT |
| | | CTTGAATTCCACCTGGTACATCTTCAGGTCCTCGTAGATGCTGCTC |
| | | AGGCACAGGGCCATCATGAAGGAGGTCTTTCTGCTGGCCAGGCAA |
| | | GAGCCGTTGGTGATGAAGCTGGTTTCCCGGCTGTTCAGGCAGCTCT |
| | | CGTTCTTGGTCAGTTCCAGAGGCAGGCAGGCTTCCACGGTGCTGGT |
| | | CTTATCCTTGGTGATGTCCTCGTGGTCGATTTCCTCGCTGGTGCAGG |
| | | GGTAGAATTCCAGGGTCTGTCTGGCCTTCTGCAGCATGTTGGACAC |
| | | GGCTCTCAGCAGGTTCTGGCTGTGGTGCAGACAAGGGAACATGCC |
| | | AGGATCAGGAGTGGCCACAGGCAGGTTTCTAGATCCGCCGCCAGA |
| | | TCCACCACCTGATCCGCCACCGCTTCCTCCGCCAGAACATGGCACG |
| | | CTGGCCCATTCGCTCCAAGAGCTGCTGTAGTACCGGTCCTGGGCTC |
| | | TGACGCTGATGCTGGCGTTCTTTCTGCAGATCACGGTGGCGCTGGT |
| | | CTTGTCGGTGAACACCCGGTCCTTTTTCTCGCGCTTGGACTTGCCCT |
| | | GCACTTGCACGCAAAAGGTCAGGCTGAAGTAGCTGTGGGGTGTAG |
| | | ACCAGGTGTCGGGGTACTCCCAGGACACTTCCACCTGTCTGCTGTT |
| | | CTTCAGAGGCTTCAGCTGCAGGTTCTTTGGAGGATCGGGCTTGATG |
| | | ATGTCCCGGATGAAAAAGCTGGAGGTGTAGTTCTCGTACTTCAGCT |
| | | TGTGCACGGCGTCCACCATCACTTCGATAGGCAGAGACTCTTCGGC |
| | | GGCTGGACAGGCGCTGTCCTCTTGGCATTCCACGCTGTACTCGTAT |
| | | TCTTTGTTGTCGCCCCGCACTCTTTCGGCAGACAGTGTAGCGGCGC |
| | | CACATGTAACGCCCTGAGGATCACTGCTGCCTCTGCTGGACTTCAC |
| | | GCTGAAGGTCAGGTCGGTGCTGATGGTGGTCAGCCACCAACATGT |
| | | GAACCGGCCGCTGTAGTTCTTGGCCTCGCATCTCAGGAAGGTCTTG |
| | | TTCTTGGGCTCTTTCTGGTCCTTCAGGATGTCGGTGCTCCAAATGCC |
| | | ATCCTCTTTCTTGTGGAGCAGCAGCAGGCTGTGGCTCAGCACTTCT |
| | | CCGCCTTTGTGACAGGTGTACTGGCCGGCGTCGCCAAACTCTTTCA |
| | | CTTGGATGGTCAGGGTCTTGCCGCTGCCGAGCACCTCGCTAGACTG |
| | | ATCCAGTGTCCAGGTGATGCCGTCCTCTTCAGGGGTATCGCAGGTC |
| | | AGCACCACCATCTCGCCAGGAGCATCGGGATACCAGTCCAGTTCC |
| | | ACCACGTACACGTCTTTCTTCAGCTCCCAGATGGCCACCAGAGGAG |
| | | AGGCCAGGAACACCAGGCTGAACCAGCTGATGACCAGCTGCTGGT |
| | | GACACATCATGGTGGCGACACCGGTACGCGTTGGCCCCCATTATAT |
| | | ACCCTCTAGAACTAGTtatccactccgtgtaagggagagtgagcctcttacgaatcTTCGG |
| | | CGTCGACTGCTTCattccgaggcgactgataccTTCGGCGTCGACTGCTTCatacg |
| | | aaggcagtccgattcTTCGGCGTCGACTGCTTCaacctttactgagacgggacTTCGGC |
| | | GTCGACTGCTTCaaaggcgttgcgaatcctcatgcgattgttacgaaacccgTTAATTAAA |
| | | GAGCGAGATTCCGTCTCAAAGAAAAAAAAAGTAATGAAATGAATA |
| | | AAATGAGTCCTAGAGCCAGTAAATGTCGTAAATGTCTCAGCTAGTC |
| | | AGGTAGTAAAAGGTCTCAACTAGGCAGTGGCAGAGCAGGATTCAA |
| | | ATTCAGGGCTGTTGTGATGCCTCCGCAGACTCTGAGCGCCACCTGG |
| | | TGGTAATTTGTCTGTGCCTCTTCTGACGTGGAAGAACAGCAACTAA |
| | | CACACTAACACGGCATTTACTATGGGCCAGCCATTGTCCATCTAGA |
| | | TGGccgataaaataaaagattttatttagtctccagaaaaaggggggaatgaaagaccccacctgtaggtttg |
| | | gcaagctagctgcaGTGTGTCAGTTAGGGTGTGGAAAGTCCCCAGGCTCCC |
| | | CAGCAGGCAGAAGTATGCAAAGCATGCATCTCAATTAGTCAGCAA |
| | | CCAGGTGTGGAAAGTCCCCAGGCTCCCCAGCAGGCAGAAGTATGC |
| | | AAAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCTGCCTCTGAGCTATTCCAGAAGTAGTGAGGAGGCTTTTTTGG |
| | | AGGCCTAGGCTTTTGCAAAGGATCCGCCACCATGTCTAGACCTGGC |
| | | GAGAGGCCCTTCCAGTGCCGGATCTGCATGCGGAACTTCAGCAAC |
| | | ATGAGCAACCTGACCAGACACACCCGGACACACACAGGCGAGAA |
| | | GCCTTTTCAGTGCAGAATCTGTATGCGCAATTTCTCCGACAGAAGC |
| | | GTGCTGCGGAGACACCTGAGAACCCACACCGGCAGCCAGAAACCA |
| | | TTCCAGTGTCGCATCTGTATGAGAAACTTTAGCGACCCCTCCAATC |
| | | TGGCCCGGCACACCAGAACACATACCGGGGAAAAACCCTTTCAGT |
| | | GTAGGATATGCATGAGGAATTTTTCCGACCGGTCCAGCCTGAGGC |
| | | GGCACCTGAGGACACATACTGGCTCCCAAAAGCCGTTCCAATGTC |
| | | GGATATGTATGCGCAACTTTAGCCAGAGCGGCACCCTGCACAGAC |
| | | ACACAAGAACCCATACTGGCGAGAAACCTTTCCAATGTAGAATCT |
| | | GCATGCGAAATTTTTCCCAGCGGCCTAATCTGACCAGGCATCTGAG |
| | | GACCCACCTGAGAGGATCTaCCTGCAGGGATGAGTTTCCCACCATG |
| | | GTGTTTCCTTCTGGGCAGATCAGCCAGGCCTCGGCCTTGGCCCCGG |
| | | CCCCTCCCCAAGTCCTGCCCCAGGCTCCAGCCCCTGCCCCTGCTCC |
| | | AGCCATGGTATCAGCTCTGGCCCAGGCCCCAGCCCCTGTCCCAGTC |
| | | CTAGCCCCAGGCCCTCCTCAGGCTGTGGCCCCACCTGCCCCCAAGC |
| | | CCACCCAGGCTGGGGAAGGAACGCTGTCAGAGGCCCTGCTGCAGC |
| | | TGCAGTTTGATGATGAAGACCTGGGGGCCTTGCTTGGCAACAGCA |
| | | CAGACCCAGCTGTGTTCACAGACCTGGCATCCGTCGACAACTCCGA |
| | | GTTTCAGCAGCTGCTGAACCAGGGCATACCTGTGGCCCCCCACACA |
| | | ACTGAGCCCATGCTGATGGAGTACCCTGAGGCTATAACTCGCCTAG |
| | | TGACAGGGGCCCAGAGGCCCCCCGACCCAGCTCCTGCTCCACTGG |
| | | GGGCCCCGGGGCTCCCCAATGGCCTCCTTTCAGGAGATGAAGACTT |
| | | CTCCTCCATTGCGGACATGGACTTCTCAGCCCTGCTGAGTCAGATC |
| | | AGCTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgccttgttg |
| | | gatgctgagccccccatactctattccgagtatgatcctaccagacccttcagtgaagcttcgatgatgggct |
| | | tactgaccaacctggcagacagggagCTGGTTCACATGATCAACTGGGCGAAGAGG |
| | | GTGCCAGGCTTTGTGGATTTGACCCTCCATGATCAGGTCCACCTTC |
| | | TAGAATGTGCCTGGCTAGAGATCCTGATGATTGGTCTCGTCTGGCG |
| | | CTCCATGGAGCACCCAGTGAAGCTACTGTTTGCTCCTAACTTGGTG |
| | | TTGGACAGGGACGAGGGAAAATGTGTAGAGGGCCTGGTGGAGATC |
| | | TTCGACATGCTGCTGGCTACATCATCTCGGTTCCGCATGATGAATC |
| | | TGCAGGGAGAGGAGTTTGTGTGCCTCAAATCTATTATTTTGCTTAA |
| | | TTCTGGAGTGTACACATTTCTGCCCAGCACCCTGAAGTCTCTGGAA |
| | | GAGAAGGACCATATCCACCGAGTCCTGGACAAGATCACAGACACT |
| | | TTGATCCACCTGATGGCCAAGGCAGGCCTGACCCTGCAGCAGCAG |
| | | CACCAGCGGCTGGCCCAGCTCCTCCTCATCCTCTCCCACATCAGGC |
| | | ACATGAGTAACAAAGGCATGGAGCTGctgtacagcatgaagtgcaagaacgtggtg |
| | | cccctctatGaCctgctgctggaggcggcggacgcccaccgcctacatgcgcccactagccgtggagggg |
| | | catccgtggaggagacggaccaaagccacttggccactgcgggctctacttcatcgcattccttgcaaaagtat |
| | | tacatcacgggggaggcagagggtttccctgccacaTaAGTCGACAATCAACCTCtggatta |
| | | caaaatttgtgaaagattgactggtattcttaactatgttgctccttttacgctatgtggatacgctgcttt |
| | | aatgcctttgtatcatgctattgcttcccgtatggctttcattttctcctccttgtataaatcctggttgct |
| | | gtctctttatgaggagttgtggcccgttgtcaggcaacgtggcgtggtgtgcactgtgtttgctgacgcaacc |
| | | cccactggttggggcattgccaccacctgtcagctcctttccgggactttcgctttccccctccctattgcca |
| | | cggcggaactcatcgccgcctgccttgcccgctgctggacaggggctcggctgttgggcactgacaattccgt |
| | | ggtgttgtcggggaaatcatcgtcctttccttggctgctcgcctgtgttgccacctggattctgcgcgggacg |
| | | tccttctgctacgtcccttcggccctcaatccagcggaccttccttcccgcggcctgctgccggctctgcggc |
| | | ctcttccgcgtctacgccttcgccctcagacgagtcggatctccctttgggccgcctccccgcgatatcagtg |
| | | gtccaggctctagttttgactcaacaatatcaccagctgaagcctatagagtacgagccatagataaaataaa |
| | | agattttatttagtctccagaaaaaggggggaatgaaagaccccacctgtaggtttggcaagctagcaataaa |
| | | agagcccacaacccctcactcggggcgccagtcctccgattgactgagtcgcccggccgcttcgagcagacat |
| | | gataagatacattgatgagtttggacaaaccacaactagaatgcagtgaaaaaaatgctttatttgtgaaatt |
| | | tgtgatgctattgctttatttgtaaccattataagctgcaataaacaagttaacaacaacaattgcattcat |
| | | tttatgtttcaggttcagggggagatgtgggaggttttttaaagcaagtaaaacctctacaaatgtggtaaa |
| | | atcgataaggatcgggtacccgtgtatccaataaaccctcttgcagttgcatccgacttgtggtctcgctgtt |
| | | ccttgggagggtctcctctgagtgattgactacccgtcagcgggggtctttcacacatgcagcatgtatcaaa |
| | | attaatttggttttttttcttaagctgtgccttctagttgccagccatctgttgtttgcccctcccccgtgcc |
| | | ttccttgaccctggaaggtgccactcccactgtcctttcctaataaaatgaggaaattgcatcgcattgtctg |
| | | agtaggtgtcattctattctggggggtggggtggggcaggacagcaagggggaggattgggaagacaatagca |
| | | ggcatgctggggatgcggtgggctctatggagatcccgcggtacctcgcgaatgcatctagatccaatggcct |
| | | ttttggcccagacatgataagatacattgatgagtttggacaaaccacaactagaatgcagtgaaaaaaatgc |
| | | tttatttgtgaaatttgtgatgctattgctttatttgtaaccattataagctgcaataaacaagttgcggccg |
| | | cttagccctcccacacataaccagagggcagcaattcacgaatcccaactgccgtcggctgtccatcactgtc |
| | | cttcactatggctttgatcccaggatgcagatcgagaagcacctgtcggcaccgtccgcaggggctcaag |
| | | atgcccctgttctcatttccgatcgcgacgatacaagtcaggttgccagctgccgcagcagcagcagtgccca |
| | | gcaccacgagttctgcacaaggtcccccagtaaaatgatatacattgacaccagtgaagatgcggccgtcgct |
| | | agagagagctgcgctggcgacgctgtagtcttcagagatggggatgctgttgattgtagccgttgctctttcaa |
| | | tgagggtggattcttcttgagacaaaggcttggccatgcggccgccgctcggtgttcgaggccacacgcgtcac |
| | | cttaatatgcgaagtggacctcggaccgcgccgccccgactgcatctgcgtgttcgaattcgccaatgacaag |
| | | acgctgggcggggtttgtgtcatcatagaactaaagacatgcaaatatatttcttccggggggtaccggcctt |
| | | tttggccATTGGatcggatctggccaaaaaggcccttaagtatttacattaaatggccatagtacttaaagtt |
| | | acattggcttccttgaaataaacatggagtattcagaatgtgtcataaatatttctaattttaagatagtatc |
| | | tccattggctttctactttttcttttatttttttttgtcctctgtcttccatttgttgttgttgttgtttgtt |
| | | tgtttgtttgttggttggttggttaatttttttttaaagatcctacactatagttcaagctagactattagct |
| | | actctgtaacccagggtgaccttgaagtcatgggtagcctgctgttttagccttcccacatctaagattacag |
| | | gtatgagctatcatttttggtatattgattgattgattgattgatgtgtgtgtgtgtgattgtgtttgtgtg |
| | | tgtgaTtgtgTaTatgtgtgtatggTtgtgtgtgaTtgtgtgtatgtatgTTtgtgtgtgaTtgTgtgtgtg |
| | | tgaTtgtgcatgtgtgtgtgtgtgaTtgtgtTtatgtgtatgaTtgtgtgtgtgtgtgtgtgtgtgtgtgtg |
| | | tgtgtgtgtgtgtgtgtgttgtgTaTaTatatttatggtagtgagagGcaacgctccggctcaggtgtcagg |
| | | ttggtttttgagacagagtctttcacttagcttggaattcactggccgtcgttttacaacgtcgtgactggga |
| | | aaaccctggcgttacccaacttaatcgccttgcagcacatccccctttcgccagctggcgtaatagcgaagag |
| | | gcccgcaccgatcgcccttcccaacagttgcgcagcctgaatggcgaatggcgcctgatgcggtattttctc |
| | | cttacgcatctgtgcggtatttcacaccgcatatggtgcactctcagtacaatctgctctgatgcc |
| | | gcatagttaagccagccccgacacccgccaacacccgctgacgcgccctgacgggcttgtctgctcccggca |
| | | tccgcttacagacaagctgtgaccgtctccgggagctgcatgtgtcagaggttttcaccgtcatcaccgaaacg |
| | | cgcgagacgaaagggcctcgtgatacgcctatttttataggttaatgtcatgataataatggtttcttagacg |
| | | tcaggtggcacttttcggggaaatgtgcgcggaacccctatttgtttatttttctaaatacattcaaatatg |
| | | tatccgctcatgagacaataaccctgataaatgcttcaataatattgaaaaaggaagagtatgagtattcaac |
| | | atttccgtgtcgcccttattcccttttttgcggcattttgccttcctgtttttgctcacccagaaacgctggt |
| | | gaaagtaaaagatgctgaagatcagttgggtgcacgagtgggttacatcgaactggatctcaacagcggtaa |
| | | gatccttgagagttttcgccccgaagaacgttttccaatgatgagcacttttaaagttctgctatgtggcgc |
| | | ggtattatcccgtattgacgccgggcaagagcaactcggtcgccgcatacactattctcagaatgacttggtt |
| | | gagtactcaccagtcacagaaaagcatcttacggatggcatgacagtaagagaattatgcagtgctgccataa |
| | | ccatgagtgataacactgcggccaacttacttctgacaacgatcggaggaccgaaggagctaaccgctttttt |
| | | gcacaacatgggggatcatgtaactcgccttgatcgttgggaaccggagctgaatgaagccataccaaacgac |
| | | gagcgtgacaccacgatgcctgtagcaatggcaacaacgttgcgcaaactattaactggcgaactacttactc |
| | | tagcttcccggcaacaattaatagactggatggaggcggataaagttgcaggaccacttctgcgctcggccc |
| | | ttccggctggctggtttattgctgataaatctggagccggtgagcgtgggtctcgcggtatcattgcagcact |
| | | ggggccagatggtaagccctcccgtatcgtagttatctacacgacggggagtcaggcaactatggatgaacga |
| | | aatagacagatcgctgagataggtgcctcactgattaagcattggtaactgtcagaccaagtttactcatata |
| | | tactttagattgatttaaaacttcatttttaatttaaaaggatctaggtgaagatcctttttgataatctcatg |
| | | accaaaatcccttaacgtgagttttcgttccactgagcgtcagaccccgtagaaaagatcaaaggatcttctt |
| | | gagatcctttttttctgcgcgtaatctgctgcttgcaaacaaaaaaaccaccgctaccagcggtggtttgttt |
| | | gccggatcaagagctaccaactctttttccgaaggtaactggcttcagcagagcgcagataccaaatactgtc |
| | | cttctagtgtagccgtagttaggccaccacttcaagaactctgtagcaccgcctacatacctcgctctgctaa |
| | | tcctgttaccagtggctgctgccagtggcgataagtcgtgtcttaccgggttggactcaagacgatagttacc |
| | | ggataaggcgcagcggtcgggctgaacggggggttcgtgcacacagcccagcttggagcgaacgacctacacc |
| | | gaactgagatacctacagcgtgagctatgagaaagcgccacgcttcccgaagggagaaaggcggacaggtatc |
| | | cggtaagcggcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcctatg |
| | | gaaaaacgccagcaacgcggcctttttacggttcctggccttttgctggccttttgctcacatgttctttcc |
| | | tgcgttatcccctgattctgtggataaccgtattaccgcctttgagtgagctgataccgctcgccgcagccg |
| | | aacgaccgagcgcagcgagtcagtgagcgaggaagcggaagagcgcccaatacgcaaaccgcctctccccgcg |
| | | cgttggccgattcattaatgcagctggcacgacaggtttcccgactggaaagcgggcagtgagcgcaacgcaa |
| | | ttaatgtgagttagctcactcattaggcaccccaggctttacactttatgcttccggctcgtatgttgtgtgg |
| | | aattgtgagcggataacaatttcacacaggaaacagctatgaccatgattacgcc |
| WRPW | 82 | WRPW |
| motif | | |
| IL-12 | 83 | ATGTGTCACCAGCAGCTGGTCATCAGCTGGTTCAGCCTGGTGTTCC |
| | | TGGCCTCTCCTCTGGTGGCCATCTGGGAGCTGAAGAAAGACGTGTA |
| | | CGTGGTGGAACTGGACTGGTATCCCGATGCTCCTGGCGAGATGGT |
| | | GGTGCTGACCTGCGATACCCCTGAAGAGGACGGCATCACCTGGAC |
| | | ACTGGATCAGTCTAGCGAGGTGCTCGGCAGCGGCAAGACCCTGAC |
| | | CATCCAAGTGAAAGAGTTTGGCGACGCCGGCCAGTACACCTGTCA |
| | | CAAAGGCGGAGAAGTGCTGAGCCACAGCCTGCTGCTGCTCCACAA |
| | | GAAAGAGGATGGCATTTGGAGCACCGACATCCTGAAGGACCAGAA |
| | | AGAGCCCAAGAACAAGACCTTCCTGAGATGCGAGGCCAAGAACTA |
| | | CAGCGGCCGGTTCACATGTTGGTGGCTGACCACCATCAGCACCGA |
| | | CCTGACCTTCAGCGTGAAGTCCAGCAGAGGCAGCAGTGATCCTCA |
| | | GGGCGTTACATGTGGCGCCGCTACACTGTCTGCCGAAAGAGTGCG |
| | | GGGCGACAACAAAGAATACGAGTACAGCGTGGAATGCCAAGAGG |
| | | ACAGCGCCTGTCCAGCCGCCGAAGAGTCTCTGCCTATCGAAGTGAT |
| | | GGTGGACGCCGTGCACAAGCTGAAGTACGAGAACTACACCTCCAG |
| | | CTTTTTCATCCGGGACATCATCAAGCCCGATCCTCCAAAGAACCTG |
| | | CAGCTGAAGCCTCTGAAGAACAGCAGACAGGTGGAAGTGTCCTGG |
| | | GAGTACCCCGACACCTGGTCTACACCCCACAGCTACTTCAGCCTGA |
| | | CCTTTTGCGTGCAAGTGCAGGGCAAGTCCAAGCGCGAGAAAAAGG |
| | | ACCGGGTGTTCACCGACAAGACCAGCGCCACCGTGATCTGCAGAA |
| | | AGAACGCCAGCATCAGCGTCAGAGCCCAGGACCGGTACTACAGCA |
| | | GCTCTTGGAGCGAATGGGCCAGCGTGCCATGTTCTGGCGGAGGAA |
| | | GCGGTGGCGGATCAGGTGGTGGATCTGGCGGCGGATCTAGAAACC |
| | | TGCCTGTGGCCACTCCTGATCCTGGCATGTTCCCTTGTCTGCACCAC |
| | | AGCCAGAACCTGCTGAGAGCCGTGTCCAACATGCTGCAGAAGGCC |
| | | AGACAGACCCTGGAATTCTACCCCTGCACCAGCGAGGAAATCGAC |
| | | CACGAGGACATCACCAAGGATAAGACCAGCACCGTGGAAGCCTGC |
| | | CTGCCTCTGGAACTGACCAAGAACGAGAGCTGCCTGAACAGCCGG |
| | | GAAACCAGCTTCATCACCAACGGCTCTTGCCTGGCCAGCAGAAAG |
| | | ACCTCCTTCATGATGGCCCTGTGCCTGAGCAGCATCTACGAGGACC |
| | | TGAAGATGTACCAGGTGGAATTCAAGACCATGAACGCCAAGCTGC |
| | | TGATGGACCCCAAGCGGCAGATCTTCCTGGACCAGAATATGCTGG |
| | | CCGTGATCGACGAGCTGATGCAGGCCCTGAACTTCAACAGCGAGA |
| | | CAGTGCCCCAGAAGTCTAGCCTGGAAGAACCCGACTTCTACAAGA |
| | | CCAAGATCAAGCTGTGCATCCTGCTGCACGCCTTCCGGATCAGAGC |
| | | CGTGACCATCGACAGAGTGATGAGCTACCTGAACGCCTCT |

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

1. A modified estrogen receptor ligand binding domain (ER-LBD) comprising an amino acid sequence corresponding to amino acids 282-595 of SEQ ID NO: 1, wherein the modified ER-LBD comprises a G400V amino acid substitution, an M543A amino acid substitution, and an L544A amino acid substitution, and one or more additional amino acid substitutions, wherein the one or more additional amino acid substitutions are within a region of SEQ ID NO: 1 selected from the group consisting of: positions 343-354, positions 380-392, positions 404-463, and positions 517-540, and position 547, and wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions,
optionally wherein the modified ER-LBD further comprises a V595A amino acid substitution, and/or
optionally wherein the non-endogenous ligand is selected from the group consisting of: 4-hydroxytamoxifen, N-desmethyltamoxifen, tamoxifen-N-oxide, and endoxifen.

2. The modified ER-LBD of claim 1, wherein the one or more additional amino acid substitutions are at one or more positions of SEQ ID NO: 1 selected from the group consisting of: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

3. The modified ER-LBD of claim 2, wherein:
a. the one or more positions comprise position 343 of SEQ ID NO: 1, optionally wherein the amino acid substitution at position 343 of SEQ ID NO: 1 is selected from the group consisting of: M343F, M343I, M343L, and M343V;
b. the one or more positions comprise position 344 of SEQ ID NO: 1, optionally wherein the amino acid substitution at position 344 of SEQ ID NO: 1 is G344M;
c. the one or more positions comprise position 345 of SEQ ID NO: 1, optionally wherein the amino acid substitution at position 345 of SEQ ID NO: 1 is L345S;
d. the one or more positions comprise position 346 of SEQ ID NO: 1, optionally wherein the amino acid substitution at position 346 of SEQ ID NO: 1 is selected from the group consisting of: L346I, L346M, L346F, and L346V;
e. the one or more positions comprise position 347 of SEQ ID NO: 1, optionally wherein the amino acid substitution at position 347 of SEQ ID NO: 1 is selected from the group consisting of: T347D, T347E, T347F, T347I, T347K, T347L, T347M, T347N, T347Q, T347R, T347S, and T347V;
f. the one or more positions comprise position 348 of SEQ ID NO: 1, optionally wherein the amino acid substitution at position 348 of SEQ ID NO: 1 is N348K;
g. the one or more positions comprise position 349 of SEQ ID NO: 1, optionally wherein the amino acid substitution at position 349 of SEQ ID NO: 1 is selected from the group consisting of: L349I, L349M, L349F, and L349V;
h. the one or more positions comprise position 350 of SEQ ID NO: 1, optionally wherein the amino acid substitution at position 350 of SEQ ID NO: 1 is selected from the group consisting of: A350F, A350I, A350L, A350M and A350V;
i. the one or more positions comprise position 351 of SEQ ID NO: 1, optionally wherein the amino acid substitution at position 351 of SEQ ID NO: 1 is selected from the group consisting of: D351E, D351F, D351I, D351L, D351M, D351N, D351Q, and D351V;
j. the one or more positions comprise position 352 of SEQ ID NO: 1, optionally wherein the amino acid substitution at position 352 of SEQ ID NO: 1 is R352K;
k. the one or more positions comprise position 354 of SEQ ID NO: 1, optionally wherein the amino acid substitution at position 354 of SEQ ID NO: 1 is selected from the group consisting of: L354I, L354M, L354F, and L354V;
l. the one or more positions comprise position 380 of SEQ ID NO: 1, optionally wherein the amino acid substitution at position 380 of SEQ ID NO: 1 is E380Q;
m. the one or more positions comprise position 384 of SEQ ID NO: 1, optionally wherein the amino acid substitution at position 384 of SEQ ID NO: 1 is selected from the group consisting of: L384I, L384M, L384F, and L384V;
n. the one or more positions comprise position 386 of SEQ ID NO: 1, optionally wherein the amino acid substitution at position 386 of SEQ ID NO: 1 is I386V;
o. the one or more positions comprise position 387 of SEQ ID NO: 1, optionally wherein the amino acid substitution at position 387 of SEQ ID NO: 1 is selected from the group consisting of: L387I, L387M, L387F, and L387V;
p. the one or more positions comprise position 388 of SEQ ID NO: 1, optionally wherein the amino acid substitution at position 388 of SEQ ID NO: 1 is selected from the group consisting of: M388I, M388L, and M388F;
q. the one or more positions comprise position 389 of SEQ ID NO: 1, optionally wherein the amino acid substitution at position 389 of SEQ ID NO: 1 is I389M;
r. the one or more positions comprise position 391 of SEQ ID NO: 1, optionally wherein the amino acid substitution at position 391 of SEQ ID NO: 1 is selected from the group consisting of: L391I, L391M, L391F, and L391V;
s. the one or more positions comprise position 392 of SEQ ID NO: 1, optionally wherein the amino acid substitution at position 392 of SEQ ID NO: 1 is V392M;
t. the one or more positions comprise position 404 of SEQ ID NO: 1, optionally wherein the amino acid substitution at position 404 of SEQ ID NO: 1 is selected from the group consisting of: F404I, F404L, F404M, and F404V;
u. the one or more positions comprise position 407 of SEQ ID NO: 1, optionally wherein the amino acid substitution at position 407 of SEQ ID NO: 1 is N407D;
v. the one or more positions comprise position 409 of SEQ ID NO: 1, optionally wherein the amino acid substitution at position 409 of SEQ ID NO: 1 is L409V;
w. the one or more positions comprise position 413 of SEQ ID NO: 1, optionally wherein the amino acid substitution at position 413 of SEQ ID NO: 1 is N413D;
x. the one or more positions comprise position 414 of SEQ ID NO: 1, optionally wherein the amino acid substitution at position 414 of SEQ ID NO: 1 is Q414E;
y. the one or more positions comprise position 417 of SEQ ID NO: 1, optionally wherein the amino acid substitution at position 417 of SEQ ID NO: 1 is C417S;
z. the one or more positions comprise position 418 of SEQ ID NO: 1, optionally wherein the amino acid substitution at position 418 of SEQ ID NO: 1 is selected from the group consisting of: V418I, V418L, V418M, and V418F;
aa. the one or more positions comprise position 420 of SEQ ID NO: 1, optionally wherein the amino acid substitution at position 420 of SEQ ID NO: 1 is selected from the group consisting of: G420I, G420M, G420F, and G420V;
bb. the one or more positions comprise position 421 of SEQ ID NO: 1, optionally wherein the amino acid substitution at position 421 of SEQ ID NO: 1 is selected from the group consisting of: M421I, M421L, M421F, and M421V;
cc. the one or more positions comprise position 422 of SEQ ID NO: 1, optionally wherein the amino acid substitution at position 422 of SEQ ID NO: 1 is V422I;
dd. the one or more positions comprise position 424 of SEQ ID NO: 1, optionally wherein the amino acid substitution at position 424 of SEQ ID NO: 1 is selected from the group consisting of: I424L, I424M, I424F, and I424V;
ee. the one or more positions comprise position 428 of SEQ ID NO: 1, optionally wherein the amino acid substitution at position 428 of SEQ ID NO: 1 is selected from the group consisting of: L428I, L428M, L428F, and L428V;
ff. wherein the one or more positions comprise position 463 of SEQ ID NO: 1, optionally wherein the amino acid substitution at position 463 of SEQ ID NO: 1 is S463P;
gg. the one or more positions comprise position 517 of SEQ ID NO: 1, optionally wherein the amino acid substitution at position 517 of SEQ ID NO: 1 is M517A;
hh. the one or more positions comprise position 521 of SEQ ID NO: 1, optionally wherein the amino acid substitution at position 521 of SEQ ID NO: 1 is selected from the group consisting of: G521A, G521F, G521I, G521L, G521M, and G521V;
ii. the one or more positions comprise position 522 of SEQ ID NO: 1, optionally wherein the amino acid substitution at position 522 of SEQ ID NO: 1 is selected from the group consisting of: M522I, M522L, and M522V;
jj. the one or more positions comprise position 524 of SEQ ID NO: 1, optionally wherein the amino acid substitution at position 524 of SEQ ID NO: 1 is selected from the group consisting of: H524A, H524I, H524L, H524F, and H524V;
kk. the one or more positions comprise position 525 of SEQ ID NO: 1, optionally wherein the amino acid substitution at position 525 of SEQ ID NO: 1 is selected from the group consisting of: L525F, L525I, L525M, L525N, L525Q, L525S, L525T, and L525V;
ll. the one or more positions comprise position 526 of SEQ ID NO: 1, optionally wherein the amino acid substitution at position 526 of SEQ ID NO: 1 is Y526L;
mm. the one or more positions comprise position 527 of SEQ ID NO: 1, optionally wherein the amino acid substitution at position 527 of SEQ ID NO: 1 is S527N;
nn. wherein the one or more positions comprise position 528 of SEQ ID NO: 1, optionally wherein the amino acid substitution at position 528 of SEQ ID NO: 1 is selected from the group consisting of: M528F, M528I, and M528V;
oo. the one or more positions comprise position 533 of SEQ ID NO: 1, optionally wherein the amino acid substitution at position 533 of SEQ ID NO: 1 is selected from the group consisting of: V533F and V533W;
pp. the one or more positions comprise position 534 of SEQ ID NO: 1, optionally wherein the amino acid substitution at position 534 of SEQ ID NO: 1 is selected from the group consisting of: V534Q and V534R;
qq. the one or more positions comprise position 536 of SEQ ID NO: 1, optionally wherein the amino acid substitution at position 536 of SEQ ID NO: 1 is selected from the group consisting of: L536F, and L536M, L536R, and L536Y;
rr. the one or more positions comprise position 537 of SEQ ID NO: 1, optionally wherein the amino acid substitution at position 537 of SEQ ID NO: 1 is selected from the group consisting of: Y537E and Y537S;
ss. the one or more positions comprise position 538 of SEQ ID NO: 1, optionally wherein the amino acid substitution at position 538 of SEQ ID NO: 1 is selected from the group consisting of: D538G and D538K;
tt. the one or more positions comprise position 539 of SEQ ID NO: 1, optionally wherein the amino acid substitution at position 539 of SEQ ID NO: 1 is selected from the group consisting of: L539A and L539R;
uu. the one or more positions comprise position 540 of SEQ ID NO: 1, optionally wherein the amino acid substitution at position 540 of SEQ ID NO: 1 is selected from the group consisting of: L540A and L540F; or
vv. the one or more positions comprise position 547 of SEQ ID NO: 1, optionally wherein the amino acid substitution at position 547 of SEQ ID NO: 1 is H547A.

4. The modified ER-LBD of claim 1, wherein:
a. the one or more additional amino acid substitutions are two amino acid substitutions, optionally wherein each of the two amino acid substitutions are at a position of SEQ ID NO: 1 selected from the group consisting of: 343, 345, 347, 348, 351, 354, 384, 387, 388, 389, 391, 392, 404, 418, 421, 521, 524, and 525,
optionally wherein the two amino acid substitutions are at positions 345 and 348 of SEQ ID NO: 1 and wherein the amino acid substitution at position 345 of SEQ ID NO: 1 is L345S and the amino acid substitution at position 348 of SEQ ID NO: 1 is N348K,
optionally wherein the two amino acid substitutions are at positions 384 and 389 of SEQ ID NO: 1 and wherein the amino acid substitution at position 384 of SEQ ID NO: 1 is L384M and the amino acid substitution at position 389 of SEQ ID NO: 1 is I389M,
optionally wherein the two amino acid substitutions are at positions 421 and 392 of SEQ ID NO: 1 and wherein the amino acid substitution at position 421 of SEQ ID NO: 1 is M421I and the amino acid substitution at position 392 of SEQ ID NO: 1 is V392M,
optionally wherein the two amino acid substitutions are at positions 354 and 391 of SEQ ID NO: 1 and wherein the amino acid substitution at position 354 of SEQ ID NO: 1 is L354I and the amino acid substitution at position 391 of SEQ ID NO: 1 is L391F,
optionally the two amino acid substitutions are at positions 354 and 384 of SEQ ID NO: 1 and wherein the amino acid substitution at position 354 of SEQ ID NO: 1 is L354I and the amino acid substitution at position 384 of SEQ ID NO: 1 is L384M,
optionally wherein the two amino acid substitutions are at positions 354 and 387 of SEQ ID NO: 1 and wherein the amino acid substitution at position 354 of SEQ ID NO: 1 is L354I and the amino acid substitution at position 387 of SEQ ID NO: 1 is L387M,
optionally wherein the two amino acid substitutions are at positions 387 and 391 and wherein the amino acid substitution at position 387 of SEQ ID NO: 1 is L387M and the amino acid substitution at position 391 of SEQ ID NO: 1 is L391F,
optionally wherein the two amino acid substitutions are at positions 384 and 387 of SEQ ID NO: 1 and wherein the amino acid substitution at position 384 of SEQ ID NO: 1 is L384M and the amino acid substitution at position 387 of SEQ ID NO: 1 is L387M,
optionally wherein the two amino acid substitutions are at positions 384 and 391 of SEQ ID NO: 1 and wherein the amino acid substitution at position 384 of SEQ ID NO: 1 is L384M and the amino acid substitution at position 391 of SEQ ID NO: 1 is L391F; and/or
b. the one or more additional amino acid substitutions are three amino acid substitutions, optionally wherein each of the three amino acid substitutions are at a position of SEQ ID NO: 1 selected from the group consisting of: 343, 347, 351, 354, 388, 391, 404, 414, 418, 463, 521, 524, and 525,
optionally wherein the three amino acid substitutions are at positions 354, 384, and 391 of SEQ ID NO: 1 and wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F,
optionally wherein the three amino acid substitutions are at positions 414, 463, and 524 of SEQ ID NO: 1 and wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L; and/or
c. the one or more additional amino acid substitutions are four amino acid substitutions, optionally wherein each of the four amino acid substitutions are at a position of SEQ ID NO: 1 selected from the group consisting of: 343, 347, 351, 354, 384, 388, 391, 404, 413, 418, 463, 521, 524, and 525,
optionally wherein the four amino acid substitutions are at positions 354, 384, 391, and 418 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I,
optionally wherein the four amino acid substitutions are at positions 343, 388, 521, and 404 of SEQ ID NO: 1 and wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L,
optionally wherein the four amino acid substitutions are at positions 524, 347, 351, and 525 of SEQ ID NO: 1 and wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N,
optionally wherein the four amino acid substitutions are at positions 354, 384, 391, and 463 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P,
optionally wherein the four amino acid substitutions are at positions 384, 391, 413, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F; and/or
d. the one or more additional amino acid substitutions are five amino acid substitutions, optionally wherein each of the five amino acid substitutions are at a position of SEQ ID NO: 1 selected from the group consisting of: 354, 384, 391, 409, 413, 414, 421, 463, and 524, optionally wherein the five amino acid substitutions are at positions 384, 409, 413, 463, and 524 of SEQ ID NO: 1 and wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L,
optionally wherein the five amino acid substitutions are at positions 391, 413, 414, 463, and 524 of SEQ ID NO: 1 and wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F,
optionally wherein the five amino acid substitutions are at positions 391, 414, 421, 463, and 524 of SEQ ID NO: 1 and wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F,
optionally wherein the five amino acid substitutions are at positions 354, 409, 413, 421, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L,
optionally wherein the five amino acid substitutions are at positions 354, 409, 421, 463, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L; and/or
e. the one or more additional amino acid substitutions are six amino acid substitutions, optionally wherein each of the six amino acid substitutions are at a position of SEQ ID NO: 1 selected from the group consisting of: 354, 384, 391, 409, 413, 414, 421, 463, and 524, optionally wherein the six amino acid substitutions are at positions 384, 391, 413, 421, 463, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L, optionally wherein the six amino acid substitutions are at positions 409, 413, 414, 421, 463, and 524 of SEQ ID NO: 1 and wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L, optionally wherein the six amino acid substitutions are at positions 354, 391, 409, 413, 414, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L; and/or
f. the one or more additional amino acid substitutions are seven amino acid substitutions, optionally wherein each of the seven amino acid substitutions are at a position of SEQ ID NO: 1 selected from the group consisting of: 354, 384, 391, 409, 413, 414, 421, 463, 517, and 524, optionally wherein the seven amino acid substitutions are at positions 354, 384, 409, 413, 421, 463, and 524 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F,
optionally wherein the seven amino acid substitutions are at positions 354, 391, 413, 421, 463, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L,
optionally wherein the seven amino acid substitutions are at positions 354, 391, 413, 414, 421, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F; and/or
g. the one or more additional amino acid substitutions are eight amino acid substitutions, optionally wherein the eight amino acid substitutions are at positions 384, 391, 409, 413, 421, 463, 517, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

5. A chimeric protein comprising a polypeptide of interest fused to the modified ER-LBD of claim 1,
optionally wherein the polypeptide of interest comprises a nucleic acid binding domain, optionally wherein the nucleic acid binding domain comprises a zinc finger domain, optionally wherein the nucleic acid binding domain comprises a zinc finger domain, optionally wherein the zinc finger domain comprises the sequence
(SEQ ID NO: 62)
MSRPGERPFQCRICMRNFSNMSNLTRHTRTHTGEKPFQCRICMRNFSDR
SVLRRHLRTHTGSQKPFQCRICMRNFSDPSNLARHTRTHTGEKPFQCRI
CMRNFSDRSSLRRHLRTHTGSQKPFQCRICMRNFSQSGTLHRHTRTHTG
EKPFQCRICMRNFSQRPNLTRHLRTHLRGS,
optionally wherein the chimeric protein comprises a chimeric transcription factor, and wherein the polypeptide of interest comprises a nucleic acid binding domain and a transcriptional modulator domain, optionally wherein the transcriptional modular domain is a transcriptional activator, optionally wherein the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain), or
optionally wherein the transcriptional modular domain is a p65 transcriptional activator comprising the amino acid sequence of
(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

6. An isolated polynucleotide molecule comprising a nucleotide sequence encoding the modified ER-LBD of claim 1.

7. A heterologous construct comprising a promoter operatively linked to the polynucleotide molecule of claim 6.

8. A plasmid comprising the heterologous construct of claim 7.

9. A cell comprising the heterologous construct of claim 7.

10. A genetic switch for modulating transcription of a gene of interest, comprising:
(a) the chimeric protein of claim 5, wherein the chimeric protein binds to a chimeric transcription factor-responsive (CTF-responsive) promoter operably linked to the gene of interest; and
(b) a non-endogenous ligand, wherein binding of the non-endogenous ligand to the modified ER-LBD induces the chimeric protein to modulate transcription of the gene of interest.

11. The genetic switch of claim 10, wherein:
a. the non-endogenous ligand is selected from the group consisting of: 4-hydroxytamoxifen, N-desmethyltamoxifen, tamoxifen-N-oxide, and endoxifen; and/or
b. the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme; and/or
c. the gene of interest encodes a cytokine selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha; and/or
d. the gene of interest encodes an IL12p70 fusion protein comprising the amino acid sequence of
(SEQ ID NO: 58)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSGGGSGGGSGGGSGGG
SRNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS.

12. A method of modulating transcription of a gene of interest, comprising:
transforming a cell with (i) a heterologous construct encoding the chimeric protein of claim 5 and (ii) a target expression cassette comprising a chimeric transcription factor-responsive (CTF-responsive) promoter operably linked to the gene of interest, and inducing the chimeric protein to modulate transcription of the gene of interest by contacting the transformed cell with a non-endogenous ligand,
optionally wherein the method further comprises culturing the transformed cell under conditions suitable for expression of the chimeric protein prior to inducing the chimeric protein to modulate transcription,
optionally wherein modulating transcription comprises activating transcription of the gene of interest,
optionally wherein the target expression cassette is encoded by the heterologous construct encoding the chimeric protein of claim 5 or the target expression cassette is encoded by a second heterologous construct, and
optionally wherein the non-endogenous ligand is selected from the group consisting of: 4-hydroxytamoxifen, N-desmethyltamoxifen, tamoxifen-N-oxide, and endoxifen.

13. A method of modulating localization of a chimeric protein comprising transforming a cell with a heterologous construct encoding the chimeric protein of claim 5, and inducing nuclear localization of the chimeric protein by contacting the transformed cell with a non-endogenous ligand,
optionally wherein the method further comprising culturing the transformed cell under conditions suitable for expression of the chimeric protein prior to inducing the nuclear localization, and
optionally wherein the non-endogenous ligand is selected from the group consisting of: 4-hydroxytamoxifen, N-desmethyltamoxifen, tamoxifen-N-oxide, and endoxifen.

14. The method of claim 12, wherein the transformed cell is in a human or animal, and wherein contacting the transformed cell with the non-endogenous ligand comprises administering a pharmacological dose of the ligand to the human or animal.

15. The method of claim 14, wherein the non-endogenous ligand is administered at a concentration at which the non-endogenous ligand is substantially inactive on a wild-type estrogen receptor alpha of SEQ ID NO: 1.

16. The method of claim 13, wherein the transformed cell is in a human or animal, and wherein contacting the transformed cell with the non-endogenous ligand comprises administering a pharmacological dose of the ligand to the human or animal.

17. The method of claim 16, wherein the non-endogenous ligand is administered at a concentration at which the non-endogenous ligand is substantially inactive on a wild-type estrogen receptor alpha of SEQ ID NO: 1.


SEQUENCE LISTING (83 sequences)

SEQ ID NO: 1 - protein, 595 aa, Homo sapiens [sequence omitted]

SEQ ID NO: 2 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 3 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 4 - protein, 19 aa, synthetic construct
  REGION 1..19: note = Description of Artificial Sequence: Synthetic peptide
GGGGSGGGGSGGGGSVDGF

SEQ ID NO: 5 - protein, 9 aa, synthetic construct
  REGION 1..9: note = Description of Artificial Sequence: Synthetic peptide
ASGGGGSAS

SEQ ID NO: 6 - protein, 176 aa, synthetic construct
  REGION 1..176: note = Description of Artificial Sequence: Synthetic polypeptide
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 7 - DNA, 31 bp, synthetic construct
  misc_feature 1..31: note = Description of Artificial Sequence: Synthetic oligonucleotide
  source 1..31: mol_type = other DNA
agagggtatataatggaagctcgacttccag

SEQ ID NO: 8 - DNA, 52 bp, synthetic construct
  misc_feature 1..52: note = Description of Artificial Sequence: Synthetic oligonucleotide
  source 1..52: mol_type = other DNA
gggaatttccggggactttccgggaatttccggggactttccgggaatttcc

SEQ ID NO: 9 - DNA, 85 bp, synthetic construct
  misc_feature 1..85: note = Description of Artificial Sequence: Synthetic oligonucleotide
  source 1..85: mol_type = other DNA
caccagacagtgacgtcagctgccagatcccatggccgtcatactgtgacgtctttcagacaccccattgacgtcaatgggagaa

SEQ ID NO: 10 - DNA, 90 bp, synthetic construct
  misc_feature 1..90: note = Description of Artificial Sequence: Synthetic oligonucleotide
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 11 - DNA, 115 bp, synthetic construct
  misc_feature 1..115: note = Description of Artificial Sequence: Synthetic polynucleotide
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 12 - DNA, 115 bp, synthetic construct
  misc_feature 1..115: note = Description of Artificial Sequence: Synthetic polynucleotide
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 13 - DNA, 48 bp, synthetic construct
  misc_feature 1..48: note = Description of Artificial Sequence: Synthetic oligonucleotide
  source 1..48: mol_type = other DNA
tgagtcagtgactcagtgagtcagtgactcagtgagtcagtgactcag

SEQ ID NO: 14 - DNA, 120 bp, synthetic construct
  misc_feature 1..120: note = Description of Artificial Sequence: Synthetic polynucleotide
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 15 - DNA, 32 bp, synthetic construct
  misc_feature 1..32: note = Description of Artificial Sequence: Synthetic oligonucleotide
  source 1..32: mol_type = other DNA
gtctagacgtctagacgtctagacgtctagac

SEQ ID NO: 16 - DNA, 24 bp, synthetic construct
  misc_feature 1..24: note = Description of Artificial Sequence: Synthetic oligonucleotide
  source 1..24: mol_type = other DNA
cagacacagacacagacacagaca

SEQ ID NO: 17 - DNA, 65 bp, synthetic construct
  misc_feature 1..65: note = Description of Artificial Sequence: Synthetic oligonucleotide
  source 1..65: mol_type = other DNA
ggatccggtactcgagatctgcgatctaagtaagcttggcattccggtactgttggtaaagccac

SEQ ID NO: 18 - DNA, 588 bp, synthetic construct
  misc_feature 1..588: note = Description of Artificial Sequence: Synthetic polynucleotide
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 19 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 20 - DNA, 544 bp, synthetic construct
  misc_feature 1..544: note = Description of Artificial Sequence: Synthetic polynucleotide
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 21 - DNA, 399 bp, synthetic construct
  misc_feature 1..399: note = Description of Artificial Sequence: Synthetic polynucleotide
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 22 - DNA, 511 bp, synthetic construct
  misc_feature 1..511: note = Description of Artificial Sequence: Synthetic polynucleotide
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 23 - DNA, 408 bp, synthetic construct
  misc_feature 1..408: note = Description of Artificial Sequence: Synthetic polynucleotide
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 24 - DNA, 344 bp, synthetic construct
  misc_feature 1..344: note = Description of Artificial Sequence: Synthetic polynucleotide
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 25 - DNA, 1229 bp, synthetic construct
  misc_feature 1..1229: note = Description of Artificial Sequence: Synthetic polynucleotide
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 26 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 27 - DNA, 1718 bp, synthetic construct
  misc_feature 1..1718: note = Description of Artificial Sequence: Synthetic polynucleotide
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 28 - DNA, 212 bp, synthetic construct
  misc_feature 1..212: note = Description of Artificial Sequence: Synthetic polynucleotide
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 29 - DNA, 2379 bp, synthetic construct
  misc_feature 1..2379: note = Description of Artificial Sequence: Synthetic polynucleotide
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 30 - DNA, 529 bp, synthetic construct
  misc_feature 1..529: note = Description of Artificial Sequence: Synthetic polynucleotide
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 31 - DNA, 794 bp, synthetic construct
  misc_feature 1..794: note = Description of Artificial Sequence: Synthetic polynucleotide
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 32 - DNA, 532 bp, synthetic construct
  misc_feature 1..532: note = Description of Artificial Sequence: Synthetic polynucleotide
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 33 - DNA, 701 bp, synthetic construct
  misc_feature 1..701: note = Description of Artificial Sequence: Synthetic polynucleotide
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 34 - DNA, 465 bp, synthetic construct
  misc_feature 1..465: note = Description of Artificial Sequence: Synthetic polynucleotide
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 35 - DNA, 538 bp, synthetic construct
  misc_feature 1..538: note = Description of Artificial Sequence: Synthetic polynucleotide
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 36 - DNA, 1091 bp, synthetic construct
  misc_feature 1..1091: note = Description of Artificial Sequence: Synthetic polynucleotide
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 37 - DNA, 243 bp, synthetic construct
  misc_feature 1..243: note = Description of Artificial Sequence: Synthetic polynucleotide
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 38 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 39 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 40 - protein, 314 aa, synthetic construct; identical to SEQ ID NO: 3
  REGION 1..314: note = Description of Artificial Sequence: Synthetic polypeptide

SEQ ID NO: 41 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 42 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPVKLLFAPNLVLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 43 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRNQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 44 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATA

SEQ ID NO: 45 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATA

SEQ ID NO: 46 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 47 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRNEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATA

SEQ ID NO: 48 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLVLDRDQGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 49 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLVLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 50 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPATA

SEQ ID NO: 51 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLVLDRNQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 52 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 53 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 54 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPATA

SEQ ID NO: 55 - protein, 314 aa, synthetic construct
  REGION 1..314: note = Description of Artificial Sequence: Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATA

SEQ ID NO: 56 - DNA, 243 bp, synthetic construct
  misc_feature 1..243: note = Description of Artificial Sequence: Synthetic polynucleotide
  source 1..243: mol_type = other DNA
cgggtttcgtaacaatcgcatgaggattcgcaacgcctttgaagcagtcgacgccgaagtcccgtctcagtaaaggttgaagcagtcgacgccgaagaatcggactgccttcgtatgaagcagtcgacgccgaaggtatcagtcgcctcggaatgaagcagtcgacgccgaagattcgtaagaggctcactctcccttacacggagtggataactagttctagagggtatataatgggggcca

SEQ ID NO: 57 - DNA, 1623 bp, synthetic construct
  misc_feature 1..1623: note = Description of Artificial Sequence: Synthetic polynucleotide
  source 1..1623: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 58 - protein, 541 aa, synthetic construct
  REGION 1..541: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - DNA, 51 bp, synthetic construct
  misc_feature 1..51: note = Description of Artificial Sequence: Synthetic oligonucleotide
  source 1..51: mol_type = other DNA
atttatttatttatttatttaacatcggttccctgtttaatatttaaacag

SEQ ID NO: 60 - DNA, 266 bp, synthetic construct
  misc_feature 1..266: note = Description of Artificial Sequence: Synthetic polynucleotide
  source 1..266: mol_type = other DNA
agagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 61 - DNA, 531 bp, synthetic construct
  misc_feature 1..531: note = Description of Artificial Sequence: Synthetic polynucleotide
  source 1..531: mol_type = other DNA
atgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatct

SEQ ID NO: 62 - protein, 177 aa, synthetic construct
  REGION 1..177: note = Description of Artificial Sequence: Synthetic polypeptide
MSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGS

SEQ ID NO: 63 - DNA, 573 bp, synthetic construct
  misc_feature 1..573: note = Description of Artificial Sequence: Synthetic polynucleotide
  source 1..573: mol_type = other DNA
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 64 - protein, 191 aa, synthetic construct
  REGION 1..191: note = Description of Artificial Sequence: Synthetic polypeptide
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 65 - DNA, 936 bp, synthetic construct
  misc_feature 1..936: note = Description of Artificial Sequence: Synthetic polynucleotide
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 66 - protein, 312 aa, synthetic construct
  REGION 1..312: note = Description of Artificial Sequence: Synthetic polypeptide
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 67 - DNA, 11809 bp, synthetic construct
  misc_feature 1..11809: note = Description of Artificial Sequence: Synthetic polynucleotide
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 68 - DNA, 936 bp, synthetic construct
  misc_feature 1..936: note = Description of Artificial Sequence: Synthetic polynucleotide
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 69 - protein, 312 aa, synthetic construct
  REGION 1..312: note = Description of Artificial Sequence: Synthetic polypeptide
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPAT

SEQ ID NO: 70 - DNA, 11809 bp, synthetic construct
  misc_feature 1..11809: note = Description of Artificial Sequence: Synthetic polynucleotide
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 71 - DNA, 936 bp, synthetic construct
  misc_feature 1..936: note = Description of Artificial Sequence: Synthetic polynucleotide
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 72 - protein, 312 aa, synthetic construct
  REGION 1..312: note = Description of Artificial Sequence: Synthetic polypeptide
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLVLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 73 - DNA, 11809 bp, synthetic construct
  misc_feature 1..11809: note = Description of Artificial Sequence: Synthetic polynucleotide
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 74 - DNA, 123 bp, synthetic construct
  misc_feature 1..123: note = Description of Artificial Sequence: Synthetic polynucleotide
  source 1..123: mol_type = other DNA
agcggcggaggtggtagcggaggcggaggatctggaattacacagggactcgccgtgtctacaatctccagcttctttggtggcggtagtggcggcggtggcagtggcggtggatctcttcaa

SEQ ID NO: 75 - protein, 41 aa, synthetic construct
  REGION 1..41: note = Description of Artificial Sequence: Synthetic polypeptide
SGGGGSGGGGSGITQGLAVSTISSFFGGGSGGGGSGGGSLQ

SEQ ID NO: 76 - DNA, 138 bp, synthetic construct
  misc_feature 1..138: note = Description of Artificial Sequence: Synthetic polynucleotide
  source 1..138: mol_type = other DNA
ctgctgccaagctgggccatcacactgatctccgtgaacggcatcttcgtgatctgttgcctgacctactgcttcgcccctcggtgcagagagcggagaagaaacgaacggctgcggagagaatctgtgcggcctgtg

SEQ ID NO: 77 - protein, 46 aa, synthetic construct
  REGION 1..46: note = Description of Artificial Sequence: Synthetic polypeptide
LLPSWAITLISVNGIFVICCLTYCFAPRCRERRRNERLRRESVRPV

SEQ ID NO: 78 - DNA, 330 bp, synthetic construct
  misc_feature 1..330: note = Description of Artificial Sequence: Synthetic polynucleotide
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 79 - DNA, 11892 bp, synthetic construct
  misc_feature 1..11892: note = Description of Artificial Sequence: Synthetic polynucleotide
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 80 - DNA, 11892 bp, synthetic construct
  misc_feature 1..11892: note = Description of Artificial Sequence: Synthetic polynucleotide
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 81 - DNA, 11892 bp, synthetic construct
  misc_feature 1..11892: note = Description of Artificial Sequence: Synthetic polynucleotide
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 82 - protein, 4 aa, synthetic construct
  REGION 1..4: note = Description of Artificial Sequence: Synthetic peptide
WRPW

SEQ ID NO: 83 - DNA, 1623 bp, synthetic construct
  misc_feature 1..1623: note = Description of Artificial Sequence: Synthetic polynucleotide
  source 1..1623: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggcggaggaagcggtggcggatcaggtggtggatctggcggcggatctagaaacctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctct